(19) United States
(12) Patent Application Publication
(10) Pub. No.: US 2023/0365963 A1
(43) Pub. Date: Nov. 16, 2023

(54) METHODS FOR TREATING NEUROLOGICAL DISEASE

(71) Applicant: ASKLEPIOS BIOPHARMACEUTICAL, INC., Research Triangle Park, NC (US)

(72) Inventors: Anna TRETIAKOVA, Research Triangle Park, NC (US); Lester Suarez, Research Triangle Park, NC (US); Anne Braae, Mildlothian (GB); Michael L. Roberts, Mildlothian (GB); Philippe Moullier, Research Triangle Park, NC (US)

(73) Assignee: ASKLEPIOS BIOPHARMACEUTICAL, INC., Research Triangle Park, NC (US)

(21) Appl. No.: 18/027,293

(22) PCT Filed: Sep. 21, 2021
(86) PCT No.: PCT/US2021/071534

§ 371 Date: Mar. 20, 2023

Related U.S. Application Data
(60) Provisional application No. 63/080,925, filed on Sep. 21, 2020.
(60) Provisional application No. 63/121,152, filed on Dec. 3, 2020.
(60) Provisional application No. 63/139,410, filed on Jan. 20, 2021.
(60) Provisional application No. 63/140,440, filed on Jan. 22, 2021.
(60) Provisional application No. 63/180,407, filed on Apr. 27, 2021.

(51) Int. Cl. C12N 15/113; C12N 9/02; C12N 15/86; A61K 48/00

(52) U.S. Cl. CPC C12N 15/113; C12N 9/0073; C12N 15/86; A61K 48/0058; C12Y 114/13098; C12N 2310/141; C12N 2830/008; C12N 2750/14143

(57) ABSTRACT

Aspects of the disclosure relate to compositions and methods useful for treating neurological diseases and disorders. In some embodiments, the disclosure provides a method for treating a neurological disease or disorder comprising administration of both a viral vector comprising interfering nucleic acids (e.g., artificial miRNAs) and a viral vector comprising a CYP46A1 protein. In some embodiments, the disclosure provides a method for treating Huntington's disease comprising administration of both a viral vector comprising interfering nucleic acids (e.g., artificial miRNAs) targeting the huntingtin gene (HTT) and a viral vector comprising a CYP46A1 protein. In some embodiments, the viral vector comprises a modified viral capsid, such as for preferentially targeting cells in the CNS or PNS.

### CROSS-REFERENCE TO RELATED APPLICATIONS

This application is a 35 U.S.C. § 371 National Phase Entry Application of International Application No. PCT/US2021/071534 filed Sep. 21, 2021, which claims benefit under 35 U.S.C. § 119(e) of U.S. Provisional Application No. 63/080,925 filed Sep. 21, 2020, U.S. Provisional Application No. 63/121,152 filed Dec. 3, 2020, U.S. Provisional Application No. 63/139,410 filed Jan. 10, 2021, U.S. Provisional Application No. 63/140,440 filed Jan. 22, 2021, U.S. Provisional Application No. 63/180,407 filed Apr. 27, 2021, the contents of each of which are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Oct. 25, 2021, is named 046192-098000WOPT_SL.txt and is 159,759 bytes in size.

### TECHNICAL FIELD

The technology described herein relates to methods for treating neurological diseases or disorders, e.g., Huntington's disease.

### BACKGROUND

Huntington's disease (HD) is a devastating inherited neurodegenerative disease caused by an expansion of the CAG repeat region in exon 1 of the huntingtin gene. While the Huntingtin protein (HTT) is expressed throughout the body, the polyglutamine expanded protein is especially toxic to medium spiny neurons in the striatum and their cortical connections. Patients struggle with emotional symptoms including depression and anxiety and with characteristic movement disturbances and chorea. There is currently no cure for Huntington's disease; therapeutic options are limited to ameliorating disease symptoms.

### SUMMARY

One aspect provided herein describes a method for treating a neurological disease or disorder in a subject in need thereof, the method comprising administering to a subject having or at risk of developing the neurological disease or disorder a therapeutically effective amount of (a) a nucleic acid encoding at least one miRNA; and (b) a nucleic acid encoding a CYP46A1 protein.

In one aspect, described herein is a composition or combination comprising (a) an isolated nucleic acid encoding a transgene encoding one or more miRNAs; and (b) an isolated nucleic acid encoding a CYP46A1 protein. In one aspect, described herein is a composition or combination comprising: (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and (b) a recombinant viral vector comprising an isolated nucleic acid encoding the CYP46A1 protein.

In one aspect, described herein is a method for treating a neurological disease or disorder in a subject in need thereof, the method comprising administering to a subject having or at risk of developing the neurological disease or disorder a therapeutically effective amount of (a) an isolated nucleic acid encoding a transgene encoding one or more miRNAs; and (b) an isolated nucleic acid encoding a CYP46A1 protein. In one aspect, described herein is a method for treating a neurological disease or disorder in a subject in need thereof, the method comprising administering to a subject having or at risk of developing the neurological disease or disorder a therapeutically effective amount of (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and (b) a recombinant viral vector comprising an isolated nucleic acid encoding the CYP46A1 protein.

In some embodiments, the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders. In some embodiments, the neurological disease or disorder is a central nervous system (CNS) disease or disorder. In some embodiments, the CNS disease or disorder is selected from Huntington's disease, Alzheimer's disease, Polyglutamine repeat spinocerebellar ataxias, Amyotrophic lateral sclerosis and Parkinson's disease.

In some embodiments, the CNS disease or disorder is Alzheimer's disease and the at least one miRNA comprises a seed sequence complementary to Amyloid Precursor Protein (APP), Presenilin 1, Presenilin 2, ABCA7, SORL1, and disease-associated alleles thereof.

In some embodiments, the CNS disease or disorder is Parkinson's disease and the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

In some embodiments, the CNS disease is Huntington's disease and at least one miRNA comprises a seed sequence complementary to SEQ ID NO: 4, or wherein at least one miRNA comprises the sequence of any one of SEQ ID NOs: 6-17, 40-44, or 50-66 flanked by a miRNA backbone sequence. In some embodiments, the CNS disease is Huntington's disease and at least one miRNA comprises the sequence of any one of SEQ ID NOs: 6-17, 40-44, or 50-66. In some embodiments, at least one of the miRNAs hybridizes with and inhibits expression of human huntingtin. In some embodiments, the subject comprises a huntingtin gene having more than 36 CAG repeats, more than 40 repeats, or more than 100 repeats. In some embodiments, the subject is less than 20 years of age.

In some embodiments, the recombinant viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector.

In some embodiments, the recombinant viral vector comprising (a) is the same as the recombinant viral vector comprising (b). In some embodiments, the isolated nucleic acid of (a) and (b) are comprised in separate recombinant viral vectors. In some embodiments, the isolated nucleic acid of (a) and (b) are comprised in the same recombinant viral vector.

In some embodiments, (a) and (b) are administered at substantially the same time. In some embodiments, (a) and (b) are administered at different time points. In some embodiments, the different time points are spaced by at least 1 min, at least 1 hour, at least 1 day, at least 1 week, at least 1 month, at least 1 year, or more. In some embodiments, (a) is administered prior to the administration of (b). In some embodiments, (b) is administered prior to the administration of (a). In some embodiments, the administration of (a), (b), or (a) and (b) is repeated at least once.

In some embodiments, the transgene comprises two miRNAs in tandem that are flanked by introns. In some embodiments, the flanking introns are identical. In some embodiments, the flanking introns are from the same species. In some embodiments, the flanking introns are hCG introns.

In some embodiments, the transgene comprises a promoter. In some embodiments, the promoter is a synapsin (Syn1) promoter, or a promoter of Tables 10-13.

In some embodiments, the one or more miRNAs are located in an untranslated portion of the transgene. In some embodiments, the untranslated portion is an intron. In some embodiments, the untranslated portion is between the last codon of the nucleic acid sequence encoding a protein and a poly-A tail sequence, or between the last nucleotide base of a promoter sequence and a poly-A tail sequence. In some embodiments, the untranslated portion is a 5′ untranslated region (5′ UTR).

In some embodiments, the nucleic acid or viral vector further comprises a third region comprising a second adeno-associated virus (AAV) inverted terminal repeat (ITR), or a variant thereof.

In some embodiments, the ITR variant lacks a functional terminal resolution site (TRS), optionally wherein the ITR variant is a ATRS ITR.

In some embodiments, the administration results in delivery of the viral vector or isolated nucleic acid to the central nervous system (CNS) of the subject. In some embodiments, the administration is via injection, optionally intravenous injection or intrastriatal injection.

In some embodiments, the viral vector is AAV1, AAV2, AAV3b, AAV4, AAV5, AAV6, AAV7, AAV8, AAV9, AAV10, AAV 11, or, AAV12, or a chimera thereof. In some embodiments, the viral vector comprises a capsid protein from AAV serotype AAV1, AAV2, AAV3b, AAV4, AAV5, AAV6, AAV7, AAV8, AAV9, AAV10, AAV 11, or, AAV12, or a chimera thereof. In some embodiments, the capsid protein is an AAV9 capsid protein. In some embodiments, the viral vector is a self-complementary AAV (scAAV). In some embodiments, the viral vector is formulated for delivery to the central nervous system (CNS).

In some embodiments of any of the aspects, the viral vector comprises a modified viral capsid.

In some embodiments of any of the aspects, the viral vector comprises a modification to a viral capsid.

In some embodiments of any of the aspects, the modification is a chemical, non-chemical or amino acid modification of the viral capsid.

In some embodiments of any of the aspects, at least one of the capsid modifications preferentially targets cells in the CNS or PNS.

In some embodiments of any of the aspects, the chemical modification comprises a chemically-modified tyrosine residue modified to comprise a covalently-linked mono- or polysaccharide moiety.

In some embodiments of any of the aspects, the chemically-modified tyrosine residue comprises a mono-saccharide selected from galactose, mannose, N-acetylgalactosamine, bridge GalNac, and mannose-6-phosphate.

In some embodiments of any of the aspects, the chemical modification comprises a ligand covalently linked to a primary amino group of a capsid polypeptide via a —CSNH— bond.

In some embodiments of any of the aspects, the ligand comprises an arylene or heteroarylene radical covalently bound to the ligand.

In some embodiments of any of the aspects, the modified viral capsid is a chimeric capsid or a haploid capsid.

In some embodiments of any of the aspects, the modified viral capsid is a haploid capsid.

In some embodiments of any of the aspects, the modified viral capsid is a chimeric or haploid capsid further comprising a modification.

In some embodiments of any of the aspects, the modified viral capsid is an AAV serotype AAV1, AAV2, AAV3b, AAV4, AAV5, AAV6, AAV7, AAV8, AAV9, AAV10, AAV11, AAV12, or a mutant modified form, a chimera, a mosaic, or a rational haploid thereof.

In some embodiments of any of the aspects, the modification changes the antigenic profile of the modified viral capsid as compared to the unmodified viral capsid.

In some embodiments of any of the aspects, the modified viral capsid can be used for repeat administration.

### BRIEF DESCRIPTION OF THE DRAWINGS

FIG. 1 is a schematic showing an HD plasmid map of pJAL130-CYP46A1, 7314 bp, see e.g., SEQ ID NO: 111 and Table 16, which shows the ITR to ITR sequence of the CYP46 variant sequence (see e.g., SEQ ID NO: 110) from the plasmid.

FIG. 2 shows the intracranial biodistribution in sagittal sections of the transgene GFP under the control of CNS-1 (see e.g., SEQ ID NO: 112), CNS-2 (see e.g., SEQ ID NO: 113), CNS-3 (see e.g., SEQ ID NO: 114), CNS-4 (see e.g., SEQ ID NO: 115), CNS-5 (see e.g., SEQ ID NO: 122), CNS-6 (see e.g., SEQ ID NO: 123), CNS-7 (see e.g., SEQ ID NO: 124) and CNS-8 (see e.g., SEQ ID NO: 125) and the control promoter hSyn1 (see e.g., SEQ ID NO: 152) delivered by intracerebroventricular (ICV) and intravenous (IV) injection. Scale bar is 1 mm.

FIG. 3A-3B show images of coronal bran sections. FIG. 3A shows the intracranial biodistribution in coronal sections of the transgene GFP under the control of CNS-1 (see e.g., SEQ ID NO: 112), CNS-2 (see e.g., SEQ ID NO: 113), CNS-3 (see e.g., SEQ ID NO: 114) and CNS-4 (see e.g., SEQ ID NO: 115) delivered by ICV. Scale bar is 1 mm. FIG. 3B shows the intracranial biodistribution in coronal sections of the transgene GFP under the control of CNS-5 (see e.g., SEQ ID NO: 122), CNS-6 (see e.g., SEQ ID NO: 123), CNS-7 (see e.g., SEQ ID NO: 124) and CNS-8 (see e.g., SEQ ID NO: 125) and the control promoter hSyn1 (see e.g., SEQ ID NO: 152) delivered by ICV. Scale bar is 1 mm.

FIG. 4 shows percentage GFP immunoreactivity in different brain regions following ICV or IV delivery of GFP driven by CNS 1-8 (see e.g., SEQ ID NOs: 112-115, 122-125) or Synapsin-1 (see e.g., SEQ ID NO: 152). The data was obtained by quantitative measurement of 10 non-overlapping RGB images of GFP staining intensity by thresholding analysis in cortex, hippocampus, striatum, midbrain and cerebellum (mean±SEM). Images were taken at ×40 magnification through discrete brain regions keeping constant settings. The foreground immunostaining was defined by averaging of the highest and lowest signals. Data is represented as the mean percentage area of immunoreactivity per field for each region of interest (n=3). With ICV delivery, expression is highest in cortex and hippocampal brain regions. CNS 1-8 (see e.g., SEQ ID NO: 112-115, 122-125) show higher expression in the hippocampus than hSyn1 control. CNS-1 (see e.g., SEQ ID NO: 112) shows higher expression in hippocampus, midbrain and cerebellum compared to hSyn1 with ICV delivery.

FIG. 5A-5B show the tissue expression pattern for the faf1 and pitx3 genes from which the CRE/proximal promoter from CNS-5, CNS-5_v2, CNS-2, CNS-3 and CNS-4 were designed. FIG. 5A shows the expression pattern of the faf1 gene in mouse PNS neurones from single cell transcriptomic data (Zeisel et al., 2018). Dark grey denotes high expression, white denotes no expression and light grey denotes low expression. faf1 is expressed in many PNS neurones. FIG. 5B shows the expression pattern of the pitx3 gene in PNS neurones from single cell transcriptomic data (Zeisel et al., 2018). Dark grey denotes high expression, white denotes no expression and light grey denotes low expression. pixt3 is expressed in sympathetic PNS neurones. faf1 is expressed in many PNS neurones so a synthetic promoter comprising CRE or proximal promoter designed from the faf1 gene such as CNS-5 and CNS-5_v2 is expected to have strong expression in the PNS. pitx3 is expressed in sympathetic PNS neurones so a synthetic promoter comprising CRE designed from the pitx3 gene such as CNS-2, CNS-3 or CNS-4 is expected to have expression in PNS sympathetic neurones. Similar analysis for lmx1b and pitx2 revealed no expression in PNS above the cut off score for the analysis (trinization score of less than 0.95; data not shown) so CNS-1, CNS-6, CNS-6_v2, CNS-7, CNS-7_v2, CNS-8 and CNS-8_v2 are not expected to be active in PNS neurones.

FIG. 6A shows the expression pattern of the HTT gene in a sagittal section from an adult mouse brain (taken from the Allen Mouse brain atlas; mouse.brain-map.org). HTT (huntingtin) is highly expressed in throughout the brain.

FIG. 6B shows the expression pattern of the CYP46A1 gene in a coronal section from an adult mouse brain (taken from the Allen Mouse brain atlas; mouse.brain-map.org). CYP46A1 is widely expressed in the brain.

FIG. 7A shows the median GFP expression of synthetic NS-specific promoters SP0013, SP0014, SP0030, SP0031, SP0032, SP0019, SP0020, SP0021, SP0022, SP0011, SP0034, SP0035, SP0036 and control promoters Synapsin-1 relative to control promoter CAG in neuroblastoma-derived SH-SY5Y cells. NTC denotes non-transfected cells. The data is collected from three biological replicates, each of which is the average of two technical replicates. Error bars are standard error.

FIG. 7B shows the transfection efficiency in neuroblastoma-derived SH-SY5Y cells when transfected with synthetic NS-specific promoters SP0013, SP0014, SP0030, SP0031, SP0032, SP0019, SP0020, SP0021, SP0022, SP0011, SP0034, SP0035, SP0036 or control promoters Synapsin-1 and CAG, operably linked to GFP. NTC denotes non-transfected cells. The data is collected from three biological replicates, each of which is the average of two technical replicates. Error bars are standard error. GFP positive % denotes the % of all cells which were GFP positive.

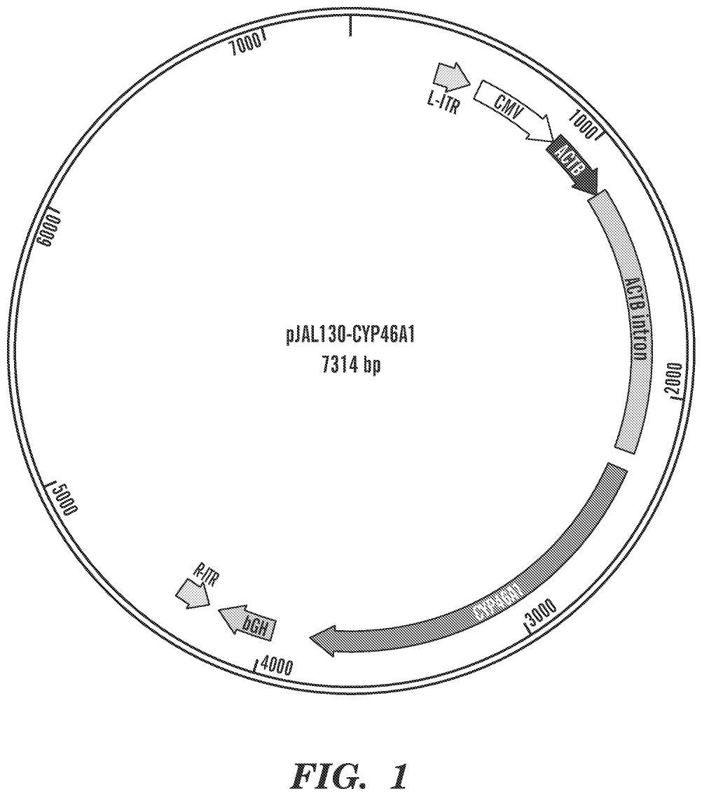

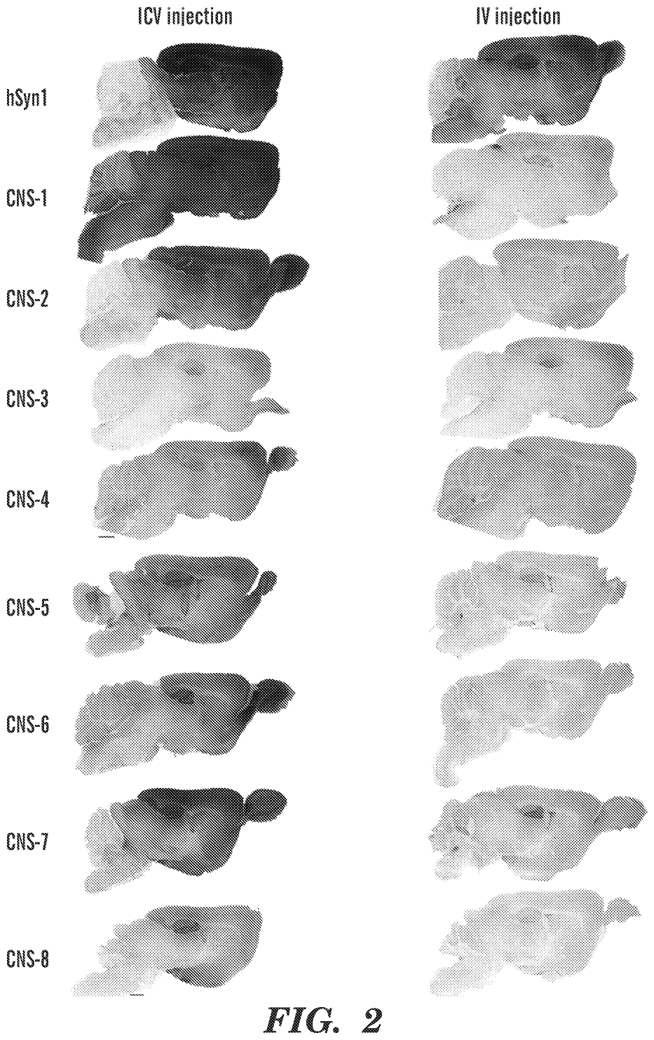

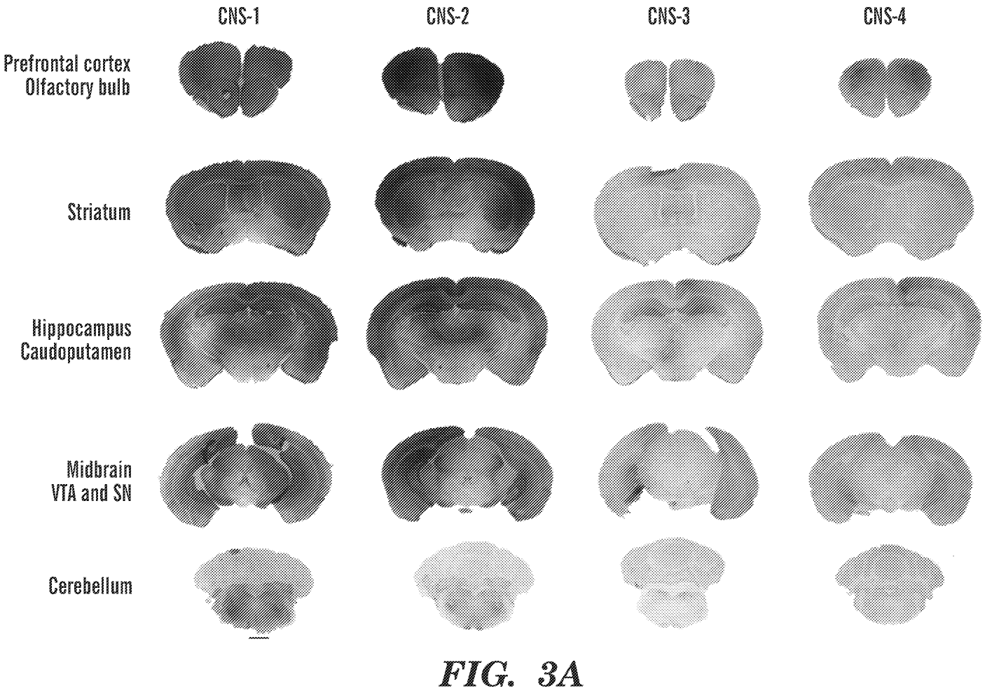

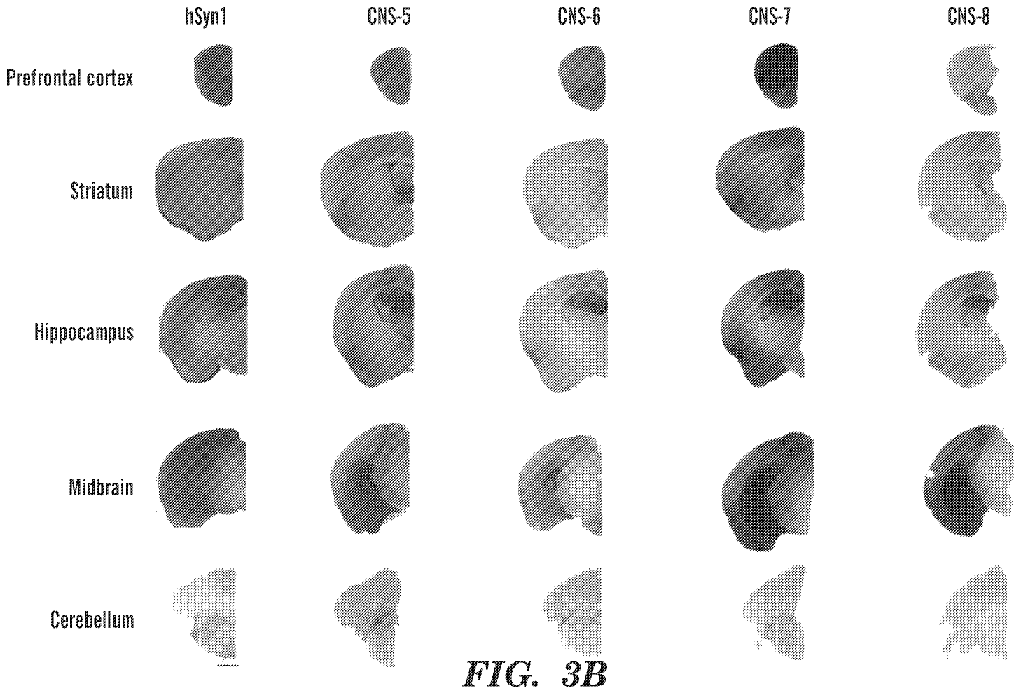

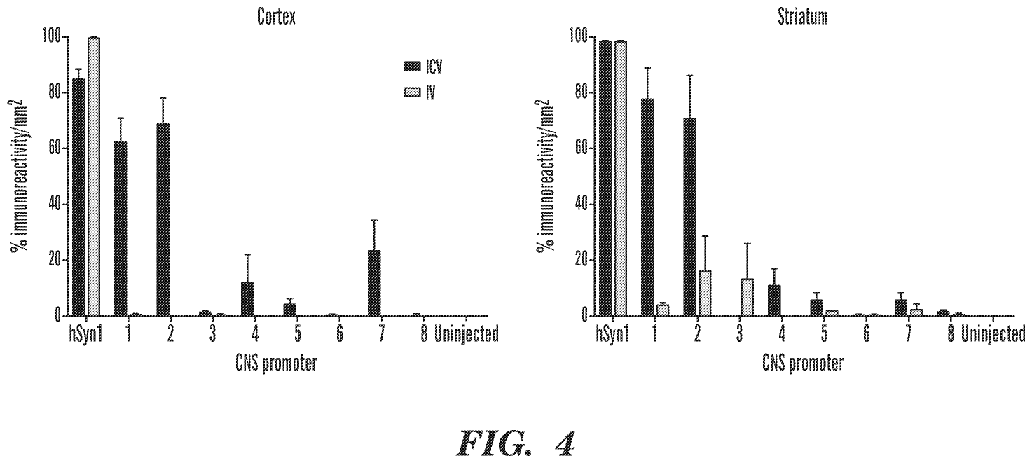

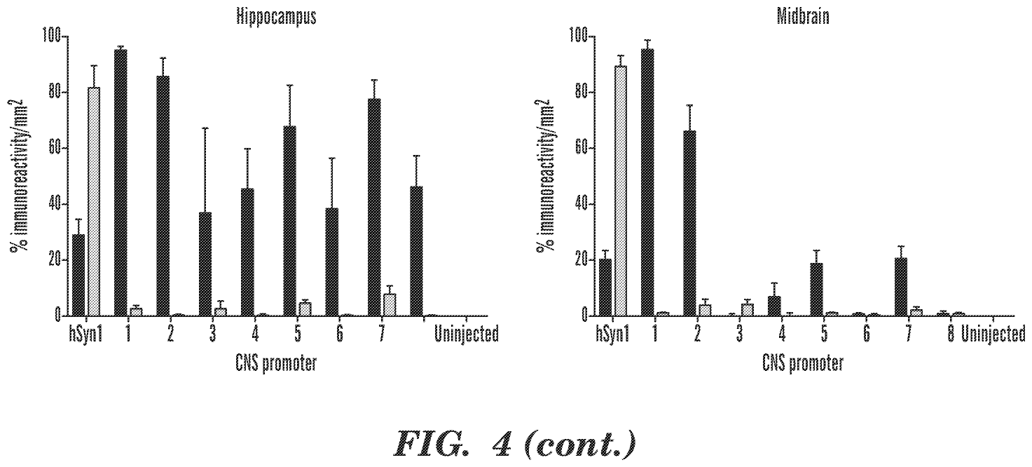

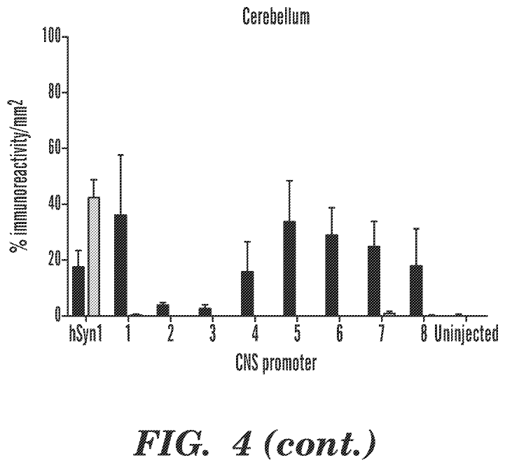

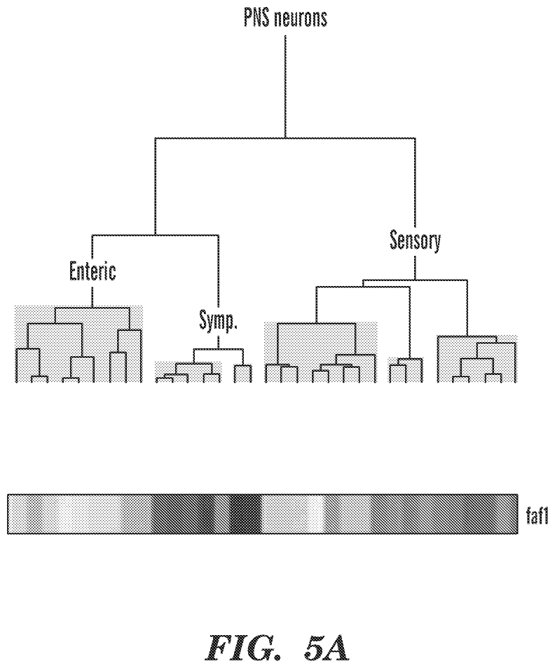

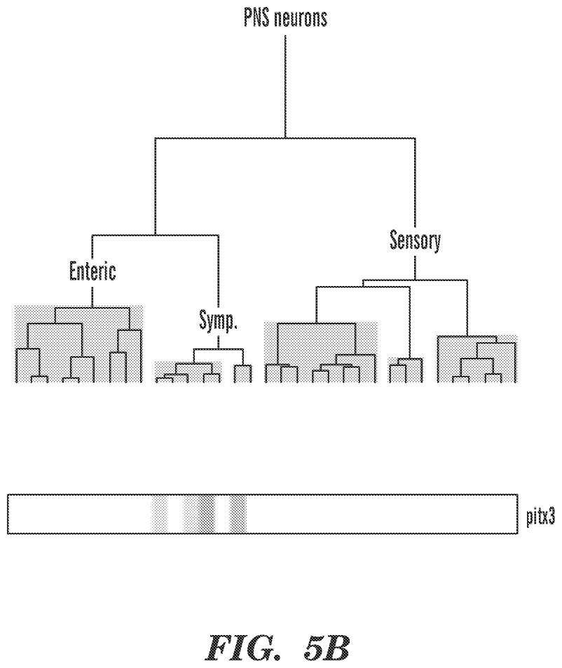

### DETAILED DESCRIPTION

Aspects of the invention relate to administration of both an interfering RNA (e.g., miRNAs, such as artificial miRNAs) that when delivered to a subject are effective for reducing the expression of a pathogenic gene in the subject, and a nucleic acid encoding a CYP46A1 protein. Accordingly, methods and compositions described by the disclosure are useful, in some embodiments, for the treatment of neurological diseases or disorders.

### Treatment Methods

Methods for delivering a nucleic acid and/or a transgene (e.g., an inhibitory RNA, such as a miRNA and/or a nucleic acid encoding CYP46A1) to a subject are provided by the disclosure. The methods typically involve administering to a subject an effective amount of a nucleic acid encoding at least one interfering RNA/inhibitory nucleic acid capable of reducing expression of a target gene, e.g., a pathogenic gene associated with a neurological disease or disorder (e.g., huntingtin (htt) protein) and a nucleic acid encoding CYP46A1. In some embodiments, one or both of the nucleic acids are provided in a viral vector and/or in a viral particle, e.g., a rAAV.

As used herein, “neurological disease or disorder” can refer to any disease, disorder, or condition affecting or associated with the nervous system, i.e. those that affect the central nervous system (brain and spinal cord), the peripheral nervous system (PNS; e.g., peripheral nerves and cranial nerves), and the autonomic nervous system (parts of which are located in both central and peripheral nervous systems). More than 600 neurological diseases have been identified in humans. By way of non-limiting examples, the neurological disease or disorder can be Alzheimer's disease, Parkinson's disease, Huntington's disease, Canavan disease, Leigh's disease, spinal cerebral ataxia, polyglutamine repeat spinocerebellar ataxias, Krabbe's disease, Batten's disease, Refsum disease, Tourette syndrome, primary lateral sclerosis, amyotrophic lateral sclerosis, progressive muscular atrophy, Pick's disease, Niemann Pick's disease, muscular dystrophy, multiple sclerosis, myasthenia gravis, Binswanger's disease, trauma due to spinal cord or head injury, ophthalmic diseases and disorders, Tay-Sachs disease, Rett syndrome, Neuropathic pain, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, a psychosexual disorder, a sleeping disorder, a pain disorder, and/or a eating or weight disorder. In some embodiments, the neurological disease or disorder is a central nervous system (CNS) disease or disorder, e.g., Huntington's disease, Parkinson's disease, or Alzheimer's disease.

As used herein, “Huntington's disease”, or “HD”, refers to a neurodegenerative disease characterized by progressively worsening movement, cognitive and behavioral changes caused by a tri-nucleotide repeat expansion (e.g., CAG, which is translated into a poly-Glutamine, or PolyQ, tract) in the HTT gene that results in production of pathogenic mutant huntingtin protein (HTT, or mHTT).

As used herein, “HTT” or “huntingtin” refers to the gene which encodes the huntingtin protein. Normal huntingtin proteins function in nerve cells, and the normal HTT gene usually has from about 7 to about 35 CAG repeats at the 5′ end. The HTT gene is often mutated in patients with Huntington Disease, or at risk of developing Huntington Disease. In some embodiments, mutant huntingtin protein accelerates the rate of neuronal cell death in certain regions of the brain. Generally, the severity of HD is correlated to the size of the tri-nucleotide repeat expansion in a subject. For example, a subject having a CAG repeat region comprising between 36 and 39 repeats (SEQ ID NO: 157) is characterized as having “reduced penetrance” HD, whereas a subject having greater than 40 repeats is characterized as having “full penetrance” HD. Thus, in some embodiments, a subject having or at risk of having HD has a HTT gene comprising between about 36 and about 39 CAG repeats (e.g., 36, 37, 38 or 39 repeats). In some embodiments, a subject having or at risk of having HD has a HTT gene comprising 40 or more (e.g., 40, 45, 50, 60, 70, 80, 90, 100, 200, or more) CAG repeats (SEQ ID NO: 156). In some embodiments, a subject having a HTT gene comprising more than 100 CAG repeats develops HD earlier than a subject having fewer than 100 CAG repeats. In some embodiments, a subject having a HTT gene comprising more than 100 CAG repeats may develop HD symptoms before the age of about 20 years, and is referred to as having juvenile HD (also referred to as akinetic-rigid HD, or Westphal variant HD). The number of CAG repeats in a HTT gene allele of a subject can be determined by any suitable modality known in the art. For example, nucleic acids (e.g., DNA) can be isolated from a biological sample (e.g., blood) of a subject and the number of CAG repeats of a HTT allele can be determined by a hybridization-based method, such as PCR or nucleic acid sequencing (e.g., Illumina sequencing, Sanger sequencing, SMRT sequencing, etc.). The sequences of the HTT genes are known in a number of species, e.g., human HTT (NCBI Gene ID: 3064) mRNA sequences (NCBI Ref Seq: NM_002111.8, SEQ ID NO: 4) and protein sequences (NCBI Ref Seq: NP_0021012.4, SEQ ID NO: 5). Accordingly, in some embodiments relating to the treatment of Huntington's disease the one or more inhibitory nucleic acids (e.g., miRNAs) can hybridize to and/or reduce expression of HTT.

As used herein, “Alzheimer's disease”, or “AD”, refers to a neurodegenerative disease characterized by progressively worsening memory, disorientation, mood swings, as well as increasing difficulty with language, motivation and self-care. A number of genes can contribute to or increase the risk of AD, including Amyloid Precursor Protein (APP; NCBI Gene ID: 351), Presenilin 1 (PSEN1; NCBI Gene ID 5663), Presenilin 2 (PSEN2; NCBI Gene ID 5664), ATP binding cassette subfamily A member 7 (ABCA7; NCBI Gene ID 10347), and sortilin related receptor 1 (SORL1; NCBI Gene ID 6653). The sequences of such AD-associated genes are known in a number of species, e.g., human mRNAs and protein sequences are available in the NCBI database using the provided Gene ID numbers. These AD-associated genes and others, as well as AD-associated alleles thereof (e.g. mutations, SNPs, etc.) are known in the art and described further in, e.g., Sims et al. Nature Neuroscience 2020 23:311-22; Bellenguez et al. Current Opinion in Neurobiology 2020 61:40-48; Tabuas-Pereira et al. 2020 Neurogenetics and Psychiatric Genetics 8:1-16; and Porter et al. Chapter 15 of “Neurodegeneration and Alzheimer's Disease” 2019; each of which is incorporated by reference herein in its entirety. Accordingly, in some embodiments relating to the treatment of Alzheimer's disease, the one or more inhibitory nucleic acids (e.g., miRNAs) can hybridize to and/or reduce expression of APP, PSEN1, PSEN2, ABCA7, and/or SORL1

As used herein, “Parkinson's disease”, or “PD”, refers to a neurodegenerative disease characterized by progressively worsening shaking and stiffness and increasing problems with balance, walking, and coordination. A number of genes can contribute to or increase the risk of PD, including synuclein alpha (SNCA; NCBI Gene ID: 6622), leucine rich repeat kinase 2 (LRRK2/PARK8; NCBI Gene ID 120892), glucosylceramidase beta (GBA1; NCBI Gene ID 2629), parkin RBR E3 ubiquitin (PRKN; NCBI Gene ID 5071), PTEN induced kinase 1 (PINK1; NCBI Gene ID 65018), Parkinsonism associated deglycase (DJ1/PARK7; NCBI Gene ID 11315), VPS35 retromer complex component (VPS35; NCBI Gene ID 55737), eukaryotic translation initiation factor 4 gamma 1 (EIF4G1; NCBI Gene ID 1981), DnaJ heat shock protein family member C13 (DNAJC13; NCBI Gene ID 23317), coiled-coil-helix-coiled-coil-helix domain containing 2 (CHCHD2; NCBI Gene ID 51142), and/or ubiquitin C-terminal hydrolase L1 (UCHL1; NCBI Gene ID 7345). The sequences of such PD-associated genes are known in a number of species, e.g., human mRNAs and protein sequences are available in the NCBI database using the provided Gene ID numbers. These PD-associated genes and others, as well as PD-associated alleles thereof (e.g. mutations, SNPs, etc.) are known in the art and described further in, e.g., D'Souza et al. Acta Neuropsychiatrica 2020 32:10-22; Sardi et al. Parkinsonism & Related Disorders 2019 59:32-38; Hardy et al. Current Opinion in Genetics & Development 2009 19:254-65; Ferreria et al. Neurologica 2017 135:273-84; Jain et al. Clinical Science 2005 109:355-64; Fagan et al. European Journal of Neurology 2017 24:561-e20; Campelo et al. Parkinson's Disease 2017 4318416; and Porter et al. Chapter 15 of “Neurodegeneration and Alzheimer's Disease” 2019; each of which is incorporated by reference herein in its entirety. Accordingly, in some embodiments relating to the treatment of Parkinson's disease the one or more inhibitory nucleic acids (e.g., miRNAs) can hybridize to and/or reduce expression of SNCA, LRRK2/PARK8, PRKN, PINK1, DJ1/PARK7, VPS35, EIF4G1, DNAJC13, CHCHD2, UCHL1, and/or GBA1.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to reduce the expression of a pathogenic gene or protein (e.g., HTT), to extend the lifespan of a subject, to improve in the subject one or more symptoms of disease (e.g., a symptom of Huntington's disease), etc. The effective amount will depend on a variety of factors such as, for example, the species, age, weight, health of the subject, and the tissue to be targeted, and may thus vary among subject and tissue as described elsewhere in the disclosure.

### Inhibitory RNAs

In some aspects, the disclosure provides inhibitory nucleic acids, e.g., miRNA, that specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a target, e.g., human huntingtin mRNA (e.g., SEQ ID NO: 4). In some embodiments, the disclosure provides inhibitory nucleic acids, e.g., miRNA, that specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of exon 1 of human huntingtin mRNA (e.g., SEQ ID NO: 3). As used herein “continuous bases” refers to two or more nucleotide bases that are covalently bound (e.g., by one or more phosphodiester bond, etc.) to each other (e.g. as part of a nucleic acid molecule). In some embodiments, the at least one miRNA is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of the target, e.g., SEQ ID NOs 3 or 4. In some embodiments, the inhibitory RNA is a miRNA which is comprises or is encoded by the sequence set forth in any one of SEQ ID NOs: 6-17, 40-44, or 50-66.

In one aspect described herein are inhibitory RNAs that can be used for the treatment of a neurological disease or disorder. In some embodiments of any of the aspects, the nucleic acid sequence of the inhibitory RNA comprises one of SEQ ID NO: 6-17, 40-44, or 50-66 or a sequence that is at least 95% (e.g., at least 95%, at least 96%, at least 97%, at least 98%, or at least 99%) identical to the sequence of at least one of SEQ ID NO: 6-17, 40-44, or 50-66 that maintains the same functions as SEQ ID NO: 3 or 4 (e.g., HTT inhibition).

In some embodiments, the vector described herein comprises at least one miRNA, each miRNA comprising a sequence set forth in any one of SEQ ID NOs: 6-17, 40-44, or 50-66. In some embodiments, the vector described herein comprises at least one miRNA, each miRNA comprising a sequence set forth in any one of SEQ ID NOs: 6-17, 40-44, or 50-66 flanked by a miRNA backbone sequence.

In some embodiments, the vector described herein comprises at least one miRNA, each miRNA comprising a seed sequence complementary to one of SEQ ID NO: 3, 4, 18-39, or 46-49. In some embodiments, the vector described herein comprises at least one miRNA, each miRNA comprising a seed sequence complementary to one of SEQ ID NO: 3, 4, 18-39, or 46-49 flanked by a miRNA backbone sequence. In some embodiments, the vector described herein comprises at least one miRNA, each miRNA comprising a seed sequence substantially complementary to one of SEQ ID NO: 3, 4, 18-39, or 46-49. In some embodiments, the vector described herein comprises at least one miRNA, each miRNA comprising a seed sequence substantially complementary to one of SEQ ID NO: 3, 4, 18-39, or 46-49 flanked by a miRNA backbone sequence.

TABLE 1
first RNA sequences substantially complementary
to SEQ ID NO: 4

| | SEQ ID |
|---|---|
| miRNA sequence | NO: |
| 5′-AAGGACUUGAGGGACUCGA-3′ | 6 |
| 5′-AAGGACUUGAGGGACUCGAA-3′ | 7 |
| 5′-AAGGACUUGAGGGACUCGAAG-3′ | 8 |
| 5′-AAGGACUUGAGGGACUCGAAGG-3′ | 9 |
| 5′-AAGGACUUGAGGGACUCGAAGGC-3′ | 10 |

TABLE 2
second RNA sequences substantially complementary
to one or more first RNA sequences provided in
Table 1

| | SEQ ID |
|---|---|
| miRNA sequence | NO: |
| 5′-UCGAGUCCCUCAAGUCCUU-3′ | 11 |
| 5′-UUCGAGUCCCUCAAGUCCUU-3′ | 12 |
| 5′-CUUCGAGUCCCUCAAGUCCUU-3′ | 13 |
| 5′-CCUUCGAGUCCCUCAAGUCCUU-3′ | 14 |
| 5′-GCCUUCGAGUCCCUCAAGUCCUU-3′ | 15 |
| 5′-CUUCGAGUCUCAAGUCCUU-3′ | 16 |
| 5′-ACGAGUCCCUCAAGUCCUC-3′ | 17 |

TABLE 3
Target Sequences in Exon 1 of human HTT gene,
targeted by the miRNAs provided by Tables 1 and 2

| Target Sequence | SEQ ID NO: |
|---|---|
| aaggacuuga gggacucgaa | 18 |
| tccaagatgg acggccgctc a | 19 |
| ccaagatgga cggccgctca g | 20 |
| agatggacgg ccgctcaggt t | 21 |
| atggacggcc gctcaggttc t | 22 |
| gacggccgct caggttctgc t | 23 |
| cggccgctca ggttctgctt t | 24 |
| gtgctgagcg gcgccgcgag t | 25 |
| cgccgcgagt cggcccgagg c | 26 |
| accgccatgg cgaccctgga a | 27 |
| ccgccatggc gaccctggaa a | 28 |
| gaaggccttc gagtccctca a | 29 |
| cttcgagtcc ctcaagtcct t | 30 |
| ccgccgccgc ctcctcagct t | 31 |
| gccgcctcct cagcttcctc a | 32 |
| tcagccgccg ccgcaggcac a | 33 |
| gccgcaggca cagccgctgc t | 34 |
| ggcacagccg ctgctgcctc a | 35 |
| gccgctgctg cctcagccgc a | 36 |
| cggcccggct gtggctgagg a | 37 |
| ctgtggctga ggagccgctg c | 38 |
| tgtggctgag gagccgctgc a | 39 |

In some embodiments, an miRNA comprises SEQ ID NOs: 6 and 11, SEQ ID NOs: 7 and 12; SEQ ID NOs: 8 and 11; SEQ ID NOs: 8 and 16; SEQ ID NOs: 8 and 17; SEQ ID NOs: 9 and 14; or SEQ ID NOs: 10 and 15.

In some embodiments, the vector comprises a pre-miRNA having the sequence of SEQ ID NO: 40 or 41. These pre-miRNAs include scaffolds comprising SEQ ID NO: 8. Alternative first RNA sequences disclosed herein can be substituted for SEQ ID NO: 8 in either of SEQ ID NOs: 40 and 41.

In some embodiments, the vector comprises a pri-miRNA having the sequence of SEQ ID NO: 42 or 43. The pri-miRNA of SEQ ID NO: 42 includes scaffolds comprising SEQ ID NO: 8 and 16. Alternative RNA sequences disclosed herein can be substituted for SEQ ID NO: 8 and 16 in SEQ ID NO: 42. The pri-miRNA of SEQ ID NOs: 43 and 44 include scaffolds comprising SEQ ID NO: 8 and 17. Alternative RNA sequences disclosed herein can be substituted for SEQ ID NO: 8 and 17 in either of SEQ ID NOs: 43 and 44.

TABLE 4
pre-and pri-miRNAs comprising miRNAs provided in Tables 1 and 2

| | | SEQ ID |
|---|---|---|
| Name | Sequence | NO: |
| Pre- | 5′- | 40 |
| miR451a | CUUGGGAAUGGCAAGGAAGGACUUGAGGGACUCGAAGACGA | |
| | GUCCCUCAAGUCCUCUCUUGCUAUACCCAGA-3′ | |
| Pre- | 5′-UGCUGAAGGACUUGAGGGACUCGAAGGUUUUGGCCACUGACU | 41 |
| miR155 | GACCUUCGAGUCUCAAGUCCUUCAGGA-3′ | |
| | gcuaagcacu ucguggccgu cgaucguuua aagggaggua gugagucgac | 42 |
| | caguggaucc uggaggcuug cugaaggcug uaugcugaag gacuugaggg | |
| | acucgaaggu uuuggccacu gacugaccuu cgagucucaa guccuucagg | |
| | acacaaggcc uguuacuagc acucacaugg aacaaauggc ccagaucugg | |
| | ccgcacucga gauaucuaga cccagcuuuc uuguacaaag ugguugaucu | |
| | agagggcccg cgguucgcug au | |
| | gcuccugggc aacgugcugg uuauugugcu gucucaucau uuuggcaaag | 43 |
| | aauuaagggc gaauucgagc ucgguaccuc gcgaaugcau cuagauaucg | |
| | gcgcuaugcu uccugugccc ccaguggggc ccuggcuggg auuucaucau | |
| | auacuguaag gggcaccccc agcccaagaa cacuugggaa aaguccucuc | |
| | gggcaccccc agcucuggag ccugacaagg aggacaggag agaugcugca | |
| | agcccaagaa gcucucugcu cagccuguca caaccuacug acugccaggg | |
| | cacuugggaa uggcaaggaa ggacuugagg gacucgaaga cgagucccuc | |
| | aaguccucuc uugcuauacc cagaaaacgu gccaggaaga gaacucagga | |
| | cccugaagca gacuacugga agggagacuc cagcucaaac aaggcagggg | |
| | ugggggcgug ggauuggggg uaggggaggg aauagauaca uuuucucuuu | |
| | ccuguuguaa agaaauaaag auaagccagg cacaguggcu cacgccugua | |
| | aucccaccac uuucagaggc caaggcgcug gauccagauc ucgagcggcc | |
| | gcccg | |
| | agucucgugc agauggacag caccgcugag caauggaagc ggguaggccu | 44 |
| | uuggggcagc ggccaauagc agcuuugcuc cuucgcuuuc ugggcucaga | |
| | ggcugggaag gggugggucc gggggcgggc ucaggggcgg gcucaggggc | |
| | ggggcgggcg cccgaagguc cuccggaggc ccggcauucu gcacgcuuca | |
| | aaagcgcacg ucugccgcgc uguucuccuc uuccucaucu ccgggccuuu | |
| | cgacccggau cccccgggcu gcaggaauuc gagcucggua ccucgcgaau | |
| | gcaucuagau aucggcgcua ugcuuccugu gcccccagug gggcccuggc | |
| | ugggauuuca ucauauacug uaaguuugcg augagacacu acaguauaga | |
| | ugauguacua guccgggcac ccccagcucu ggagccugac aaggaggaca | |
| | ggagagaugc ugcaagccca agaagcucuc ugcucagccu gucacaaccu | |
| | acugacugcc agggcacuug ggaauggcaa ggaaggacuu gagggacucg | |
| | aagacgaguc ccucaagucc ucucuugcua uacccagaaa acgugccagg | |
| | aagagaacuc aggacccuga agcagacuac uggaagggag acuccagcuc | |
| | aaacaaggca gggguggggg cgugggauug gggguagggg agggaauaga | |
| | uacauuuucu cuuuccuguu guaaagaaau aaagauaagc caggcacagu | |
| | ggcucacgcc uguaauccca ccacuuucag aggccaaggc gcuggaucca | |
| | gaucucgagc ggccgcccg | |

In some embodiments, the inhibitory nucleic acid can comprise one or more of SEQ ID NOs: 1-102 and/or 103-249 of International Patent Publication WO2017/201258. In some embodiments, the inhibitory nucleic acid can comprise one or more of the duplex combinations selected from SEQ ID NOs: 1-249 of International Patent Publication WO2017/201258 which are provided in Tables 3-5 of International Patent Publication WO2017/201258. In some embodiments, the vector can comprise one or more of the pri-miRNAs which are provided in Table 9 or the pri-raiRNAs which are provided in Table 10 of International Patent Publication WO2017/201258. The contents of International Patent Publication WO2017/201258 are incorporated by reference herein in their entirety.

In some embodiments, the inhibitory nucleic acid can comprise one or more of SEQ ID NOs: 914-1013 and/or 1014-1160 of International Patent Publication WO2018/204803. In some embodiments, the inhibitory nucleic acid can comprise one or more of the duplex combinations selected from SEQ ID NOs: 914-1160 of International Patent Publication WO2018/204803 which are provided in Tables 4-6 of International Patent Publication WO2018/204803. The contents of International Patent Publication WO2018/204803 are incorporated by reference herein in their entirety.

In some embodiments, the inhibitory nucleic acid can comprise one or more of SEQ ID NOs: 916-1015 and/or 1016-1162, of International Patent Publication WO2018/204797. In some embodiments, the inhibitory nucleic acid can comprise one or more of SEQ ID NOs: 916-1015, 1016-1162, 1164-1332, and/or 1333-1501 of International Patent Publication WO2018/204797. In some embodiments, the inhibitory nucleic acid can comprise one or more of the duplex combinations selected from SEQ ID NOs: 916-1162 of International Patent Publication WO2018/204797 which are provided in Tables 4-6 of International Patent Publication WO2018/204797. In some embodiments, the inhibitory nucleic acid can comprise one or more of the duplex combinations selected from SEQ ID NOs: 1164-1501 of International Patent Publication WO2018/204797 which are provided in Table 9 of International Patent Publication WO2018/204797. The contents of International Patent Publication WO2018/204797 are incorporated by reference herein in their entirety.

In some embodiments, the inhibitory nucleic acid can target, e.g., comprise a sequence complementary or substantially complementary to, a heterozygous SNP within a gene encoding a gain-of-function mutant huntingtin protein. In some embodiments, the SNP has an allelic frequency of at least 10% in a sample population. In some embodiments, the SNP present at a genomic site selected from the group consisting of RS362331, RS4690077, RS363125, RS363075, RS362268, RS362267, RS362307, RS362306, RS362305, RS362304, RS362303, and RS7685686. Such SNPs are described in more detail in, e.g., U.S. Pat. No. 9,343,943 which is incorporated by reference herein in its entirety. In some embodiments, the target sequence is one of SEQ ID NOs: 45-49. In some embodiments, the inhibitory nucleic acid sequence comprises one or more of SEQ ID NOs: 50-61. In some embodiments, the inhibitory nucleic acid sequence comprises at least SEQ ID NOs: 50 and 51, e.g., in a duplex. In some embodiments, the inhibitory nucleic acid sequence comprises at least SEQ ID NOs: 52 and 53, e.g., in a duplex. In some embodiments, the inhibitory nucleic acid sequence comprises at least SEQ ID NOs: 54 and 55, e.g., in a duplex. In some embodiments, the inhibitory nucleic acid sequence comprises at least SEQ ID NOs: 56 and 57, e.g., in a duplex. In some embodiments, the inhibitory nucleic acid sequence comprises at least SEQ ID NOs: 58 and 59, e.g., in a duplex. In some embodiments, the inhibitory nucleic acid sequence comprises at least SEQ ID NOs: 60 and 61, e.g., in a duplex.

TABLE 5

| Target Sequence | SEQ ID NO: |
|---|---|
| ccacgccugc ucccucaucc acugugugca cuucauccug | 45 |
| ccacgccugc ucccucaucu acugugugca cuucauccug | 46 |
| uaagagaugg ggacaguaau ucaacgcuag aagaaca | 47 |
| uaagagaugg ggacaguacu ucaacgcuag aagaaca | 48 |
| cagatgcc atggcctgtgct gggccag | 49 |

TABLE 6
sense and antisense (or first and second RNA sequences) that target SNPs in
human HTT gene

| Sense sequence | SEQ ID NO: | Antisense sequence | SEQ ID NO: |
|---|---|---|---|
| ucccucaucc acugugugaa c | 50 | gcacacagug gaugagggag c | 51 |
| ucccucaucu acugugugaa c | 52 | cgagggagua gaugacacac g | 53 |
| gggacaguaa uucaacgcgu c | 54 | agcguugaau uacugucccc a | 55 |
| gggacaguac uucaacgcgu c | 56 | accccuguca ugaaguugcg a | 57 |
| ugccauggcc ugugcugguc c | 58 | cccagcacag gccauggca c | 59 |
| ugccauggca ugugcugguc c | 60 | cccagcacau gccuaggcau c | 61 |

In some embodiments, an inhibitory nucleic acid, e.g., miRNA, can hybridize specifically to, or target a polymorphism, mutation, or SNP in one of the genes disclosed herein. Methods of selecting inhibitory nucleic acid sequences that target polymorphisms, e.g., SNPs, in a HTT gene are known in the art. For example, such methods are disclosed in U.S. Pat. Nos. 8,679,750 and 7,947,658, each of which is incorporated by reference herein in its entirety. In some embodiments, the inhibitory nucleic acid can comprise a sequence, e.g., one or more of SEQ ID NOs: 1-342 of U.S. Pat. No. 8,679,750 or 7,947,658.

In some embodiments, the inhibitory nucleic acid can comprise one or more of SEQ ID NOs: 62-66.

TABLE 7
In some embodiments, the capitalized letters
comprise 2′-O-(2-methoxy)ethyl modifications.

| | SEQ ID NO |
|---|---|
| 5′-CTCAGtaacattgacACCAC-3′ | 62 |
| 5′-CTCGActaaagcaggATTTC-3 | 63 |
| 5′-CCTTCcctgaaggttCCTCC-3′ | 64 |
| 5′-GCAGGgttaccgccaTCCCC-3′ | 65 |
| 5′-CGAGAcagtcgcttcCACTT-3′ | 66 |

Further suitable sequences are known in the art, e.g., in U.S. Pat. No. 7,951,934, Miniarikova et al. Molecular Therapy—Nucleic Acids 2015 5:e297; and Kordasiweicz et al. Neuron 2012 74:1031-1044; each of which is incorporated by reference herein in its entirety.

In some embodiments of any of the aspects, the inhibitory RNA (e.g., miRNA) binds and/or targets the 5′ untranslated region (UTR) of the target. In some embodiments of any of the aspects, the inhibitory RNA (e.g., miRNA) binds and/or targets one or more exons of the target. In some embodiments of any of the aspects, the inhibitory RNA (e.g., miRNA) binds and/or targets the 5′ UTR, exon 1, CAG repeats, the CAG 5′-jumper, or a CAG 3′jumper of HTT.

In some embodiments, the inhibitory RNA and/or vector does not comprise a sequence of any of SEQ ID NOs: 67-73. In some embodiments, the inhibitory RNA and/or vector does not comprise a sequence having more than 80%, more than 85%, more than 90%, more than 95%, or more than 98% sequence identity with any of any of SEQ ID NOs: 67-73.

In some embodiments, the inhibitory RNA and/or vector does not comprise a sequence of any of SEQ ID NOs: 67-73. In some embodiments, the inhibitory RNA and/or vector does not comprise a sequence of any of SEQ ID NOs: 135-151. In some embodiments, the inhibitory RNA and/or vector does not comprise a sequence having more than 80%, more than 85%, more than 90%, more than 95%, or more than 98% sequence identity with any of any of SEQ ID NOs: 67-73. In some embodiments, the inhibitory RNA and/or vector does not comprise a sequence having more than 80%, more than 85%, more than 90%, more than 95%, or more than 98% sequence identity with any of any ofSSEQ ID NOs: 135-151.

In some embodiments, the inhibitory RNA and/or vector does comprise a sequence of any of SEQ ID NOs: 67-73. In some embodiments, the inhibitory RNA and/or vector does comprise a sequence having more than 80%, more than 85%, more than 90%, more than 95%, or more than 98% sequence identity with any ofany ofSEQ ID NOs: 67-73.

In some embodiments, the inhibitory RNA and/or vector does comprise a sequence of any of SEQ ID NOs: 67-73 or 135-151. In some embodiments, the inhibitory RNA and/or vector does comprise a sequence having more than 80%, more than 85%, more than 90%, more than 95%, or more than 98% sequence identity with any of any of SEQ ID NOs: 67-73 or 135-151. See e.g., International Patent Application WO 2021/127455, the contents of which are incorporated herein by reference in their entireties.

TABLE 8

| | SEQ ID NO: |
|---|---|
| CGAGGCCGGGGCGGGGCACA | 67 |
| CGGGGCGGGGCCGTGGAGGG | 68 |
| ACTGTGCCACTATGTTTTCA | 69 |
| GCCTTCATCAGCTTTTCCAG | 70 |
| GCTGCTGCTGCTGCTGCTGC | 71 |
| TGCTGGAAGGACTTGAGGGA | 72 |
| TGTTGCTGCTGCTGCTGCTG | 73 |
| TGCTGCTGCTGCTGCTGCTG | 135 |
| GGCGGCGGCGGCGGCGGCGG | 136 |
| GAGGGGTGGGGAGGCTGGGG | 137 |
| TCCTTGACCTGCTGCTGCAG | 138 |
| CCTTCCACTGGCCATGATGC | 139 |
| ACTGTGCCACTATGTTTTCA | 140 |
| TGAGGTATCAGATTGTCTAG | 141 |
| AAAttAATCTCTTACCTGAT | 142 |
| CCCAGGGCTAGCAAGGAACA | 143 |
| AATTCAGTAGCTTCCCTTAA | 144 |
| CTGGGCCCGCAGCGGAAGGG | 145 |
| TTATTGCTGTCTACTATCCG | 146 |
| TCAGTCCTTCCCAAAGCTCT | 147 |
| TAATCTCTTTACTGATATAA | 148 |
| TCAGCAGTGTTATTTCTTAC | 149 |
| AAACCGttACCAttACtGAGtt | 150 |
| AAAtCGCtGAtttGtGtAGtC | 151 |

Suitable sequences for use in inhibitory nucleic acids (e.g., miRNAs) that target AD and/or PD associated targets are known in the art, e.g., see International Patent Publication WO2011/133890, WO2012/036433, WO2013/007874; U.S. Patent Publications US2016/0264965; U.S. Pat. Nos. 7,829,694, 8,415,319, 10,125,363, 10,011,835 The contents of the foregoing references are incorporated by reference herein in their entirety.

In some embodiments of any of the aspects, the agent that treats a neurological disease or disorder is or comprises an inhibitory nucleic acid. In some embodiments of any of the aspects, inhibitors of the expression of a given gene can be an inhibitory nucleic acid. As used herein, “inhibitory nucleic acid” refers to a nucleic acid molecule which can inhibit the expression of a target, e.g., double-stranded RNAs (dsRNAs), inhibitory RNAs (iRNAs), and the like.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject can result in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length. Exemplary embodiments of types of inhibitory nucleic acids can include, e.g., siRNA, shRNA, miRNA, and/or amiRNA, which are well known in the art.

In some embodiments of any of the aspects, the inhibitory is a miRNA. MicroRNAs (miRNAs) are small RNAs of 17-25 nucleotides, which function as regulators of gene expression in eukaryotes. A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The duplex structure comprises a) a first RNA sequence a region of complementarity that is substantially complementary, and generally fully complementary, to a target sequence and b) second RNA sequence region that is complementary to the first RNA sequence strand, such that the two sequences hybridize and form a duplex structure when combined under suitable conditions. The target sequence can be derived from the sequence of an mRNA formed during the expression of the target, e.g., it can span one or more intron boundaries. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive.

miRNAs are initially expressed in the nucleus as part of long primary transcripts called primary miRNAs (pri-miRNAs). The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Inside the nucleus, pri-miRNAs are partially digested by the enzyme Drosha, to form 65-120 nucleotide-long hairpin precursor miRNAs (pre-miRNAs) that are exported to the cytoplasm for further processing by Dicer into shorter, mature miRNAs, which are the active molecules. In animals, these short RNAs comprise a 5′ proximal “seed” region (nucleotides 2 to 8) which appears to be the primary determinant of the pairing specificity of the miRNA to the 3′ untranslated region (3′-UTR) of a target mRNA. Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length. In some embodiments, an isolated nucleic acid of the disclosure comprises a sequence encoding a pri-miRNA, a pre-miRNA, or a mature miRNA comprising a sequence set forth in any one of SEQ ID NOs: 6-17, 40-44, or 50-66.

In the context of the invention, a miRNA molecule or an equivalent or a mimic or an isomiR thereof may be a synthetic or natural or recombinant or mature or part of a mature miRNA or a human miRNA or derived from a human miRNA as further defined in the part dedicated to the general definitions. A human miRNA molecule is a miRNA molecule which is found in a human cell, tissue, organ or body fluids (i.e. endogenous human miRNA molecule). A human miRNA molecule may also be a human miRNA molecule derived from an endogenous human miRNA molecule by substitution, deletion and/or addition of a nucleotide. A miRNA molecule or an equivalent or a mimic thereof may be a single stranded or double stranded RNA molecule. Preferably a miRNA molecule or an equivalent, or a mimic thereof is from 6 to 30 nucleotides in length, preferably 12 to 30 nucleotides in length, preferably 15 to 28 nucleotides in length, more preferably said molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

In a preferred embodiment, a miRNA molecule or equivalent or mimic or isomiR thereof comprises at least 6 of the 7 nucleotides present in the seed sequence of said miRNA molecule or equivalent or mimic or isomiR thereof. Preferably in this embodiment, a miRNA molecule or an equivalent or a mimic or isomiR thereof is from 6 to 30 nucleotides in length and more preferably comprises at least 6 of the 7 nucleotides present in the seed sequence of said miRNA molecule or equivalent thereof. Even more preferably a miRNA molecule or an equivalent or a mimic or isomiR thereof is from 15 to 28 nucleotides in length and more preferably comprises at least 6 of the 7 nucleotides present in the seed sequence, even more preferably a miRNA molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Accordingly, a preferred miRNA molecule or equivalent or mimic or isomiR thereof comprises at least 6 of the 7 nucleotides present in the seed sequence identified in or as SEQ ID NO: 6-17, 40-44, or 50-66 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Delivery vehicles for miRNA include but are not limited to the following: liposomes, polymeric nanoparticles, viral systems, conjugation of lipids or receptor-binding molecules, exosomes, and bacteriophage; see e.g., Baumann and Winkler, miRNA-based therapies: Strategies and delivery platforms for oligonucleotide and non-oligonucleotide agents, Future Med Chem. 2014, 6(17): 1967-1984; U.S. Pat. Nos. 8,900,627; 9,421,173; 9,555,060; WO 2019/177550; the contents of each of which are incorporated herein by reference in their entireties.

A microRNA sequence comprises a “seed” region, i.e., a sequence in the region of positions 2-8 of the mature microRNA, which sequence has perfect Watson-Crick complementarity to the miRNA target sequence of the nucleic acid. In one embodiment, the viral genome may be engineered to include, alter or remove at least one miRNA binding site, sequence or seed region.

The term substantial complementarity means that is not required to have the first and second RNA sequence to be fully complementary, or to have the first RNA sequence and a reference or target sequence (e.g., SEQ ID NO: 3 or 4) to be fully complementary. In one embodiment, the substantial complementarity between a RNA sequence and the target consists of having no mismatches, one mismatched nucleotide, or two mismatched nucleotides. It is understood that one mismatched nucleotide means that over the entire length of the RNA sequence that base pairs with the target one nucleotide does not base pair with the target. Having no mismatches means that all nucleotides base pair with the target, and having 2 mismatches means two nucleotides do not base pair with the target.

The miRNAs and/or the transgene comprising one or more miRNAs can be provided in or comprise a scaffold sequence. As used herein, “scaffold” refers to portions of the miRNA-encoding sequence that are external to the mature duplex structure. For example, the scaffold can comprise loops and/or stem regions. Accordingly, scaffolds are useful in producing, encoding, and/or expressing the miRNAs described herein. Scaffolds used in the compositions and methods described herein can be sequences of, obtained from, and/or derived from endogenous and/or naturally-occurring miRNA scaffolds, e.g., human miRNAs. In some embodiments, the scaffold sequence is obtained used in the compositions and methods described herein can be sequences of, obtained from, and/or derived from endogenous and/or naturally-occurring miRNA scaffolds of miRNAs that are overexpressed in one or more NS and/or CNS diseases.

### Nucleic Acids

In some aspects, the disclosure provides isolated nucleic acids that are useful for reducing (e.g., inhibiting) expression of a pathogenic gene (e.g., HTT) and/or which encode CYP46A1. A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F.M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more inhibitory RNAs (e.g., miRNAs) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAV 11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV 11, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10): 1648-1656. In some embodiments of any of the aspects disclosed herein, at least one or more ITRs are less than 145 bp length, e.g., 130 bp length.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency {i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

In some aspects, the disclosure provides an isolated nucleic acid comprising a transgene, wherein the transgene comprises a nucleic acid sequence encoding one or more microRNAs (e.g., miRNAs).

It should be appreciated that an isolated nucleic acid or vector (e.g., rAAV vector), in some embodiments comprises a nucleic acid sequence encoding more than one (e.g., a plurality, such as 2, 3, 4, 5, 10, or more) miRNAs. In some embodiments, each of the more than one miRNAs targets (e.g., hybridizes or binds specifically to) the same target gene (e.g., an isolated nucleic acid encoding three unique miRNAs, where each miRNA targets the HTT gene). In some embodiments, each of the more than one miRNAs targets (e.g., hybridizes or binds specifically to) a different target gene.

In some aspects, the disclosure provides isolated nucleic acids and vectors (e.g., rAAV vectors) that encode one or more artificial miRNAs. As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. For example, in some embodiments an artificial miRNA comprises a miR-155 pri-miRNA backbone into which a sequence encoding a mature HTT-specific miRNA (e.g., any one of SEQ ID NOs: 6-17, 40-44, or 50-66) has been inserted in place of the endogenous miR-155 mature miRNA-encoding sequence. In some embodiments, miRNA (e.g., an artificial miRNA) as described by the disclosure comprises a miR-155 backbone sequence, a miR-30 backbone sequence, a mir-64 backbone sequence, or a miR-122 backbone sequence.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon. In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base.

It should be appreciated that in cases where a transgene encodes more than one miRNA, each miRNA may be positioned in any suitable location within the transgene. For example, a nucleic acid encoding a first miRNA may be positioned in an intron of the transgene and a nucleic acid sequence encoding a second miRNA may be positioned in another untranslated region (e.g., between the last codon of a protein coding sequence and the first base of the poly-A tail of the transgene).

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the p-actin promoter, the phosphoglycerol kinase (PGK) promoter, and the EF 1 a promoter [Invitrogen]. In some embodiments, a promoter is an enhanced chicken R-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionein (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268: 1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3: 1002-9 (1996); alpha-fetoprotein (AFP) promoter, Arbuthnot et al., Hum. Gene Ther., 7: 1503-14 (1996)), bone osteocalcin promoter (Stein et al., Mol. Biol. Rep., 24: 185-96 (1997)); bone sialoprotein promoter (Chen et al., J. Bone Miner. Res., 11:654-64 (1996)), CD2 promoter (Hansal et al., J. Immunol., 161: 1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan. NS-specific promoters contemplated for use in the present methods and compositions also include those described in Patent Application GB2013940.8 filed Sep. 4, 2020 and GB2005732.9 filed Apr. 20, 2020, which are incorporated by reference herein in their entireties. In some embodiments, the NS-specific promoter is a promoter of Table 10, or a promoter having at least 80%, at least 85%, at least 90%, at least 95%, at least 98% identity to a promoter of Table 10. In some embodiments, the NS-specific promoter is a promoter of Table 10, or a promoter having at least 80%, at least 85%, at least 90%, at least 95%, at least 98% identity to a promoter of Table 10 and retaining the NS-specific promoter activity of the promoter of Table 10.

CNS-specific promoters contemplated for use in the present methods and compositions also include those described in International Patent Application PCT/GB2021/050939 filed Apr. 19, 2021, the contents of which are incorporated by reference herein in their entireties. In some embodiments, the CNS-specific promoter is a promoter of Tables 11-13, or a promoter having at least 80%, at least 85%, at least 90%, at least 95%, at least 98% identity to a promoter of Tables 11-13. In some embodiments, the CNS-specific promoter is a promoter of Tables 11-13, or a promoter having at least 80%, at least 85%, at least 90%, at least 95%, at least 98% identity to a promoter of Tables 11-13 and retaining the CNS-specific promoter activity of the promoter of Tables 11-13.

In some embodiments, the nucleic acid comprises one or more CREs. In some embodiments, the nucleic acid comprises one or more NS-specific CREs or CNS-specific CREs. In some embodiments, the nucleic acid comprises one or more CREs of Tables 13-15, or a CRE having at least 80%, at least 85%, at least 90%, at least 95%, at least 98% identity to a CRE of Tables 13-15. In some embodiments, the CRE is a CRE of Tables 13-15, or a CRE having at least 80%, at least 85%, at least 90%, at least 95%, at least 98% identity to a CRE of Tables 13-15 and retaining the activity of the CRE of Tables 13-15.

In some embodiments, the CRE can comprise one or more CREs known in the art. For example, in one embodiment, the one or more CREs may be selected from SEQ ID NOs: 19-24, 27, 28, 37, 38 in Patent Application GB2013940.8 filed Sep. 4, 2020. For example, in one embodiment, the one or more CREs may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. The foregoing references are incorporated by reference herein in their entireties.

TABLE 10
NS-specific promoters

| NAME | SEQUENCE | Length |
|---|---|---|
| SP0013 | AACATATCCTGGTGTGGAGTAGGGGACGCTGCTCTGACAGAGGCTCGGGGGC | 795 |
| (SEQ ID | CTGAGCTGGCTCTGTGAGCTGGGGAGGAGGCAGACAGCCAGGCCTTGTCTGCA | |
| NO: 74) | AGCAGACCTGGCAGCATTGGGCTGGCCGCCCCCCAGGGCCTCCTCTTCATGCC | |
| | CAGTGAATGACTCACCTTGGCACAGACACAATGTTCGGGGTGGGCACAGTGCC | |
| | TGCTTCCCGCCGCACCCCAGCCCCCCTCAAATGCCTTCCGAGAAGCCCATTGA | |
| | GCAGGGGGCTTGCATTGCACCCCAGCCTGACAGCCTGGCATCTTGGGATAAAA | |
| | GCAGCACAGCCCCCTAGGGGCTGCCCTTGCTGTGTGGCGCCACCGGCGGTGGA | |
| | GAACAAGGCTCTATTCAGCCTGTGCCCAGGAAAGGGGATCAGGGGATGCCCA | |
| | GGCATGGACAGTGGGTGGCAGGGGGGGAGAGGAGGGCTGTCTGCTTCCCAGA | |
| | AGTCCAAGGACACAAATGGGTGAGGGGAGGGCTAGGCCTGCGCACCCACCCA | |
| | CCGACCCCTCACCCACCGACCCGTCACCCACCGACCAAGGGGCACCCTGGCCT | |
| | AGAGGGGATGCTGAGCGGGACCCGCCTCCTGCCTCTGGCAGTCCCAGATGGG | |
| | ACTTGGACCCCGCAGTTGCTCTCTCGGACCCTAAGTTTCTACCCCTGGATCTAA | |
| | GGCGGAGCTGGGTTTGCGGATCCCACGGTTCCCGGCGGGGCGGGGCCCGGTC | |
| | GCCCCTCCCCCTCCCCGCCCTCCTGCGCCGGGAGCAGTGCATTGTGGGAAACT | |
| | CCCGA | |
| SP0014 | AACATATCCTGGTGTGGAGTAGGGGACGCTGCTCTGACAGAGGCTCGGGGGC | 810 |
| (SEQ ID | CTGAGCTGGCTCTGTGAGCTGGGGAGGAGGCAGACAGCCAGGCCTTGTCTGCA | |
| NO: 75) | AGCAGACCTGGCAGCATTGGGCTGGCCGCCCCCCAGGGCCTCCTCTTCATGCC | |
| | CAGTGAATGACTCACCTTGGCACAGACACAATGTTCGGGGTGGGCACAGTGCC | |
| | TGCTTCCCGCCGCACCCCAGCCCCCCTCAAATGCCTTCCGAGAAGCCCATTGA | |
| | GCAGGGGGCTTGCATTGCACCCCAGCCTGACAGCCTGGCATCTTGGGATAAAA | |
| | GCAGCACAGCCCCCTAGGGGCTGCCCTTGCTGTGTGGCGCCACCGGCGGTGGA | |
| | GAACAAGGCTCTATTCAGCCTGTGCCCAGGAAAGGGGATCAGGGGATGCCCA | |
| | GGCATGGACAGTGGGTGGCAGGGGGGGAGAGGAGGGCTGTCTGCTTCCCAGA | |
| | AGTCCAAGGACACAAATGGGTGAGGGGATGCGGCGAGGCGCGTGCGCACTGC | |
| | CAGCTTCAGCACCGCGGACAGTGCCTTCGCCCCCGCCTGGCGGCGCGCGCCAC | |
| | CGCCGCCTCAGCACTGAAGGCGCGCTGACGTCACTCGCCGGTCCCCCGCAAAC | |
| | TCCCCTTCCCGGCCACCTTGGTCGCGTCCGCGCCGCCGCCGGCCCAGCCGGAC | |
| | CGCACCACGCGAGGCGCGAGATAGGGGGGCACGGGCGCGACCATCTGCGCTG | |
| | CGGCGCCGGCGACTCAGCGCTGCCTCAGTCTGCGGTGGGCAGCGGAGGAGTC | |
| | GTGTCGTGCCTGAGAGCGCAG | |
| SP0026 | AACATATCCTGGTGTGGAGTAGGGGACGCTGCTCTGACAGAGGCTCGGGGGC | 916 |
| (SEQ ID | CTGAGCTGGCTCTGTGAGCTGGGGAGGAGGCAGACAGCCAGGCCTTGTCTGCA | |
| NO: 76) | AGCAGACCTGGCAGCATTGGGCTGGCCGCCCCCCAGGGCCTCCTCTTCATGCC | |
| | CAGTGAATGACTCACCTTGGCACAGACACAATGTTCGGGGTGGGCACAGTGCC | |
| | TGCTTCCCGCCGCACCCCAGCCCCCCTCAAATGCCTTCCGAGAAGCCCATTGA | |
| | GCAGGGGGCTTGCATTGCACCCCAGCCTGACAGCCTGGCATCTTGGGATAAAA | |
| | GCAGCACAGCCCCCTAGGGGCTGCCCTTGCTGTGTGGCGCCACCGGCGGTGGA | |
| | GAACAAGGCTCTATTCAGCCTGTGCCCAGGAAAGGGGATCAGGGGATGCCCA | |
| | GGCATGGACAGTGGGTGGCAGGGGGGGAGAGGAGGGCTGTCTGCTTCCCAGA | |
| | AGTCCAAGGACACAAATGGGTGAGGGGAAGCGCGCAGAGTCTGCATGCGTGA | |
| | GGAAGCTCCTGGGCGCGTCACAGCCGCGCTATTCTCAGCGTCTCTCCTTTTATG | |
| | GCTCCGGAAGTGAGCTGGGGTTGCTGGCAGCCTGGCTGGCACTGGGCTAGGCC | |
| | TGCGCACCCACCCACCGACCCCTCACCCACCGACCCGTCACCCACCGACCAAG | |
| | GGGCACCCTGGCCTAGAGGGGATGCTGAGCGGGACCCGCCTCCTGCCTCTGGC | |
| | AGTCCCAGATGGGACTTGGACCCCGCAGTTGCTCTCTCGGACCCTAAGTTTCT | |
| | ACCCCTGGATCTAAGGCGGAGCTGGGTTTGCGGATCCCACGGTTCCCGGCGGG | |
| | GCGGGGCCCGGTCGCCCCTCCCCCTCCCCGCCCTCCTGCGCCGGGAGCAGTGC | |
| | ATTGTGGGAAACTCCCGA | |
| SP0027 | AACATATCCTGGTGTGGAGTAGGGGACGCTGCTCTGACAGAGGCTCGGGGGC | 931 |
| (SEQ ID | CTGAGCTGGCTCTGTGAGCTGGGGAGGAGGCAGACAGCCAGGCCTTGTCTGCA | |
| NO: 77) | AGCAGACCTGGCAGCATTGGGCTGGCCGCCCCCCAGGGCCTCCTCTTCATGCC | |
| | CAGTGAATGACTCACCTTGGCACAGACACAATGTTCGGGGTGGGCACAGTGCC | |
| | TGCTTCCCGCCGCACCCCAGCCCCCCTCAAATGCCTTCCGAGAAGCCCATTGA | |
| | GCAGGGGGCTTGCATTGCACCCCAGCCTGACAGCCTGGCATCTTGGGATAAAA | |
| | GCAGCACAGCCCCCTAGGGGCTGCCCTTGCTGTGTGGCGCCACCGGCGGTGGA | |
| | GAACAAGGCTCTATTCAGCCTGTGCCCAGGAAAGGGGATCAGGGGATGCCCA | |
| | GGCATGGACAGTGGGTGGCAGGGGGGGAGAGGAGGGCTGTCTGCTTCCCAGA | |
| | AGTCCAAGGACACAAATGGGTGAGGGGAAGCGCGCAGAGTCTGCATGCGTGA | |
| | GGAAGCTCCTGGGCGCGTCACAGCCGCGCTATTCTCAGCGTCTCTCCTTTTATG | |
| | GCTCCGGAAGTGAGCTGGGGTTGCTGGCAGCCTGGCTGGCACTTGCGGCGAGG | |
| | CGCGTGCGCACTGCCAGCTTCAGCACCGCGGACAGTGCCTTCGCCCCCGCCTG | |
| | GCGGCGCGCGCCACCGCCGCCTCAGCACTGAAGGCGCGCTGACGTCACTCGCC | |
| | GGTCCCCCGCAAACTCCCCTTCCCGGCCACCTTGGTCGCGTCCGCGCCGCCGC | |
| | CGGCCCAGCCGGACCGCACCACGCGAGGCGCGAGATAGGGGGGCACGGGCGC | |
| | GACCATCTGCGCTGCGGCGCCGGCGACTCAGCGCTGCCTCAGTCTGCGGTGGG | |
| | CAGCGGAGGAGTCGTGTCGTGCCTGAGAGCGCAG | |
| SP0030 | AACATATCCTGGTGTGGAGTAGGGGACGCTGCTCTGACAGAGGCTCGGGGGC | 617 |
| (SEQ ID | CTGAGCTGGCTCTGTGAGCTGGGGAGGAGGCAGACAGCCAGGCCTTGTCTGCA | |
| NO: 78) | AGCAGACCTGGCAGCATTGGGCTGGCCGCCCCCCAGGGCCTCCTCTTCATGCC | |
| | CAGTGAATGACTCACCTTGGCACAGACACAATGTTCGGGGTGGGCACAGTGCC | |
| | TGCTTCCCGCCGCACCCCAGCCCCCCTCAAATGCCTTCCGAGAAGCCCATTGA | |
| | GCAGGGGGCTTGCATTGCACCCCAGCCTGACAGCCTGGCATCTTGGGATAAAA | |
| | GCAGCACGGGCTAGGCCTGCGCACCCACCCACCGACCCCTCACCCACCGACCC | |
| | GTCACCCACCGACCAAGGGGCACCCTGGCCTAGAGGGGATGCTGAGCGGGAC | |
| | CCGCCTCCTGCCTCTGGCAGTCCCAGATGGGACTTGGACCCCGCAGTTGCTCTC | |
| | TCGGACCCTAAGTTTCTACCCCTGGATCTAAGGCGGAGCTGGGTTTGCGGATC | |
| | CCACGGTTCCCGGCGGGGCGGGGCCCGGTCGCCCCTCCCCCTCCCCGCCCTCC | |
| | TGCGCCGGGAGCAGTGCATTGTGGGAAACTCCCGA | |
| SP0031 | AACATATCCTGGTGTGGAGTAGGGGACGCTGCTCTGACAGAGGCTCGGGGGC | 632 |
| (SEQ ID | CTGAGCTGGCTCTGTGAGCTGGGGAGGAGGCAGACAGCCAGGCCTTGTCTGCA | |
| NO: 79) | AGCAGACCTGGCAGCATTGGGCTGGCCGCCCCCCAGGGCCTCCTCTTCATGCC | |
| | CAGTGAATGACTCACCTTGGCACAGACACAATGTTCGGGGTGGGCACAGTGCC | |
| | TGCTTCCCGCCGCACCCCAGCCCCCCTCAAATGCCTTCCGAGAAGCCCATTGA | |
| | GCAGGGGGCTTGCATTGCACCCCAGCCTGACAGCCTGGCATCTTGGGATAAAA | |
| | GCAGCACTGCGGCGAGGCGCGTGCGCACTGCCAGCTTCAGCACCGCGGACAG | |
| | TGCCTTCGCCCCCGCCTGGCGGCGCGCGCCACCGCCGCCTCAGCACTGAAGGC | |
| | GCGCTGACGTCACTCGCCGGTCCCCCGCAAACTCCCCTTCCCGGCCACCTTGGT | |
| | CGCGTCCGCGCCGCCGCCGGCCCAGCCGGACCGCACCACGCGAGGCGCGAGA | |
| | TAGGGGGGCACGGGCGCGACCATCTGCGCTGCGGCGCCGGCGACTCAGCGCT | |
| | GCCTCAGTCTGCGGTGGGCAGCGGAGGAGTCGTGTCGTGCCTGAGAGCGCAG | |
| SP0032 | TTCAGAGGTCGGATCTGAATCCAGCTCCAAGGCCCCAGCACCCAAGCCCTGAC | 701 |
| (SEQ ID | CCCATGGCTGCCTGCTGGCTGGGAGTGGCATTCTTTAGAACAGGAAAGGCAGT | |
| NO: 80) | GAGTTCTCATTGCATCAATACTTGCATTTGCTACAACAGAAGCTTTTTGTCTCT | |
| | TCCTCACATTCCTTTAGAACACAAGCCTCCTTTTCTGCCTGTGGCCATCGGTCA | |
| | CTGGAGTCAGCCTCGTGAGTGGCTTGGTGGCGGATGGCACCGTCCTTTGTGGG | |
| | AGAAAACAATGTTGCTGCCCAGGCCTTTCTGGAATGACCCCTTCCCACCCCCT | |
| | GACCAGCCCCAGCAAGGCCCGGGGCTGGCTGCCTAGTTGATTGGAGCCAAGA | |
| | GTTTGCTGAATGGATGAAGGGAGAAGGGACATCCAAGGGCTAGGCCTGCGCA | |
| | CCCACCCACCGACCCCTCACCCACCGACCCGTCACCCACCGACCAAGGGGCAC | |
| | CCTGGCCTAGAGGGGATGCTGAGCGGGACCCGCCTCCTGCCTCTGGCAGTCCC | |
| | AGATGGGACTTGGACCCCGCAGTTGCTCTCTCGGACCCTAAGTTTCTACCCCTG | |
| | GATCTAAGGCGGAGCTGGGTTTGCGGATCCCACGGTTCCCGGCGGGGCGGGG | |
| | CCCGGTCGCCCCTCCCCCTCCCCGCCCTCCTGCGCCGGGAGCAGTGCATTGTG | |
| | GGAAACTCCCGA | |
| SP0033 | TTCAGAGGTCGGATCTGAATCCAGCTCCAAGGCCCCAGCACCCAAGCCCTGAC | 716 |
| (SEQ ID | CCCATGGCTGCCTGCTGGCTGGGAGTGGCATTCTTTAGAACAGGAAAGGCAGT | |
| NO: 81) | GAGTTCTCATTGCATCAATACTTGCATTTGCTACAACAGAAGCTTTTTGTCTCT | |
| | TCCTCACATTCCTTTAGAACACAAGCCTCCTTTTCTGCCTGTGGCCATCGGTCA | |
| | CTGGAGTCAGCCTCGTGAGTGGCTTGGTGGCGGATGGCACCGTCCTTTGTGGG | |
| | AGAAAACAATGTTGCTGCCCAGGCCTTTCTGGAATGACCCCTTCCCACCCCCT | |
| | GACCAGCCCCAGCAAGGCCCGGGGCTGGCTGCCTAGTTGATTGGAGCCAAGA | |
| | GTTTGCTGAATGGATGAAGGGAGAAGGGACATCCAATGCGGCGAGGCGCGTG | |
| | CGCACTGCCAGCTTCAGCACCGCGGACAGTGCCTTCGCCCCCGCCTGGCGGCG | |
| | CGCGCCACCGCCGCCTCAGCACTGAAGGCGCGCTGACGTCACTCGCCGGTCCC | |
| | CCGCAAACTCCCCTTCCCGGCCACCTTGGTCGCGTCCGCGCCGCCGCCGGCCC | |
| | AGCCGGACCGCACCACGCGAGGCGCGAGATAGGGGGGCACGGGCGCGACCAT | |
| | CTGCGCTGCGGCGCCGGCGACTCAGCGCTGCCTCAGTCTGCGGTGGGCAGCGG | |
| | AGGAGTCGTGTCGTGCCTGAGAGCGCAG | |
| SP0019 | TCAGGGGTGCAGCTTTTTTTCTGTCTTTTACTCAGCCTGAGAAAGGTTGTCGTT | 792 |
| (SEQ ID | TGACAAGGTTTGTTCAGAGGTCGGATCTGAATCCAGCTCCAAGGCCCCAGCAC | |
| NO: 82) | CCAAGCCCTGACCCCATGGCTGCCTGCTGGCTGGGAGTGGCATTCTTTAGAAC | |
| | AGGAAAGGCAGTGAGTTCTCATTGCATCAATACTTGCATTTGCTACAACAGAA | |
| | GCTTTTTGTCTCTTCCTCACATTCCTTTAGAACACAAGCCTCCTTTTCTGCCTGT | |
| | GGCCATCGGTCACTGGAGTCAGCCTCGTGAGTGGCTTGGTGGCGGATGGCACC | |
| | GTCCTTTGTGGGAGAAAACAATGTTGCTGCCCAGGCCTTTCTGGAATGACCCC | |
| | TTCCCACCCCCTGACCAGCCCCAGCAAGGCCCGGGGCTGGCTGCCTAGTTGAT | |
| | TGGAGCCAAGAGTTTGCTGAATGGATGAAGGGAGAAGGGACATCCAAGTGAC | |
| | TGAAGAAGATCTTAACAGAAGGGCTAGGCCTGCGCACCCACCCACCGACCCCT | |
| | CACCCACCGACCCGTCACCCACCGACCAAGGGGCACCCTGGCCTAGAGGGGA | |
| | TGCTGAGCGGGACCCGCCTCCTGCCTCTGGCAGTCCCAGATGGGACTTGGACC | |
| | CCGCAGTTGCTCTCTCGGACCCTAAGTTTCTACCCCTGGATCTAAGGCGGAGCT | |
| | GGGTTTGCGGATCCCACGGTTCCCGGCGGGGCGGGGCCCGGTCGCCCCTCCCC | |
| | CTCCCCGCCCTCCTGCGCCGGGAGCAGTGCATTGTGGGAAACTCCCGA | |
| SP0020 | CTTCCTCTTATATTTCACCAAGACTCAGTTCCTGAGCAAGAAACCACAGGCAC | 686 |
| (SEQ ID | AGCAAGTGCCATGAAAAGCGGCTTTGTGTGGGGTGGGCTCTTCACACTCCAAT | |
| NO: 83) | CTCCACTTCCTTCTCAAGGCCTCAAAAAAAGTTGAAAAATGAAAACAAAAGCC | |
| | CTGCTGTGTTGAGCTGGGCTCTGGCGTTGCCATGGACCCAGGGCAAACAGCGG | |
| | TGCTCCTGCTCTGCCCCCGGCTCAGCTCATGCTGGGCCTGCACTTCTGGAAGGG | |
| | AGCATGGACTTTGGAATGACTGGTTAGAACCCAAATGAATTAATGGAATTTGA | |
| | CATAGTTCAAAAATAATAAAATGTGATACCCATGAAATGCTGATATTCTGCCT | |
| | TAATTTGCCAGATTGGGGGCCGGGCTAGGCCTGCGCACCCACCCACCGACCCC | |
| | TCACCCACCGACCCGTCACCCACCGACCAAGGGGCACCCTGGCCTAGAGGGG | |
| | ATGCTGAGCGGGACCCGCCTCCTGCCTCTGGCAGTCCCAGATGGGACTTGGAC | |
| | CCCGCAGTTGCTCTCTCGGACCCTAAGTTTCTACCCCTGGATCTAAGGCGGAG | |
| | CTGGGTTTGCGGATCCCACGGTTCCCGGCGGGGCGGGGCCCGGTCGCCCCTCC | |
| | CCCTCCCCGCCCTCCTGCGCCGGGAGCAGTGCATTGTGGGAAACTCCCGA | |
| SP0021 | TCAGGGGTGCAGCTTTTTTTCTGTCTTTTACTCAGCCTGAGAAAGGTTGTCGTT | 807 |
| (SEQ ID | TGACAAGGTTTGTTCAGAGGTCGGATCTGAATCCAGCTCCAAGGCCCCAGCAC | |
| NO: 84) | CCAAGCCCTGACCCCATGGCTGCCTGCTGGCTGGGAGTGGCATTCTTTAGAAC | |
| | AGGAAAGGCAGTGAGTTCTCATTGCATCAATACTTGCATTTGCTACAACAGAA | |
| | GCTTTTTGTCTCTTCCTCACATTCCTTTAGAACACAAGCCTCCTTTTCTGCCTGT | |
| | GGCCATCGGTCACTGGAGTCAGCCTCGTGAGTGGCTTGGTGGCGGATGGCACC | |
| | GTCCTTTGTGGGAGAAAACAATGTTGCTGCCCAGGCCTTTCTGGAATGACCCC | |
| | TTCCCACCCCCTGACCAGCCCCAGCAAGGCCCGGGGCTGGCTGCCTAGTTGAT | |
| | TGGAGCCAAGAGTTTGCTGAATGGATGAAGGGAGAAGGGACATCCAAGTGAC | |
| | TGAAGAAGATCTTAACAGAATGCGGCGAGGCGCGTGCGCACTGCCAGCTTCA | |
| | GCACCGCGGACAGTGCCTTCGCCCCCGCCTGGCGGCGCGCGCCACCGCCGCCT | |
| | CAGCACTGAAGGCGCGCTGACGTCACTCGCCGGTCCCCCGCAAACTCCCCTTC | |
| | CCGGCCACCTTGGTCGCGTCCGCGCCGCCGCCGGCCCAGCCGGACCGCACCAC | |
| | GCGAGGCGCGAGATAGGGGGGCACGGGCGCGACCATCTGCGCTGCGGCGCCG | |
| | GCGACTCAGCGCTGCCTCAGTCTGCGGTGGGCAGCGGAGGAGTCGTGTCGTGC | |
| | CTGAGAGCGCAG | |
| SP0022 | CTTCCTCTTATATTTCACCAAGACTCAGTTCCTGAGCAAGAAACCACAGGCAC | 701 |
| (SEQ ID | AGCAAGTGCCATGAAAAGCGGCTTTGTGTGGGGTGGGCTCTTCACACTCCAAT | |
| NO: 85) | CTCCACTTCCTTCTCAAGGCCTCAAAAAAAGTTGAAAAATGAAAACAAAAGCC | |
| | CTGCTGTGTTGAGCTGGGCTCTGGCGTTGCCATGGACCCAGGGCAAACAGCGG | |
| | TGCTCCTGCTCTGCCCCCGGCTCAGCTCATGCTGGGCCTGCACTTCTGGAAGGG | |
| | AGCATGGACTTTGGAATGACTGGTTAGAACCCAAATGAATTAATGGAATTTGA | |
| | CATAGTTCAAAAATAATAAAATGTGATACCCATGAAATGCTGATATTCTGCCT | |
| | TAATTTGCCAGATTGGGGGCCTGCGGCGAGGCGCGTGCGCACTGCCAGCTTCA | |
| | GCACCGCGGACAGTGCCTTCGCCCCCGCCTGGCGGCGCGCGCCACCGCCGCCT | |
| | CAGCACTGAAGGCGCGCTGACGTCACTCGCCGGTCCCCCGCAAACTCCCCTTC | |
| | CCGGCCACCTTGGTCGCGTCCGCGCCGCCGCCGGCCCAGCCGGACCGCACCAC | |
| | GCGAGGCGCGAGATAGGGGGGCACGGGCGCGACCATCTGCGCTGCGGCGCCG | |
| | GCGACTCAGCGCTGCCTCAGTCTGCGGTGGGCAGCGGAGGAGTCGTGTCGTGC | |
| | CTGAGAGCGCAG | |
| SP0028 | TCAGGGGTGCAGCTTTTTTTCTGTCTTTTACTCAGCCTGAGAAAGGTTGTCGTT | 913 |
| (SEQ ID | TGACAAGGTTTGTTCAGAGGTCGGATCTGAATCCAGCTCCAAGGCCCCAGCAC | |
| NO: 86) | CCAAGCCCTGACCCCATGGCTGCCTGCTGGCTGGGAGTGGCATTCTTTAGAAC | |
| | AGGAAAGGCAGTGAGTTCTCATTGCATCAATACTTGCATTTGCTACAACAGAA | |
| | GCTTTTTGTCTCTTCCTCACATTCCTTTAGAACACAAGCCTCCTTTTCTGCCTGT | |
| | GGCCATCGGTCACTGGAGTCAGCCTCGTGAGTGGCTTGGTGGCGGATGGCACC | |
| | GTCCTTTGTGGGAGAAAACAATGTTGCTGCCCAGGCCTTTCTGGAATGACCCC | |
| | TTCCCACCCCCTGACCAGCCCCAGCAAGGCCCGGGGCTGGCTGCCTAGTTGAT | |
| | TGGAGCCAAGAGTTTGCTGAATGGATGAAGGGAGAAGGGACATCCAAGTGAC | |
| | TGAAGAAGATCTTAACAGAAAGCGCGCAGAGTCTGCATGCGTGAGGAAGCTC | |
| | CTGGGCGCGTCACAGCCGCGCTATTCTCAGCGTCTCTCCTTTTATGGCTCCGGA | |
| | AGTGAGCTGGGGTTGCTGGCAGCCTGGCTGGCACTGGGCTAGGCCTGCGCACC | |
| | CACCCACCGACCCCTCACCCACCGACCCGTCACCCACCGACCAAGGGGCACCC | |
| | TGGCCTAGAGGGGATGCTGAGCGGGACCCGCCTCCTGCCTCTGGCAGTCCCAG | |
| | ATGGGACTTGGACCCCGCAGTTGCTCTCTCGGACCCTAAGTTTCTACCCCTGGA | |
| | TCTAAGGCGGAGCTGGGTTTGCGGATCCCACGGTTCCCGGCGGGGCGGGGCCC | |
| | GGTCGCCCCTCCCCCTCCCCGCCCTCCTGCGCCGGGAGCAGTGCATTGTGGGA | |
| | AACTCCCGA | |
| SP0029 | TCAGGGGTGCAGCTTTTTTTCTGTCTTTTACTCAGCCTGAGAAAGGTTGTCGTT | 928 |
| (SEQ ID | TGACAAGGTTTGTTCAGAGGTCGGATCTGAATCCAGCTCCAAGGCCCCAGCAC | |
| NO: 87) | CCAAGCCCTGACCCCATGGCTGCCTGCTGGCTGGGAGTGGCATTCTTTAGAAC | |
| | AGGAAAGGCAGTGAGTTCTCATTGCATCAATACTTGCATTTGCTACAACAGAA | |
| | GCTTTTTGTCTCTTCCTCACATTCCTTTAGAACACAAGCCTCCTTTTCTGCCTGT | |
| | GGCCATCGGTCACTGGAGTCAGCCTCGTGAGTGGCTTGGTGGCGGATGGCACC | |
| | GTCCTTTGTGGGAGAAAACAATGTTGCTGCCCAGGCCTTTCTGGAATGACCCC | |
| | TTCCCACCCCCTGACCAGCCCCAGCAAGGCCCGGGGCTGGCTGCCTAGTTGAT | |
| | TGGAGCCAAGAGTTTGCTGAATGGATGAAGGGAGAAGGGACATCCAAGTGAC | |
| | TGAAGAAGATCTTAACAGAAAGCGCGCAGAGTCTGCATGCGTGAGGAAGCTC | |
| | CTGGGCGCGTCACAGCCGCGCTATTCTCAGCGTCTCTCCTTTTATGGCTCCGGA | |
| | AGTGAGCTGGGGTTGCTGGCAGCCTGGCTGGCACTTGCGGCGAGGCGCGTGCG | |
| | CACTGCCAGCTTCAGCACCGCGGACAGTGCCTTCGCCCCCGCCTGGCGGCGCG | |
| | CGCCACCGCCGCCTCAGCACTGAAGGCGCGCTGACGTCACTCGCCGGTCCCCC | |
| | GCAAACTCCCCTTCCCGGCCACCTTGGTCGCGTCCGCGCCGCCGCCGGCCCAG | |
| | CCGGACCGCACCACGCGAGGCGCGAGATAGGGGGGCACGGGCGCGACCATCT | |
| | GCGCTGCGGCGCCGGCGACTCAGCGCTGCCTCAGTCTGCGGTGGGCAGCGGA | |
| | GGAGTCGTGTCGTGCCTGAGAGCGCAG | |
| SP0011 | CTAGCCCACAGGAAATGTCTGTCTATATCCAGGCAAGTACCTTGCTCATTGGA | 529 |
| (SEQ ID | CCAACCCGAAACTGTTCAGGGAAGATCAGGGAAATCAACTCAGTTACAAATG | |
| NO: 88) | GGATAATCATGCCCAGTAAAAACTACCTGTGGTGAATAAAGAGTTAACCCCTG | |
| | TTCCATCTTAGGTCACTATGCAGAGTACCAATGAGTACAAGAGATGGTGCCAA | |
| | AGAGGGTGGCCCCTCCCTAGCTGGGAACAGTCAACCCTTAGGAACTAGACTGT | |
| | CAACACATCAGCCAGCCAGAGACAAGGGAAACCGTGGCAACCAAGTGTTGCT | |
| | GGCACATTGTGAGGTGGTGATGGGAACTGCAGAGGCCCTGCACAGCATGCTA | |
| | ATGAGCCCAGGCAAACATGAGCTCTCCCCATAGCTGGGCTGCGGCCCAACCCC | |
| | ACCCCCTCAGGCTATGCCAGGGGGTGTTGCCAGGGGCACCCGGGCATCGCCAG | |
| | TCTAGCCCACTCCTTCATAAAGCCCTCGCATCCCAGGAGCGAGCAGAGCCAGA | |
| | GC | |
| SP0034 | TCCAAAGAAAAGCCAGATAAGTAGCTGATTATTGCATAGAGCTGACAGTATCA | 646 |
| (SEQ ID | CAGGAAGATCAGTAGTAGCAGCTCAAGTACAAAAAGGTTAATTAGCAATACT | |
| NO: 89) | TAATAAGAAAAACTACCTCTGGCAGGTGAAGAGTTAATCCCTGGTCAATTTTA | |
| | AGCTACTCTGCTGAGAGTACTAATAAGTGTAGGGGTTGGAGCCAATGAGGGTG | |
| | ACCCCTTCCTTGATGGGAACAGTCATCCCTTAGGAACTGCCCTGGAAAGCATC | |
| | AGCCAGCCAGAAAACAGGGAAAGAGGCTGAGAAACCGTGGTAACCAAGTTTT | |
| | GCTGGCACTTTGTAAAATGGTAACTGCAACTGCCGAGGCTGTGCAGAGAATGC | |
| | TAATAAGCCTAGGACAACCTGTAAAGAGTGGACCTAGAAAATGTCCACCCGCT | |
| | AGAGAGAGGGAGCGAGCATGTGCGATGAGCAATAGCTGTGGACCTTACAGTT | |
| | GCTGCTAACTGCCCTGGTGTGTGTGAGGGAGAGAGAGGGAGGGAGGGAGAGA | |
| | GAGCGCGCTAGCGCGAGAGAGCGAGTGAGCAAGCGAGCAGAAAAGAGGTGG | |
| | AGAGGGGGGGAATAAGAAAGAGAGAGAAGGAAAGGAGAGAAGGCAGGAAG | |
| | AAGGCAAGGGACGAGACAA | |
| SP0035 | CTAGCCCACAGGAAATGTCTGTCTATATCCAGGCAAGTACCTTGCTCATTGGA | 614 |
| (SEQ ID | CCAACCCGAAACTGTTCAGGGAAGATCAGGGAAATCAACTCAGTTACAAATG | |
| NO: 90) | GGATAATCATGCCCAGTAAAAACTACCTGTGGTGAATAAAGAGTTAACCCCTG | |
| | TTCCATCTTAGGTCACTATGCAGAGTACCAATGAGTACAAGAGATGGTGCCAA | |
| | AGAGGGTGGCCCCTCCCTAGCTGGGAACAGTCAACCCTTAGGAACTAGACTGT | |
| | CAACACATCAGCCAGCCAGAGACAAGGGAAACCGTGGCAACCAAGTGTTGCT | |
| | GGCACATTGTGAGGTGGTGATGGGAACTGCAGAGGCCCTGCACAGCATGCTA | |
| | ATGAGCCCAGGCAAACATCGCTAGAGAGAGGGAGCGAGCATGTGCGATGAGC | |
| | AATAGCTGTGGACCTTACAGTTGCTGCTAACTGCCCTGGTGTGTGTGAGGGAG | |
| | AGAGAGGGAGGGAGGGAGAGAGAGCGCGCTAGCGCGAGAGAGCGAGTGAGC | |
| | AAGCGAGCAGAAAAGAGGTGGAGAGGGGGGGAATAAGAAAGAGAGAGAAG | |
| | GAAAGGAGAGAAGGCAGGAAGAAGGCAAGGGACGAGACAA | |
| SP0036 | CTAGCCCACAGGAAATGTCTGTCTATATCCAGGCAAGTACCTTG | 695 |
| (SEQ | CTCATTGGACCAACCCGAAACTGTTCAGGGAAGATCAGGGAAAT | |
| ID NO: | CAACTCAGTTACAAATGGGATAATCATGCCCAGTAAAAACTACC | |
| 154) | TGTGGTGAATAAAGAGTTAACCCCTGTTCCATCTTAGGTCACTAT | |
| | GCAGAGTACCAATGAGTACAAGAGATGGTGCCAAAGAGGGTGG | |
| | CCCCTCCCTAGCTGGGAACAGTCAACCCTTAGGAACTAGACTGT | |
| | CAACACATCAGCCAGCCAGAGACAAGGGAAACCGTGGCAACCA | |
| | AGTGTTGCTGGCACATTGTGAGGTGGTGATGGGAACTGCAGAGG | |
| | CCCTGCACAGCATGCTAATGAGCCCAGGCAAACATTCGAGTTGG | |
| | CTGGACAAGGTTATGAGCATCCGTGTACTTATGGGGTTGCCAGC | |
| | TTGGTCCTGGATCGCCCGGGCCCTTCCCCCACCCGTTCGGTTCCC | |
| | CACCACCACCCGCGCTCGTACGTGCGTCTCCGCCTGCAGCTCTTG | |
| | ACTCATCGGGGCCCCCCGGGTCACATGCGCTCGCTCGGCTCTAT | |
| | AGGCGCCGCCCCCTGCCCACCCCCCGCCCGCGCTGGGAGCCGCA | |
| | GCCGCCGCCACTCCTGCTCTCTCTGCGCCGCCGCCGTCACCACCG | |
| | CCACCGCCACCGGCTGAGTCTGCAGTCCTCGAG | |
| CNS-1 | CTGGGCAGAGAGGGGGCATCGGGGGCATGGCTAGGGGCCAGCACTGT | 696 |
| (SEQ ID | GCTTCCTGGGCGCCTCACCTCCTCCCTGACTCCTGGAGACTCCCAGCCC | |
| NO: 91) | CTGTCTGGGAGATGAGCATTTAGGAATCTGCTTGTGCAGGGGTGGTGG | |
| | GAGGGGCCGGGGTGGAGGGCGCATCCCCACGGGGAGATTGGATGGAA | |
| | ATGGCCTGCCAGTGTGTGTGTGAGTGTGCGCCTGTGGCAGCAGCAGAG | |
| | TAAACAGCCGCTGCCCTGTCCTCTCTGCGGCCGTGGCCAGGTACACAG | |
| | GCCTGTTTGGACAGCTGCCTTGTCTGTCCGTCTGTTTGGGAGATGCTGG | |
| | CTGATAGATGGGGATGGGCGGACTGTTAACCCCTCGTTGCCTGCACTG | |
| | CTATGTGCTTCCTGCCTCATCCATGGGGTAGAAGGTAGCCAGAAGGTG | |
| | GTCCTGGCTGTGCCCCCAGCTCCTCTCTAGGGGGGAAACCTCTAGTTCT | |
| | GAGTCAGGGACAGAGTGAGGAGGGCTCCAGGGCATCAAGAGCTTGCT | |
| | CCTCCCCGCACCAGGGAGCCAAGGACAGAGGAGAAGGGGGTCTTCCC | |
| | CAGTGGTGACTAGGGGCAGAATATGTCTCTGAGTGAGTGTCTGGAGCC | |
| | CTCCTCACCCCAACACCATGGGGCTGGGCATAAAAGTCAGGGCAGAGC | |
| | CATCTATTGCTTACATTTGCTTCTG | |
| CNS-2 | GGTGTGTGGAAGGGTGAGAGGCACACACACAGACACTGAAAGAATCC | 709 |
| (SEQ ID | TAGGCCTGGTAGGCACTTAACAAATGTCTGTTACAGACCAGAATTTTA | |
| NO: 92) | TTGCTGTTAGAGACCCAAGCCCCTCATAGGAACAGTGAGAAACAGGTG | |
| | CAGAAAGGCGGAGTAACTTTATCTAAAGTCATAGGCTCCCTGAATAGC | |
| | AGAGCTGACACCTACAAGGAAGCGTTGGAGACCAGATCTACCAGCTA | |
| | GCCTCCCTGAGACCACGAGGTGGCGCCGCAGCACCGGCTGTGGCCGAT | |
| | GCCAGCCAGGTAGCCGGTTTCCCACGTCCCCCGCACGCACGCACCTCT | |
| | TTGCTGCAGGAATCCCGGGCTGCCCCGACCTGGAGTAGGGGGGGTGGT | |
| | GAGTGGGACTGAGTCCCTAGAAGCCTGGACCCTCACTTCGTTCCTGTA | |
| | CATCCAGCTCGCCTGTAGACAGTGGGGGAGGATGAAGGGAAGAGGAC | |
| | TCAAGCGCAACTTTGAATCATCACGCCTTCGACAGTCCGCGCACGTTTA | |
| | TTTCATTTATCTTTGAAAACGAGGGAGGGGAAGCCTGGAGAAGGCGGG | |
| | ATGGGCCAAGGGTGAGTTGGCCCCCGGGGAGCTGGTCCCTGTTCCTGG | |
| | CTTTAGTCCCAGGGGCGCGGTCTGTGTGTAGGGCGGGCTGGGCATAAA | |
| | AGTCAGGGCAGAGCCATCTATTGCTTACATTTGCTTCTG | |
| CNS-3 | TGCTACCAGAGCCGGGAGAGCTGCTCGGAGACGCCTCCGGGGTGCGG | |
| (SEQ ID | GCTGGACATGAGCAGCGGCTGCCGGTCCTGGGACTAGGCCCCGCCATT | |
| NO: 93) | TTGGATCCGCTGACAGGTCAGCGAAGTCTCTTCCTAGAGTTCCGGTGTC | |
| | GTGAAGGCCGCCCTGACATCGCAATAGGGAATTAGTGGGAAGGGCCCT | |
| | TAAATTGGGCGAGCCAAGGTGGGGGGAGGATTGGAACAGAGACAAAA | |
| | GGGAGGAGAGACGGACAGCGACAAGTGGAGAAAATCGGCGAAACTTG | |
| | AGTGGCAGAGAAGTCTGAGCGCTGAGACCCGGCGGCCCCGTGCGCCTT | |
| | CCCACCTGGCGCCGATCCACTTTCCTCGGGGTAGCGGCCCAACCCACT | |
| | TCGCTGCCAGCCGATCCCTTTTACCCGTGGCTACCGGGACCACTCTACT | |
| | CTCGCCCACTTGGCTCTGCCTAAGCGTCCTAGCCGGAGCGCGGTCTCTG | |
| | CCACGTGGGGAGGGGCGCGGCCGAGTTGCTGAAGAGCGCTTCTGATTG | |
| | GCCAGAGGGCGGGGTTCTTGGCGTCTCGCCGGCCAGACCCCTCCCTCA | |
| | AAGGCGGGGCCTGGAGATCCACAGCTGGAAAGGGCGGAGCCCCAGCA | |
| | GGGCAGCTGGAAAGGGGCGGGGCCTGACGCGCGCGGCTCGCCGCGGC | |
| | GGGCTGGGGGCGCCCTGGTCTGCCATAAAGTGAATGGGCGCCGGCTGG | |
| | GGGTGGCAGTACGCGGTGAGGCTCACTCCCTCCGAGAGTCCAGGAGCG | |
| | CC | |
| CNS-4 | AAGGAGAATGGTAAACAGCAGGAGCGAAGCGGCTGAGGAGAAAGAA | |
| (SEQ ID | GAGGAAAGAAAGGCGAGACGTGGGAGGATTGGAACAGAGACAAAAG | |
| NO: 94) | GGAGGAGAGACGGACAGCGACAAGTGGAGAAAATCGGCGAAACTTGA | |
| | GTGGCAGAGAAGTCTGAGCGCTGAGACCCGGCGGCCCCGTGCGCCTTC | |
| | CCACCTGGCGCCGATCCACTTTCCTCGGGGTAGCGGCCCAACCCACTT | |
| | CGCTGCCAGCCGATCCCTTTTACCCGTGGCTACCGGGACCACTCTACTC | |
| | TCGCCCACTTGGCTCTGCCTAAGCGTCCTAGCCGGAGCGCGGTCTCTGC | |
| | CACGTGGGGAGGGGCGCGGCCGAGTTGCTGAAGAGCGCTTCTGATTGG | |
| | CCAGAGGGCGGGGTTCTTGGCGTCTCGCCGGCCAGACCCCTCCCTCAA | |
| | AGGCGGGGCCTGGAGATCCACAGCTGGAAAGGGCGGAGCCCCAGCAG | |
| | GGCAGCTGGAAAGGGGCGGGGCCTGACGCGCGCGGCTCGCCGCGGCG | |
| | GGCTGGGGGCGCCCTGGTCTGCCATAAAGTGAATGGGCGCCGGCTGGG | |
| | GGTGGCAGTACGCGGTGAGGCTCACTCCCTCCGAGAGTCCAGGAGCGC | |
| | CCGAGCGGAGAGGCGGCCCGGGAGCAGGGGGGCGGCCCCCACTCCGG | |
| | CCGGGTGCCCGGCCCCTGGCCCCTGCCTGCCCTCTAGATCGCCGCCGC | |
| | AGCCGCCGCTACTGGGAGTCTG | |
| CNS-5 | ttgtAATGGGAATAAGGGCAGGACtcctgggtataagtagctcagctgatcccaccctgcttctatgt | 1744 |
| (SEQ ID | gttaattcatttattcattcattcaacaagcatttgttgaaatgctctttgtgtcaggctcagcaggaagcagtggcaa | |
| NO: 95) | taaaatggtgaacaagaaagactcggggtttcttcatctatgttgatgtctgcagagaacagtatcagccttctaggaa | |
| | gtttgtaatcagatacattgttagagagatacttatctagtaaattcctactcatcctataaggctcaaaacaaatgcc | |
| | tctatgaaaccttccgtgattccctcaggcagagttaagagcttcctttcctgggcctctatctccttccattagtatt | |
| | ataactgtttaccagtttcccctctagactaaatttctcaaaagagagaatgaggtctctttcagtcttctttgcatct | |
| | ttaaactagcctgggtcccagcctgtttgatgaaagaaacaagaacactgatacaagccacagccccttggcaaaaaag | |
| | atacccaatagcaatggcaatgtaaaatcagttttagtaaatgaatcaagaattctgatgctttagggaaagtaatgtg | |
| | aacctggcaccattaacaaattcagaactcttcttcttaggagctctctaactgaacagacagagggatgtcaacccct | |
| | aattcagcttgatcgtatctcagcaactacatttaatgagacagtgggaaaaagagagctgtccacttttaaatcagca | |
| | tatttctaactaaacaatggcaatggctaaatctttaaaatgcctatttctctcaagaacactgcaatggaacatttag | |
| | actttgggaaagagattagtgatttacattgctatctcactgatttaatttaaatgctcttccaaaccaaacacacatg | |
| | tgccgaagaggctactaagaaacccaacatgcagagttctctataagtgcagccgacagtgttgactgaaactaaactt | |
| | ggaaatccagggcactaatgcacaatatcaagcaataaaacggcatctctttggcaatatttaatttaaaaaagaagaa | |
| | agagacaggcgaagatcaggcactgtctgttttggaggatcaaccattctgcatttcaaagcattggtccctgcaatat | |
| | ccaggttactgtgctagaatctcgactattatatcgcagttgtgagagggagggcaaagatgtgtttactcagtgatta | |
| | ggcccttagaataagcctctagctcctagagagacagctcaccacttattcatttgggccaattcacaaagcctaggaa | |
| | gattaaacatccatgctgagaagacaagcgaatgcagacggtgaaaaagaaataaaaattctttaaaaactctgagatg | |
| | acttcattatttttccacaaggaaactttaggaaagtgtttagttagagaaaaacccacattgacctctctctaaaccc | |
| | ttaatctttcctttgtggtggcactgctttgtggtaagcgactggctcgcctcgcccctcttttcactggaagctgaga | |
| | gaaaaaagactctggagaaacagttttcgttccagggacacaaacccctgacactgttaaacatgagatgccaggaaaa | |
| | cacacttaaaaaaaaaattcccactttaagctttagactgaatgtgagaaaggagatgataaaaagagtatcacaaGAG | |
| | AATCTTCAGGCTGTGGG | |
| CNS-6 | TTTGGCACTGTGAGCAGTTTACttgacaaattctgtcaaatatttgctttctgaaatctcgagaattgg | 1104 |
| (SEQ ID | ttgaatataattgtacttaatgtttgcaaaataaataaatatgggactaaggacgttctatcattaatttgtcagaaaa | |
| NO: 96) | gagagttgtcatttctgaaaatttaatgtcattgaagctctatttccaatagcaaaggagcactattgctaatagactt | |
| | cagagcttgaaataaataaatctttggaatcctgttgcatctcttggggtgtgacatttgacagtcttttatagcacag | |
| | aacgaaacaagtttgtgagctggaattcaattgtggcgtattgattccttgcatcagtcattattccctgctgattgac | |
| | aggtgaaaattggttacgttaagtatttcatatgttatattggctgacatttgcttgcctgctcttgtgtcaatattgt | |
| | tgtaaagatctccagctttatgagatagcaatagacactgactgtggcttttgtgtgatgttccagtgtttttcctgac | |
| | ataatttaagacatattaaaaaccagcagcatcttccctcttgagaagcttaatgccaatattattgtcttccagggga | |
| | agatcatgtatgctcataatcgggtgctaatttccaccagtacgctcatgtttaggcattaggcactataactgtaaaa | |
| | ttgagccttcttgattgattcatgtcaagcctcatctcggctcctgcaggggaagtcatccggctgaccctttttacac | |
| | taaaagaagagatttgtgttcctttctttcacctggaaccatcaaattgactgaataatctgtaatacattagtgctga | |
| | catttgttagggagaattaaacaagacacagtaatcattccccagaataaaaattgtgtttgatttccagcagagttct | |
| | attaaagggaggacagaatctgtctcttccaaggtggaaaagatagggaggctctgatttaTGGTCAACACAGATTTGT | |
| | AACCC | |
| CNS-7 | TCAACATGGATAACCAAAGTTCTtaaaactacgctttcaatgaacacatatcctttgagcaagact | 1941 |
| (SEQ ID | aataatgaggaatgggagccagctcctgtgatatttatgcaactactaaattctcactgaagtcaatgggagtttgctt | |
| NO: 97) | acgtaagggctgcaaactttagcctccagagattaaaggggaaaaaaatccttaaactctttcaacattaatattgcct | |
| | gtaaggaatccagccatgacctaagccatggagctttctgaacctagcaagtagaagggtaaacagtaaacaccagtta | |
| | ttttaagcacaatctaatcagagttcaatgagaagcaatattatatttgatctctaaggtattaatacttgtatatcac | |
| | tattagacatctttatgtagtccattatccaaacaatggcttaagtctgtggtatttaataaatcaagtttccatggcc | |
| | gtgagactgagtgggagtggggatgaagccttttttcttcatttttttttcctcaggtgcaattctgtgttaatataag | |
| | agaagtgtggccttccttctcatagcactaaaagtgagataatccctgtgtaagaaatcagtaagtacggtctgcttaa | |
| | tctagtcccagtgtgaaactgttgacatttgttcttttttctatcattatgtgactgggcctgttttgtgctggattag | |
| | gcacaaatctcctatgcagcacatttggcatgttactagtagtttaacttcattaataatgtatgaagaaaatgtaatc | |
| | catgacaaggaagcaaagaaaagtatttttttttttttttgcttctcccaaatcctttggaatgagtaattattcaaca | |
| | ttttatgtttgatgttatattttacaattcaacttccatagtgatatttaaaaaagaaactttggcaaatgcttgcaaa | |
| | aaacacaccttttacaattttaaatgtgatttactgatggccagaacttgttaaacatagtaggaaattaaatatttat | |
| | tcatcttatttcattttcagggccgtaaacgctccttctgagtcattcccaataacaagaatttctaccagtaaagcta | |
| | ttaacaggcatcaaaataggggagtgctaaattaagatgagattgtaaaagcaaataagaacatacgcagactcgcata | |
| | ggagtgcaaatgatcgtttctgattgaaatgtttatagctaaatgagtttggctgaattaaacacaaatgttccaaaag | |
| | ataagccgtagctggtgcttcttttttctgttttttaagctgctttacagacgaaaatggaactatatttggaacaatg | |
| | ctttctgtttttccatactattgatatttgtggaaagtcacaaaatggcctaaggaagctaagctcgccccaagcagtg | |
| | gtcacttacaagtacttttgtactctgtactcctgtcacatttgggcgatcagagcaacagctggggagactttttcaa | |
| | caaagatgagtgtcagataatcctgatgagattccacatccaacatcttttgtaattatgtcacattcagctgtaatgg | |
| | aataattcaagctgaaagaacaagctttgatcctttcttaaacctttccctgtggactggctatctaaaagatttaaag | |
| | atatttctgttacaagatctagtgtttcctcagagaagtcatgcttctgaagcatcgtgatctacaagaacaatatcaa | |
| | gtttgccaaacacatttctgaaagcatcgtgttttggggggaggggttgtatttaatgaagatatcaataatatgctat | |
| | gcttcaattttcatctaggtgatcaagattcattttcttgttctgtcatccaaataggcagacagaaaagtgattgaaa | |
| | tacattaTGGAGATGTGTCATTGCACA | |
| CNS-8 | GCTGGTGCTTCTTTTTTCTGTTTTTTAAGCTGCTTTACAGACGAAAATG | 540 |
| (SEQ ID | GAACTATATTTGGAACAATGCTTTCTGTTTTTCCATACTATTGATATTTG | |
| NO: 98) | TGGAAAGTCACAAAATGGCCTAAGGAAGCTAAGCTCGCCCCAAGCAG | |
| | TGGTCACTTACAAGTACTTTTGTACTCTGTACTCCTGTCACATTTGGGC | |
| | GATCAGAGCAACAGCTGGGGAGACTTTTTCAACAAAGATGAGTGTCAG | |
| | ATAATCCTGATGAGATTCCACATCCAACATCTTTTGTAATTATGTCACA | |
| | TTCAGCTGTAATGGAATAATTCAAGCTGAAAGAACAAGCTTTGATCCT | |
| | TTCTTAAACCTTTCCCTGTGGACTGGCTATCTAAAAGATTTAAAGATAT | |
| | TTCTGTTACAAGATCTAGTGTTTCCTCAGAGAAGTCATGCTTCTGAAGC | |
| | ATCGTGATCTACAAGAACAATATCAAGTTTGCCAAACACATTTCTGAA | |
| | AGCATCGTGTTTTGGGGGGAGGGGTTGTATTTAATGAAGATATCAATA | |
| | ATATGC | |

TABLE 11
CNS-specific promoters

| NAME | SEQUENCE | Length |
|---|---|---|
| CNS-1 | CTGGGCAGAGAGGGGGCATCGGGGGCATGGCTAGGGGCCAGCACTGT | 696 |
| (SEQ | GCTTCCTGGGCGCCTCACCTCCTCCCTGACTCCTGGAGACTCCCAGCCC | |
| ID NO: | CTGTCTGGGAGATGAGCATTTAGGAATCTGCTTGTGCAGGGGTGGTGG | |
| 112) | GAGGGGCCGGGGTGGAGGGCGCATCCCCACGGGGAGATTGGATGGAA | |
| | ATGGCCTGCCAGTGTGTGTGTGAGTGTGCGCCTGTGGCAGCAGCAGAG | |
| | TAAACAGCCGCTGCCCTGTCCTCTCTGCGGCCGTGGCCAGGTACACAG | |
| | GCCTGTTTGGACAGCTGCCTTGTCTGTCCGTCTGTTTGGGAGATGCTGG | |
| | CTGATAGATGGGGATGGGCGGACTGTTAACCCCTCGTTGCCTGCACTG | |
| | CTATGTGCTTCCTGCCTCATCCATGGGGTAGAAGGTAGCCAGAAGGTG | |
| | GTCCTGGCTGTGCCCCCAGCTCCTCTCTAGGGGGGAAACCTCTAGTTCT | |
| | GAGTCAGGGACAGAGTGAGGAGGGCTCCAGGGCATCAAGAGCTTGCT | |
| | CCTCCCCGCACCAGGGAGCCAAGGACAGAGGAGAAGGGGGTCTTCCC | |
| | CAGTGGTGACTAGGGGCAGAATATGTCTCTGAGTGAGTGTCTGGAGCC | |
| | CTCCTCACCCCAACACCATGGGGCTGGGCATAAAAGTCAGGGCAGAGC | |
| | CATCTATTGCTTACATTTGCTTCTG | |
| CNS-2 | GGTGTGTGGAAGGGTGAGAGGCACACACACAGACACTGAAAGAATCC | 709 |
| (SEQ | TAGGCCTGGTAGGCACTTAACAAATGTCTGTTACAGACCAGAATTTTA | |
| ID NO: | TTGCTGTTAGAGACCCAAGCCCCTCATAGGAACAGTGAGAAACAGGTG | |
| 113) | CAGAAAGGCGGAGTAACTTTATCTAAAGTCATAGGCTCCCTGAATAGC | |
| | AGAGCTGACACCTACAAGGAAGCGTTGGAGACCAGATCTACCAGCTA | |
| | GCCTCCCTGAGACCACGAGGTGGCGCCGCAGCACCGGCTGTGGCCGAT | |
| | GCCAGCCAGGTAGCCGGTTTCCCACGTCCCCCGCACGCACGCACCTCT | |
| | TTGCTGCAGGAATCCCGGGCTGCCCCGACCTGGAGTAGGGGGGGTGGT | |
| | GAGTGGGACTGAGTCCCTAGAAGCCTGGACCCTCACTTCGTTCCTGTA | |
| | CATCCAGCTCGCCTGTAGACAGTGGGGGAGGATGAAGGGAAGAGGAC | |
| | TCAAGCGCAACTTTGAATCATCACGCCTTCGACAGTCCGCGCACGTTTA | |
| | TTTCATTTATCTTTGAAAACGAGGGAGGGGAAGCCTGGAGAAGGCGGG | |
| | ATGGGCCAAGGGTGAGTTGGCCCCCGGGGAGCTGGTCCCTGTTCCTGG | |
| | CTTTAGTCCCAGGGGCGCGGTCTGTGTGTAGGGCGGGCTGGGCATAAA | |
| | AGTCAGGGCAGAGCCATCTATTGCTTACATTTGCTTCTG | |
| CNS-3 | TGCTACCAGAGCCGGGAGAGCTGCTCGGAGACGCCTCCGGGGTGCGG | |
| (SEQ | GCTGGACATGAGCAGCGGCTGCCGGTCCTGGGACTAGGCCCCGCCATT | |
| ID NO: | TTGGATCCGCTGACAGGTCAGCGAAGTCTCTTCCTAGAGTTCCGGTGTC | |
| 114) | GTGAAGGCCGCCCTGACATCGCAATAGGGAATTAGTGGGAAGGGCCCT | |
| | TAAATTGGGCGAGCCAAGGTGGGGGGAGGATTGGAACAGAGACAAAA | |
| | GGGAGGAGAGACGGACAGCGACAAGTGGAGAAAATCGGCGAAACTTG | |
| | AGTGGCAGAGAAGTCTGAGCGCTGAGACCCGGCGGCCCCGTGCGCCTT | |
| | CCCACCTGGCGCCGATCCACTTTCCTCGGGGTAGCGGCCCAACCCACT | |
| | TCGCTGCCAGCCGATCCCTTTTACCCGTGGCTACCGGGACCACTCTACT | |
| | CTCGCCCACTTGGCTCTGCCTAAGCGTCCTAGCCGGAGCGCGGTCTCTG | |
| | CCACGTGGGGAGGGGCGCGGCCGAGTTGCTGAAGAGCGCTTCTGATTG | |
| | GCCAGAGGGCGGGGTTCTTGGCGTCTCGCCGGCCAGACCCCTCCCTCA | |
| | AAGGCGGGGCCTGGAGATCCACAGCTGGAAAGGGCGGAGCCCCAGCA | |
| | GGGCAGCTGGAAAGGGGCGGGGCCTGACGCGCGCGGCTCGCCGCGGC | |
| | GGGCTGGGGGCGCCCTGGTCTGCCATAAAGTGAATGGGCGCCGGCTGG | |
| | GGGTGGCAGTACGCGGTGAGGCTCACTCCCTCCGAGAGTCCAGGAGCG | |
| | CC | |
| CNS-4 | AAGGAGAATGGTAAACAGCAGGAGCGAAGCGGCTGAGGAGAAAGAA | |
| (SEQ | GAGGAAAGAAAGGCGAGACGTGGGAGGATTGGAACAGAGACAAAAG | |
| ID NO: | GGAGGAGAGACGGACAGCGACAAGTGGAGAAAATCGGCGAAACTTGA | |
| 115) | GTGGCAGAGAAGTCTGAGCGCTGAGACCCGGCGGCCCCGTGCGCCTTC | |
| | CCACCTGGCGCCGATCCACTTTCCTCGGGGTAGCGGCCCAACCCACTT | |
| | CGCTGCCAGCCGATCCCTTTTACCCGTGGCTACCGGGACCACTCTACTC | |
| | TCGCCCACTTGGCTCTGCCTAAGCGTCCTAGCCGGAGCGCGGTCTCTGC | |
| | CACGTGGGGAGGGGCGCGGCCGAGTTGCTGAAGAGCGCTTCTGATTGG | |
| | CCAGAGGGCGGGGTTCTTGGCGTCTCGCCGGCCAGACCCCTCCCTCAA | |
| | AGGCGGGGCCTGGAGATCCACAGCTGGAAAGGGCGGAGCCCCAGCAG | |
| | GGCAGCTGGAAAGGGGCGGGGCCTGACGCGCGCGGCTCGCCGCGGCG | |
| | GGCTGGGGGCGCCCTGGTCTGCCATAAAGTGAATGGGCGCCGGCTGGG | |
| | GGTGGCAGTACGCGGTGAGGCTCACTCCCTCCGAGAGTCCAGGAGCGC | |
| | CCGAGCGGAGAGGCGGCCCGGGAGCAGGGGGGCGGCCCCCACTCCGG | |
| | CCGGGTGCCCGGCCCCTGGCCCCTGCCTGCCCTCTAGATCGCCGCCGC | |
| | AGCCGCCGCTACTGGGAGTCTG | |
| CNS-5_ | ttgtAATGGGAATAAGGGCAGGACtcctgggtataagtagctcagctgatcccaccctgcttctatgt | 1744 |
| v2 | gttaattcatttattcattcattcaacaagcatttgttgaaatgctctttgtgtcaggctcagcaggaagcagtggcaataaa | |
| (SEQ | atggtgaacaagaaagactcggggtttcttcatctatgttgatgtctgcagagaacagtatcagccttctaggaagtttgt | |
| ID NO: | aatcagatacattgttagagagatacttatctagtaaattcctactcatcctataaggctcaaaacaaatgcctctatgaaa | |
| 116) | ccttccgtgattccctcaggcagagttaagagcttcctttcctgggcctctatctccttccattagtattataactgtttacc | |
| | agtttcccctctagactaaatttctcaaaagagagaatgaggtctctttcagtcttctttgcatctttaaactagcctgggtc | |
| | ccagcctgtttgatgaaagaaacaagaacactgatacaagccacagccccttggcaaaaaagatacccaatagcaatg | |
| | gcaatgtaaaatcagttttagtaaatgaatcaagaattctgatgctttagggaaagtaatgtgaacctggcaccattaaca | |
| | aattcagaactcttcttcttaggagctctctaactgaacagacagagggatgtcaacccctaattcagcttgatcgtatct | |
| | cagcaactacatttaatgagacagtgggaaaaagagagctgtccacttttaaatcagcatatttctaactaaacaatggc | |
| | aatggctaaatctttaaaatgcctatttctctcaagaacactgcaatggaacatttagactttgggaaagagattagtgatt | |
| | tacattgctatctcactgatttaatttaaatgctcttccaaaccaaacacacatgtgccgaagaggctactaagaaaccca | |
| | acatgcagagttctctataagtgcagccgacagtgttgactgaaactaaacttggaaatccagggcactaatgcacaat | |
| | atcaagcaataaaacggcatctctttggcaatatttaatttaaaaaagaagaaagagacaggcgaagatcaggcactgt | |
| | ctgttttggaggatcaaccattctgcatttcaaagcattggtccctgcaatatccaggttactgtgctagaatctcgactatt | |
| | atatcgcagttgtgagagggagggcaaagatgtgtttactcagtgattaggcccttagaataagcctctagctcctaga | |
| | gagacagctcaccacttattcatttgggccaattcacaaagcctaggaagattaaacatccatgctgagaagacaagc | |
| | gaatgcagacggtgaaaaagaaataaaaattctttaaaaactctgagatgacttcattatttttccacaaggaaactttag | |
| | gaaagtgtttagttagagaaaaacccacattgacctctctctaaacccttaatctttcctttgtggtggcactgctttgtggt | |
| | aagcgactggctcgcctcgcccctcttttcactggaagctgagagaaaaaagactctggagaaacagttttcgttccag | |
| | ggacacaaacccctgacactgttaaacatgagatgccaggaaaacacacttaaaaaaaaaattcccactttaagcttta | |
| | gactgaatgtgagaaaggagatgataaaaagagtatcacaaGAGAATCTTCAGGCTGTGGG | |
| CNS-6_ | TTTGGCACTGTGAGCAGTTTACttgacaaattctgtcaaatatttgctttctgaaatctcgagaattgg | 1104 |
| v2 | ttgaatataattgtacttaatgtttgcaaaataaataaatatgggactaaggacgttctatcattaatttgtcagaaaagaga | |
| (SEQ | gttgtcatttctgaaaatttaatgtcattgaagctctatttccaatagcaaaggagcactattgctaatagacttcagagctt | |
| ID NO: | gaaataaataaatctttggaatcctgttgcatctcttggggtgtgacatttgacagtcttttatagcacagaacgaaacaa | |
| 117) | gtttgtgagctggaattcaattgtggcgtattgattccttgcatcagtcattattccctgctgattgacaggtgaaaattggt | |
| | tacgttaagtatttcatatgttatattggctgacatttgcttgcctgctcttgtgtcaatattgttgtaaagatctccagctt | |
| | tatgagatagcaatagacactgactgtggcttttgtgtgatgttccagtgtttttcctgacataatttaagacatattaaaaa | |
| | ccagcagcatcttccctcttgagaagcttaatgccaatattattgtcttccaggggaagatcatgtatgctcataatcgggtg | |
| | ctaatttccaccagtacgctcatgtttaggcattaggcactataactgtaaaattgagccttcttgattgattcatgtcaagc | |
| | ctcatctcggctcctgcaggggaagtcatccggctgaccctttttacactaaaagaagagatttgtgttcctttctttcacct | |
| | ggaaccatcaaattgactgaataatctgtaatacattagtgctgacatttgttagggagaattaaacaagacacagtaatcat | |
| | tccccagaataaaaattgtgtttgatttccagcagagttctattaaagggaggacagaatctgtctcttccaaggtggaaa | |
| | atcgtgaatattccctgcattaatgaaccaagttaacactttaattgcttatagaaccgagttctccaatgacagcattaaa | |
| | agatagggaggctctgatttaTGGTCAACACAGATTTGTAACCC | |
| CNS-7_ | TCAACATGGATAACCAAAGTTCTtaaaactacgctttcaatgaacacatatcctttgagcaagact | 1941 |
| v2 | aataatgaggaatgggagccagctcctgtgatatttatgcaactactaaattctcactgaagtcaatgggagtttgcttac | |
| (SEQ | gtaagggctgcaaactttagcctccagagattaaaggggaaaaaaatccttaaactctttcaacattaatattgcctgtaa | |
| ID NO: | ggaatccagccatgacctaagccatggagctttctgaacctagcaagtagaagggtaaacagtaaacaccagttatttt | |
| 118) | aagcacaatctaatcagagttcaatgagaagcaatattatatttgatctctaaggtattaatacttgtatatcactattagac | |
| | atctttatgtagtccattatccaaacaatggcttaagtctgtggtatttaataaatcaagtttccatggccgtgagactgagt | |
| | gggagtggggatgaagccttttttcttcatttttttttcctcaggtgcaattctgtgttaatataagagaagtgtggccttcc | |
| | ttctcatagcactaaaagtgagataatccctgtgtaagaaatcagtaagtacggtctgcttaatctagtcccagtgtgaaac | |
| | tgttgacatttgttcttttttctatcattatgtgactgggcctgttttgtgctggattaggcacaaatctcctatgcagcaca | |
| | tttggcatgttactagtagtttaacttcattaataatgtatgaagaaaatgtaatccatgacaaggaagcaaagaaaagtatt | |
| | tttttttttttttgcttctcccaaatcctttggaatgagtaattattcaacattttatgtttgatgttatattttacaattca | |
| | acttccatagtgatatttaaaaaagaaactttggcaaatgcttgcaaaaaacacaccttttacaattttaaatgtgatttact | |
| | gatggccagaacttgttaaacatagtaggaaattaaatatttattcatcttatttcattttcagggccgtaaacgctccttct | |
| | gagtcattcccaataacaagaatttctaccagtaaagctattaacaggcatcaaaataggggagtgctaaattaagatgagat | |
| | tgtaaaagcaaataagaacatacgcagactcgcataggagtgcaaatgatcgtttctgattgaaatgtttatagctaaatgag | |
| | tttggctgaattaaacacaaatgttccaaaagataagccgtagctggtgcttcttttttctgttttttaagctgctttacaga | |
| | cgaaaatggaactatatttggaacaatgctttctgtttttccatactattgatatttgtggaaagtcacaaaatggcctaagg | |
| | aagctaagctcgccccaagcagtggtcacttacaagtacttttgtactctgtactcctgtcacatttgggcgatcagagcaac | |
| | agctggggagactttttcaacaaagatgagtgtcagataatcctgatgagattccacatccaacatcttttgtaattatgtca | |
| | cattcagctgtaatggaataattcaagctgaaagaacaagctttgatcctttcttaaacctttccctgtggactggctatcta | |
| | aaagatttaaagatatttctgttacaagatctagtgtttcctcagagaagtcatgcttctgaagcatcgtgatctacaagaa | |
| | caatatcaagtttgccaaacacatttctgaaagcatcgtgttttggggggaggggttgtatttaatgaagatatcaataata | |
| | tgctatgcttcaattttcatctaggtgatcaagattcattttcttgttctgtcatccaaataggcagacagaaaagtgattga | |
| | aatacattaTGGAGATGTGTCATTGCACA | |
| CNS-8_ | GCTGGTGCTTCTTTTTTCTGTTTTTTAAGCTGCTTTACAGACGAAAATG | 540 |
| v2 | GAACTATATTTGGAACAATGCTTTCTGTTTTTCCATACTATTGATATTTG | |
| (SEQ | TGGAAAGTCACAAAATGGCCTAAGGAAGCTAAGCTCGCCCCAAGCAG | |
| ID NO: | TGGTCACTTACAAGTACTTTTGTACTCTGTACTCCTGTCACATTTGGGC | |
| 119) | GATCAGAGCAACAGCTGGGGAGACTTTTTCAACAAAGATGAGTGTCAG | |
| | ATAATCCTGATGAGATTCCACATCCAACATCTTTTGTAATTATGTCACA | |
| | TTCAGCTGTAATGGAATAATTCAAGCTGAAAGAACAAGCTTTGATCCT | |
| | TTCTTAAACCTTTCCCTGTGGACTGGCTATCTAAAAGATTTAAAGATAT | |
| | TTCTGTTACAAGATCTAGTGTTTCCTCAGAGAAGTCATGCTTCTGAAGC | |
| | ATCGTGATCTACAAGAACAATATCAAGTTTGCCAAACACATTTCTGAA | |
| | AGCATCGTGTTTTGGGGGGAGGGGTTGTATTTAATGAAGATATCAATA | |
| | ATATGC | |
| CNS-1 + | CTGGGCAGAGAGGGGGCATCGGGGGCATGGCTAGGGGCCAGCACTGT | 982 |
| CMV- | GCTTCCTGGGCGCCTCACCTCCTCCCTGACTCCTGGAGACTCCCAGCCC | |
| IE UTR | CTGTCTGGGAGATGAGCATTTAGGAATCTGCTTGTGCAGGGGTGGTGG | |
| and | GAGGGGCCGGGGTGGAGGGCGCATCCCCACGGGGAGATTGGATGGAA | |
| intron | ATGGCCTGCCAGTGTGTGTGTGAGTGTGCGCCTGTGGCAGCAGCAGAG | |
| (SEQ | TAAACAGCCGCTGCCCTGTCCTCTCTGCGGCCGTGGCCAGGTACACAG | |
| ID NO: | GCCTGTTTGGACAGCTGCCTTGTCTGTCCGTCTGTTTGGGAGATGCTGG | |
| 120) | CTGATAGATGGGGATGGGCGGACTGTTAACCCCTCGTTGCCTGCACTG | |
| | CTATGTGCTTCCTGCCTCATCCATGGGGTAGAAGGTAGCCAGAAGGTG | |
| | GTCCTGGCTGTGCCCCCAGCTCCTCTCTAGGGGGGAAACCTCTAGTTCT | |
| | GAGTCAGGGACAGAGTGAGGAGGGCTCCAGGGCATCAAGAGCTTGCT | |
| | CCTCCCCGCACCAGGGAGCCAAGGACAGAGGAGAAGGGGGTCTTCCC | |
| | CAGTGGTGACTAGGGGCAGAATATGTCTCTGAGTGAGTGTCTGGAGCC | |
| | CTCCTCACCCCAACACCATGGGGCTGGGCATAAAAGTCAGGGCAGAGC | |
| | CATCTATTGCTTACATTTGTCAGATCGCCTGGAGACGCCATCCACGCTG | |
| | TTTTGACCTCCATAGAAGACACCGGGACCGATCCAGCCTCCGCGGCCG | |
| | GGAACGGTGCATTGGAACGCGGATTCCCCGTGCCAAGAGTGACGTAAG | |
| | TACCGCCTATAGACTCTATAGGCACACCCCTTTGGCTCTTATGCATGAA | |
| | CGGTGGAGGGCAGTGTAGTCTGAGCAGTACTCGTTGCTGCCGCGCGCG | |
| | CCACCAGACATAATAGCTGACAGACTAACAGACTGTTCCTTTCCATGG | |
| | GTCTTTTCTGCAGATGCCACC | |
| CNS-4 + | AAGGAGAATGGTAAACAGCAGGAGCGAAGCGGCTGAGGAGAAAGAA | 876 |
| CMV- | GAGGAAAGAAAGGCGAGACGTGGGAGGATTGGAACAGAGACAAAAG | |
| IE UTR | GGAGGAGAGACGGACAGCGACAAGTGGAGAAAATCGGCGAAACTTGA | |
| and | GTGGCAGAGAAGTCTGAGCGCTGAGACCCGGCGGCCCCGTGCGCCTTC | |
| intron | CCACCTGGCGCCGATCCACTTTCCTCGGGGTAGCGGCCCAACCCACTT | |
| (SEQ | CGCTGCCAGCCGATCCCTTTTACCCGTGGCTACCGGGACCACTCTACTC | |
| ID NO: | TCGCCCACTTGGCTCTGCCTAAGCGTCCTAGCCGGAGCGCGGTCTCTGC | |
| 121) | CACGTGGGGAGGGGCGCGGCCGAGTTGCTGAAGAGCGCTTCTGATTGG | |
| | CCAGAGGGCGGGGTTCTTGGCGTCTCGCCGGCCAGACCCCTCCCTCAA | |
| | AGGCGGGGCCTGGAGATCCACAGCTGGAAAGGGCGGAGCCCCAGCAG | |
| | GGCAGCTGGAAAGGGGCGGGGCCTGACGCGCGCGGCTCGCCGCGGCG | |
| | GGCTGGGGGCGCCCTGGTCTGCCATAAAGTGAATGGGCGCCGGCTGGG | |
| | GGTGGCAGTACGCTCAGATCGCCTGGAGACGCCATCCACGCTGTTTTG | |
| | ACCTCCATAGAAGACACCGGGACCGATCCAGCCTCCGCGGCCGGGAA | |
| | CGGTGCATTGGAACGCGGATTCCCCGTGCCAAGAGTGACGTAAGTACC | |
| | GCCTATAGACTCTATAGGCACACCCCTTTGGCTCTTATGCATGAACGGT | |
| | GGAGGGCAGTGTAGTCTGAGCAGTACTCGTTGCTGCCGCGCGCGCCAC | |
| | CAGACATAATAGCTGACAGACTAACAGACTGTTCCTTTCCATGGGTCTT | |
| | TTCTGCAGATGCCACC | |
| CNS-5 | GGAACATTTAGACTTTGGGAAAGAGATTAGTGATTTACATTGCTATCTC | 835 |
| (SEQ | ACTGATTTAATTTAAATGCTCTTCCAAACCAAACACACATGTGCCGAA | |
| ID NO: | GAGGCTACTAAGAAACCCAACATGCAGAGTTCTCTATAAGTGCAGCCG | |
| 122) | ACAGTGTTGACTGAAACTAAACTTGGAAATCCAGGGCACTAATGCACA | |
| | ATATCAAGCAATAAAACGGCATCTCTTTGGCAATATTTAATTTAAAAA | |
| | AGAAGAAAGAGACAGGCGAAGATCAGGCACTGTCTGTTTTGGAGGAT | |
| | CAACCATTCTGCATTTCAAAGCATTGGTCCCTGCAATATCCAGGTTACT | |
| | GTGCTAGAATCTCGACTATTATATCGCAGTIGTGAGAGGGAGGGCAAA | |
| | GATGTGTTTACTCAGTGATTAGGCCCTTAGAATAAGCCTCTAGCTCCTA | |
| | GAGAGACAGCTCACCACTTATTCATTTGGGCCAATTCACAAAGCCTAG | |
| | GAAGATTAAACATCCATGCTGAGAAGACAAGCGAATGCAGACGGTGA | |
| | AAAAGAAATAAAAATTCTTTAAAAACTCTGAGATGACTTCATTATTTTT | |
| | CCACAAGGAAACTTTAGGAAAGTGTTTAGTTAGAGAAAAACCCACATT | |
| | GACCTCTCTCTAAACCCTTAATCTTTCCTTTGTGGTGGCACTGCTTTGTG | |
| | GTAAGCGACTGGCTCGCCTCGCCCCTCTTTTCACTGGAAGCTGAGAGA | |
| | AAAAAGACTCTGGAGAAACAGTTTTCGTTCCAGGGACACAAACCCCTG | |
| | ACACTGTTAAGGGCTGGGCATAAAAGTCAGGGCAGAGCCATCTATTGC | |
| | TTACATTTGCTTCTG | |
| CNS-6 | GAAAATTTAATGTCATTGAAGCTCTATTTCCAATAGCAAAGGAGCACT | 812 |
| (SEQ | ATTGCTAATAGACTTCAGAGCTTGAAATAAATAAATCTTTGGAATCCT | |
| ID NO: | GTTGCATCTCTTGGGGTGTGACATTTGACAGTCTTTTATAGCACAGAAC | |
| 123) | GAAACAAGTTTGTGAGCTGGAATTCAATTGTGGCGTATTGATTCCTTGC | |
| | ATCAGTCATTATTCCCTGCTGATTGACAGGTGAAAATTGGTTACGTTAA | |
| | GTATTTCATATGTTATATTGGCTGACATTTGCTTGCCTGCTCTTGTGTCA | |
| | ATATTGTTGTAAAGATCTCCAGCTTTATGAGATAGCAATAGACACTGA | |
| | CTGTGGCTTTTGTGTGATGTTCCAGTGTTTTTCCTGACATAATTTAAGA | |
| | CATATTAAAAACCAGCAGCATCTTCCCTCTTGAGAAGCTTAATGCCAA | |
| | TATTATTGTCTTCCAGGGGAAGATCATGTATGCTCATAATCGGGTGCTA | |
| | ATTTCCACCAGTACGCTCATGTTTAGGCATTAGGCACTATAACTGTAAA | |
| | ATTGAGCCTTCTTGATTGATTCATGTCAAGCCTCATCTCGGCTCCTGCA | |
| | GGGGAAGTCATCCGGCTGACCCTTTTTACACTAAAAGAAGAGATTTGT | |
| | GTTCCTTTCTTTCACCTGGAACCATCAAATTGACTGAATAATCTGTAAT | |
| | ACATTAGTGCTGACATTTGTTAGGGAGAATTAAACAAGACACAGTAAT | |
| | CATTCCCCAGAATAAAAATTGTGTTTGATGGGCTGGGCATAAAAGTCA | |
| | GGGCAGAGCCATCTATTGCTTACATTTGCTTCTG | |
| CNS-7 | TGAGACTGAGTGGGAGTGGGGATGAAGCCTTTTTTCTTCATTTTTTTTT | 487 |
| (SEQ | CCTCAGGTGCAATTCTGTGTTAATATAAGAGAAGTGTGGCCTTCCTTCT | |
| ID NO: | CATAGCACTAAAAGTGAGATAATCCCTGTGTAAGAAATCAGTAAGTAC | |
| 124) | GGTCTGCTTAATCTAGTCCCAGTGTGAAACTGTTGACATTTGTTCTTTTT | |
| | TCTATCATTATGTGACTGGGCCTGTTTTGTGCTGGATTAGGCACAAATC | |
| | TCCTATGCAGCACATTTGGCATGTTACTAGTAGTTTAACTTCATTAATA | |
| | ATGTATGAAGAAAATGTAATCCATGACAAGGAAGCAAAGAAAAGTAT | |
| | TTTTTTTTTTTTTTGCTTCTCCCAAATCCTTTGGAATGAGTAATTATTCA | |
| | ACATTTTATGTTTGATGTTATATTTTACAATTCAACTTCCATAGGGCTG | |
| | GGCATAAAAGTCAGGGCAGAGCCATCTATTGCTTACATTTGCTTCTG | |
| CNS-8 | GCTGGTGCTTCTTTTTTCTGTTTTTTAAGCTGCTTTACAGACGAAAATG | 593 |
| (SEQ | GAACTATATTTGGAACAATGCTTTCTGTTTTTCCATACTATTGATATTTG | |
| ID NO: | TGGAAAGTCACAAAATGGCCTAAGGAAGCTAAGCTCGCCCCAAGCAG | |
| 125) | TGGTCACTTACAAGTACTTTTGTACTCTGTACTCCTGTCACATTTGGGC | |
| | GATCAGAGCAACAGCTGGGGAGACTTTTTCAACAAAGATGAGTGTCAG | |
| | ATAATCCTGATGAGATTCCACATCCAACATCTTTTGTAATTATGTCACA | |
| | TTCAGCTGTAATGGAATAATTCAAGCTGAAAGAACAAGCTTTGATCCT | |
| | TTCTTAAACCTTTCCCTGTGGACTGGCTATCTAAAAGATTTAAAGATAT | |
| | TTCTGTTACAAGATCTAGTGTTTCCTCAGAGAAGTCATGCTTCTGAAGC | |
| | ATCGTGATCTACAAGAACAATATCAAGTTTGCCAAACACATTTCTGAA | |
| | AGCATCGTGTTTTGGGGGGAGGGGTTGTATTTAATGAAGATATCAATA | |
| | ATATGCGGGCTGGGCATAAAAGTCAGGGCAGAGCCATCTATTGCTTAC | |
| | ATTTGCTTCTG | |

TABLE 12
Minimal/Proximal Promoters comprised in the promoters of Table 11

| Name | SEQUENCE |
|---|---|
| SYNP_CRE151 | GGGCTGGGCATAAAAGTCAGGGCAGAGCCATCTATTGCTTACATTTGCTTCTG |
| (SEQ ID NO: | |
| 126) | |
| CRE0001v1_ | GGGAGGATTGGAACAGAGACAAAAGGGAGGAGAGACGGACAGCGACAAGTGG |
| Pitx3 | AGAAAATCGGCGAAACTTGAGTGGCAGAGAAGTCTGAGCGCTGAGACCCGGCG |
| (SEQ ID NO: | GCCCCGTGCGCCTTCCCACCTGGCGCCGATCCACTTTCCTCGGGGTAGCGGCCC |
| 127) | AACCCACTTCGCTGCCAGCCGATCCCTTTTACCCGTGGCTACCGGGACCACTCTA |
| | CTCTCGCCCACTTGGCTCTGCCTAAGCGTCCTAGCCGGAGCGCGGTCTCTGCCAC |
| | GTGGGGAGGGGCGCGGCCGAGTTGCTGAAGAGCGCTTCTGATTGGCCAGAGGG |
| | CGGGGTTCTTGGCGTCTCGCCGGCCAGACCCCTCCCTCAAAGGCGGGGCCTGGA |
| | GATCCACAGCTGGAAAGGGCGGAGCCCCAGCAGGGCAGCTGGAAAGGGGCGG |
| | GGCCTGACGCGCGCGGCTCGCCGCGGCGGGCTGGGGGCGCCCTGGTCTGCCATA |
| | AAGTGAATGGGCGCCGGCTGGGGGTGGCAGTACGCGGTGAGGCTCACTCCCTCC |
| | GAGAGTCCAGGAGCGCC |

TABLE 13
Synthetic CNS-specific promoter overview

| | Minimal/proximal | | |
|---|---|---|---|
| Promoter name | promoter | CRE | UTR |
| CNS-1 | SYNP_CRE151 | CRE0004_Lmx1b | |
| CNS-2 | SYNP_CRE151 | CRE0003_Pitx3 | |
| CNS-3 | CRE0001v1_Pitx3 | CRE0002_Gbf1 | |
| CNS-4 | CRE0001_Pitx3 | | |
| CNS-5_v2 | CRE0005_faf1 | | |
| CNS-6_v2 | CRE0006_Pitx2 | | |
| CNS-7_v2 | CRE0007_Pitx2 | | |
| CNS-8_v2 | CRE0008_Pitx2 | | |
| CNS-1 + CMV-IE | SYNP_CRE151 | CRE0004_Lmx1b | CMV-IE UTR and |
| UTR and intron | | | intron |
| CNS-4 + CMV-IE | CRE0001_Pitx3 | | CMV-IE UTR and |
| UTR and intron | | | intron |
| CNS-5 | SYNP_CRE151 | CRE0005_faf1_short | |
| CNS-6 | SYNP_CRE151 | CRE0006_Pitx2_short | |
| CNS-7 | SYNP_CRE151 | CRE0007_Pitx2_short | |
| CNS-8 | SYNP_CRE151 | CRE0008_Pitx2_short | |

TABLE 14
Exemplary CREs

| | |
|---|---|
| Name | SEQUENCE |

| | |
|---|---|
| CRE0006_ | AACATATCCTGGTGTGGAGTAGGGGACGCTGCTCTGACAGAGGCTCGGGGGC |
| GFAP | CTGAGCTGGCTCTGTGAGCTGGGGAGGAGGCAGACAGCCAGGCCTTGTCTGCA |
| (SEQ ID | AGCAGACCTGGCAGCATTGGGCTGGCCGCCCCCCAGGGCCTCCTCTTCATGCC |
| NO: 99) | CAGTGAATGACTCACCTTGGCACAGACACAATGTTCGGGGTGGGCACAGTGCC |
| | TGCTTCCCGCCGCACCCCAGCCCCCCTCAAATGCCTTCCGAGAAGCCCATTGA |
| | GCAGGGGGCTTGCATTGCACCCCAGCCTGACAGCCTGGCATCTTGGGATAAAA |
| | GCAGCACAGCCCCCTAGGGGCTGCCCTTGCTGTGTGGCGCCA |
| CRE0008_ | AACATATCCTGGTGTGGAGTAGGGGACGCTGCTCTGACAGAGGCTCGGGGGC |
| GFAP | CTGAGCTGGCTCTGTGAGCTGGGGAGGAGGCAGACAGCCAGGCCTTGTCTGCA |
| (SEQ ID | AGCAGACCTGGCAGCATTGGGCTGGCCGCCCCCCAGGGCCTCCTCTTCATGCC |
| NO: 100) | CAGTGAATGACTCACCTTGGCACAGACACAATGTTCGGGGTGGGCACAGTGCC |
| | TGCTTCCCGCCGCACCCCAGCCCCCCTCAAATGCCTTCCGAGAAGCCCATTGA |
| | GCAGGGGGCTTGCATTGCACCCCAGCCTGACAGCCTGGCATCTTGGGATAAAA |
| | GCACCGGCGGTGGAGAACAAGGCTCTATTCAGCCTGTGCCCAGGAAAGGGGA |
| | TCAGGGGATGCCCAGGCATGGACAGTGGGTGGCAGGGGGGGAGAGGAGGGCT |
| | GTCTGCTTCCCAGAAGTCCAAGGACACAAATGGGTGAGGGGA |
| CRE0006_ | CTAGCCCACAGGAAATGTCTGTCTATATCCAGGCAAGTACCTTGCTCATTGGA |
| AQP4 | CCAACCCGAAACTGTTCAGGGAAGATCAGGGAAATCAACTCAGTTACAAATG |
| (SEQ ID | GGATAATCATGCCCAGTAAAAACTACCTGTGGTGAATAAAGAGTTAACCCCTG |
| NO: 101) | TTCCATCTTAGGTCACTATGCAGAGTACCAATGAGTACAAGAGATGGTGCCAA |
| | AGAGGGTGGCCCCTCCCTAGCTGGGAACAGTCAACCCTTAGGAACTAGACTGT |
| | CAACACATCAGCCAGCCAGAGACAAGGGAAACCGTGGCAACCAAGTGTTGCT |
| | GGCACATTGTGAGGTGGTGATGGGAACTGCAGAGGCCCTGCACAGCATGCTA |
| | ATGAGCCCAGGCAAACAT |
| CRE0008_ | TCCAAAGAAAAGCCAGATAAGTAGCTGATTATTGCATAGAGCTGACAGTATCA |
| AQP4 | CAGGAAGATCAGTAGTAGCAGCTCAAGTACAAAAAGGTTAATTAGCAATACT |
| (SEQ ID | TAATAAGAAAAACTACCTCTGGCAGGTGAAGAGTTAATCCCTGGTCAATTTTA |
| NO: 102) | AGCTACTCTGCTGAGAGTACTAATAAGTGTAGGGGTTGGAGCCAATGAGGGTG |
| | ACCCCTTCCTTGATGGGAACAGTCATCCCTTAGGAACTGCCCTGGAAAGCATC |
| | AGCCAGCCAGAAAACAGGGAAAGAGGCTGAGAAACCGTGGTAACCAAGTTTT |
| | GCTGGCACTTTGTAAAATGGTAACTGCAACTGCCGAGGCTGTGCAGAGAATGC |
| | TAATAAGCCTAGGACAACCTGTAAAGAGTGGACCTAGAAAATGTCCACC |
| CRE0005_ | AACATATCCTGGTGTGGAGTAGGGGACGCTGCTCTGACAGAGGCTCGGGGGC |
| GFAP | CTGAGCTGGCTCTGTGAGCTGGGGAGGAGGCAGACAGCCAGGCCTTGTCTGCA |
| (SEQ ID | AGCAGACCTGGCAGCATTGGGCTGGCCGCCCCCCAGGGCCTCCTCTTCATGCC |
| NO: 103) | CAGTGAATGACTCACCTTGGCACAGACACAATGTTCGGGGTGGGCACAGTGCC |
| | TGCTTCCCGCCGCACCCCAGCCCCCCTCAAATGCCTTCCGAGAAGCCCATTGA |
| | GCAGGGGGCTTGCATTGCACCCCAGCCTGACAGCCTGGCATCTTGGGATAAAA |
| | GCAGCACAGCCCCCTAGGGGCTGCCCTTGCTGTGTGGCGCCACCGGCGGTGGA |
| | GAACAAGGCTCTATTCAGCCTGTGCCCAGGAAAGGGGATCAGGGGATGCCCA |
| | GGCATGGACAGTGGGTGGCAGGGGGGGAGAGGAGGGCTGTCTGCTTCCCAGA |
| | AGTCCAAGGACACAAATGGGTGAGGGGA |
| CRE0007_ | AACATATCCTGGTGTGGAGTAGGGGACGCTGCTCTGACAGAGGCTCGGGGGC |
| GFAP | CTGAGCTGGCTCTGTGAGCTGGGGAGGAGGCAGACAGCCAGGCCTTGTCTGCA |
| (SEQ ID | AGCAGACCTGGCAGCATTGGGCTGGCCGCCCCCCAGGGCCTCCTCTTCATGCC |
| NO: 104) | CAGTGAATGACTCACCTTGGCACAGACACAATGTTCGGGGTGGGCACAGTGCC |
| | TGCTTCCCGCCGCACCCCAGCCCCCCTCAAATGCCTTCCGAGAAGCCCATTGA |
| | GCAGGGGGCTTGCATTGCACCCCAGCCTGACAGCCTGGCATCTTGGGATAAAA |
| | GCAGCAC |
| CRE0012_ | AGCGCGCAGAGTCTGCATGCGTGAGGAAGCTCCTGGGCGCGTCACAGCCGCG |
| Arc | CTATTCTCAGCGTCTCTCCTTTTATGGCTCCGGAAGTGAGCTGGGGTTGCTGGC |
| (SEQ ID | AGCCTGGCTGGCACT |
| NO: 105) | |
| CRE0001_ | CTTCCTCTTATATTTCACCAAGACTCAGTTCCTGAGCAAGAAACCACAGGCAC |
| S100B | AGCAAGTGCCATGAAAAGCGGCTTTGTGTGGGGTGGGCTCTTCACACTCCAAT |
| (SEQ ID | CTCCACTTCCTTCTCAAGGCCTCAAAAAAAGTTGAAAAATGAAAACAAAAGCC |
| NO: 106) | CTGCTGTGTTGAGCTGGGCTCTGGCGTTGCCATGGACCCAGGGCAAACAGCGG |
| | TGCTCCTGCTCTGCCCCCGGCTCAGCTCATGCTGGGCCTGCACTTCTGGAAGGG |
| | AGCATGGACTTTGGAATGACTGGTTAGAACCCAAATGAATTAATGGAATTTGA |
| | CATAGTTCAAAAATAATAAAATGTGATACCCATGAAATGCTGATATTCTGCCT |
| | TAATTTGCCAGATTGGGGGCC |
| CRE0009_ | TTCAGAGGTCGGATCTGAATCCAGCTCCAAGGCCCCAGCACCCAAGCCCTGAC |
| S100B | CCCATGGCTGCCTGCTGGCTGGGAGTGGCATTCTTTAGAACAGGAAAGGCAGT |
| (SEQ ID | GAGTTCTCATTGCATCAATACTTGCATTTGCTACAACAGAAGCTTTTTGTCTCT |
| NO: 107) | TCCTCACATTCCTTTAGAACACAAGCCTCCTTTTCTGCCTGTGGCCATCGGTCA |
| | CTGGAGTCAGCCTCGTGAGTGGCTTGGTGGCGGATGGCACCGTCCTTTGTGGG |
| | AGAAAACAATGTTGCTGCCCAGGCCTTTCTGGAATGACCCCTTCCCACCCCCT |
| | GACCAGCCCCAGCAAGGCCCGGGGCTGGCTGCCTAGTTGATTGGAGCCAAGA |
| | GTTTGCTGAATGGATGAAGGGAGAAGGGACATCCAA |
| CRE0002_ | TCAGGGGTGCAGCTTTTTTTCTGTCTTTTACTCAGCCTGAGAAAGGTTGTCGTT |
| S100B | TGACAAGGTTTGTTCAGAGGTCGGATCTGAATCCAGCTCCAAGGCCCCAGCAC |
| (SEQ ID | CCAAGCCCTGACCCCATGGCTGCCTGCTGGCTGGGAGTGGCATTCTTTAGAAC |
| NO: 108) | AGGAAAGGCAGTGAGTTCTCATTGCATCAATACTTGCATTTGCTACAACAGAA |
| | GCTTTTTGTCTCTTCCTCACATTCCTTTAGAACACAAGCCTCCTTTTCTGCCTGT |
| | GGCCATCGGTCACTGGAGTCAGCCTCGTGAGTGGCTTGGTGGCGGATGGCACC |
| | GTCCTTTGTGGGAGAAAACAATGTTGCTGCCCAGGCCTTTCTGGAATGACCCC |
| | TTCCCACCCCCTGACCAGCCCCAGCAAGGCCCGGGGCTGGCTGCCTAGTTGAT |
| | TGGAGCCAAGAGTTTGCTGAATGGATGAAGGGAGAAGGGACATCCAAGTGAC |
| | TGAAGAAGATCTTAACAGAA |

TABLE 15
Cis-regulatory elements (CRE) comprised in the promoters of Table 11

| Name | SEQUENCE |
|---|---|
| CRE0004_Lmx1b | CTGGGCAGAGAGGGGGCATCGGGGGCATGGCTAGGGGCCAGCACTGTGCTTC |
| (SEQ ID NO: | CTGGGCGCCTCACCTCCTCCCTGACTCCTGGAGACTCCCAGCCCCTGTCTGGGA |
| 128) | GATGAGCATTTAGGAATCTGCTTGTGCAGGGGTGGTGGGAGGGGCCGGGGTG |
| | GAGGGCGCATCCCCACGGGGAGATTGGATGGAAATGGCCTGCCAGTGTGTGT |
| | GTGAGTGTGCGCCTGTGGCAGCAGCAGAGTAAACAGCCGCTGCCCTGTCCTCT |
| | CTGCGGCCGTGGCCAGGTACACAGGCCTGTTTGGACAGCTGCCTTGTCTGTCC |
| | GTCTGTTTGGGAGATGCTGGCTGATAGATGGGGATGGGCGGACTGTTAACCCC |
| | TCGTTGCCTGCACTGCTATGTGCTTCCTGCCTCATCCATGGGGTAGAAGGTAGC |
| | CAGAAGGTGGTCCTGGCTGTGCCCCCAGCTCCTCTCTAGGGGGGAAACCTCTA |
| | GTTCTGAGTCAGGGACAGAGTGAGGAGGGCTCCAGGGCATCAAGAGCTTGCT |
| | CCTCCCCGCACCAGGGAGCCAAGGACAGAGGAGAAGGGGGTCTTCCCCAGTG |
| | GTGACTAGGGGCAGAATATGTCTCTGAGTGAGTGTCTGGAGCCCTCCTCACCC |
| | CAACACCATG |
| CRE0003_Pitx3 | GGTGTGTGGAAGGGTGAGAGGCACACACACAGACACTGAAAGAATCCTAGGC |
| (SEQ ID NO: | CTGGTAGGCACTTAACAAATGTCTGTTACAGACCAGAATTTTATTGCTGTTAG |
| 129) | AGACCCAAGCCCCTCATAGGAACAGTGAGAAACAGGTGCAGAAAGGCGGAGT |
| | AACTTTATCTAAAGTCATAGGCTCCCTGAATAGCAGAGCTGACACCTACAAGG |
| | AAGCGTTGGAGACCAGATCTACCAGCTAGCCTCCCTGAGACCACGAGGTGGC |
| | GCCGCAGCACCGGCTGTGGCCGATGCCAGCCAGGTAGCCGGTTTCCCACGTCC |
| | CCCGCACGCACGCACCTCTTTGCTGCAGGAATCCCGGGCTGCCCCGACCTGGA |
| | GTAGGGGGGGTGGTGAGTGGGACTGAGTCCCTAGAAGCCTGGACCCTCACTTC |
| | GTTCCTGTACATCCAGCTCGCCTGTAGACAGTGGGGGAGGATGAAGGGAAGA |
| | GGACTCAAGCGCAACTTTGAATCATCACGCCTTCGACAGTCCGCGCACGTTTA |
| | TTTCATTTATCTTTGAAAACGAGGGAGGGGAAGCCTGGAGAAGGCGGGATGG |
| | GCCAAGGGTGAGTTGGCCCCCGGGGAGCTGGTCCCTGTTCCTGGCTTTAGTCC |
| | CAGGGGCGCGGTCTGTGTGTAGGGC |
| CRE0002_Gbf1 | TGCTACCAGAGCCGGGAGAGCTGCTCGGAGACGCCTCCGGGGTGCGGGCTGG |
| (SEQ ID NO: | ACATGAGCAGCGGCTGCCGGTCCTGGGACTAGGCCCCGCCATTTTGGATCCGC |
| 130) | TGACAGGTCAGCGAAGTCTCTTCCTAGAGTTCCGGTGTCGTGAAGGCCGCCCT |
| | GACATCGCAATAGGGAATTAGTGGGAAGGGCCCTTAAATTGGGCGAGCCAAG |
| | GTGGG |
| CRE0005_faf1_ | GGAACATTTAGACTTTGGGAAAGAGATTAGTGATTTACATTGCTATCTCACTG |
| short | ATTTAATTTAAATGCTCTTCCAAACCAAACACACATGTGCCGAAGAGGCTACT |
| (SEQ ID NO: | AAGAAACCCAACATGCAGAGTTCTCTATAAGTGCAGCCGACAGTGTTGACTGA |
| 131) | AACTAAACTTGGAAATCCAGGGCACTAATGCACAATATCAAGCAATAAAACG |
| | GCATCTCTTTGGCAATATTTAATTTAAAAAAGAAGAAAGAGACAGGCGAAGA |
| | TCAGGCACTGTCTGTTTTGGAGGATCAACCATTCTGCATTTCAAAGCATTGGTC |
| | CCTGCAATATCCAGGTTACTGTGCTAGAATCTCGACTATTATATCGCAGTTGTG |
| | AGAGGGAGGGCAAAGATGTGTTTACTCAGTGATTAGGCCCTTAGAATAAGCCT |
| | CTAGCTCCTAGAGAGACAGCTCACCACTTATTCATTTGGGCCAATTCACAAAG |
| | CCTAGGAAGATTAAACATCCATGCTGAGAAGACAAGCGAATGCAGACGGTGA |
| | AAAAGAAATAAAAATTCTTTAAAAACTCTGAGATGACTTCATTATTTTTCCAC |
| | AAGGAAACTTTAGGAAAGTGTTTAGTTAGAGAAAAACCCACATTGACCTCTCT |
| | CTAAACCCTTAATCTTTCCTTTGTGGTGGCACTGCTTTGTGGTAAGCGACTGGC |
| | TCGCCTCGCCCCTCTTTTCACTGGAAGCTGAGAGAAAAAAGACTCTGGAGAAA |
| | CAGTTTTCGTTCCAGGGACACAAACCCCTGACACTGTTAA |
| CRE0006_Pitx2_ | GAAAATTTAATGTCATTGAAGCTCTATTTCCAATAGCAAAGGAGCACTATTGC |
| short (SEQ ID | TAATAGACTTCAGAGCTTGAAATAAATAAATCTTTGGAATCCTGTTGCATCTCT |
| NO: 132) | TGGGGTGTGACATTTGACAGTCTTTTATAGCACAGAACGAAACAAGTTTGTGA |
| | GCTGGAATTCAATTGTGGCGTATTGATTCCTTGCATCAGTCATTATTCCCTGCT |
| | GATTGACAGGTGAAAATTGGTTACGTTAAGTATTTCATATGTTATATTGGCTGA |
| | CATTTGCTTGCCTGCTCTTGTGTCAATATTGTTGTAAAGATCTCCAGCTTTATG |
| | AGATAGCAATAGACACTGACTGTGGCTTTTGTGTGATGTTCCAGTGTTTTTCCT |
| | GACATAATTTAAGACATATTAAAAACCAGCAGCATCTTCCCTCTTGAGAAGCT |
| | TAATGCCAATATTATTGTCTTCCAGGGGAAGATCATGTATGCTCATAATCGGG |
| | TGCTAATTTCCACCAGTACGCTCATGTTTAGGCATTAGGCACTATAACTGTAAA |
| | ATTGAGCCTTCTTGATTGATTCATGTCAAGCCTCATCTCGGCTCCTGCAGGGGA |
| | AGTCATCCGGCTGACCCTTTTTACACTAAAAGAAGAGATTTGTGTTCCTTTCTT |
| | TCACCTGGAACCATCAAATTGACTGAATAATCTGTAATACATTAGTGCTGACA |
| | TTTGTTAGGGAGAATTAAACAAGACACAGTAATCATTCCCCAGAATAAAAATT |
| | GTGTTTGAT |
| CRE0007_Pitx2_ | TGAGACTGAGTGGGAGTGGGGATGAAGCCTTTTTTCTTCATTTTTTTTTCCTCA |
| short (SEQ ID | GGTGCAATTCTGTGTTAATATAAGAGAAGTGTGGCCTTCCTTCTCATAGCACTA |
| NO: 133) | AAAGTGAGATAATCCCTGTGTAAGAAATCAGTAAGTACGGTCTGCTTAATCTA |
| | GTCCCAGTGTGAAACTGTTGACATTTGTTCTTTTTTCTATCATTATGTGACTGG |
| | GCCTGTTTTGTGCTGGATTAGGCACAAATCTCCTATGCAGCACATTTGGCATGT |
| | TACTAGTAGTTTAACTTCATTAATAATGTATGAAGAAAATGTAATCCATGACA |
| | AGGAAGCAAAGAAAAGTATTTTTTTTTTTTTTTGCTTCTCCCAAATCCTTTGGA |
| | ATGAGTAATTATTCAACATTTTATGTTTGATGTTATATTTTACAATTCAACTTCC |
| | ATA |
| CRE0008 _Pitx2_ | GCTGGTGCTTCTTTTTTCTGTTTTTTAAGCTGCTTTACAGACGAAAATGGAACT |
| short (SEQ ID | ATATTTGGAACAATGCTTTCTGTTTTTCCATACTATTGATATTTGTGGAAAGTC |
| NO: 134) | ACAAAATGGCCTAAGGAAGCTAAGCTCGCCCCAAGCAGTGGTCACTTACAAG |
| | TACTTTTGTACTCTGTACTCCTGTCACATTTGGGCGATCAGAGCAACAGCTGGG |
| | GAGACTTTTTCAACAAAGATGAGTGTCAGATAATCCTGATGAGATTCCACATC |
| | CAACATCTTTTGTAATTATGTCACATTCAGCTGTAATGGAATAATTCAAGCTGA |
| | AAGAACAAGCTTTGATCCTTTCTTAAACCTTTCCCTGTGGACTGGCTATCTAAA |
| | AGATTTAAAGATATTTCTGTTACAAGATCTAGTGTTTCCTCAGAGAAGTCATGC |
| | TTCTGAAGCATCGTGATCTACAAGAACAATATCAAGTTTGCCAAACACATTTC |
| | TGAAAGCATCGTGTTTTGGGGGGAGGGGTTGTATTTAATGAAGATATCAATAA |
| | TATGC |

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (polIII) promoter sequence. Non-limiting examples of polIII promoter sequences include U6 and HI promoter sequences. In some embodiments, the second promoter sequence (e.g., the promoter sequence driving expression of the inhibitory RNA) is a RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polII promoter sequence drives expression of a protein coding region.

In some embodiments, the nucleic acid comprises a transgene that encodes a protein. The protein can be a therapeutic protein (e.g., a peptide, protein, or polypeptide useful for the treatment or prevention of disease states in a mammalian subject) or a reporter protein. In some embodiments, the protein is CYP46A1. In some embodiments, the protein is human CYP46A1. In some embodiments, the protein encodes SEQ ID NO; 2 or a protein comprising SEQ ID NO: 2. In some embodiments, the protein encodes a protein with a sequence identity of at least 80%, at least 85%, at least 90%, at least 95%, at least 98% to SEQ ID NO: 2. In some embodiments, the therapeutic protein is useful for treatment or prevention of Huntington's disease, for example Polyglutamine binding peptide 1 (QBP1), PTD-QBP1, ED 11, C4 intrabody, VL12.3 intrabody, MW7 intrabody, HappI antibodies, Happ3 antibodies, mEM48 intrabody, certain monoclonal antibodies (e.g., 1C2), and peptide P42 and variants thereof, as described in Marelli et al. (2016) Orphanet Journal of Rare Disease 11:24; doi: 10.1186/s 13023-016-0405-3. In some embodiments, the therapeutic protein is wild-type huntingtin protein (e.g., huntingtin protein having a PolyQ repeat region comprising less than 36 repeats).

### CYP46A1

Cholesterol 24-hydroxylase is a neuronal enzyme that is coded by the CYP46A1 gene. It converts cholesterol into 24-hydroxycholesterol and has a critical role in the efflux of cholesterol from the brain (Dietschy, J. M. et al., 2004). Brain cholesterol is essentially produced—but cannot be degraded-in situ, and intact blood-brain barrier restricts direct transportation of cholesterol from the brain (Dietschy, J. M. et al., 2004). 24-hydroxycholesterol is able to cross the plasma membrane and the blood-brain barrier and reaches the liver where it is degraded.

CYP46A1 is neuroprotective in a cellular model of HD (see, e.g., WO2012/049314). Moreover, there is a reduction of CYP46A1 mRNAs in the striatum, the more vulnerable brain structure in the disease, of the R6/2 transgenic HD mouse model.

During the early stages of AD, 24-hydroxycholesterol concentrations are high in CSF and in peripheral circulation. In later stages of AD, concentrations of 24-hydroxycholesterol may fall likely reflecting neuronal loss (Kolsch, H. et al., 2004). CYP46A1 is expressed around the amyloid core of the neuritic plaques in the brain of AD patients (Brown, J., 3rd et al., 2004).

Agonism of cholesterol 24-hydroxylase, encoded by CYP46A1, provided marked decrease of neuropathology and an improvement of cognitive deficits in mouse models of CNS disease. For example, co-expression of CYP46A1 with ExpHtt in a Huntington's disease model promoted a strong and significant decrease of ExpHtt aggregates formation (58% versus 27.5%)) (WO2012/049314). (see also, International Patent Publication WO2009/034127; which is incorporated by reference herein in its entirety). The methods described herein relate to agonism of CYP46A1 in combination with the administration of miRNAs targeting certain other targets. For example, the methods can relate to administration of a viral vector for the treatment of a neurological disease or disorder, wherein the vector expresses CYP46A1 in cells of the central nervous system.

In some embodiments, described herein is a viral vector for treating a neurological disease or disorder, which vector comprises a cholesterol 24-hydroxylase encoding nucleic acid. In some embodiments, the viral vector comprises a nucleic acid sequence that encodes the amino acid sequence SEQ ID NO:2. In some embodiments, the viral vector comprises a nucleic acid sequence that encodes an amino acid sequence with at least 80%, at least 85%, at least 90%, at least 95%, at least 98%, or more sequence identity to SEQ ID NO:2. In some embodiments, the viral vector comprises a sequence with at least 80%, at least 85%, at least 90%, at least 95%, at least 98%, or more sequence identity to SEQ ID NO: 1. In some embodiments, the viral vector comprises the sequence of SEQ ID NO: 1. In some embodiments, the viral vector may be an Adeno-Associated-Virus (AAV) vector.

Further description of CY46A1 and its therapeutic uses (e.g., for Alzheimer's disease, ALS, and ataxia) are described in the art, e.g., in WO 2012/049314, WO 2009/034127, WO 2018/138371, and WO2020/089154. The sequences, methods, and compositions described therein can be utilized in the methods and compositions described herein. The foregoing references are incorporated by reference herein in their entireties. The term “gene” refers to a polynucleotide containing at least one open reading frame that is capable of encoding a particular polypeptide or protein after being transcribed or translated.

The terms “coding sequence” or “a sequence which encodes a particular protein”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

A cDNA sequence for CYP46A1 is disclosed in Genbank Access Number NM_006668 (SEQ ID NO: 1). The amino acid sequence is shown in SEQ ID NO:2. The invention makes use of a nucleic acid construct comprising sequence SEQ ID NO:1 or a variant thereof for the treatment of a neurological disease or disorder. The variants include, for instance, naturally-occurring variants due to allelic variations between individuals (e.g., polymorphisms), alternative splicing forms, etc. The term variant also includes CYP46A1 gene sequences from other sources or organisms. Variants are preferably substantially homologous to SEQ ID NO:1 and/or 2, i.e., exhibit a nucleotide sequence identity of typically at least about 75%, preferably at least about 85%, more preferably at least about 90%, more preferably at least about 95% with SEQ ID NO:1 or 2. In some embodiments, the nucleic acid construct comprises a sequence with at least 95% sequence identity to SEQ ID NO: 1 and which retains the activity of SEQ ID NO: 1 or 2 (e.g., the ability to convert cholesterol into 24-hydroxycholesterol). Variants of a CYP46A1 gene also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

An exemplary CYP46A1 variant contemplated for use herein is provided in SEQ ID NOs: 109 and 110. In some embodiments, the viral vector comprises a nucleic acid sequence that encodes the amino acid sequence SEQ ID NO:109. In some embodiments, the viral vector comprises a nucleic acid sequence that encodes an amino acid sequence with at least 80%, at least 85%, at least 90%, at least 95%, at least 98%, or more sequence identity to SEQ ID NO: 109. In some embodiments, the viral vector comprises the nucleic acid sequence of SEQ ID NO:110. In some embodiments, the viral vector comprises a nucleic acid sequence with at least 80%, at least 85%, at least 90%, at least 95%, at least 98%, or more sequence identity to the sequence of SEQ ID NO: 110.

In one aspect, provided herein is a composition comprising an isolated nucleic acid comprising a sequence at least 80% identical, e.g., at least 85%, at least 90%, at least 95%, at least 98%, at least 99% or 100% identical, to SEQ ID NO: 110. In one aspect, provided herein is a composition comprising a recombinant viral vector comprising an isolated nucleic acid comprising a sequence at least 80% identical, e.g., at least 85%, at least 90%, at least 95%, at least 98%, at least 99% or 100% identical, to SEQ ID NO: 110. In some embodiments, an isolated nucleic acid encoding a CYP46A1 protein comprises at least 1, at least 2, at least 3, at least 4, at least 5, at least 6, at least 7, at least 8, at least 9, at least 10, at least 11, at least 12, at least 13, at least 14, or at least 15 of the mutations as compared to SEQ ID NO: 1. In some embodiments, the mutation comprises deletion and/or, addition, and/or, substitution of at least one nucleic acid as compared to the sequence set forth in SEQ ID NO: 1. The mutations can result in, e.g., removing bacterial sequence, and/or, removing alternating reading frames, and/or, removing CpG, and or, removing restriction enzyme sites. In several embodiments, the foregoing compositions can be used, e.g., in the absence of an administered miRNA to treat a neurological disease or disorder as described herein. In various embodiments, the foregoing compositions can be used, e.g., in the presence of an administered miRNA to treat a neurological disease or disorder as described herein. In some embodiments, recombinant viral vector, e.g., recombinant AAV comprising an isolated nucleic acid as set forth in SEQ ID NO: 110 is administered to a subject in need thereof for expressing the CYP46A1 protein and/or, for treating a neurological disease or disorder as described herein. In some embodiments, recombinant viral vector, e.g., recombinant AAV comprising an isolated nucleic acid sequence at least 80% identical, e.g., at least 85%, at least 90%, at least 95%, at least 98%, at least 99% or 100% identical to SEQ ID NO: 110, is administered to a subject in need therof for expressing the CYP46A1 protein and/or, for treating a neurological disease or disorder as described herein. In some embodiments, recombinant viral vector, e.g recombinant AAV comprising an isolated nucleic acid sequence at least 80% identical, e.g., at least 85%, at least 90%, at least 95%, at least 98%, at least 99% or 100% identical to SEQ ID NO: 111, is administered to a subject in need therof for expressing the CYP46A1 protein and/or, for treating a neurological disease or, disorder as described herein.

SEQ ID NO: 1 CYP46A1 mRNA atg agc ccc ggg ctg ctg ctg ctc ggc agc gcc gtc ctg ctc gcc ttc 48 ggc ctc tgc tgc acc ttc gtg cac cgc gct cgc agc cgc tac gag cac 96 atc ccc ggg ccg ccg cgg ccc agt ttc ctt cta gga cac ctc ccc tgc 144 ttt tgg aaa aag gat gag gtt ggt ggc cgt gtg ctc caa gat gtg ttt 192 ttg gat tgg get aag aag tat gga cct gtt gtg cgg gtc aac gtc ttc 240 cac aaa acc tca gtc atc gtc acg agt cct gag tcg gtt aag aag ttc 288 ctg atg tca acc aag tac aac aag gac tcc aag atg tac cgt gcg ctc 336 cag act gtg ttt ggt gag aga ctc ttc ggc caa ggc ttg gtg tcc gaa 384 tgc aac tat gag cgc tgg cac aag cag cgg aga gtc ata gac ctg gcc 432 ttc agc cgg agc tcc ttg gtt agc tta atg gaa aca ttc aac gag aag 480 gct gag cag ctg gtg gag att cta gaa gcc aag gca gat ggg cag acc 528 cca gtg tcc atg cag gac atg ctg acc tac acc gcc atg gac atc ctg 576

| CYP46A1 mRNA | |
|---|---|
| SEQ ID NO: 1 | |
| atg agc ccc ggg ctg ctg ctg ctc ggc agc gcc gtc ctg ctc gcc ttc | 48 |
| ggc ctc tgc tgc acc ttc gtg cac cgc gct cgc agc cgc tac gag cac | 96 |
| atc ccc ggg ccg ccg cgg ccc agt ttc ctt cta gga cac ctc ccc tgc | 144 |
| ttt tgg aaa aag gat gag gtt ggt ggc cgt gtg ctc caa gat gtg ttt | 192 |
| ttg gat tgg gct aag aag tat gga cct gtt gtg cgg gtc aac gtc ttc | 240 |
| cac aaa acc tca gtc atc gtc acg agt cct gag tcg gtt aag aag ttc | 288 |
| ctg atg tca acc aag tac aac aag gac tcc aag atg tac cgt gcg ctc | 336 |
| cag act gtg ttt ggt gag aga ctc ttc ggc caa ggc ttg gtg tcc gaa | 384 |
| tgc aac tat gag cgc tcg cac aag cag cgg aga gtc ata gac ctg gcc | 432 |
| ttc agc cgg agc tcc ttg gtt agc tta atg gaa aca ttc aac gag aag | 480 |
| gct gag cag ctg gtg gag att cta gaa gcc aag gca gat ggg cag acc | 528 |
| cca gtg tcc atg cag gac atg ctg acc tac acc gcc atg gac atc ctg | 576 |
| gcc aag gca gct ttt ggg atg gag acc agt atg ctg ctg ggt gcc cag | 624 |
| aag cct ctg tcc cag gca gtg aaa ctt atg ttg gag gga atc act gcg | 672 |
| tcc cgc aac act ctg gca aag ttc ctg cca ggg aag agg aag cag ctc | 720 |
| cgg gag gtc cgg gag agc att cgc ttc ctg cgc cag gtg ggc agg gac | 768 |
| tgg gtc cag cgc cgc cgg gaa gcc ctg aag agg ggc gag gag gtt cct | 816 |
| gcc gac atc ctc aca cag att ctg aaa gct gaa gag gga gcc cag gac | 864 |
| gac gag ggt ctg ctg gac aac ttc gtc acc ttc ttc att gct ggt cac | 912 |
| gag acc tct gcc aac cac ttg gcg ttc aca gtg atg gag ctg tct cgc | 960 |
| cag cca gag atc gtg gca agg ctg cag gcc gag gtg gat gag gtc att | 1008 |
| ggt tct aag agg tac ctg gat ttc gag gac ctg ggg aga ctg cag tac | 1056 |
| ctg tcc cag gtc ctc aaa gag tcg ctg agg ctg tac cca cca gca tgg | 1104 |
| ggc acc ttt cgc ctg ctg gaa gag gag acc ttg att gat ggg gtc aga | 1152 |
| gtc ccc ggc aac acc ccg ctc ttg ttc agc acc tat gtc atg ggg cgg | 1200 |
| atg gac aca tac ttt gag gac ccg ctg act ttc aac ccc gat cgc ttc | 1248 |
| ggc cct gga gca ccc aag cca cgg ttc acc tac ttc ccc ttc tcc ctg | 1296 |
| ggc cac cgc tcc tgc atc ggg cag cag ttt gct cag atg gag gtg aag | 1344 |
| gtg gtc atg gca aag ctg ctg cag agg ctg gag ttc cgg ctg gtg ccc | 1392 |
| ggg cag cgc ttc ggg ctg cag gag cag gcc aca ctc aag cca ctg gac | 1440 |
| ccc gtg ctg tgc acc ctg cgg ccc cgc ggc tgg cag ccc gca ccc cca | 1488 |
| cca ccc ccc tgc tga gggggcctcc aggcaggacg agactcctcg ggcaagggcc | 1543 |
| gtgcccgccc acctctgctg cccacggcca cccacccttc tcccctgccc cgtcccctgg | 1603 |
| gccacccttc acgctggctt ccagcgggcc ctctgccgac cgcctgcttc acacccctca | 1663 |
| gcgctccctg tcgcctgcgg actccatggc ccttcctgga ctggcccttg cccaactccc | 1723 |
| agccaccacc actgtcccta ccactgagcc cttgcacagg ccacttgctc agacgagaca | 1783 |
| ccctaactct tgctcactcc ctaaagccct cttcaggggt cacctcctcc aagaagccct | 1843 |
| ccttgccacc ccccgccggc aggggcccct cctctgtgct ccctcggtca cctgtgctac | 1903 |
| ctctaacacc acactgacca cactgtatcg tgagtgtccg ttgacgtgac caattgccct | 1963 |
| gccaggctgt cagcgcctca agggtagggt ctgcgtgtga tttgtctctg agccccctgt | 2023 |
| gcccacccag ggcccggcac agagtcgatg ctcaataaat gtgtgttgac tgcaaaaaaa | 2083 |
| aaaaaaaaaa aaaaaaaaaa aaaaaaaaaa aaaaaaaaaa aaaaaaaaaa aaaaa | 2138 |

```
CYP46A1 amino acid sequence
SEQ ID NO: 2
Met Ser Pro Gly Leu Leu Leu Leu Gly Ser Ala Val Leu Leu Ala Phe
1 5 10 15
Gly Leu Cys Cys Thr Phe Val His Arg Ala Arg Ser Arg Tyr Glu His
                        20 25 30
Ile Pro Gly Pro Pro Arg Pro Ser Phe Leu Leu Gly His Leu Pro Cys
                35 40 45
Phe Trp Lys Lys Asp Glu Val Gly Gly Arg Val Leu Gln Asp Val Phe
        50 55 60
Leu Asp Trp Ala Lys Lys Tyr Gly Pro Val Val Arg Val Asn Val Phe
65 70 75 80
His Lys Thr Ser Val Ile Val Thr Ser Pro Glu Ser Val Lys Lys Phe
                                85 90 95
Leu Met Ser Thr Lys Tyr Asn Lys Asp Ser Lys Met Tyr Arg Ala Leu
                        100 105 110
Gln Thr Val Phe Gly Glu Arg Leu Phe Gly Gln Gly Leu Val Ser Glu
                115 120 125
Cys Asn Tyr Glu Arg Trp His Lys Gln Arg Arg Val Ile Asp Leu Ala
        130 135 140
Phe Ser Arg Ser Ser Leu Val Ser Leu Met Glu Thr Phe Asn Glu Lys
145 150 155 160
Ala Glu Gln Leu Val Glu Ile Leu Glu Ala Lys Ala Asp Gly Gln Thr
                                165 170 175
Pro Val Ser Met Gln Asp Met Leu Thr Tyr Thr Ala Met Asp Ile Leu
                        180 185 190
Ala Lys Ala Ala Phe Gly Met Glu Thr Ser Met Leu Leu Gly Ala Gln
                195 200 205
Lys Pro Leu Ser Gln Ala Val Lys Leu Met Leu Glu Gly Ile Thr Ala
        210 215 220
Ser Arg Asn Thr Leu Ala Lys Phe Leu Pro Gly Lys Arg Lys Gln Leu
225 230 235 240
Arg Glu Val Arg Glu Ser Ile Arg Phe Leu Arg Gln Val Gly Arg Asp
                                245 250 255
Trp Val Gln Arg Arg Arg Glu Ala Leu Lys Arg Gly Glu Glu Val Pro
                        260 265 270
Ala Asp Ile Leu Thr Gln Ile Leu Lys Ala Glu Glu Gly Ala Gln Asp
                275 280 285
Asp Glu Gly Leu Leu Asp Asn Phe Val Thr Phe Phe Ile Ala Gly His
        290 295 300
Glu Thr Ser Ala Asn His Leu Ala Phe Thr Val Met Glu Leu Ser Arg
305 310 315 320
Gln Pro Glu Ile Val Ala Arg Leu Gln Ala Glu Val Asp Glu Val Ile
                                325 330 335
Gly Ser Lys Arg Tyr Leu Asp Phe Glu Asp Leu Gly Arg Leu Gln Tyr
                        340 345 350
Leu Ser Gln Val Leu Lys Glu Ser Leu Arg Leu Tyr Pro Pro Ala Trp
                355 360 365
Gly Thr Phe Arg Leu Leu Glu Glu Glu Thr Leu Ile Asp Gly Val Arg
        370 375 380
Val Pro Gly Asn Thr Pro Leu Leu Phe Ser Thr Tyr Val Met Gly Arg
385 390 395 400
Met Asp Thr Tyr Phe Glu Asp Pro Leu Thr Phe Asn Pro Asp Arg Phe
                                405 410 415
Gly Pro Gly Ala Pro Lys Pro Arg Phe Thr Tyr Phe Pro Phe Ser Leu
                        420 425 430
Gly His Arg Ser Cys Ile Gly Gln Gln Phe Ala Gln Met Glu Val Lys
                435 440 445
Val Val Met Ala Lys Leu Leu Gln Arg Leu Glu Phe Arg Leu Val Pro
        450 455 460
Gly Gln Arg Phe Gly Leu Gln Glu Gln Ala Thr Leu Lys Pro Leu Asp
465 470 475 480
Pro Val Leu Cys Thr Leu Arg Pro Arg Gly Trp Gln Pro Ala Pro Pro
                                485 490 495
Pro Pro Pro Cys
                        500
```

### Vectors

Without wishing to be bound by any particular theory, allele-specific silencing of a pathogenic gene, e.g., mutant huntingtin (HTT), may provide an improved safety profile in a subject compared to non-allele specific silencing (e.g., silencing of both wild-type and mutant HTT alleles) because wild-type expression and function is preserved in the cells. For example, aspects of the invention relate to the inventors' recognition and appreciation that isolated nucleic acids and vectors that incorporate one or more inhibitory RNA (e.g., miRNA) sequences targeting the HTT gene in a non-allele-specific manner while driving the expression of hardened wild-type HTT gene (a wild-type HTT gene that is not targeted by the miRNA) are capable of achieving concomitant mutant HTT knockdown e.g., in the CNS tissue, with increased expression of wildtype HTT. Generally, the sequence of the nucleic acid encoding endogenous wild-type and mutant HTT mRNAs, and the nucleic acid of the transgene encoding the “hardened” wild-type HTT mRNA are sufficiently different such that the “hardened” wild-type HTT transgene mRNA is not targeted by the one or more inhibitory RNAs (e.g., miRNAs). This may be accomplished, for example, by introducing one or more silent mutations into the HTT transgene sequence such that it encodes the same protein as the endogenous wild-type HTT gene but has a different nucleic acid sequence. In this case, the exogenous mRNA may be referred to as “hardened.” Alternatively, the inhibitory RNA (e.g., miRNA) can target the 5′ and/or 3′ untranslated regions of the endogenous wild-type HTT mRNA. These 5′ and/or 3′ regions can then be removed or replaced in the transgene mRNA such that the transgene mRNA is not targeted by the one or more inhibitory RNAs.

Reporter sequences (e.g., nucleic acid sequences encoding a reporter protein) that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of a nucleic acid. Recombinant adeno-associated viruses (rAAVs).

In some embodiments, the vector is adeno-associated virus (AAV) or recombinant AAV. In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, a recombinant AAV (rAAV) comprises a AAV capsid protein selected from the group consisting of AAV2, AAV3, AAV4, AAV5, AAV6, AAV8, AAVrh8, AAVrh10, AAV 2G9, AAV 2.5G9, AAV9, and AAV10. In some embodiments, recombinant AAV capsid (rAAV) protein is of a serotype derived from a non-human primate, for example AAVrh10 serotype. In some embodiments, rAAV is AAV PhP.eB or, AAV PhP.B, as described in US Publication nos and US granted patents US20170166926A1, U.S. Pat. No. 9,585,971, U.S. Ser. No. 10/301,360, U.S. Pat. No. 9,957,303, U.S. Ser. No. 10/202,425, U.S. Ser. No. 10/519,198, US20190292230A1, US20200087353A1, which are incorporated herein by reference in tier entirety. In some embodiments, rAAV comprises an AAV comprising a surface bound peptide e.g., PB5-3, PB5-5, PB5-14 as described in international publication WO201912635, which is incorporated by reference in its entirety. In some embodiments, rAAV is an AAV9 serotype. In some embodiments, the rAAV is an AAV1, AAV2, AAV3b, AAV4, AAV5, AAV6, AAV7, AAV8, AAV9, AAV10, AAV 11, AAV12, or AAV13 serotype or, a chimera thereof. In some embodiments, the rAAV comprises a capsid protein from serotype AAV1, AAV2, AAV3a, AAV3b, AAV4, AAV5, AAV6, AAV7, AAV8, AAV9, AAV 2G9, AAV 2.5G9, AAV rh8, AAV rhl0, AAV rh74, AAV10, or, AAV 11 or, a chimera thereof. In certain embodiments, the rAAV comprises a chemically modified capsid as disclosed in WO 2017/212019 e.g., mannose ligand is chemically coupled to AAV2. The rAAVs with chemically modified capsids disclosed in WO 2017/212019 is incorporated herein by reference in its entirety. As a further embodiment, the rAAV comprises AAV capsid proteins of this invention that can be polyploid (also referred to as haploid, or, rational haploid or, rational polyploid) in that they can comprise VP1, VP2 and VP3 capsid proteins from more than one AAV serotypes in a single AAV virion as described in PCT/US18/22725, PCT/US2018/044632, or U.S. Pat. No. 10,550,405, which are incorporated by reference. In some embodiments rAAV comprises a capsid protein selected from AAV serotypes listed in Table 17.

TABLE 17
Table 17: AAV Serotypes and exemplary published corresponding capsid sequence

| Serotype and where capsid sequence is published | Serotype and where capsid sequence is published |
|---|---|
| AAV3.3b (See SEQ ID NO: 72 in US20030138772) | AAV3-3 (See SEQ ID NO: 200 US20150315612) |
| AAV3-3 (See SEQ ID NO: 217 US20150315612) | AAV3a ((See SEQ ID NO: 5 in U.S. Pa. No. 6156303) |
| AAV3a (See SEQ ID NO: 9 in U.S. Pa. No. 6156303) | AAV3b (See SEQ ID NO: 6 in U.S. Pa. No. 6156303) |
| AAV3b (See SEQ ID NO: 10 in U.S. Pa. No. 6156303) | AAV3b (See SEQ ID NO: 1 in U.S. Pa. No. 6156303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO: 63 in US20030138772) |
| | and US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in US20030138772) |
| AAV42.2 (See SEQ ID NO: 102 in US20030138772) | AAV42.3b (See SEQ ID NO: 36 in |
| | US20030138772) |
| AAV42.3B (See SEQ ID NO: 107 in US20030138772) | AAV42.4 (See SEQ ID NO: 33 in US20030138772) |
| AAV42.4 (See SEQ ID NO: 88 in US20030138772) | AAV42.8 (See SEQ ID NO: 27 in US20030138772) |
| AAV42.8 (See SEQ ID NO: 85 in US20030138772) | AAV43.1 (See SEQ ID NO: 39 in US20030138772) |
| AAV43.1 (See SEQ ID NO: 92 in US20030138772) | AAV43.12 (See SEQ ID NO: 41 in |
| | US20030138772) |
| AAV43.12 (See SEQ ID NO: 93 in US20030138772) | AAV8 (See SEQ ID NO: 15 in US20150159173) |
| AAV8 (See SEQ ID NO: 7 in US20150376240) | AAV8 (See SEQ ID NO: 4 in |
| | US20030138772; US20150315612 SEQ |
| ID NO: 182 | AAV8 (See SEQ ID NO: 95 in US20030138772), |
| | US20140359799 SEQ |
| AAV8 (See SEQ ID NO: 31 in US20150159173) | AAV8 (See, e.g., SEQ ID NO: 8 in US20160017295, |
| | or SEQ ID NO: 7 in U.S. Pa. No. 7198951, or SEQ ID NO: 223 |
| | in US20150315612) |
| AAV8 (See SEQ ID NO: 8 in US20150376240) | AAV8 (See SEQ ID NO: 214 in US20150315612) |
| AAV-8b (See SEQ ID NO: 5 in US20150376240) | AAV-8b (See SEQ ID NO: 3 in US20150376240) |
| AAV-8h (See SEQ ID NO: 6 in US20150376240) | AAV-8h (See SEQ ID NO: 4 in US20150376240) |
| AAV9 (See SEQ ID NO: 5 in US20030138772) | AAV9 (See SEQ ID NO: 1 in U.S. Pa. No. 7,198,951) |
| AAV9 (See SEQ ID NO: 9 in US20160017295) | AAV9 (See SEQ ID NO: 100 in US20030138772), |
| | U.S. Pat. No. 7,198,951 SEQ ID NO: 2 |
| | AAV9 (See SEQ ID NO: 3 in U.S. Pat. No. 7,198,951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in US20030138772) | AAVA3.3 (See SEQ ID NO: 57 in |
| | US20030138772) |
| AAVA3.3 (See SEQ ID NO: 66 in US20030138772) | AAVA3.4 (See SEQ ID NO: 54 in |
| | US20030138772) |
| AAVA3.4 (See SEQ ID NO: 68 in US20030138772) | AAVA3.5 (See SEQ ID NO: 55 in |
| | US20030138772) |
| AAVA3.5 (See SEQ ID NO: 69 in US20030138772) | AAVA3.7 (See SEQ ID NO: 56 in |
| | US20030138772) |
| AAVA3.7 (See SEQ ID NO: 67 in US20030138772) | AAV29. (See SEQ ID NO: 11 in (AAVbb. 1) 161 |
| | US20030138772) |
| AAVC2 (See SEQ ID NO: 61 in US20030138772) | AAVCh.5 (See SEQ ID NO: 46 in |
| | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in US20030138772) | AAVcy.3 (AAV24.1) (See SEQ ID NO: 16 in |
| | US20030138772) |
| AAV27.3 (See SEQ ID NO: 104 in US20030138772) | AAVcy.4 (AAV27.3) (See SEQ ID NO: 17 in |
| | US20030138772) |
| AAVcy.5 (See SEQ ID NO: 227 in US20150315612) | AAV7.2 (See SEQ ID NO: 103 in US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in US20150159173) |
| US20030138772) | |
| AAVcy.5 (See SEQ ID NO: 24 in US20150159173) | AAVCy.5R1 (See SEQ ID NO: in US20150159173 |
| AAVCy.5R2 (See SEQ ID NO: in US20150159173) | AAVCy.5R3 (See SEQ ID NO: in |
| | US20150159173 |
| AAVCy.5R4 (See SEQ ID NO: in US20150159173) | AAVDJ (See SEQ ID NO: 3 in US20140359799) |
| | and SEQ ID NO: 2 in U.S. Pat. No. 7,588,772) |
| | AAVDJ (See SEQ ID NO: 2 in US20140359799; |
| | and SEQ ID NO: 1 in U.S. Pat. No. 7,588,772) |
| | AAVDJ-8 (See SEQ ID NO: in U.S. Pat. No. 7,588,772; |
| | Grimm et al 2008 |
| AAVDJ-8 (See SEQ ID NO: in U.S. Pat. No. 7,588,772; Grimm et | AAVF5 (See SEQ ID NO: 110 in US20030138772) |
| al 2008 | |
| AAVH2 (See SEQ ID NO: 26 in US20030138772) | AAVH6 (See SEQ ID NO: 25 in US20030138772) |
| AAVhEl. l (See SEQ ID NO: 44 in U.S. Pat. No. 9,233,131) | AAVhErl.14 (See SEQ ID NO: 46 in U.S. Pat. No. 9,233,131) |
| AAVhErl.16 (See SEQ ID NO: 48 in U.S. Pat. No. 9,233,131) | AAVhErl.18 (See SEQ ID NO: 49 in U.S. Pat. No. 9,233,131) |
| AAVhErl.23 (AAVhEr2.29) (See SEQ ID NO: 53 in | AAVhErl.35 (See SEQ ID NO: 50 in U.S. Pat. No. 9,233,131) |
| U.S. Pat. No. 9,233,131) | |
| AAVhErl.36 (See SEQ ID NO: 52 in U.S. Pat. No. 9,233,131) | AAVhErl.5 (See SEQ ID NO: 45 in U.S. Pat. No. 9,233,131) |
| AAVhErl.7 (See SEQ ID NO: 51 in U.S. Pat. No. 9,233,131) | AAVhErl.8 (See SEQ ID NO: 47 in U.S. Pat. No. 9,233,131) |
| AAVhEr2.16 (See SEQ ID NO: 55 in U.S. Pat. No. 9,233,131) | AAVhEr2.30 (See SEQ ID NO: 56 in U.S. Pat. No. 9,233,131) |
| AAVhEr2.31 (See SEQ ID NO: 58 in U.S. Pat. No. 9,233,131) | AAVhEr2.36 (See SEQ ID NO: 57 in U.S. Pat. No. 9,233,131) |
| AAVhEr2.4 (See SEQ ID NO: 54 in U.S. Pat. No. 9,233,131) | AAVhEr3.1 (See SEQ ID NO: 59 in U.S. Pat. No. 9,233,131) |
| AAVhu.l (See SEQ ID NO: 46 in US20150315612) | AAVhu.l (See SEQ ID NO: 144 in |
| | US20150315612) |
| AAVhu.lO (AAV16.8) (See SEQ ID NO: 56 in | AAVhu.lO (AAV16.8) (See SEQ ID NO: 156 in |
| US20150315612) | US20150315612) |
| AAVhu.l l (AAV16.12) (See SEQ ID NO: 57 in | AAVhu.l l (AAV16.12) (See SEQ ID NO: 153 in |
| US20150315612) | US20150315612) |
| AAVhu.12 (See SEQ ID NO: 59 in US20150315612) | AAVhu.12 (See SEQ ID NO: 154 in |
| | US20150315612) |
| AAVhu.13 (See SEQ ID NO: 16 in US2015015917 and | |
| ID NO: 71 in US20150315612) | |
| AAVhu.13 (See SEQ ID NO: 32 in US20150159173 | |
| and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in US20150315612) | AAVhu.15 (AAV33.4) (See SEQ ID NO: 50 in |
| | US20150315612) |
| AAVhu.156.1 (See SEQ ID NO: 179 in | AAVhu.16 (See SEQ ID NO: 148 in |
| US20150315612) | US20150315612) |
| AAVhu.l6 (AAV33.8) (See SEQ ID NO: 51 in | AAVhu.17 (See SEQ ID NO: 83 in |
| US20150315612) | US20150315612) |
| AAVhu.17 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in US20150315612) | AAVhu.19 (See SEQ ID NO: 62 in |
| | US20150315612) |
| AAVhu.19 (See SEQ ID NO: 133 in US20150315612) | AAVhu.2 (See SEQ ID NO: 48 in |
| | US20150315612) |
| AAVhu.2 (See SEQ ID NO: 143 in US20150315612) | AAVhu.20 (See SEQ ID NO: 63 in |
| | US20150315612) |
| AAVhu.20 (See SEQ ID NO: 134 in US20150315612) | AAVhu.21 (See SEQ ID NO: 65 in |
| | US20150315612) |
| AAVhu.21 (See SEQ ID NO: 135 in US20150315612) | AAVhu.22 (See SEQ ID NO: 67 in |
| | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in US20150315612) | AAVhu.24 (See SEQ ID NO: 66 in |
| | US20150315612) |
| AAVhu.24 (See SEQ ID NO: 136 in US20150315612) | AAVhu.25 (See SEQ ID NO: 49 in |
| | US20150315612) |
| AAVhu.25 (See SEQ ID NO: 146 in US20150315612) | AAVhu.26 (See SEQ ID NO: 17 in |
| | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in US20150315612) | AAVhu.28 (See SEQ ID NO: 68 in |
| | US20150315612) |
| AAVhu.28 (See SEQ ID NO: 130 in US20150315612) | AAVhu.29 (See SEQ ID NO: 69 in |
| | US20150315612) |
| AAVhu.29 (See SEQ ID NO: 42 in US20150159173 | |
| and SEQ ID NO: 132 in US20150315612) | |
| AAVhu.29 (See SEQ ID NO: 225 in US20150315612) | AAVhu.29R (See SEQ ID NO: in US20150159173 |
| AAVhu.3 (See SEQ ID NO: 44 in US20150315612) | AAVhu.3 (See SEQ ID NO: 145 in |
| | US20150315612) |
| AAVhu.30 (See SEQ ID NO: 70 in US20150315612) | AAVhu.30 (See SEQ ID NO: 131 in |
| | US20150315612) |
| AAVhu.31 (See SEQ ID NO: 1 in US20150315612) | AAVhu.31 (See SEQ ID NO: 121 in |
| | US20150315612) |
| AAVhu.32 (See SEQ ID NO: 2 in US20150315612) | AAVhu.32 (See SEQ ID NO: 122 in |
| | US20150315612) |
| AAVhu.33 (See SEQ ID NO: 75 in US20150315612) | AAVhu.33 (See SEQ ID NO: 124 in |
| | US20150315612) |
| AAVhu.34 (See SEQ ID NO: 72 in US20150315612) | AAVhu.34 (See SEQ ID NO: 125 in |
| | US20150315612) |
| AAVhu.35 (See SEQ ID NO: 73 in US20150315612) | AAVhu.35 (See SEQ ID NO: 164 in |
| | US20150315612) |
| AAVhu.36 (See SEQ ID NO: 74 in US20150315612) | AAVhu.36 (See SEQ ID NO: 126 in |
| | US20150315612) |
| AAVhu.37 (See SEQ ID NO: 34 in US20150159173 | |
| and SEQ ID NO: 88 in US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in US20150315612) | AAVhu.39 (See SEQ ID NO: 102 in |
| | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in US20150315612) | AAVhu.40 (See SEQ ID NO: 87 in |
| | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO: 8 in | AAVhu.43 (See SEQ ID NO: 160 in |
| US20150315612) | US20150315612) |
| AAVhu.43 (See SEQ ID NO: 236 in US20150315612) | AAVhu.43 (AAV128.1) (See SEQ ID NO: 80 in |
| | US20150315612) |
| AAVhu.44 (See SEQ ID NO: 45 in US20150159173 | |
| and SEQ ID NO: 158 in US20150315612) | |
| AAVhu.44 (AAV128.3) (See SEQ ID NO: 81 in | AAVhu.44R1 (See SEQ ID NO: in |
| US20150315612) | US20150159173 |
| AAVhu.44R2 (See SEQ ID NO: in US20150159173 | AAVhu.44R3 (See SEQ ID NO: in |
| | US20150159173 |
| AAVhu.45 (See SEQ ID NO: 76 in US20150315612) | AAVhu.45 (See SEQ ID NO: 127 in |
| | US20150315612) |
| AAVhu.46 (See SEQ ID NO: 82 in US20150315612) | AAVhu.46 (See SEQ ID NO: 159 in |
| | US20150315612) |
| AAVhu.46 (See SEQ ID NO: 224 in US20150315612) | AAVhu.47 (See SEQ ID NO: 77 in |
| | US20150315612) |
| AAVhu.47 (See SEQ ID NO: 128 in US20150315612) | AAVhu.48 (See SEQ ID NO: 38 in |
| | US20150159173) |
| AAVhu.48 (See SEQ ID NO: 157 in US20150315612) | AAVhu.48 (AAV130.4) (See SEQ ID NO: 78 in |
| | US20150315612) |
| AAVhu.48Rl (See SEQ ID NO: in US20150159173 | AAVhu.48R2 (See SEQ ID NO: in |
| | US20150159173 |
| AAVhu.48R3 (See SEQ ID NO: in US20150159173 | AAVhu.49 (See SEQ ID NO: 209 in |
| | US20150315612) |
| AAVhu.49 (See SEQ ID NO: 189 in US20150315612) | AAVhu.5 (See SEQ ID NO: 45 in |
| | US20150315612) |
| AAVhu.5 (See SEQ ID NO: 142 in US20150315612) | AAVhu.51 (See SEQ ID NO: 208 in |
| | US20150315612) |
| AAVhu.51 (See SEQ ID NO: 190 in US20150315612) | AAVhu.52 (See SEQ ID NO: 210 in |
| | US20150315612) |
| AAVhu.52 (See SEQ ID NO: 191 in US20150315612) | AAVhu.53 (See SEQ ID NO: 19 in |
| | US20150159173) |
| AAVhu.53 (See SEQ ID NO: 35 in US20150159173) | AAVhu.53 (AAV145.1) (See SEQ ID NO: 176 in |
| | US20150315612) |
| AAVhu.54 (See SEQ ID NO: 188 in US20150315612) | AAVhu.54 (AAV145.5) (See SEQ ID NO: 177 in |
| | US20150315612) |
| AAVhu.55 (See SEQ ID NO: 187 in US20150315612) | AAVhu.56 (See SEQ ID NO: 205 in |
| | US20150315612) |
| AAVhu.56 (AAV145.6) (See SEQ ID NO: 168 in | AAVhu.56 (AAV145.6) (See SEQ ID NO: 192 in |
| US20150315612) | US20150315612) |
| AAVhu.57 (See SEQ ID NO: 206 in US20150315612) | AAVhu.57 (See SEQ ID NO: 169 in |
| | US20150315612) |
| AAVhu.57 (See SEQ ID NO: 193 in US20150315612) | AAVhu.58 (See SEQ ID NO: 207 in |
| | US20150315612) |
| AAVhu.58 (See SEQ ID NO: 194 in US20150315612) | AAVhu.6 (AAV3.1) (See SEQ ID NO: 5 in |
| | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.61 (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in US20150315612) | AAVhu.64 (See SEQ ID NO: 212 in |
| | US20150315612) |
| AAVhu.64 (See SEQ ID NO: 196 in US20150315612) | AAVhu.66 (See SEQ ID NO: 197 in |
| | US20150315612) |
| AAVhu.67 (See SEQ ID NO: 215 in US20150315612) | AAVhu.67 (See SEQ ID NO: 198 in |
| | US20150315612) |
| AAVhu.7 (See SEQ ID NO: 226 in US20150315612) | AAVhu.7 (See SEQ ID NO: 150 in |
| | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in US20150315612) | AAVhu.8 (See SEQ ID NO: 12 in |
| | US20150315612) |
| AAVhu.8 (See SEQ ID NO: 151 in US20150315612) | AAVhu.9 (AAV3.1) (See SEQ ID NO: 58 in |
| | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in US20150376607) | AAV-LK02 (See SEQ ID NO: 3 in |
| | US20150376607) |
| AAV-LK02 (See SEQ ID NO: 30 in US20150376607) | AAV-LK03 (See SEQ ID NO: 4 in |
| | US20150376607) |
| AAV-LK03 (See SEQ ID NO: 12 in WO2015121501 | |
| and SEQ ID NO: 31 in US20150376607) | |
| AAV-LK04 (See SEQ ID NO: 5 in US20150376607) | AAV-LK04 (See SEQ ID NO: 32 in |
| | US20150376607) |
| AAV-LK05 (See SEQ ID NO: 6 in US20150376607) | AAV-LK05 (See SEQ ID NO: 33 in |
| | US20150376607) |
| AAV-LK06 (See SEQ ID NO: 7 in US20150376607) | AAV-LK06 (See SEQ ID NO: 34 in |
| | US20150376607) |
| AAV-LK07 (See SEQ ID NO: 8 in US20150376607) | AAV-LK07 (See SEQ ID NO: 35 in |
| | US20150376607) |
| AAV-LK08 (See SEQ ID NO: 9 in US20150376607) | AAV-LK08 (See SEQ ID NO: 36 in |
| | US20150376607) |
| AAV-LK09 (See SEQ ID NO: 10 in US20150376607) | AAV-LK09 (See SEQ ID NO: 37 in |
| | US20150376607) |
| AAV-LK10 (See SEQ ID NO: 11 in US20150376607) | AAV-LK10 (See SEQ ID NO: 38 in |
| | US20150376607) |
| AAV-LK11 (See SEQ ID NO: 12 in US20150376607) | AAV-LK11 (See SEQ ID NO: 39 in |
| | US20150376607) |
| AAV-LK12 (See SEQ ID NO: 13 in US20150376607) | AAV-LK12 (See SEQ ID NO: 40 in |
| | US20150376607) |
| AAV-LK13 (See SEQ ID NO: 14 in US20150376607) | AAV-LK13 (See SEQ ID NO: 41 in |
| | US20150376607) |
| AAV-LK14 (See SEQ ID NO: 15 in US20150376607) | AAV-LK14 (See SEQ ID NO: 42 in |
| | US20150376607) |
| AAV-LK15 (See SEQ ID NO: 16 in US20150376607) | AAV-LK15 (See SEQ ID NO: 43 in |
| | US20150376607) |
| AAV-LK16 (See SEQ ID NO: 17 in US20150376607) | AAV-LK16 (See SEQ ID NO: 44 in |
| | US20150376607) |
| AAV-LK17 (See SEQ ID NO: 18 in US20150376607) | AAV-LK17 (See SEQ ID NO: 45 in |
| | US20150376607) |
| AAV-LK18 (See SEQ ID NO: 19 in US20150376607) | AAV-LK18 (See SEQ ID NO: 46 in |
| | US20150376607) |
| AAV-LK19 (See SEQ ID NO: 20 in US20150376607) | AAV-LK19 (See SEQ ID NO: 47 in |
| | US20150376607) |
| AAV-PAEC (See SEQ ID NO: 1 in US20150376607) | AAV-PAEC (See SEQ ID NO: 48 in |
| | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in US20150376607) | AAV-PAEC2 (See SEQ ID NO: 56 in |
| | US20150376607) |
| AAV-PAEC4 (See SEQ ID NO: 22 in US20150376607) | AAV-PAEC4 (See SEQ ID NO: 55 in |
| | US20150376607) |
| AAV-PAEC6 (See SEQ ID NO: 23 in US20150376607) | AAV-PAEC6 (See SEQ ID NO: 52 in |
| | US20150376607) |
| AAV-PAEC7 (See SEQ ID NO: 24 in US20150376607) | AAV-PAEC7 (See SEQ ID NO: 53 in |
| | US20150376607) |
| AAV-PAEC8 (See SEQ ID NO: 25 in US20150376607) | AAV-PAEC8 (See SEQ ID NO: 50 in |
| | US20150376607) |
| AAVpi.l (See SEQ ID NO: 28 in US20150315612) | AAVpi.l (See SEQ ID NO: 93 in US20150315612; |
| | AAVpi.2 408, see SEQ ID NO: 30 in |
| | US20150315612) |
| AAVpi.2 (See SEQ ID NO: 95 in US20150315612) | AAVpi.3 (See SEQ ID NO: 29 in US20150315612) |
| AAVpi.3 (See SEQ ID NO: 94 in US20150315612) | AAVrh.10 (See SEQ ID NO: 9 in US20150159173) |
| AAVrh.10 (See SEQ ID NO: 25 in US20150159173) | AAV44.2 (See SEQ ID NO: 59 in |
| | US20030138772) |
| AAVrh.10 (AAV44.2) (See SEQ ID NO: 81 in | AAV42.1B (See SEQ ID NO: 90 in |
| US20030138772) | US20030138772) |
| AAVrh.l2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in US20150159173) | AAVrh.13 (See SEQ ID NO: 228 in |
| | US20150315612) |
| AAVrh.l3R (See SEQ ID NO: in US20150159173 | AAV42.3A (See SEQ ID NO: 87 in |
| | US20030138772) |
| AAVrh.l4 (AAV42.3a) (See SEQ ID NO: 32 in | AAV42.5A (See SEQ ID NO: 89 in |
| US20030138772) | US20030138772) |
| AAVrh.l7 (AAV42.5a) (See SEQ ID NO: 34 in | AAV42.5B (See SEQ ID NO: 91 in |
| US20030138772) | US20030138772) |
| AAVrh.l8 (AAV42.5b) (See SEQ ID NO: 29 in | AAV42.6B (See SEQ ID NO: 112 in |
| US20030138772) | US20030138772) |
| AAVrh.l9 (AAV42.6b) (See SEQ ID NO: 38 in | AAVrh.2 (See SEQ ID NO: 39 in US20150159173) |
| US20030138772) | |
| AAVrh.2 (See SEQ ID NO: 231 in US20150315612) | AAVrh.20 (See SEQ ID NO: 1 in US20150159173) |
| AAV42.10 (See SEQ ID NO: 106 in US20030138772) | AAVrh.21 (AAV42.10) (See SEQ ID NO: 35 in |
| | US20030138772) |
| AAV42.11 (See SEQ ID NO: 108 in US20030138772) | AAVrh.22 (AAV42.11) (See SEQ ID NO: 37 in |
| | US20030138772) |
| AAV42.12 (See SEQ ID NO: 113 in US20030138772) | AAVrh.23 (AAV42.12) (See SEQ ID NO: 58 in |
| | US20030138772) |
| AAV42.13 (See SEQ ID NO: 86 in US20030138772) | AAVrh.24 (AAV42.13) (See SEQ ID NO: 31 in |
| | US20030138772) |
| AAV42.15 (See SEQ ID NO: 84 in US20030138772) | AAVrh.25 (AAV42.15) (See SEQ ID NO: 28 in |
| | US20030138772) |
| AAVrh.2R (See SEQ ID NO: in US20150159173 | AAVrh.31 (AAV223.1) (See SEQ ID NO: 48 in |
| | US20030138772) |
| AAVC1 (See SEQ ID NO: 60 in US20030138772) | AAVrh.32 (AAVC1) (See SEQ ID NO: 19 in 446 |
| | US20030138772) |
| AAVrh.32/33 (See SEQ ID NO: 2 in US20150159173) | AAVrh.51 (AAV2-5) (See SEQ ID NO: 104 in |
| | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in US20150159173 and | |
| SEQ ID NO: 116 in US20150315612) | |
| AAVrh.55 (See SEQ ID NO: 37 in US20150315612) | AAVrh.55 (AAV4-19) (See SEQ ID NO: 117 in |
| | US20150315612) |
| AAVrh.56 (See SEQ ID NO: 54 in US20150315612) | AAVrh.56 (See SEQ ID NO: 152 in |
| | US20150315612) |
| AAVrh.57 (See SEQ ID NO: in 497 US20150315612 | AAVrh.57 (See SEQ ID NO: 105 in |
| SEQ ID NO: 26 | US20150315612) |
| AAVrh.58 (See SEQ ID NO: 27 in US20150315612) | AAVrh.58 (See SEQ ID NO: 48 in |
| | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in US20150315612) | AAVrh.59 (See SEQ ID NO: 110 in |
| | US20150315612) |
| AAVrh.60 (See SEQ ID NO: 31 in US20150315612) | AAVrh.60 (See SEQ ID NO: 120 in |
| | US20150315612) |
| AAVrh.61 (See SEQ ID NO: 107 in US20150315612) | AAVrh.61 (AAV2-3) (See SEQ ID NO: 21 in |
| | US20150315612) |
| AAVrh.62 (AAV2-15) (See SEQ ID NO: 33 in | AAVrh.62 (AAV2-15) (See SEQ ID NO: 114 in |
| US20150315612) | US20150315612) |
| AAVrh.64 (See SEQ ID NO: 15 in US20150315612) | AAVrh.64 (See SEQ ID NO: 43 in |
| | US20150159173 and SEQ ID NO: 99 in |
| | US20150315612) |
| | AAVrh.64 (See SEQ ID NO: 233 in |
| | US20150315612) |
| AAVRh.64R1 (See SEQ ID NO: in US20150159173 | AAVRh.64R2 (See SEQ ID NO: in |
| | US20150159173 |
| AAVrh.65 (See SEQ ID NO: 35 in US20150315612) | AAVrh.65 (See SEQ ID NO: 112 in |
| | US20150315612) |
| AAVrh.67 (See SEQ ID NO: 36 in US20150315612) | AAVrh.67 (See SEQ ID NO: 230 in |
| | US20150315612) |
| AAVrh.67 (See SEQ ID NO: 47 in US20150159173 and | |
| SEQ ID NO: 47 in US20150315612) | |
| AAVrh.68 (See SEQ ID NO: 16 in US20150315612) | AAVrh.68 (See SEQ ID NO: 100 in |
| | US20150315612) |
| AAVrh.69 (See SEQ ID NO: 39 in US20150315612) | AAVrh.69 (See SEQ ID NO: 119 in |
| | US20150315612) |
| AAVrh.70 (See SEQ ID NO: 20 in US20150315612) | AAVrh.70 (See SEQ ID NO: 98 in |
| | US20150315612) |
| AAVrh.71 (See SEQ ID NO: 162 in US20150315612) | AAVrh.72 (See SEQ ID NO: 9 in US20150315612) |
| AAVrh.73 (See SEQ ID NO: 5 in US20150159173) | AAVrh.74 (See SEQ ID NO: 6 in US20150159173) |
| AAVrh.8 (See SEQ ID NO: 41 in US20150159173) | AAVrh.8 (See SEQ ID NO: 235 in |
| | US20150315612) |
| AAVrh.8R (See SEQ ID NO: 9 in US20150159173, | AAVrh.8R A586R mutant (See SEQ ID NO: 10 in |
| WO2015168666) | WO2015168666) |
| AAVrh.8R R533A mutant (See SEQ ID NO: 11 in | BAAV (bovine AAV) (See SEQ ID NO: 8 in |
| WO2015168666) | U.S. Pat. No. 9,193,769) |
| BAAV (bovine AAV) (See SEQ ID NO: 10 in | BAAV (bovine AAV) (See SEQ ID NO: 4 in |
| U.S. Pat. No. 9,193,769) | U.S. Pat. No. 9,193,769) |
| BAAV (bovine AAV) (See SEQ ID NO: 2 in | BAAV (bovine AAV) (See SEQ ID NO: 6 in |
| U.S. Pat. No. 9,193,769) | U.S. Pat. No. 9,193,769) |
| BAAV (bovine AAV) (See SEQ ID NO: 1 in | BAAV (bovine AAV) (See SEQ ID NO: 5 in |
| U.S. Pat. No. 9,193,769) | U.S. Pat. No. 9,193,769) |
| BAAV (bovine AAV) (See SEQ ID NO: 3 in | BAAV (bovine AAV) (See SEQ ID NO: 11 in |
| U.S. Pat. No. 9,193,769) | U.S. Pat. No. 9,193,769) |
| BAAV (bovine AAV) (See SEQ ID NO: 5 in | BAAV (bovine AAV) (See SEQ ID NO: 6 in |
| U.S. Pat. No. 7,427,396) | U.S. Pat. No. 7,427,396) |
| BAAV (bovine AAV) (See SEQ ID NO: 7 in | BAAV (bovine AAV) (See SEQ ID NO: 9 in |
| U.S. Pat. No. 9,193,769) | U.S. Pat. No. 9,193,769) |
| BNP61 AAV (See SEQ ID NO: 1 in US20150238550) | BNP61 AAV (See SEQ ID NO: 2 in |
| | US20150238550) |
| BNP62 AAV (See SEQ ID NO: 3 in US20150238550) | BNP63 AAV (See SEQ ID NO: 4 in |
| | US20150238550) |
| caprine AAV (See SEQ ID NO: 3 in U.S. Pat. No. 7,427,396) | caprine AAV (See SEQ ID NO: 4 in U.S. Pat. No. 7,427,396) |
| true type AAV (ttAAV) (See SEQ ID NO: 2 in | AAAV (Avian AAV) (See SEQ ID NO: 12 in |
| WO2015121501) | U.S. Pat. No. 9,238,800) |
| AAAV (Avian AAV) (See SEQ ID NO: 2 in | AAAV (Avian AAV) (See SEQ ID NO: 6 in |
| U.S. Pat. No. 9,238,800) | U.S. Pat. No. 9,238,800) |
| AAAV (Avian AAV) (See SEQ ID NO: 4 in | AAAV (Avian AAV) (See SEQ ID NO: 8 in |
| U.S. Pat. No. 9,238,800) | U.S. Pat. No. 9,238,800) |
| AAAV (Avian AAV) (See SEQ ID NO: 14 in | AAAV (Avian AAV) (See SEQ ID NO: 10 in |
| U.S. Pat. No. 9,238,800) | U.S. Pat. No. 9,238,800) |
| AAAV (Avian AAV) (See SEQ ID NO: 15 in | AAAV (Avian AAV) (See SEQ ID NO: 5 in |
| U.S. Pat. No. 9,238,800) | U.S. Pat. No. 9,238,800) |
| AAAV (Avian AAV) (See SEQ ID NO: 9 in | AAAV (Avian AAV) (See SEQ ID NO: 3 in |
| U.S. Pat. No. 9,238,800) | U.S. Pat. No. 9,238,800) |
| AAAV (Avian AAV) (See SEQ ID NO: 7 in | AAAV (Avian AAV) (See SEQ ID NO: 11 in |
| U.S. Pat. No. 9,238,800) | U.S. Pat. No. 9,238,800) |
| AAAV (Avian AAV) (See SEQ ID NO: in | AAAV (Avian AAV) (See SEQ ID NO: 1 in |
| U.S. Pat. No. 9,238,800) | U.S. Pat. No. 9,238,800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in WO2016065001) | AAV CBr-7.1 (See SEQ ID NO: 54 in |
| | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in WO2016065001) | AAV CBr-7.2 (See SEQ ID NO: 55 in |
| | WO2016065001) |
| AAV CBr-7.3 (See SEQ ID NO: 6 in WO2016065001) | AAV CBr-7.3 (See SEQ ID NO: 56 in |
| | WO2016065001) |
| AAV CBr-7.4 (See SEQ ID NO: 7 in WO2016065001) | AAV CBr-7.4 (See SEQ ID NO: 57 in |
| | WO2016065001) |
| AAV CBr-7.5 (See SEQ ID NO: 8 in WO2016065001) | AAV CHt-6.6 (See SEQ ID NO: 35 in |
| | WO2016065001) |
| AAV CHt-6.6 (See SEQ ID NO: 85 in WO2016065001) | AAV CHt-6.7 (See SEQ ID NO: 36 in |
| | WO2016065001) |
| AAV CHt-6.7 (See SEQ ID NO: 86 in WO2016065001) | AAV CHt-6.8 (See SEQ ID NO: 37 in |
| | WO2016065001) |
| AAV CHt-6.8 (See SEQ ID NO: 87 in WO2016065001) | AAV CHt-Pl (See SEQ ID NO: 29 in |
| | WO2016065001) |
| AAV CHt-Pl (See SEQ ID NO: 79 in WO2016065001) | AAV CHt-P2 (See SEQ ID NO: 1 in |
| | WO2016065001) |
| AAV CHt-P2 (See SEQ ID NO: 51 in WO2016065001) | AAV CHt-P5 (See SEQ ID NO: 2 in |
| | WO2016065001) |
| AAV CHt-P5 (See SEQ ID NO: 52 in WO2016065001) | AAV CHt-P6 (See SEQ ID NO: 30 in |
| | WO2016065001) |
| AAV CHt-P6 (See SEQ ID NO: 80 in WO2016065001) | AAV CHt-P8 (See SEQ ID NO: 31 in |
| | WO2016065001) |
| AAV CHt-P8 (See SEQ ID NO: 81 in WO2016065001) | AAV CHt-P9 (See SEQ ID NO: 3 in |
| | WO2016065001) |
| AAV CHt-P9 (See SEQ ID NO: 53 in WO2016065001) | AAV CKd-1 (See SEQ ID NO: 57 in U.S. Pat. No. 8,734,809) |
| AAV CKd-1 (See SEQ ID NO: 131 in U.S. Pat. No. 8,734,809) | AAV CKd-10 (See SEQ ID NO: 58 in U.S. Pat. No. 8,734,809) |
| AAV CKd-10 (See SEQ ID NO: 132 in U.S. Pat. No. 8,734,809) | AAV CKd-2 (See SEQ ID NO: 59 in U.S. Pat. No. 8,734,809) |
| AAV CKd-2 (See SEQ ID NO: 133 in U.S. Pat. No. 8,734,809) | AAV CKd-3 (See SEQ ID NO: 60 in U.S. Pat. No. 8,734,809) |
| AAV CKd-3 (See SEQ ID NO: 134 in U.S. Pat. No. 8,734,809) | AAV CKd-4 (See SEQ ID NO: 61 in U.S. Pat. No. 8,734,809) |
| AAV CKd-4 (See SEQ ID NO: 135 in U.S. Pat. No. 8,734,809) | AAV CKd-6 (See SEQ ID NO: 62 in U.S. Pat. No. 8,734,809) |
| AAV CKd-6 (See SEQ ID NO: 136 in U.S. Pat. No. 8,734,809) | AAV CKd-7 (See SEQ ID NO: 63 in U.S. Pat. No. 8,734,809) |
| AAV CKd-7 (See SEQ ID NO: 137 in U.S. Pat. No. 8,734,809) | AAV CKd-8 (See SEQ ID NO: 64 in U.S. Pat. No. 8,734,809) |
| AAV CKd-8 (See SEQ ID NO: 138 in U.S. Pat. No. 8,734,809) | AAV CKd-B 1 (See SEQ ID NO: 73 in |
| | U.S. Pat. No. 8,734,809) |
| AAV CKd-B 1 (See SEQ ID NO: 147 in U.S. Pat. No. 8,734,809) | AAV CKd-B2 (See SEQ ID NO: 74 in U.S. Pat. No. 8,734,809) |
| AAV CKd-B2 (See SEQ ID NO: 148 in U.S. Pat. No. 8,734,809) | AAV CKd-B3 (See SEQ ID NO: 75 in U.S. Pat. No. 8,734,809) |
| AAV CKd-B3 (See SEQ ID NO: in U.S. Pat. No. 8,734,809 | AAV CKd-B3 (See SEQ ID NO: 149 in |
| | U.S. Pat. No. 8,734,809) |
| AAV CLv-1 (See SEQ ID NO: 65 in U.S. Pat. No. 8,734,809) | AAV CLv-1 (See SEQ ID NO: 139 in U.S. Pat. No. 8,734,809) |
| AAV CLvl-1 (See SEQ ID NO: 171 in U.S. Pat. No. 8,734,809) | AAV Civ 1-10 (See SEQ ID NO: 178 in |
| | U.S. Pat. No. 8,734,809) |
| AAV CLvl-2 (See SEQ ID NO: 172 in U.S. Pat. No. 8,734,809) | AAV CLv-12 (See SEQ ID NO: 66 in U.S. Pat. No. 8,734,809) |
| AAV CLv-12 (See SEQ ID NO: 140 in U.S. Pat. No. 8,734,809) | AAV CLvl-3 (See SEQ ID NO: 173 in U.S. Pat. No. 8,734,809) |
| AAV CLv-13 (See SEQ ID NO: 67 in U.S. Pat. No. 8,734,809) | AAV CLv-13 (See SEQ ID NO: 141 in |
| | U.S. Pat. No. 8,734,809) |
| AAV CLvl-4 (See SEQ ID NO: 174 in U.S. Pat. No. 8,734,809) | AAV Civ 1-7 (See SEQ ID NO: 175 in |
| | U.S. Pat. No. 8,734,809) |
| AAV Civ 1-8 (See SEQ ID NO: 176 in U.S. Pat. No. 8,734,809) | AAV Civ 1-9 (See SEQ ID NO: 177 in |
| | U.S. Pat. No. 8,734,809) |
| AAV CLv-2 (See SEQ ID NO: 68 in U.S. Pat. No. 8,734,809) | AAV CLv-2 (See SEQ ID NO: 142 in U.S. Pat. No. 8,734,809) |
| AAV CLv-3 (See SEQ ID NO: 69 in U.S. Pat. No. 8,734,809) | AAV CLv-3 (See SEQ ID NO: 143 in U.S. Pat. No. 8,734,809) |
| AAV CLv-4 (See SEQ ID NO: 70 in U.S. Pat. No. 8,734,809) | AAV CLv-4 (See SEQ ID NO: 144 in U.S. Pat. No. 8,734,809) |
| AAV CLv-6 (See SEQ ID NO: 71 in U.S. Pat. No. 8,734,809) | AAV CLv-6 (See SEQ ID NO: 145 in U.S. Pat. No. 8,734,809) |
| AAV CLv-8 (See SEQ ID NO: 72 in U.S. Pat. No. 8,734,809) | AAV CLv-8 (See SEQ ID NO: 146 in U.S. Pat. No. 8,734,809) |
| AAV CLv-Dl (See SEQ ID NO: 22 in U.S. Pat. No. 8,734,809) | AAV CLv-Dl (See SEQ ID NO: 96 in U.S. Pat. No. 8,734,809) |
| AAV CLv-D2 (See SEQ ID NO: 23 in U.S. Pat. No. 8,734,809) | AAV CLv-D2 (See SEQ ID NO: 97 in U.S. Pat. No. 8,734,809) |
| AAV CLv-D3 (See SEQ ID NO: 24 in U.S. Pat. No. 8,734,809) | AAV CLv-D3 (See SEQ ID NO: 98 in U.S. Pat. No. 8,734,809) |
| AAV CLv-D4 (See SEQ ID NO: 25 in U.S. Pat. No. 8,734,809) | AAV CLv-D4 (See SEQ ID NO: 99 in U.S. Pat. No. 8,734,809) |
| AAV CLv-D5 (See SEQ ID NO: 26 in U.S. Pat. No. 8,734,809) | AAV CLv-D5 (See SEQ ID NO: 100 in |
| | U.S. Pat. No. 8,734,809) |
| AAV CLv-D6 (See SEQ ID NO: 27 in U.S. Pat. No. 8,734,809) | AAV CLv-D6 (See SEQ ID NO: 101 in |
| | U.S. Pat. No. 8,734,809) |
| AAV CLv-D7 (See SEQ ID NO: 28 in U.S. Pat. No. 8,734,809) | AAV CLv-D7 (See SEQ ID NO: 102 in |
| | U.S. Pat. No. 8,734,809) |
| AAV CLv-D8 (See SEQ ID NO: 29 in U.S. Pat. No. 8,734,809) | AAV CLv-D8 (See SEQ ID NO: 103 in |
| | U.S. Pat. No. 8,734,809); AAV CLv-K1 762, see SEQ ID NO: 18 |
| | in WO2016065001) |
| AAV CLv-Kl (See SEQ ID NO: 68 in WO2016065001) | AAV CLv-K3 (See SEQ ID NO: 19 in |
| | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in WO2016065001) | AAV CLv-L5 (See SEQ ID NO: 16 in |
| | WO2016065001) |
| AAV CLv-L5 (See SEQ ID NO: 66 in WO2016065001) | AAV CLv-L6 (See SEQ ID NO: 17 in |
| | WO2016065001) |
| AAV CLv-L6 (See SEQ ID NO: 67 in WO2016065001) | AAV CLv-Ml (See SEQ ID NO: 21 in |
| | WO2016065001) |
| AAV CLv-Ml (See SEQ ID NO: 71 in WO2016065001) | AAV CLv-Mll (See SEQ ID NO: 22 in |
| | WO2016065001) |
| AAV CLv-Ml 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-Rl (See SEQ ID NO: 30 in U.S. Pat. No. 8,734,809) |
| WO2016065001) | |
| AAV CLv-Rl (See SEQ ID NO: 104 in U.S. Pat. No. 8,734,809) | AAV CLv-R2 (See SEQ ID NO: 31 in U.S. Pat. No. 8,734,809) |
| AAV CLv-R2 (See SEQ ID NO: 105 in U.S. Pat. No. 8,734,809) | AAV CLv-R3 (See SEQ ID NO: 32 in U.S. Pat. No. 8,734,809) |
| AAV CLv-R3 (See SEQ ID NO: 106 in U.S. Pat. No. 8,734,809) | AAV CLv-R4 (See SEQ ID NO: 33 in U.S. Pat. No. 8,734,809) |
| AAV CLv-R4 (See SEQ ID NO: 107 in U.S. Pat. No. 8,734,809) | AAV CLv-R5 (See SEQ ID NO: 34 in U.S. Pat. No. 8,734,809) |
| AAV CLv-R5 (See SEQ ID NO: 108 in U.S. Pat. No. 8,734,809) | AAV CLv-R6 (See SEQ ID NO: 35 in U.S. Pat. No. 8,734,809) |
| AAV CLv-R6 (See SEQ ID NO: 109 in U.S. Pat. No. 8,734,809); | AAV CLv-R7 (See SEQ ID NO: 110 in |
| AAV CLv-R7 802 (see SEQ ID NO: 36 in U.S. Pat. No. 8,734,809) | U.S. Pat. No. 8,734,809) |
| AAV CLv-R8 (See SEQ ID NO: 37 in U.S. Pat. No. 8,734,809) | AAV CLv-R8 (See SEQ ID NO: 111 in |
| | U.S. Pat. No. 8,734,809) |
| AAV CLv-R9 (See SEQ ID NO: 38 in U.S. Pat. No. 8,734,809) | AAV CLv-R9 (See SEQ ID NO: 112 in |
| | U.S. Pat. No. 8,734,809) |
| AAV CSp-1 (See SEQ ID NO: 45 in U.S. Pat. No. 8,734,809) | AAV CSp-1 (See SEQ ID NO: 119 in U.S. Pat. No. 8,734,809) |
| AAV CSp-10 (See SEQ ID NO: 46 in U.S. Pat. No. 8,734,809) | AAV CSp-10 (See SEQ ID NO: 120 in |
| | U.S. Pat. No. 8,734,809) |
| AAV CSp-11 (See SEQ ID NO: 47 in U.S. Pat. No. 8,734,809) | AAV CSp-11 (See SEQ ID NO: 121 in |
| | U.S. Pat. No. 8,734,809) |
| AAV CSp-2 (See SEQ ID NO: 48 in U.S. Pat. No. 8,734,809) | AAV CSp-2 (See SEQ ID NO: 122 in U.S. Pat. No. 8,734,809) |
| AAV CSp-3 (See SEQ ID NO: 49 in U.S. Pat. No. 8,734,809) | AAV CSp-3 (See SEQ ID NO: 123 in U.S. Pat. No. 8,734,809) |
| AAV CSp-4 (See SEQ ID NO: 50 in U.S. Pat. No. 8,734,809) | AAV CSp-4 (See SEQ ID NO: 124 in U.S. Pat. No. 8,734,809) |
| AAV CSp-6 (See SEQ ID NO: 51 in U.S. Pat. No. 8,734,809) | AAV CSp-6 (See SEQ ID NO: 125 in U.S. Pat. No. 8,734,809) |
| AAV CSp-7 (See SEQ ID NO: 52 in U.S. Pat. No. 8,734,809) | AAV CSp-7 (See SEQ ID NO: 126 in U.S. Pat. No. 8,734,809) |
| AAV CSp-8 (See SEQ ID NO: 53 in U.S. Pat. No. 8,734,809) | AAV CSp-8 (See SEQ ID NO: 127 in U.S. Pat. No. 8,734,809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in WO2016065001) | AAV CSp-8.2 (See SEQ ID NO: 89 in |
| | WO2016065001) |
| AAV CSp-8.4 (See SEQ ID NO: 40 in WO2016065001) | AAV CSp-8.4 (See SEQ ID NO: 90 in |
| | WO2016065001) |
| AAV CSp-8.5 (See SEQ ID NO: 41 in WO2016065001) | AAV CSp-8.5 (See SEQ ID NO: 91 in |
| | WO2016065001) |
| AAV CSp-8.6 (See SEQ ID NO: 42 in WO2016065001) | AAV CSp-8.6 (See SEQ ID NO: 92 in |
| | WO2016065001) |
| AAV CSp-8.7 (See SEQ ID NO: 43 in WO2016065001) | AAV CSp-8.7 (See SEQ ID NO: 93 in |
| | WO2016065001) |
| AAV CSp-8.8 (See SEQ ID NO: 44 in WO2016065001) | AAV CSp-8.8 (See SEQ ID NO: 94 in |
| | WO2016065001) |
| AAV CSp-8.9 (See SEQ ID NO: 45 in WO2016065001) | AAV CSp-8.9 (See SEQ ID NO: 95 in |
| | WO2016065001) |
| AAV CSp-9 842 (See SEQ ID NO: 54 in U.S. Pat. No. 8,734,809) | AAV CSp-9 (See SEQ ID NO: 128 in U.S. Pat. No. 8,734,809) |
| AAV.hu.48R3 (See SEQ ID NO: 183 in U.S. Pat. No. 8,734,809) | AAV.VR-355 (See SEQ ID NO: 181 in |
| | U.S. Pat. No. 8,734,809) |
| AAV3B (See SEQ ID NO: 48 in WO2016065001) | AAV3B (See SEQ ID NO: 98 in WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

The components to be cultured in the host cell to package a rAAV vector in an AAV capsid may be provided to the host cell in trans. Alternatively, any one or more of the required components {e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art. In some embodiments, the instant disclosure relates to a host cell containing a nucleic acid that comprises a coding sequence encoding a protein (e.g., wild-type huntingtin protein, optionally “hardened” wild-type huntingtin protein). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13: 197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium orjust a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., 0-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

In some embodiments, useful vectors are contemplated to be those vectors in which the nucleic acid segment to be transcribed is positioned under the transcriptional control of a promoter. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

A “promoter” refers to a DNA sequence recognized by the synthetic machinery of the cell, or introduced synthetic machinery, required to initiate the specific transcription of a gene. When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The foregoing methods for packaging recombinant vectors in desired AAV capsids to produce the rAAVs of the disclosure are not meant to be limiting and other suitable methods will be apparent to the skilled artisan.

In some embodiments, any one or more thymidine (T) nucleotides or uridine (U) nucleotides in a sequence provided herein, including a sequence provided in the sequence listing, may be replaced with any other nucleotide suitable for base pairing (e.g., via a Watson-Crick base pair) with an adenosine nucleotide. For example, in some embodiments, any one or more thymidine (T) nucleotides in a sequence provided herein, including a sequence provided in the sequence listing, may be suitably replaced with a uridine (U) nucleotide or vice versa.

In some embodiments of any of the aspects, a nucleic acid (e.g., miRNA) is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2—NH—CH2—, —CH2—N(CH3)—O—CH2—[known as a methylene (methylimino) or MMI backbone], —CH2—O—N(CH3)—CH2—, —CH2—N(CH3)—N(CH3)—CH2—and —N(CH3)—CH2—CH2—[wherein the native phosphodiester backbone is represented as —O—P—O—CH2—].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol. Canc. Ther. 6(3):833-843; Grunweller, A. et al., (2003) Nucleic Acids Research 31(12):3185-3193).

Modified nucleic acids can also contain one or more substituted sugar moieties. The nucleic acids described herein can include one of the following at the 2′ position: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2)nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, nucleic acids include one of the following at the 2′ position: C1 to C10 lower alkyl, substituted lower alkyl, alkaryl, aralkyl, O-alkaryl or O-aralkyl, SH, SCH₃, OCN, C1, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments of any of the aspects, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2—O—CH2—N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases can include other synthetic and natural nucleobases including but not limited to as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc.2008, 130, 7, 2336-2343; Malyshev et. al. PNAS. 2012. 109 (30) 12005-12010). In some embodiments of any of the aspects, oligonucleotide tags (e.g., Oligopaint) comprise any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of any of the aspects, the vector is pEMBL. In some embodiments of any of the aspects, the vector is pEMBL-D(+)Syn1. In some embodiments of any of the aspects, the vector is pEMBL-D(+)Syn1-hCG intron only. In some embodiments of any of the aspects, the vector is pEMBL-D(+)Syn1-hCGin-2× control pre-miR. In some embodiments of any of the aspects, the vector is pEMBL-D(+)Syn1-hCGin-2× artificial pre-miR. In some embodiments of any of the aspects, the vector is pEMBL-D(+)Syn1-CYP46A1-hCGin-2× artificial pre-miR. In some embodiments of any of the aspects, the vector is pEMBL-D(+)Syn1-luc-HTT-3′UTR/mutant. In some embodiments of any of the aspects, the vector comprises at least one of the following: at least one (e.g., 2) ITRs; Syn1 promoter; at least one (e.g., 2) hCG intron; at least one (e.g., 2) copy of a premiR (e.g., control pre-miR; artificial pre-miR; SEQ ID NO: 6-17, 40-44, or 50-66); small polyA; CYP46A1; luciferase; HTT targeting sequences; and/or HTT-3′UTR/mutant. In some embodiments, the vector comprises a neuron specific synthetic promoter selected from Tables 10-13, and/or a CRE selected from Tables 13-15. In certain aspects of embodiments, the miRNA targets wild type HTT allele. In other aspects of the embodiments, the miRNA targets mutant HTT allele. In yet another embodiment, the miRNA targets both wild type and mutant HTT alleles. In yet another embodiment, the miRNA targets any HTT mRNA.

In some embodiments, one or more of the recombinantly expressed gene can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments, the promoter is a synapsin (Syn1) promoter (see e.g., SEQ ID NO: 152). In one aspect, the promoter comprises a nucleic acid sequence at least 80% identical, e.g., at least 85%, at least 90%, at least 95%, at least 98%, at least 99% or 100% identical, to SEQ ID NO: 152. In one aspect, provided herein is a composition comprising a recombinant viral vector comprising a promoter comprising a nucleic acid sequence at least 80% identical, e.g., at least 85%, at least 90%, at least 95%, at least 98%, at least 99% or 100% identical, to SEQ ID NO: 152.

Synapsin-1
(SEQ ID NO: 152)
GAGGGCCCTGCGTATGAGTGCAAGTGGGTTTTAGGACCAGGATGAGGCG
GGGTGGGGGTGCCTACCTGACGACCGACCCCGACCCACTGGACAAGCAC
CCAACCCCCATTCCCCAAATTGCGCATCCCCTATCAGAGAGGGGGAGGG
GAAACAGGATGCGGCGAGGCGCGTGCGCACTGCCAGCTTCAGCACCGCG
GACAGTGCCTTCGCCCCCGCCTGGCGGCGCGCGCCACCGCCGCCTCAGC
ACTGAAGGCGCGCTGACGTCACTCGCCGGTCCCCCGCAAACTCCCCTTC
CCGGCCACCTTGGTCGCGTCCGCGCCGCCGCCGGCCCAGCCGGACCGCA
CCACGCGAGGCGCGAGATAGGGGGGCACGGGCGCGACCATCTGCGCTGC
GGCGCCGGCGACTCAGCGCTGCCTCAGTCTGCGGTGGGCAGCGGAGGAG
TCGTGTCGTGCCTGAGAGCGCAGTCG

In one aspect, provided herein is a composition comprising an isolated nucleic acid PGP-comprising a sequence at least 80% identical, e.g., at least 85%, at least 90%, at least 95%, at least 98%, at least 99% or 100% identical, to SEQ ID NO: 111. In one aspect, provided herein is a composition comprising a recombinant viral vector comprising an isolated nucleic acid comprising a sequence at least 80% identical, e.g., at least 85%, at least 90%, at least 95%, at least 98%, at least 99% or 100% identical, to SEQ ID NO: 111. In some embodiments, the vector (e.g., rAAV) comprises a promoter (e.g., a synthetic nervous system specific promoter; see e.g., Tables 10-13) or fragments thereof, and/or, an enhancer, and/or cis-regulatory elements (CREs; see e.g., Tables 13-15) that replaces the promoter and/or enhancer of SEQ ID NO:111. In some embodiments, the vector (e.g., rAAV) comprising an isolated nucleic acid comprising a sequence at least 80% identical, e.g., at least 85%, at least 90%, at least 95%, at least 98%, at least 99% or 100% identical, to SEQ ID NO: 110, further comprises a promoter (e.g., a synthetic nervous system specific promoter; see e.g., Tables 10-13) or fragments thereof, and/or, an enhancer, and/or cis-regulatory elements (CREs; see e.g., Tables 13-15). In some embodiments, the enhancer is a CMV enhancer. In some embodiments, the promoter is an ACTB proximal promoter. In some embodiments, the vector further comprises an intron. In some embodiments, the intron comprises an ACTB intron/chimeric ACTB-HBB2 intron. See, e.g., SEQ ID NO: 111, Table 16. In several embodiments, the foregoing compositions can be used, e.g., in the absence of an administered miRNA to treat a neurological disease or disorder as described herein. In various embodiments, the foregoing compositions can be used, e.g., in the presence of an administered miRNA to treat a neurological disease or disorder as described herein. In some embodiments, recombinant viral vector, e.g., recombinant AAV comprising an isolated nucleic acid sequence at least 80% identical, e.g., at least 85%, at least 90%, at least 95%, at least 98%, at least 99% or 100% identical to SEQ ID NO: 111, is administered to a subject in need therof for expressing the CYP46A1 protein and/or, for treating a neurological disease or disorder as described herein. In some embodiments, recombinant viral vector, e.g recombinant AAV comprising an isolated nucleic acid sequence SEQ ID NO: 111, is administered to a subject in need thereof for expressing the CYP46A1 protein and/or, for treating a neurological disease or disorder as described herein; and wherein the CMV enhancer and/or, ACTB proximal promoter and/or, chimeric ACTB-HBB2 intron of SEQ ID NO:111, is replaced by one or, more of synthetic nervous system specific promoter selected from Tables 10-13 or, fragments thereof, and/or, an enhancer, and/or cis-regulatory elements (CREs) selected from the Tables 13-15.

SEQ ID NO: 111, 4036 bp, ITRto ITR sequence comprising CYP46A1 variant sequence (see e.g., SEQ ID NO: 110).
Bolded text (e.g., nucleotides (nt) 1-130 of SEQ ID NO: 111) indicates the left ITR.
Italicized text (e.g., nt 182-436 of SEQ ID NO: 111) indicates the enhancer.
Bold italicized text (e.g., nt 550-804 of SEQ ID NO: 111) indicates the promoter.
Double underlined text (e.g., nt 824-1892 of SEQ ID NO: 111) indicates the intron.
Bolded double underlined text (e.g., nt 1966-3465 of SEQ ID NO: 111) indicates the coding sequence (CDS) of CYP46A1 variant sequence (see e.g., SEQ ID NO: 110).
Italicized double underlined text (e.g., nt 3629-3853 of SEQ ID NO: 111) indicates the polyA.
Bolded italicized double underlined text (e.g., nt 3907-4036 of SEQ ID NO: 111) indicates the right ITR.

(SEQ ID NO: 111)
CTGCGCGCTCGCTCGCTCACTGAGGCCGCCCGGGCAAAGCCCGGGCGTCGGGCGACCTTTGGTCGCCCGGCCTCA
GTGAGCGAGCGAGCGCGCAGAGAGGGAGTGGCCAACTCCATCACTAGGGGTTCCTTGTAGTTAATGATTAACCCG
CCATGCTACTTATCTACGGCGCGCCacgcgtgactagttattaatagtaatcaattacggggtcattagttcata
gcccatatatggagttccgcgttacataacttacggtaaatggcccgcctggctgaccgcccaacgacccccgcc
cattgacgtcaataatgacgtatgttcccatagtaacgccaatagggactttccattgacgtcaatgggtggagt
atttacggtaaactgcccacttggcagtacatcaagtgtatcatatgccaagtacgccccctattgacgtcaatg
acggtaaatggcccgcctggcattatgcccagtacatgaccttatgggactttcctacttggcagtacatctacg
tattagtcatcgctattaccatgg

[symbol]

[symbol]

[symbol]

[symbol]

[symbol]

[symbol]

[symbol]

[symbol]

[symbol]

[symbol]

aaagcgaagcgcgcggcgggc
gggagtcgctgcgcgctgccttcgccccgtgccccgctccgccgccgcctcgcgccgcccgccccggctctgact
gaccgcgttactcccacaggtgagcgggcgggacggcccttctcctccgggctgtaattagcgcttggtttaatg
acggcttgtttcttttctgtggctgcgtgaaagccttgaggggctccgggagggccctttgtgcggggggagcgg
ctcggggggtgcgtgcgtgtgtgtgtgcgtggggagcgccgcgtgcggctccgcgctgcccggcggctgtgagcg
ctgcgggcgcggcgcggggctttgtgcgctccgcagtgtgcgcgaggggagcgcggccgggggcggtgccccgcg
gtgcggggggggctgcgaggggaacaaaggctgcgtgcggggtgtgtgcgtgggggggtgagcagggggtgtggg
cgcgtcggtcgggctgcaaccccccctocacccccctccccgagttgctgagcacggcccggcttcgggtgcggg
gctccgtacggggcgtggcgcggggctcgccgtgccgggcggggggtggcggcaggtgggggtgccgggcggggc
ggggccgcctcgggccggggagggctcgggggaggggcocggcggcccccggagcgccggcggctgtcgaggcgc
ggcgagccgcagccattgccttttatggtaatcgtgcgagagggcgcagggacttcctttgtcccaaatctgtgc
ggagccgaaatctgggaggcgccgccgcaccccctctagcgggcgcggggcgaagcggtgcggcgccggcaggaa
ggaaatgggcggggagggccttcgtgcgtcgccgcgccgccgtccccttctccctctccagcctcggggctgtcc
gcggggggacggctgccttcgggggggacggggcagggcggggttcggcttctggcgtgtgaccggcggctctag
agcctctgctaaccatgttcatgccttettctttttcctacagctcctgggcaacgtgetggttattgtgctgtc
tcatcattttggcaaagaattgattaattcgagcgaacgcgtcgagtcgctcggtacgatttaaattgaattcct
taagctatcatagga

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

tgagtgatagcttggtaccgagctcgatccaattgcaatgatcatcatgacagatctgcg
cgcgatcgatatcagcgctttaaatttgcgcatgcagctatagttctagagggccctattctatagtgtcaccta
aatgctagagctcgctgatcagcctcga

[symbol]

[symbol]

[symbol]

[symbol]

[symbol]

[symbol]

[symbol]

[symbol]

[symbol]

[symbol]

[symbol]

[symbol]

[symbol]

gtttaaacGCGGCCGCGTAGATAAGTAGCATGGCGGGTTAATCATTA
ACTACA

[symbol]

[symbol]

[symbol]

[symbol]

[symbol]

[symbol]

[symbol]

[symbol]

TABLE 16

| | Location in | | |
|---|---|---|---|
| | SEQ ID | | |
| Feature | NO: 111 | | Qualifier |
| misc_feature | 1 | 130 | /label=L-ITR |
| enhancer | 182 | 548 | /label=CMVe |
| | | | /label=CMVenhancer |
| | | | /note=”GenBank: KX529075.1” |
| promoter | 550 | 823 | /label=ACTBp |
| | | | /label=ACTB proximal promoter |
| misc_feature | 824 | 1892 | /label=chimeric ACTB-HBB2 intron |
| CDS | 1966 | 3468 | /codon_start=1 /product=”>NM_006668.2 Homo sapiens |
| | | | cytochrome P450 family 46 subfamily A member 1 (CYP46A1), |
| | | | mRNA” |
| polyA_signal | 3629 | 3853 | /label=bGH polyA /label=bGH\poly(A)\signal |
| | | | /note=“Bovine growth hormone gene, complete cds. GenBank: |
| | | | M57764.1 ncbi.nlm.nih.gov/nuccore/M57764.1” |
| misc_feature | 3907 | 4036 | /label=R-ITR |

In one aspect, provided herein is a composition comprising an isolated nucleic acid comprising a sequence at least 80% identical, e.g., at least 85%, at least 90%, at least 95%, at least 98%, at least 99% or 100% identical, to SEQ ID NO: 153. In one aspect, provided herein is a composition comprising a recombinant viral vector comprising an isolated nucleic acid comprising a sequence at least 80% identical, e.g., at least 85%, at least 90%, at least 95%, at least 98%, at least 99% or 100% identical, to SEQ ID NO: 153. In some embodiments, the vector (e.g., rAAV) comprises a promoter (e.g., a synthetic nervous system specific promoter; see e.g., Tables 10-13) or fragments thereof, and/or, an enhancer, and/or cis-regulatory elements (CREs; see e.g., Tables 13-15) that replaces the promoter and/or enhancer of SEQ ID NO: 153.

| SEQ ID NO: 153 | |
|---|---|
| 1 | tctagagcta gcatatggat ccatcgattt agggataaca gggtaattat cagcacacaa |
| 61 | ttgcccatta tacgcgcgta taatggacta ttgtgtgctg atatctgtac acttaagggc |
| 121 | tagatcttag cttacgtcac tagagggtcc acgtttagtt tttaagatcc attgatctcc |
| 181 | taaacgctgc aagattcgca acctggtata cttagcctag gcgctaggtc ctagtgcagc |
| 241 | gggacttttt ttctaaagtc gttgagagga ggagtcgtca gaccagatag ctttgatgtc |
| 301 | ctgatcggaa ggatcgttgg cccccctgca ggcagctgtt aattaactgc gcgctcgctc |
| 361 | gctcactgag gccgcccggg caaagcccgg gcgtcgggcg acctttggtc gcccggcctc |
| 421 | agtgagcgag cgagcgcgca gagagggagt ggccaactcc atcactaggg gttccttgta |
| 481 | gttaatgatt aacccgccat gctacttatc tacggcgcgc cacgcgtgac tagttattaa |
| 541 | tagtaatcaa ttacggggtc attagttcat agcccatata tggagttccg cgttacataa |
| 601 | cttacggtaa atggcccgcc tggctgaccg cccaacgacc cccgcccatt gacgtcaata |
| 661 | atgacgtatg ttcccatagt aacgccaata gggactttcc attgacgtca atgggtggag |
| 721 | tatttacggt aaactgccca cttggcagta catcaagtgt atcatatgcc aagtacgccc |
| 781 | cctattgacg tcaatgacgg taaatggccc gcctggcatt atgcccagta catgacctta |
| 841 | tgggactttc ctacttggca gtacatctac gtattagtca tcgctattac catggtcgag |
| 901 | gtgagcccca cgttctgctt cactctcccc atctcccccc cctccccacc cccaattttg |
| 961 | tatttattta ttttttaatt attttgtgca gcgatggggg cggggggggg ggggggcgcg |
| 1021 | cgccaggcgg ggcggggcgg ggcgaggggc ggggcggggc gaggcggaga ggtgcggcgg |
| 1081 | cagccaatca gagcggcgcg ctccgaaagt ttccttttat ggcgaggcgg cggcggcggc |
| 1141 | ggccctataa aaagcgaagc gcgcggcggg cgggagtcgc tgcgcgctgc cttcgccccg |
| 1201 | tgccccgctc cgccgccgcc tcgcgccgcc cgccccggct ctgactgacc gcgttactcc |
| 1261 | cacaggtgag cgggcgggac ggcccttctc ctccgggctg taattagcgc ttggtttaat |
| 1321 | gacggcttgt ttcttttctg tggctgcgtg aaagccttga ggggctccgg gagggccctt |
| 1381 | tgtgcggggg gagcggctcg gggggtgcgt gcgtgtgtgt gtgcgtgggg agcgccgcgt |
| 1441 | gcggctccgc gctgcccggc ggctgtgagc gctgcgggcg cggcgcgggg cttgttgcgc |
| 1501 | tccgcagtgt gcgcgagggg agcgcggccg ggggcggtgc cccgcggtgc ggggggggct |
| 1561 | gcgaggggaa caaaggctgc gtgcggggtg tgtgcgtggg ggggtgagca gggggtgtgg |
| 1621 | gcgcgtcggt cgggctgcaa ccccccctgc acccccctcc ccgagttgct gagcacggcc |
| 1681 | cggcttcggg tgcggggctc cgtacggggc gtggcgcggg gctcgccgtg ccgggcgggg |
| 1741 | ggtggcggca ggtgggggtg ccgggcgggg cggggccgcc tcgggccggg gagggctcgg |
| 1801 | gggaggggcg cggcggcccc cggagcgccg gcggctgtcg aggcgcggcg agccgcagcc |
| 1861 | attgcctttt atggtaatcg tgcgagaggg cgcagggact tcctttgtcc caaatctgtg |
| 1921 | cggagccgaa atctgggagg cgccgccgca ccccctctag cgggcgcggg gcgaagcggt |
| 1981 | gcggcgccgg caggaaggaa atgggcgggg agggccttcg tgcgtcgccg cgccgccgtc |
| 2041 | cccttctccc tctccagcct cggggctgtc cgcgggggga cggctgcctt cgggggggac |
| 2101 | ggggcagggc ggggttcggc ttctggcgtg tgaccggcgg ctctagagcc tctgctaacc |
| 2161 | atgttcatgc cttcttcttt ttcctacagc tcctgggcaa cgtgctggtt attgtgctgt |
| 2221 | ctcatcattt tggcaaagaa ttgattaatt cgagcgaacg cgtcgagtcg ctcggtacga |
| 2281 | tttaaattga attccttaag ctatcatagg aatgagcccc gggctgctgc tgctcggtag |
| 2341 | cgccgtcctg ctcgccttcg gcctctgctg caccttcgtg caccgcgctc gcagccgcta |
| 2401 | cgagcacatc cccgggccgc cgcggcccag tttccttcta ggacacctcc cctgcttttg |
| 2461 | gaaaaaggat gaggttggtg gccgtgtgct ccaagatgtg tttctagatt gggctaagaa |
| 2521 | gtatggacct gtagtgcggg tcaacgtctt ccacaaaacc tcagtcatcg tcacgagtcc |
| 2581 | tgagtcggtt aagaagttcc tgatgtcaac caagtacaac aaggactcca agatgtaccg |
| 2641 | tgcgctccag actgtgtttg gtgagagact cttcggccaa ggcttggtgt ccgaatgcaa |
| 2701 | ctatgagcgc tggcacaagc agcggagagt gatagacctg gccttcagcc ggagctcctt |
| 2761 | ggttagctta atggaaacat tcaacgaaaa ggctgagcag ctggtggaga ttctagaagc |
| 2821 | caaggcagat gggcagaccc ctgtgagcat gcaggacatg ctgacctaca ccgccatgga |
| 2881 | catcctggcc aaggcagctt ttgggatgga gaccagtatg ctgctgggtg cccagaagcc |
| 2941 | tctgtcccag gcagtgaaac ttatgttgga gggaatcact gcgtcccgca acactctggc |
| 3001 | aaagttcctg ccagggaaga ggaagcagct ccgggaggtc cgggagagca ttcgcttcct |
| 3061 | gcgccaggtg ggcagggact gggtccagcg ccgccgggaa gccctgaaga ggggcgagga |
| 3121 | ggttcctgcc gacatcctca cacagattct gaaagctgaa gagggagccc aggacgacga |
| 3181 | gggtctgctg gacaacttcg tcaccttctt cattgctggt cacgagacct ctgccaacca |
| 3241 | cttggcgttc acagtgatgg agctgtctcg ccagccagag atcgtggcaa ggctgcaggc |
| 3301 | cgaggtggat gaagtgattg gttctaagag gtacctggat ttcgaggacc tggggagact |
| 3361 | gcagtacctg tcccaggtcc tcaaagagtc gctgaggctg tacccaccag catggggcac |
| 3421 | ctttaggctg ctggaagagg agaccttgat tgatggggtg agagtccccg gcaacacccc |
| 3481 | gctcttgttc agcacctaty tgatggggcg gatggacaca tactttgagg acccgctgac |
| 3541 | tttcaacccc gatcgcttcg gccctggagc acccaagcca cggttcacct acttcccctt |
| 3601 | ctccctgggc caccgctcct gcatcgggca gcagtttgct cagatggagg tgaaggtggt |
| 3661 | catggcaaag ctgctgcaga ggctggagtt ccggctggtg cccgggcagc gcttcgggct |
| 3721 | gcaggagcag gccacactca agccactgga ccccgtgctg tgcaccctgc ggccccgcgg |
| 3781 | ctggcagccc gcacccccac cacccccctg ctgagtgata gcttggtacc gagctcgatc |
| 3841 | caattgcaat gatcatcatg acagatctgc gcgcgatcga tatcagcgct ttaaatttgc |
| 3901 | gcatgcagct atagttctag agggccctat tctatagtgt cacctaaatg ctagagctcg |
| 3961 | ctgatcagcc tcgactgtgc cttctagttg ccagccatct gttgtttgcc cctcccccgt |
| 4021 | gccttccttg accctggaag gtgccactcc cactgtcctt tcctaataaa atgaggaaat |
| 4081 | tgcatcgcat tgtctgagta ggtgtcattc tattctgggg ggtggggtgg ggcaggacag |
| 4141 | caagggggag gattgggaag acaatagcag gcatgctggg gatgcggtgg gctctatggg |
| 4201 | tttaaacgcg gccgcgtaga taagtagcat ggcgggttaa tcattaacta caaggaaccc |
| 4261 | ctagtgatgg agttggccac tccctctctg cgcgctcgct cgctcactga ggccgggcga |
| 4321 | ccaaaggtcg cccgacgccc gggctttgcc cgggcggcct cagtgagcga gcgagcgcgc |
| 4381 | agttaattaa ggcgccctag gccgaccctt agactctgta ctcagttcta taaacgagcc |
| 4441 | attggatacg agatccgtag attgataagg gacacggaat atccccggac gcaatagaca |
| 4501 | ccggtggaca gcttggtatc ctgagcacag tcgcgcgtcc gaatctagct ctactttaga |
| 4561 | ggccccggat tctgatggtc gtagaccgca gaaccgattg gggggatgag atctactagt |
| 4621 | tatcagcaca caattgccca ttatacgcgc gtataatgga ctattgtgtg ctgatatagg |
| 4681 | gataacaggg taattctaga gctagcatat ggatccatcg atttgatgcg gtattttctc |
| 4741 | cttacgcatc tgtgcggtat ttcacaccgc atacgtcaaa gcaaccatag tacgcgccct |
| 4801 | gtagcggcgc attaagcgcg gogggtgtgg tggttacgcg cagcgtgacc gctacacttg |
| 4861 | ccagcgccct agcgcccgct cctttcgctt tcttcccttc ctttctcgcc acgttcgccg |
| 4921 | gctttccccg tcaagctcta aatcgggggc tccctttagg gttccgattt agtgctttac |
| 4981 | ggcacctcga ccccaaaaaa cttgatttgg gtgatggttc acgtagtggg ccatcgccct |
| 5041 | gatagacggt ttttcgccct ttgacgttgg agtccacgtt ctttaatagt ggactcttgt |
| 5101 | tccaaactgg aacaacactc aactctatct cgggctattc ttttgattta taagggattt |
| 5161 | tgccgatttc ggtctattgg ttaaaaaatg agctgattta acaaaaattt aacgcgaatt |
| 5221 | ttaacaaaat attaacgttt acaattttat ggtgcactct cagtacaatc tgctctgatg |
| 5281 | ccgcatagtt aagccagccc cgacacccgc caacacccgc tgacgcgccc tgacgggctt |
| 5341 | gtctgctccc ggcatccgct tacagacaag ctgtgaccgt ctccgggagc tgcatgtgtc |
| 5401 | agaggttttc accgtcatca ccgaaacgcg cgagacgaaa gggcctcgtg atacgcctat |
| 5461 | ttttataggt taatgtcatg ataataatgg tttcttagac gtcaggtggc acttttcggg |
| 5521 | gaaatgtgcg cggaacccct atttgtttat ttttctaaat acattcaaat atgtatccgc |
| 5581 | tcatgagaca ataaccctga taaatgcttc aataatattg aaaaaggaag agtatgagcc |
| 5641 | atattcaacg ggaaacgtcg aggccgcgat taaattccaa catggatgct gatttatatg |
| 5701 | ggtataaatg ggctcgcgat aatgtcgggc aatcaggtgc gacaatctat cgcttgtatg |
| 5761 | ggaagcccga tgcgccagag ttgtttctga aacatggcaa aggtagcgtt gccaatgatg |
| 5821 | ttacagatga gatggtcaga ctaaactggc tgacggaatt tatgcctctt ccgaccatca |
| 5881 | agcattttat ccgtactcct gatgatgcat ggttactcac cactgcgatc cccggaaaaa |
| 5941 | cagcattcca ggtattagaa gaatatcctg attcaggtga aaatattgtt gatgcgctgg |
| 6001 | cagtgttcct gcgccggttg cattcgattc ctgtttgtaa ttgtcctttt aacagcgatc |
| 6061 | gcgtatttcg tctcgctcag gcgcaatcac gaatgaataa cggtttggtt gatgcgagtg |
| 6121 | attttgatga cgagcgtaat ggctggcctg ttgaacaagt ctggaaagaa atgcataaac |
| 6181 | ttttgccatt ctcaccggat tcagtcgtca ctcatggtga tttctcactt gataacctta |
| 6241 | tttttgacga ggggaaatta ataggttgta ttgatgttgg acgagtcgga atcgcagacc |
| 6301 | gataccagga tcttgccatc ctatggaact gcctcggtga gttttctcct tcattacaga |
| 6361 | aacggctttt tcaaaaatat ggtattgata atcctgatat gaataaattg cagtttcatt |
| 6421 | tgatgctcga tgagtttttc taagcgtata atggtctaga gctagcatat ggatccatcg |
| 6481 | attccattat acgcctgtca gaccaagttt actcatatat actttagatt gatttaaaac |
| 6541 | ttcattttta atttaaaagg atctaggtga agatcctttt tgataatctc atgaccaaaa |
| 6601 | tcccttaacg tgagttttcg ttccactgag cgtcagaccc cgtagaaaag atcaaaggat |
| 6661 | cttcttgaga tccttttttt ctgcgcgtaa tctgctgctt gcaaacaaaa aaaccaccgc |
| 6721 | taccagcggt ggtttgtttg ccggatcaag agctaccaac tctttttccg aaggtaactg |
| 6781 | gcttcagcag agcgcagata ccaaatactg ttcttctagt gtagccgtag ttaggccacc |
| 6841 | acttcaagaa ctctgtagca ccgcctacat acctcgctct gctaatcctg ttaccagtgg |
| 6901 | ctgctgccag tggcgataag tcgtgtctta ccgggttgga ctcaagacga tagttaccgg |
| 6961 | ataaggcgca gcggtcgggc tgaacggggg gttcgtgcac acagcccagc ttggagcgaa |
| 7021 | cgacctacac cgaactgaga tacctacagc gtgagctatg agaaagcgcc acgcttcccg |
| 7081 | aagggagaaa ggcggacagg tatccggtaa gcggcagggt cggaacagga gagcgcacga |
| 7141 | gggagcttcc agggggaaac gcctggtatc tttatagtcc tgtcgggttt cgccacctct |
| 7201 | gacttgagcg tcgatttttg tgatgctcgt caggggggcg gagcctatgg aaaaacgcca |
| 7261 | gcaacgcggc ctttttacgg ttcctggcct tttgctggcc ttttgctcac atgt |

Some of the aspects provided herein is a nucleic acid sequence as set forth in SEQ ID NO: 153 is used to manufacture rAAV that lacks bacterial sequence. In some embodiments, the rAAV is manufactured from plasmid DNA template e.g, as set forth in SEQ ID NO: 111. In some embodiments, the rAAV is manufactured from close ended linear duplexed DNA e.g., as set forth in SEQ ID NO: 153 or, SEQ ID NO:111.

### Modified Capsids

In one embodiment, the capsid described herein is further modified to increase tropism for the CNS. Provided herein is a composition comprising a modified viral capsid comprising a payload, wherein the payload comprises a regulatory sequence and a nucleic acid sequence flanked by inverted terminal repeats (ITRs) that target a central nervous system disorder, and wherein the modification is a chemical, non-chemical or amino acid modification. In some embodiments, the nucleic acid sequence of the payload comprises (a) an isolated nucleic acid encoding a transgene encoding one or more miRNAs; and (b) an isolated nucleic acid encoding a CYP46A1 protein. In some embodiments, the nucleic acid sequence of the payload comprises an isolated nucleic acid encoding a transgene encoding one or more miRNAs. In some embodiments, the nucleic acid sequence of the payload comprises an isolated nucleic acid encoding a CYP46A1 protein.

Further provided herein is a composition comprising (a) a first modified viral capsid comprising a first payload, and (b) at least a second modified viral capsid comprising a second payload, wherein the payload comprises a regulatory sequence and a nucleic acid sequence flanked by inverted terminal repeats (ITRs) that target a central nervous system disorder, wherein the first and at least second modified viral capsids are the same, and the first and second payloads are different, and wherein the modification is a chemical, non-chemical or amino acid modification. In some embodiments, the nucleic acid sequence of the first or second payload comprises an isolated nucleic acid encoding a transgene encoding one or more miRNAs. In some embodiments, the nucleic acid sequence of the first or second payload comprises an isolated nucleic acid encoding a CYP46A1 protein.

Further provided herein is a composition comprising (a) a first modified capsid comprising a first payload, and (b) at least a second modified capsid comprising a second payload, wherein the payload comprises a regulatory sequence and a nucleic acid sequence flanked by inverted terminal repeats (ITRs) that target a central nervous system disorder, wherein the first and at least second modified capsids are different, and the first and second payloads can be the same or different, and wherein the modification is a chemical, non-chemical or amino acid modification. In some embodiments, the nucleic acid sequence of the first or second payload comprises an isolated nucleic acid encoding a transgene encoding one or more miRNAs. In some embodiments, the nucleic acid sequence of the first or second payload comprises an isolated nucleic acid encoding a CYP46A1 protein.

In certain embodiments, the modified viral capsid comprises modification that results in its preferential targeting of the CNS or PNS. For example, the modified viral capsid has increased tropism for the CNS, and/or decreased tropism for at least a second location, e.g., the liver. Preferential targeting of the CNS does not exclude targeting to other sites, but rather indicates that it is more highly targeted to the CNS as compared to another site.

In one embodiment, the modified viral capsid comprises modification that results in its targeting of the CNS or PNS. For example, a modification to a capsid that typically targets a non-CNS site (e.g., the liver) can redirect the capsid to now target both the CNS and the non-CNS site. In such embodiment, the CNS-targeting does not need to be preferential.

In one embodiment, the modification to the capsid is an amino acid modification, e.g., an amino acid deletion, insertion, or substitute. In one embodiment, the amino acid modification increases tropism for the CNS or PNS. In one embodiment, the amino acid modification targets the modified capsid to the CNS or PNS.

In one embodiment, the modified viral capsid has or consists of, or consists essentially of a nucleic acid sequence that is 90% identical to SEQ ID NOs 1-4 of U.S. patent application Ser. No. 16/511,913, the contents of which are incorporated herein by references in its entirety. This US patent application describes chimeric AAV capsid sequences that exhibit a dominant tropism for oligodendrocytes, and can be used to create AAV vectors that transduce oligodendrocytes in the CNS of subject.

In one embodiment, the modified viral capsid is an AAV capsid protein comprising one or more amino acids substitutions, wherein the substitutions introduce a new glycan binding site into the AAV capsid protein. In some embodiments, the amino acid substitutions are in amino acid 266, amino acids 463-475 and amino acids 499-502 in AAV2 or the corresponding amino acid positions in AAV1, AAV3, AAV4, AAV5, AAV6, AAV7, AAV8 or AAV10. Such AAV capsid protein is further described in, e.g., U.S. patent application Ser. No. 16/110,773; the contents of which are incorporated herein by references in its entirety.

In one embodiment, the modified viral capsid is an AAV capsid protein that comprises, consists of, or consists essentially of an AAV 2.5 capsid protein (SEQ ID NO: 1 of International Patent Application No. PCT/US2020/029493; the contents of which are incorporated herein by references in its entirety) comprising one or more amino acid substitutions that introduce a new glycan binding site. Such amino acid substitutions can target the capsid to neurons and glial cells, such as astrocytes. In embodiments of the capsid proteins, capsids, viral vectors and methods described in the International Patent Application No. PCT/US2020/029493, the one or more amino acid substitutions comprise A267S, SQAGASDIRDQSR464-476SX₁AGX₂SX₃X₄X₅X₆QX₇R (SEQ ID NOS 153 and 154, respectively), wherein X₁₋₇ can be any amino acid, and EYSW 500-503 (SEQ ID NO: 155) EX₈X₉W, wherein X₈₋₉ can be any amino acid. In embodiments of the capsid proteins, capsids, viral vectors and methods described herein, X₁ is V or a conservative substitution thereof, X₂ is P or a conservative substitution thereof, X₃ is N or a conservative substitution thereof, X₄ is M or a conservative substitution thereof, X₅ is A or a conservative substitution thereof; X₆ is V or a conservative substitution thereof, X₇ is G or a conservative substitution thereof, X₈ is F or a conservative substitution thereof, and/or X₉ is A or a conservative substitution thereof. In embodiments of the capsid proteins, capsids, viral vectors and methods described herein, X₁ is V, X₂ is P, X₃ is N, X₄ is M, X₅ is A, X₆ is V, X₇ is G, X₈ is F, and X₉ is A, wherein the new glycan binding site is a galactose binding site. Such AAV capsid protein is further described in, e.g., International Patent Application No. PCT/US2020/029493; the contents of which are incorporated herein by references in its entirety.

In one embodiment, the modified viral capsid is an AAV capsid protein particle comprising a surface-bound peptide, wherein the peptide bound to the surface of the AAV particle is Angiopep-2, GSH, HIV-1 TAT (48-60), ApoE (159-167)2, Leptin 30 (61-90), THR, PB5-3, PB5-5, PB5-14, or any combination thereof, as described in, e.g., U.S. patent application Ser. No. 16/956,306; the contents of which are incorporated herein by references in its entirety. Such AAV capsid permits delivery, e.g., of a payload, across the blood brain barrier.

In one embodiment, the modified viral capsid is AAV capsid protein (e.g., an AAV1, AAV5, or AAV6 capsid protein), wherein the VP3 region of the capsid protein comprises modifications (e.g., replacement of a tyrosine residue with a non-tyrosine residue and/or a threonine residue with a non-threonine residue) at positions corresponding to: one or more of, or each of Y705, Y731, and T492 of a wild-type AAV1 capsid protein (e.g., SEQ ID NO: 1 of U.S. patent application Ser. No. 16/565,191; the contents of which are incorporated herein by references in its entirety); one or more of, or each of Y436, Y693, and Y719 of a wild-type AAV5 capsid protein (e.g., SEQ ID NO: 2 of U.S. patent application Ser. No. 16/565,191); or one or more of, or each of Y705, Y731, and T492 of a wild-type AAV6 capsid protein (e.g., SEQ ID NO: 3 of U.S. patent application Ser. No. 16/565,191). Such AAV capsids target neurons and astrocytes.

In one embodiment, the modified viral capsid is AAV capsid protein (e.g., an AAV1, AAV5, or AAV6 capsid protein) comprising Y to F (tyrosine to phenylalanine) modifications or T to V (threonine to valine) modifications in the VP3 region of the capsid at positions corresponding to: one or more of or each of Y705F, Y731F, and T492V of a wild-type AAV1 capsid protein (e.g., SEQ ID NO: 1 of U.S. patent application Ser. No. 16/565,191); one or more of or each of Y436F, Y693F, and Y719F of a wild-type AAV5 capsid protein (e.g., SEQ ID NO: 2 of U.S. patent application Ser. No. 16/565,191); or one or more of or each of Y705F, Y731F, and T492V of a wild-type AAV6 capsid protein (e.g., SEQ ID NO: 3 of U.S. patent application Ser. No. 16/565,191). Such AAV capsids target neurons and astrocytes.

In one embodiment, the modified viral capsid is AAV capsid protein (e.g., an AAV1, AAV5, or AAV6 capsid protein), wherein a VP3 region of the capsid protein comprises modifications (e.g., replacement of a tyrosine residue with a non-tyrosine residue and/or a threonine residue with a non-threonine residue) at positions corresponding to: one or more of or each of Y705, Y731, and T492 of a wild-type AAV1 capsid protein (e.g., SEQ ID NO: 1 of U.S. patent application Ser. No. 16/565,191); one or more of or each of Y436, Y693, and Y719 of a wild-type AAV5 capsid protein (e.g., SEQ ID NO: 2 of U.S. patent application Ser. No. 16/565,191); or one or more of or each of Y705, Y731, and T492 of a wild-type AAV6 capsid protein (e.g., SEQ ID NO: 3 of U.S. patent application Ser. No. 16/565,191). Such AAV capsids target neurons and astrocytes.

In one embodiment, the modified viral capsid is AAV capsid protein (e.g., an AAV1, AAV5, or AAV6 capsid protein) comprising Y to F (tyrosine to phenylalanine) modifications or T to V (threonine to valine) modifications in the VP3 region of the capsid protein at positions corresponding to: one or more of or each of Y705F, Y731F, and T492V of a wild-type AAV1 capsid protein (e.g., SEQ ID NO: 1 of U.S. patent application Ser. No. 16/565,191); one or more of or each of Y436F, Y693F, and Y719F of a wild-type AAV5 capsid protein (e.g., SEQ ID NO: 2 of U.S. patent application Ser. No. 16/565,191); or one or more of or each of Y705F, Y731F, and T492V of a wild-type AAV6 capsid protein (e.g., SEQ ID NO: 3 of U.S. patent application Ser. No. 16/565,191). Such AAV capsids target neurons and astrocytes.

In one embodiment, the amino acid modification permits the modified capsid to evade neutralizing antibodies, for example, that are generated against a viral vector, e.g., of the same serotype. In one embodiment, the amino acid modification permits the modified capsid to be used for repeat administration, for example, the modification will enable the capsid to have a therapeutic effect upon re-administration.

In one embodiment, the modified viral capsid is a chimeric capsid. A “chimeric” capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

In one embodiment, the modified viral capsid is a haploid capsid. As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric protein is present.

In some embodiments of the technology described herein, a modified viral capsid comprises one or more modifications, e.g., a chemical modification, a non-chemical modification, or an amino acid modification to the capsid. Such modifications can, for example, modify the tissue-type tropism or cell-type tropism of the modified capsid, among other things.

Modifications can alter the properties of the capsid, including biochemical properties such as receptor binding, directly, such that the modification itself alters the behavior of the capsid, or can permit further modification, such as the attachment of a ligand which in turn modifies behavior of the capsid in a desired manner.

In one embodiment, chemical modification of cysteine residues, which may be naturally present or introduced by genetic modification of a capsid polypeptide coding sequence, permits the covalent attachment of a ligand via disulfide bond formation (see, e.g., WO 2005/106046, the contents of which are incorporated herein by reference).

Various ligands are contemplated, including but not limited to antibodies or antigen-binding fragments thereof that, for example, target a cell-surface protein expressed by a target cell (see, e.g., WO 2000/002654, which is incorporated herein by reference).

WO2015/062516, the contents of which are also incorporated herein by reference, describes the insertion of an amino acid comprising an azido group by genetic modification of the capsid gene, followed by chemical conjugation of a ligand via the azido group.

The modification of AAV capsid tropism by glycation, or chemical conjugation of sugar moieties, is described by Horowitz et al., Bioconjugate Chem. 22: 529-532 (2011). That approach, and similar approaches are contemplated for modification of capsids as described herein.

In other embodiments, the coating of a viral capsid with a polymer, such as polyethylene glycol (PEG) or poly-(N-hydroxypropyl)methacrylamide (pHPMA) is specifically contemplated. Such modification can, for example, reduce specific and nonspecific interactions with non-target tissues.

In other embodiments, carbodiimide coupling is specifically contemplated. See, e.g., Joo et al. ACS Nano 5, titled “Enhanced Real-time Monitoring of Adeno-Associated Virus Trafficking by Virus-Quantum Dot Conjugates” (2011).

In other embodiments, the viral capsid can be modified, e.g., as described in WO 2017/212019, see also U.S. National Phase U.S. Ser. No. 16/308,740, the contents of which are each incorporated herein by reference. The approach described therein couples a viral capsid to a ligand via bonds comprising —CSNH— and an aromatic moiety. While genetically modified viral capsids can be further modified by this approach, the modifications described therein do not require genetic modification of the viral capsid. Ligands described therein include, for example, a targeting agent, a steric shielding agent for avoiding neutralizing antibody interactions, a labeling agent or a magnetic agent. Targeting ligands described therein include, for example, a cell-type specific ligand, a protein, a mono- or polysaccharide, a steroid hormone, an RGD motif peptide (e.g., Arg-Gly-Asp, a cell adhesion motif which can mimic cell adhesion proteins and bind to integrins), a vitamin, and a small molecule.

In one embodiment, the chemical modification of the invention is a modification described in International patent application PCT/EP2017/064089, the content of which is incorporated herein by reference in its entirety.

In one embodiment, the chemical modification of the invention is a modification described in International patent application PCT/EP2020/069554, the content of which is incorporated herein by reference in its entirety.

In one embodiment, the capsid has at least one chemically-modified tyrosine residue in its capsid, wherein said chemically-modified tyrosine residue is of formula (I):

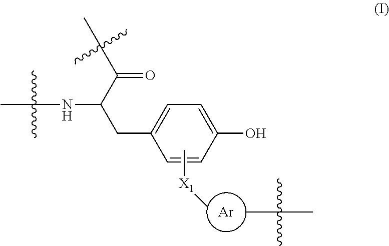

wherein:
X1 is selected from the group consisting of:

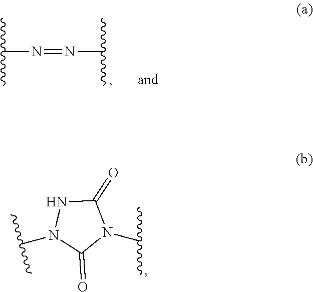

Ar is an aryl or a heteroaryl moiety optionally substituted.

In one embodiment, the capsid has at least one chemically-modified tyrosine residue is of formula (Ia):

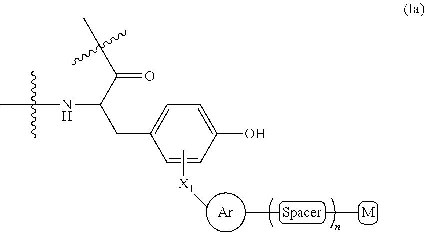

wherein:
Xi, and Ar are as defined herein above,
Spacer is a group for linking the “Ar” group to the functional moiety “M” which preferably
   comprises up to 1000 carbon atoms and which is preferably in the form of a chemical chain which optionally comprises heteroatoms and/or cyclic moieties,
n is 0 or 1; and
M is a functional moiety comprising a steric agent, a labelling agent, cell-types specific ligand or a drug moiety.

In one embodiment, Xi is of formula (a) and/or “Ar” is selected from substituted or unsubstituted phenyl, pyridyl, naphthyl, and anthracenyl.

In one embodiment, the capsid has at least one chemically-modified tyrosine is of formula (Ic):

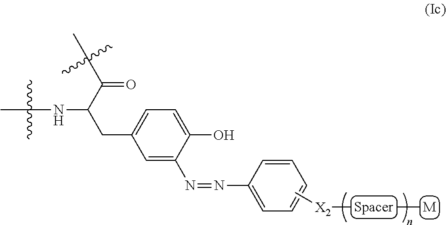

wherein:
X2 is —C(═O)—NH, —C(═O)—O, —C(═O)—O—C(═O)—, O—(C═O)—, NH—C(═O)—, NH—C(═O)—NH, —O—C═O—O—, 0, NH, —NH(C═S)—, or —(C═S)—NH—, preferably —(C═O)—NH— or —(C═O)—O—
X2 is at position para, meta or ortho, preferably at position para of the phenyl group,
Spacer, n and M are as defined herein above.

In one embodiment, “Spacer”, when present, is selected from the group consisting of saturated or unsaturated, linear or branched C2-C40 hydrocarbon chains, optionally substituted, polyethylene glycol, polypropylene glycol, pHPMA (polymer of N-(2-Hydroxypropyl)methacrylamide), Poly Lactic-co-Glycolic Acid (PLGA), polymers of alkyl diamines and combinations thereof, and/or

“M” comprises, or consists of, cell-type targeting ligand, preferably selected from a mono- or a polysaccharide, a hormone, including a steroid hormone, a peptide such as RGD peptide (e.g., Arg-Gly-Asp, a cell adhesion motif which can mimic cell adhesion proteins and bind to integrins), a muscle targeting peptide (MTP) or Angiopep-2, a protein or a fragment thereof, a membrane receptor or a fragment thereof, an aptamer, an antibody including heavy-chain antibody, and fragments thereof such as antigen-binding fragment (Fab), Fab′ (which is the antigen-binding fragment further comprising a free sulfhydryl group), and VHH, a single-chain fragment variable (ScFv), a spiegelmer, a peptide aptamer, vitamins and drugs such as Cannabinoid receptor 1 (CB1) and/or Cannabinoid receptor 2 (CB2) ligands.

In one embodiment, “Spacer” (when present) is selected from the group consisting of linear or branched C2-C20 alkyl chains, polyethylene glycol, polypropylene glycol, pHPMA, PLGA, polymer of alkyl diamine and combinations thereof, said polymers having from 2 to 20 monomers and/or “M” comprises, or consists of, a cell-type specific ligand derived from a protein selected from transferrin, Epidermal Growth Factor (EGF), and basic Fibroblast Growth Factor 13FGF, a mono- or a polysaccharide comprising one or several galactose, mannose, N-acetylgalactosamine residues, bridge GalNac, or mannose-6-phosphate, MTP selected from SEQ ID NO:1 to SEQ ID NO:7, and vitamins such as folic acid.

In one embodiment, the capsid further has at least one additional chemically modified amino acid residue in the capsid, which is different from a tyrosine residue, said amino acid residue preferably bearing an amino group chemically modified with a group of formula (V):

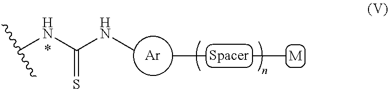

wherein:
N* being the nitrogen of the amino group of an amino acid residue, e.g. of a lysine residue or arginine residue, and
Ar, Spacer, n and M has the same definition as Ar, Spacer, n and M of formula (II) of claim 2.

In one embodiment, the capsid is incubated a chemical reagent bearing a reactive group selected from an aryl diazonium, and a 4-phenyl-1,2,4-triazole-3,5-dione (PTAD) moiety in conditions conducive for reacting said reactive group with a tyrosine residue present in the capsid so as to form a covalent bound.

In one embodiment, the capsid is incubated with a chemical reagent of formula VId to obtain the at least one chemically-modified tyrosine residue in the capsid of formula Ic.

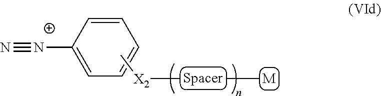

### Administration

The rAAVs of the disclosure may be delivered to a subject in compositions according to any appropriate methods known in the art. For example, an rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, rAAV as described in the disclosure are administered by intravenous injection. In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cistema magna injection. In some embodiments, the rAAV are delivered by cerebral lateral ventricle injection.

Delivery of the compositions to a mammalian subject may be by, for example, by any know mean of deliver to a desire site, e.g., the CNS. It may be desirable to deliver the composition to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Any composition described herein may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, compositions as described in the disclosure are administered by intravenous injection. In some embodiments, compositions as described in the disclosure are administered by intraspinal injection. In some embodiments, compositions as described in the disclosure are administered by intracerebro ventricular injection. In some embodiments, compositions are administered by intracerebral injection. In some embodiments, compositions are administered by intrathecal injection. In some embodiments, compositions are administered by intrastriatal injection. In some embodiments, compositions are delivered by intracranial injection. In some embodiments, compositions are delivered by cistema magna injection. In some embodiments, compositions are delivered by cerebral lateral ventricle injection.

The CNS includes, but is not limited to, certain regions of the CNS, neural pathways, somatosensory systems, visual systems, auditory systems, nerves, neuro endocrine systems, neuro vascular systems, brain neurotransmitter systems, and dural meningeal system.

Exemplary regions of the CNS include, but are not limited to Myelencephalon; Medulla oblongata; Medullary pyramids; Olivary body; Inferior olivary nucleus; Rostral ventrolateral medulla; Caudal ventrolateral medulla; Solitary nucleus (Nucleus of the solitary tract); Respiratory center-Respiratory groups Dorsal respiratory group; Ventral respiratory group or Apneustic centre Pre-Bötzinger complex; Botzinger complex; Retrotrapezoid nucleus; Nucleus retrofacialis; Nucleus retroambiguus; Nucleus para-ambiguus; Paramedian reticular nucleus; Gigantocellular reticular nucleus; Parafacial zone; Cuneate nucleus; Gracile nucleus; Perihypoglossal nuclei; Intercalated nucleus; Prepositus nucleus; Sublingual nucleus; Area postrema; Medullary cranial nerve nuclei; Inferior salivatory nucleus; Nucleus ambiguus; Dorsal nucleus of vagus nerve; Hypoglossal nucleus; Chemoreceptor trigger zone; Metencephalon; Pons; Pontine nuclei; Pontine cranial nerve nuclei; Chief or pontine nucleus of the trigeminal nerve sensory nucleus; Motor nucleus for the trigeminal nerve; Abducens nucleus (VI); Facial nerve nucleus (VII); Vestibulocochlear nuclei (vestibular nuclei and cochlear nuclei) (VIII); Superior salivatory nucleus; Pontine tegmentum; Pontine micturition center (Barrington's nucleus); Locus coeruleus; Pedunculopontine nucleus; Laterodorsal tegmental nucleus; Tegmental pontine reticular nucleus; Nucleus incertus; Parabrachial area; Medial parabrachial nucleus; Lateral parabrachial nucleus; Subparabrachial nucleus (Kolliker-Fuse nucleus); Pontine respiratory group; Superior olivary complex; Medial superior olive; Lateral superior olive; Medial nucleus of the trapezoid body; Paramedian pontine reticular formation; Parvocellular reticular nucleus; Caudal pontine reticular nucleus; Cerebellar peduncles; Superior cerebellar peduncle; Middle cerebellar peduncle; Inferior cerebellar peduncle; Fourth ventricle; Cerebellum Cerebellar vermis; Cerebellar hemispheres; Anterior lobe; Posterior lobe; Flocculonodular lobe; Cerebellar nuclei; Fastigial nucleus; Interposed nucleus; Globose nucleus; Emboliform nucleus; Dentate nucleus; Midbrain (mesencephalon); Tectum Corpora quadrigemina; Inferior colliculi; Superior colliculi; Pretectum; Tegmentum Periaqueductal gray; Rostral interstitial nucleus of medial longitudinal fasciculus; Midbrain reticular formation; Dorsal raphe nucleus; Red nucleus; Ventral tegmental area; Parabrachial pigmented nucleus; Paranigral nucleus; Rostromedial tegmental nucleus; Caudal linear nucleus; Rostral linear nucleus of the raphe; Interfascicular nucleus; Substantia nigra; Pars compacta; Pars reticulata; Interpeduncular nucleus; Cerebral peduncle; Crus cerebri; Mesencephalic cranial nerve nuclei; Oculomotor nucleus (III); Edinger-Westphal nucleus; Trochlear nucleus (IV); Mesencephalic duct (cerebral aqueduct, aqueduct of Sylvius); Forebrain (prosencephalon); Diencephalon; Epithalamus; Pineal body (pineal gland); Habenular nuclei; Stria medullaris; Taenia thalami; Third ventricle; Subcommissural organ; Thalamus; Anterior nuclear group; Anteroventral nucleus (a.k.a. ventral anterior nucleus); Anterodorsal nucleus; Anteromedial nucleus; Medial nuclear group; Medial dorsal nucleus; Midline nuclear group; Paratenial nucleus; Reuniens nucleus; Rhomboidal nucleus; Intralaminar nuclear group; Centromedian nucleus; Parafascicular nucleus; Paracentral nucleus; Central lateral nucleus; Lateral nuclear group; Lateral dorsal nucleus; Lateral posterior nucleus; Pulvinar; Ventral nuclear group Ventral anterior nucleus; Ventral lateral nucleus; Ventral posterior nucleus; Ventral posterior lateral nucleus; Ventral posterior medial nucleus; Metathalamus; Medial geniculate body; Lateral geniculate body; Thalamic reticular nucleus; Hypothalamus (limbic system) (HPA axis); Anterior Medial area Parts of preoptic area; Medial preoptic nucleus INAH 1; INAH 2; INAH 3; INAH 4; Median preoptic nucleus; Suprachiasmatic nucleus; Paraventricular nucleus; Supraoptic nucleus (mainly); Anterior hypothalamic nucleus; Lateral area; Parts of preoptic area; Lateral preoptic nucleus; Anterior part of Lateral nucleus; Part of supraoptic nucleus; Other nuclei of preoptic area; Median preoptic nucleus; Periventricular preoptic nucleus; Tuberal Medial area; Dorsomedial hypothalamic nucleus; Ventromedial nucleus; Arcuate nucleus; Lateral area Tuberal part of Lateral nucleus; Lateral tuberal nuclei; Posterior Medial area Mammillary nuclei (part of mammillary bodies); Posterior nucleus; Lateral area Posterior part of Lateral nucleus; Surface Median eminence; Mammillary bodies; Pituitary stalk (infundibulum); Optic chiasm; Subfornical organ; Periventricular nucleus; Tuber cinereum; Tuberal nucleus; Tuberomammillary nucleus; Tuberal region; Mammillary nucleus; Subthalamus (HPA axis); Subthalamic nucleus; Zona incerta; Pituitary gland (HPA axis); Neurohypophysis; Pars intermedia (Intermediate Lobe); Adenohypophysis; Telencephalon (cerebrum); Cerebral hemispheres; White matter; Centrum semiovale; Corona radiata; Internal capsule; External capsule; Extreme capsule; Subcortical; Hippocampus (Medial Temporal Lobe); Dentate gyrus; Cornu ammonis (CA fields); Cornu ammonis area 1 (CA1); Cornu ammonis area 2 (CA2); Cornu ammonis area 3 (CA3); Cornu ammonis area 4 (CA4); Amygdala (limbic system) (limbic lobe); Central nucleus (autonomic nervous system); Medial nucleus (accessory olfactory system); Cortical and basomedial nuclei (main olfactory system); Lateral and basolateral nuclei (frontotemporal cortical system); Extended amygdala; Stria terminalis Bed nucleus of the stria terminalis; Claustrum; Basal ganglia; Striatum Dorsal striatum (a.k.a. neostriatum); Putamen; Caudate nucleus; Ventral striatum; Nucleus accumbens; Olfactory tubercle; Globus pallidus (forms nucleus lentiformis with putamen); Ventral pallidum; Subthalamic nucleus; Basal forebrain; Anterior perforated substance; Substantia innominata; Nucleus basalis; Diagonal band of Broca; Septal nuclei; Medial septal nuclei; Lamina terminalis; Vascular organ of lamina terminalis; Rhinencephalon (paleocortex); Olfactory bulb; Olfactory tract; Anterior olfactory nucleus; Piriform cortex; Anterior commissure; Uncus; Periamygdaloid cortex; Cerebral cortex (neocortex); Frontal lobe; Cortex Primary motor cortex (Precentral gyrus, M1); Supplementary motor cortex; Premotor cortex; Prefrontal cortex; Orbitofrontal cortex; Dorsolateral prefrontal cortex; Gyri Superior frontal gyrus; Middle frontal gyrus; Inferior frontal gyrus; Brodmann areas: 4, 6, 8, 9, 10, 11, 12, 24, 25, 32, 33, 44, 45, 46, 47; Parietal lobe Cortex Primary somatosensory cortex (S 1); Secondary somatosensory cortex (S2); Posterior parietal cortex; Gyri Postcentral gyrus (Primary somesthetic area); Brodmann areas 1, 2, 3 (Primary somesthetic area); 5, 7, 23, 26, 29, 31, 39, 40; Occipital lobe Cortex Primary visual cortex (V1), V2, V3, V4, V5/MT; Gyri Lateral occipital gyrus; Brodmann areas 17 (V1, primary visual cortex); 18, 19; Temporal lobe Cortex Primary auditory cortex (A1); Secondary auditory cortex (A2); Inferior temporal cortex; Posterior inferior temporal cortex; Gyri Superior temporal gyrus; Middle temporal gyrus; Inferior temporal gyrus; Entorhinal cortex; Perirhinal cortex; Parahippocampal gyrus; Fusiform gyrus; Brodmann areas: 20, 21, 22, 27, 34, 35, 36, 37, 38, 41, 42; Insular cortex; Cingulate cortex Anterior cingulate; Posterior cingulate; Retrosplenial cortex; Indusium griseum; Subgenual area 25; Brodmann areas 23, 24; 26, 29, 30 (retrosplenial areas); 31, and 32.

Exemplary neural pathways include, but are not limited to Superior longitudinal fasciculus Arcuate fasciculus; Uncinate fasciculus; Perforant pathway; Thalamocortical radiations; Corpus callosum; Anterior commissure; Amygdalofugal pathway; Interthalamic adhesion; Posterior commissure; Habenular commissure; Fornix; Mammillotegmental; fasciculus; Incertohypothalamic pathway; Cerebral peduncle; Medial forebrain bundle; Medial longitudinal fasciculus; Myoclonic triangle; Solitary tract; Major dopaminergic pathways from dopaminergic cell groups; Mesocortical pathway; Mesolimbic pathway; Nigrostriatal pathway; Tuberoinfundibular pathway; Serotonergic pathways Raphe Nuclei; Norepinephrine Pathways Locus coeruleus and other noradrenergic cell groups; Epinephrine pathways from adrenergic cell groups; Glutamate and acetylcholine pathways from mesopontine nuclei; Motor systems/Descending fibers; Extrapyramidal system; Pyramidal tract; Corticospinal tract; or Cerebrospinal fibers; Lateral corticospinal tract; Anterior corticospinal tract; Corticopontine fibers; Frontopontine fibers; Temporopontine fibers; Corticobulbar tract; Corticomesencephalic tract; Tectospinal tract; Interstitiospinal tract; Rubrospinal tract; Rubro-olivary tract; Olivocerebellar tract; Olivospinal tract; Vestibulospinal tract; Lateral vestibulospinal tract; Medial vestibulospinal tract; Reticulospinal tract; Lateral raphespinal tract; Alpha system; and Gamma system.

Exemplary somatosensory systems include, but are not limited to Dorsal column-medial lemniscus pathway Gracile fasciculus; Cuneate fasciculus; Medial lemniscus; Spinothalamic tract; Lateral spinothalamic tract; Anterior spinothalamic tract; Spinomesencephalic tract; Spinocerebellar tract; Spino-olivary tract; and Spinoreticular tract.

Exemplary visual systems include, but are not limited to Optic tract; Optic radiation; Retinohypothalamic and tract.

Exemplary auditory system include, but are not limited to Medullary striae of fourth ventricle; Trapezoid body; and Lateral lemniscus

Exemplary nerves include, but are not limited to Brain stem Cranial nerves Terminal (0); Olfactory (I); Optic (II); Oculomotor (III); Trochlear (IV); Trigeminal (V); Abducens (VI); Facial (VII); Vestibulocochlear (VIII); Glossopharyngeal (IX); Vagus(X); Accessory (XI); and Hypoglossal (XII)

Exemplary neuro endocrine systems include, but are not limited to Hypothalamic-pituitary hormones; HPA axis; HPG axis; HPT axis; and GHRH-GH

Exemplary neuro vascular systems include, but are not limited to Middle cerebral artery; Posterior cerebral artery; Anterior cerebral artery; Vertebral artery; Basilar artery; Circle of Willis (arterial system); Blood-brain barrier; Glymphatic system; Venous systems; and Circumventricular organs.

Exemplary brain neurotransmitter systems; Noradrenaline system; Dopamine system; Serotonin system; Cholinergic system; GABA; Neuropeptides Opioid peptides; Endorphins; Enkephalins; Dynorphins; Oxytocin; and Substance P.

Exemplary dural meningeal system include, but are not limited to Brain-cerebrospinal fluid barrier; Meningeal coverings Dura mater; Arachnoid mater; Pia mater; Epidural space; Subdural space; Subarachnoid space Arachnoid septum; Superior cistern; Cistern of lamina terminalis; Chiasmatic cistern; Interpeduncular cistern; Pontine cistern; Cisterna magna; Spinal subarachnoid space; Ventricular system; Cerebrospinal fluid; Third ventricle; Fourth ventricle; Lateral ventricles Angular bundle; Anterior horn; Body of lateral ventricle; Inferior horn; Posterior horn Calcar avis; and Subventricular zone.

In one embodiment, the AAV is administered to the PNS. The “PNS” refers to the nerves and ganglia outside the brain and spinal cord. The main function of the PNS is to connect the CNS to the limbs and organs, essentially serving as a relay between the brain and spinal cord and the rest of the body. Unlike the CNS, the PNS is not protected by the vertebral column and skull, or by the blood-brain barrier, which leaves it exposed to, e.g., toxins and mechanical injuries.

The PNS is divided into the somatic nervous system and the autonomic nervous system. In the somatic nervous system, the cranial nerves are part of the PNS with the exception of the optic nerve (cranial nerve II), along with the retina. The second cranial nerve is not a true peripheral nerve but a tract of the diencephalon. Cranial nerve ganglia originated in the CNS. However, the remaining ten cranial nerve axons extend beyond the brain and are therefore considered part of the PNS. The autonomic nervous system exerts involuntary control over smooth muscle and glands. The connection between CNS and organs allows the system to be in two different functional states: sympathetic and parasympathetic.

The somatic nervous system is under voluntary control, and transmits signals from the brain to end organs such as muscles. The sensory nervous system is part of the somatic nervous system and transmits signals from senses such as taste and touch (including fine touch and gross touch) to the spinal cord and brain. The autonomic nervous system is a ‘self-regulating’ system which influences the function of organs outside voluntary control, such as the heart rate, or the functions of the digestive system

The PNS can be described in various sections include the cervical spinal nerves (C1-C4). The first 4 cervical spinal nerves, C1 through C4, split and recombine to produce a variety of nerves that serve the neck and back of head. The spinal nerve C1 is called the suboccipital nerve, which provides motor innervation to muscles at the base of the skull. C2 and C3 form many of the nerves of the neck, providing both sensory and motor control. These include the greater occipital nerve, which provides sensation to the back of the head, the lesser occipital nerve, which provides sensation to the area behind the ears, the greater auricular nerve and the lesser auricular nerve. The phrenic nerve is a nerve essential for our survival which arises from nerve roots C3, C4 and C5. It supplies the thoracic diaphragm, enabling breathing. If the spinal cord is transected above C3, then spontaneous breathing is not possible. The brachial plexus (C5-T1). The last four cervical spinal nerves, C5 through C8, and the first thoracic spinal nerve, T1, combine to form the brachial plexus, or plexus brachialis, a tangled array of nerves, splitting, combining and recombining, to form the nerves that subserve the upper-limb and upper back. Although the brachial plexus may appear tangled, it is highly organized and predictable, with little variation between people. The lumbosacral plexus (L1-Co1). The anterior divisions of the lumbar nerves, sacral nerves, and coccygeal nerve form the lumbosacral plexus, the first lumbar nerve being frequently joined by a branch from the twelfth thoracic. For descriptive purposes this plexus is usually divided into three parts: lumbar plexus, sacral plexus, and pudendal plexus. The autonomic nervous system. Exemplary autonomic nervous systems include the sympathetic nervous system; the parasympathetic nervous system and the enteric nervous system.

In one embodiment, administration results in delivery of the modified capsid to the CNS or PNS of the subject. In one embodiment, administration results in delivery of the payload to the CNS or PNS of the subject. In one embodiment, administration results in delivery of the modified viral capsid to a CNS or PNS cell population. In one embodiment, administration results in delivery of the payload to a CNS or PNS cell population. Exemplary CNS cell populations include, but are not limited to, Neurons, Oligodendrocytes, Astrocytes, Microglial cells, Ependymal cells, Radial glia cells, and Pituicytes. One skilled in the art can identify a particular CNS cell population using standard techniques, for example, assessing a cell population for known cellular markers. In one embodiment, administration results in delivery of the modified capsid to a cell type originating from the CNS, e.g., a cell that originates but extends away from the CNS, e.g., a nerve. In one embodiment, administration results in delivery of the payload to a cell type originating from the CNS, e.g., a cell that originates but extends away from the CNS, e.g., a nerve.

In one embodiment, when the composition of the invention is administered locally to the CNS or PNS, e.g., via a catheter, cannula or the like, administration results in a distribution of the composition that extends at least 0.5 inches from the initial site of administration. In one embodiment, administration results in a distribution of the composition that extends at least 1 inch, at least 1.5 inches, at least 2 inches, at least 2.5 inches, at least 3 inches, at least 3.5 inches, at least 4 inches, at least 4.5 inches, at least 5 inches, at least 5.5 inches, at least 6 inches, at least 6.5 inches, at least 7 inches, at least 7.5 inches, at least 8 inches, at least 8.5 inches, at least 9 inches, at least 9.5 inches, at least 10 inches or more from the initial site of administration. That is, the modified viral capsids of the composition are detectable in a cell (i.e., it has transduced a cell) that is at least 0.5 inches, at least 1 inch, at least 1.5 inches, at least 2 inches, at least 2.5 inches, at least 3 inches, at least 3.5 inches, at least 4 inches, at least 4.5 inches, at least 5 inches, at least 5.5 inches, at least 6 inches, at least 6.5 inches, at least 7 inches, at least 7.5 inches, at least 8 inches, at least 8.5 inches, at least 9 inches, at least 9.5 inches, at least 10 inches or more from the initial site of administration.

In one embodiment, when the composition of the invention is administered locally to the CNS or PNS, e.g., via a catheter, cannula or the like, administration results in expression of the modified capsid, viral vector, and/or payload in at least one cell type of the CNS or PNS. In one embodiment, when the composition of the invention is administered locally to the CNS or PNS, e.g., via a catheter, cannula or the like, administration results in expression of the modified capsid, viral vector, and/or payload in at least 2, 3, 4, 5, 6, 7, 8, 9, 10, 11, 12, 13, 14, 15, or more cell types of the CNS or PNS. In certain embodiments, the at least 2 cell types are adjacent to each other in the CNS or PNS. Alternatively, the at least cell types need not be adjacent to each other.

Aspects of the instant disclosure relate to compositions comprising a recombinant AAV comprising a capsid protein and a nucleic acid encoding a transgene, wherein the transgene comprises a nucleic acid sequence encoding one or more miRNAs. In some embodiments, each miRNA comprises a sequence set forth in any one of SEQ ID NOs: 6-17, 40-44, or 50-66. In some embodiments, the nucleic acid further comprises AAV ITRs. In some embodiments, the ITR is an AAV1, AAV2, AAV3b, AAV4, AAV5, AAV6, AAV7, AAV8, AAV9, AAV10, AAV11, AAV12, or AAV13 ITR. In some embodiments, a composition further comprises a pharmaceutically acceptable carrier. The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired. In some embodiments, all or, at least one of the nucleic acid sequences disclosed herein are delivered via non-viral DNA constructs comprising at least one DD-ITR. For example, the non viral DNA constructs as described in WO 2019/246554 can be utilized to deliver one or more of the nucleic acids described herein. WO 2019/246554 is incorporated herein by reference in its entirety.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target CNS tissue. In some cases, stable transgenic animals are produced by multiple doses of an rAAV.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., −10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1% or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 A, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the methods described herein relate to treating a subject having or diagnosed as having a neurological disease or disorder, e.g., Huntington's disease with a nucleic acid described herein. Subjects having a neurological disease or disorder, e.g., Huntington's disease can be identified by a physician using current methods of diagnosing such diseases and disorders. For example, symptoms and/or complications of Huntington's disease which characterize these conditions and aid in diagnosis are well known in the art and include but are not limited to, depression and anxiety and with characteristic movement disturbances and chorea. Tests that may aid in a diagnosis of Huntington's disease, e.g. include, but are not limited to, genetic tests. A family history of Huntington's disease can also aid in determining if a subject is likely to have Huntington's disease or in making a diagnosis of Huntington's disease.

The compositions and methods described herein can be administered to a subject having or diagnosed as having a neurological disease or disorder. In some embodiments, the methods described herein comprise administering an effective amount of compositions described herein, e.g. a nucleic acid described herein to a subject in order to alleviate a symptom of a neurological disease or disorder. As used herein, “alleviating a symptom” is ameliorating any condition or symptom associated with a neurological disease or disorder. As compared with an equivalent untreated control, such reduction is by at least 5%, 10%, 20%, 40%, 50%, 60%, 80%, 90%, 95%, 99% or more as measured by any standard technique.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for neuronal degradation or functionality among others. The dosage can be determined by a physician and adjusted, as necessary, to suit observed effects of the treatment.

### Immune Modulators

In some embodiments, the methods and compositions for treating a neurological disease or disorder, as described herein, further comprises administering an immune modulator. In some embodiments, the immune modulator can be administered at the time of rAAV vector administration, before rAAV vector administration or, after the rAAV vector administration.

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO 2010057626, U.S. Pat. Nos. 9,707,279, 8,323,908, US 20190345533, US 20190262434, and, WO 2020016318, each of which are incorporated in their entirety by reference.

In some embodiments, the immune modulator is Proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in U.S. Pat. Nos. 10,028,993, 9,592,247, and, U.S. Pat. No. 8,809,282, each of which are incorporated in their entirety by reference.

In alternative embodiments, the immune modulator is an inhibitor of the NF-kB pathway. In certain aspects of the embodiment, the immune modulator is Rapamycin or, a functional variant. Non-limiting examples of references disclosing rapamycin and its use described in U.S. Pat. No. 10,071,114, US 20160067228, US 20160074531, US 20160074532, US 20190076458, U.S. Pat. No. 10,046,064, are incorporated in their entirety. In other aspects of the embodiment, the immune modulator is synthetic nanocarriers comprising an immunosuppressant. Non limiting examples of references of immunosuppresants, immunosuppressants coupled to synthetic nanocarriers, synthetic nanocarriers comprising rapamycin, and/or, toloregenic synthetic nanocarriers, their doses, administration and use as described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. No. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In another further embodiment, the immunomodulator or adjuvant is poly-ICLC

In some embodiments, the immune modulator is a small molecule that inhibits the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a rAAV vector can also encode a negative regulators of innate immunity such as NLRX1. Accordingly, in some embodiments, a rAAV vector can also optionally encode one or more, or any combination of NLRX1, NS1, NS3/4A, or A46R. Additionally, in some embodiments, a composition comprising at least one rAAV vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211. In one embodiment, an immunosuppressive agent is cyclosporine A. Other examples include myophenylate mofetil, rapamicin, and anti-thymocyte globulin. In one embodiment, the immunosuppressive drug is administered in a composition comprising at least one rAAV vector as disclosed herein, or can be administered in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors f1 and 02, TNF and others that are publicly known).

The use of such immune modulating agents facilitates the ability to for one to use multiple dosing (e.g., multiple administration) over numerous months and/or years. This permits using multiple agents as discussed below, e.g., a rAAV vector encoding multiple genes, or multiple administrations to the subject.

### Kits

In one aspect, the instant disclosure relates to a nucleic acid, or recombinant viral vector comprising: (i) one or more inhibitory nucleic acids (e.g., miRNAs); and (ii) a nucleic acid encoding the CYP46A1 protein. In one aspect, the instant disclosure relates to the combination of (i) one or more inhibitory nucleic acids (e.g., miRNAs); and (ii) a nucleic acid encoding the CYP46A1 protein. In a combination of (i) and (ii), the two or more elements can be provided in a mixture or single formulation. Alternatively, the two or more elements can be provided in separate formulations that are packaged or provided as a set or kit.

The agents, e.g., viral vectors, described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for producing a rAAV, the kit comprising a container housing one or more of:
a) an isolated nucleic acid comprising an miRNA, e.g., comprising or encoded by the sequence set forth in any one of SEQ ID NOs: 6-17, 40-44, or 50-66 or comprising a seed sequence complementary to SEQ ID NO: 4, 18-39, or 46-49;
b) a recombinant viral vector comprising an isolated nucleic acid comprising a transgene encoding one or more miRNAs,
   e.g., wherein each miRNA comprises a seed sequence complementary to SEQ ID NO: 4, or wherein each miRNA comprises a sequence set forth in any one of SEQ ID NOs: 6-17, 40-44, or 50-66 flanked by a miRNA backbone sequence;
c) a recombinant viral vector comprising an isolated nucleic acid encoding the CYP46A1 protein; and/or
d) a recombinant viral vector comprising a nucleic acid comprising a transgene encoding one or more miRNAs,
   e.g., wherein each miRNA comprises a seed sequence complementary to SEQ ID NO: 4, or wherein each miRNA comprises a sequence set forth in any one of SEQ ID NOs: 6-17, 40-44, or 50-66 flanked by a miRNA backbone sequence; and a nucleic acid encoding the CYP46A1 protein.
      In some embodiments, the kit further comprises a container housing an isolated nucleic acid encoding an AAV capsid protein, for example an AAV9 capsid protein.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container.

Exemplary embodiments of the invention will be described in more detail by the following examples. These embodiments are exemplary of the invention, which one skilled in the art will recognize is not limited to the exemplary embodiments.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of Huntington's disease. A subject can be male or female.

A subject can be one who has been previously diagnosed with or identified as suffering from or having a condition in need of treatment (e.g. Huntington's disease) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA, miRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of any of the aspects, the miRNA described herein is exogenous. In some embodiments of any of the aspects, the miRNA described herein is ectopic. In some embodiments of any of the aspects, the miRNA described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector of this invention include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. Huntington's disease. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), WO 2018/057855A, U.S. Pat. No. 10,457,940, the contents of each of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

### EXAMPLES

### Example 1

In one aspect described herein are inhibitory RNAs that can be used for the treatment of Huntington's disease. In some embodiments of any of the aspects, the nucleic acid sequence of the inhibitory RNA comprises one of SEQ ID NO: 6-17, 40-44, or 50-66 or a sequence that is at least 95% (e.g., at least 95%, at least 96%, at least 97%, at least 98%, or at least 99%) identical to the sequence of at least one of SEQ ID NO: 6-17, 40-44, or 50-66 that maintains the same functions as SEQ ID NO: 6-17, 40-44, or 50-66 (e.g., HTT inhibition).

Described here are constructs comprising artificial miRNAs. pEMBL-D(+)-Syn1-hCG intron is a control vector, which is inserted with empty human chorionic gonadotropin (hCG) intron (hCGin) and driven with synapsin promoter. Two copies of control miRNA precursor (random sequences or non-functional mutation) are inserted into hCGin in the vector pEMBL-D(+)-Syn1-hCGin-2× control pre-miR. Two copies of artificial pre-miR (perfect match with 3′-UTR targeting sequences, including about 100-150 bp flanked upstream and downstream sequences) are cloned into between the hCG introns. The vector pEMBL-D(+)-Syn1-CYP46A1-hCGin-2× artificial pre-miR is a combo construct, which could produce both CYP46A1 and artificial miRNA at the same time. In order to identify whether the pre-miRNA could be processed into mature miRNA and combined with HTT targeting sequences including CAG expansions, which are perfectly complementary with mature miRNA, are inserted behind luciferase gene. For the limit of package size, small poly A is used in the constructs. Syn1 can be replaced by any of the CMV enhancer and/or, ACTB proximal promoter and/or, chimeric ACTB-HBB2 intron and one or, more of synthetic nervous system specific promoter selected from Tables 10-13 or, fragments thereof, and/or, an enhancer, and/or cis-regulatory elements (CREs) selected from the Tables 13-15.

The sequences of the following are known in the art: pEMBL; synapsin promoter (Syn1); ITRs (e.g., from AAV1, AAV2, AAV3b, AAV4, AAV5, AAV6, AAV7, AAV8, AAV9, AAV10, AAV 11, AAV12, or AAV13); hCG intron; small polyA; CYP46A1; luciferase; HTT targeting sequences; and/or HTT-3′UTR/mutant.

Synapsin-1 (Syn1) is a member of the synapsin gene family. Synapsins encode neuronal phosphoproteins which associate with the cytoplasmic surface of synaptic vesicles. Family members are characterized by common protein domains, and they are implicated in synaptogenesis and the modulation of neurotransmitter release, suggesting a potential role in several neuropsychiatric diseases. Syn1 plays a role in regulation of axonogenesis and synaptogenesis. Syn1 protein serves as a substrate for several different protein kinases and phosphorylation may function in the regulation of this protein in the nerve terminal. Mutations in this gene may be associated with X-linked disorders with primary neuronal degeneration such as Rett syndrome. Alternatively, spliced transcript variants encoding different isoforms have been identified. In some embodiments of any of the aspects, the Syn1 promoter can comprise a human promoter Syn1 (see e.g., the Syn1 promoter associated with NCBI ref numbers NG_008437.1 RefSeqGene Range 5001-52957; NM_006950.3; NP_008881.2; NM_133499.2; NP_598006.1).

CYP46A1 is a member of the cytochrome P450 superfamily of enzymes. The cytochrome P450 proteins are monooxygenases which catalyze many reactions involved in drug metabolism and synthesis of cholesterol, steroids and other lipids. This endoplasmic reticulum protein is expressed in the brain, where it converts cholesterol to 24S-hydroxycholesterol. While cholesterol cannot pass the blood-brain barrier, 24S-hydroxycholesterol can be secreted in the brain into the circulation to be returned to the liver for catabolism. In some embodiments of any of the aspects, CYP46A1 can comprise a human CYP46A1 (see e.g., NCBI ref numbers NG_007963.1 RefSeqGene Range 4881-47884; NM_006668.2; NP_006659.1). CYP46A1, the rate-limiting enzyme for cholesterol degradation, is neuroprotective in Huntington's disease (see e.g., Boussicault et al., CYP46A1, the rate-limiting enzyme for cholesterol degradation, is neuroprotective in Huntington's disease, Brain. 2016 March, 139(Pt 3):953-70; Kacher et al., CYP46A1 gene therapy deciphers the role of brain cholesterol metabolism in Huntington's disease, Brain. 2019 Aug. 1; 142(8):2432-2450; the contents of each of which are incorporated herein by reference in their entireties).

In some embodiments of any of the aspects, an miRNA comprises a sequence complementary to at least two (e.g., 2, 3, 4, 5, 6, 7, 8, 9, 10, 11, 12, 13, 14, 15, 16, 17, 18, 19, 20, 21, 22, 23, 24, or 25) continuous bases of the sequence set forth in SEQ ID NO: 3 or 4 flanked by a miRNA backbone sequence. In some embodiments of any of the aspects, an miRNA comprises a sequence complementary to at least two (e.g., 2, 3, 4, 5, 6, 7, 8, 9, 10, 11, 12, 13, 14, 15, 16, 17, 18, 19, 20, 21, 22, 23, 24, or 25) continuous bases of the sequence of an untranslated region (e.g., 5′ UTR, 3′UTR), exon, CAG repeat, or CAG jumper (e.g., CAG 5′ jumper, CAG 3′ jumper) associated with HTT (see e.g., NCBI Gene ID: 3064; e.g., SEQ ID NO: 4) flanked by a miRNA backbone sequence.

An isolated nucleic acid encoding a transgene encoding one or more miRNAs and an isolated nucleic acid encoding a CYP46A1 protein, when administered to the same patient can provide an improved therapeutic effect than either administered alone. An isolated nucleic acid encoding a transgene encoding one or more miRNAs and an isolated nucleic acid encoding a CYP46A1 protein, when administered to the same patient can provide a synergistically (rather than an additively) improved therapeutic effect than either administered alone. The isolated nucleic acid encoding a transgene encoding one or more miRNAs and isolated nucleic acid encoding a CYP46A1 protein can be administered sequentially or concurrently to the subject, in accordance with any of the methods described herein. It is expected that, rAAV comprising CYP46A1 variant CDS (as set forth in SEQ ID NO:110) will provide better therapeutic effect to treat neurological disease e.g Huntington's disease, than when administered rAAV comprising CYP46A1 non-variant sequence e.g as set forth in SEQ ID NO: 1. Similarly, it is expected that rAAV comprising miRNA (e.g., one or, more selected from SEQ ID NO: 6-17, or, 40-44, or 50-66) will provide better therapeutic effect to treat neurological disease e.g Huntington's disease when it is administered along with CYP46A1 variant CDS (as set forth in SEQ ID NO:110) than when it is administered along with CYP46A1 non-variant sequence e.g as set forth in SEQ ID NO:1.

| Exon 1 of human HTT gene | |
|---|---|
| SEQ ID NO: 3 | |
| uugcugugug aggcagaacc ugcgggggca ggggcgggcu gguucccugg ccagccauug | 60 |
| gcagaguccg caggcuaggg cugucaauca ugcuggccgg cguggccccg ccuccgccgg | 120 |
| cgcggccccg ccuccgccgg cgcacgucug ggacgcaagg cgccgugggg gcugccggga | 180 |
| cggguccaag auggacggcc gcucagguuc ugcuuuuacc ugcggcccag agccccauuc | 240 |
| auugccccgg ugcugagcgg cgccgcgagu cggcccgagg ccuccgggga cugccgugcc | 300 |
| gggcgggaga ccgccauggc gacccuggaa aagcugauga aggccuucga gucccucaag | 360 |
| uccuuccagc agcagcagca gcagcagcag cagcagcagc agcagcagca gcagcagcag | 420 |
| cagcagcagc aacagccgcc accgccgccg ccgccgccgc cgccuccuca gcuuccucag | 480 |
| ccgccgccgc aggcacagcc gcugcugccu cagccgcagc cgcccccgcc gccgcccccg | 540 |
| ccgccacccg gcccggcugu ggcugaggag ccgcugcacc gaccgugagu uugggcccgc | 600 |
| ugcagcuccc uguc | 614 |

| SEQ ID NO: 4: | |
|---|---|
| Human HTT mRNA sequence | |
| 1 | gctgccggga cgggtccaag atggacggcc gctcaggttc tgcttttacc tgcggcccag |
| 61 | agccccattc attgccccgg tgctgagcgg cgccgcgagt cggcccgagg cctccgggga |
| 121 | ctgccgtgcc gggcgggaga ccgccatggc gaccctggaa aagctgatga aggccttcga |
| 181 | gtccctcaag tccttccagc agcagcagca gcagcagcag cagcagcagc agcagcagca |
| 241 | gcagcagcag cagcagcagc aacagccgcc accgccgccg ccgccgccgc cgcctcctca |
| 301 | gcttcctcag ccgccgccgc aggcacagcc gctgctgcct cagccgcagc cgcccccgcc |
| 361 | gccgcccccg ccgccacccg gcccggctgt ggctgaggag ccgctgcacc gaccaaagaa |
| 421 | agaactttca gctaccaaga aagaccgtgt gaatcattgt ctgacaatat gtgaaaacat |
| 481 | agtggcacag tctgtcagaa attctccaga atttcagaaa cttctgggca tcgctatgga |
| 541 | actttttctg ctgtgcagtg atgacgcaga gtcagatgtc aggatggtgg ctgacgaatg |
| 601 | cctcaacaaa gttatcaaag ctttgatgga ttctaatctt ccaaggttac agctcgagct |
| 661 | ctataaggaa attaaaaaga atggtgcccc tcggagtttg cgtgctgccc tgtggaggtt |
| 721 | tgctgagctg gctcacctgg ttcggcctca gaaatgcagg ccttacctgg tgaaccttct |
| 781 | gccgtgcctg actcgaacaa gcaagagacc cgaagaatca gtccaggaga ccttggctgc |
| 841 | agctgttccc aaaattatgg cttcttttgg caattttgca aatgacaatg aaattaaggt |
| 901 | tttgttaaag gccttcatag cgaacctgaa gtcaagctcc cccaccattc ggcggacagc |
| 961 | ggctggatca gcagtgagca tctgccagca ctcaagaagg acacaatatt tctatagttg |
| 1021 | gctactaaat gtgctcttag gcttactcgt tcctgtcgag gatgaacact ccactctgct |
| 1081 | gattcttggc gtgctgctca ccctgaggta tttggtgccc ttgctgcagc agcaggtcaa |
| 1141 | ggacacaagc ctgaaaggca gcttcggagt gacaaggaaa gaaatggaag tctctccttc |
| 1201 | tgcagagcag cttgtccagg tttatgaact gacgttacat catacacagc accaagacca |
| 1261 | caatgttgtg accggagccc tggagctgtt gcagcagctc ttcagaacgc ctccacccga |
| 1321 | gcttctgcaa accctgaccg cagtcggggg cattgggcag ctcaccgctg ctaaggagga |
| 1381 | gtctggtggc cgaagccgta gtgggagtat tgtggaactt atagctggag ggggttcctc |
| 1441 | atgcagccct gtcctttcaa gaaaacaaaa aggcaaagtg ctcttaggag aagaagaagc |
| 1501 | cttggaggat gactctgaat cgagatcgga tgtcagcagc tctgccttaa cagcctcagt |
| 1561 | gaaggatgag atcagtggag agctggctgc ttcttcaggg gtttccactc cagggtcagc |
| 1621 | aggtcatgac atcatcacag aacagccacg gtcacagcac acactgcagg cggactcagt |
| 1681 | ggatctggcc agctgtgact tgacaagctc tgccactgat ggggatgagg aggatatctt |
| 1741 | gagccacagc tccagccagg tcagcgccgt cccatctgac cctgccatgg acctgaatga |
| 1801 | tgggacccag gcctcgtcgc ccatcagcga cagctcccag accaccaccg aagggcctga |
| 1861 | ttcagctgtt accccttcag acagttctga aattgtgtta gacggtaccg acaaccagta |
| 1921 | tttgggcctg cagattggac agccccagga tgaagatgag gaagccacag gtattcttcc |
| 1981 | tgatgaagcc toggaggcct tcaggaactc ttccatggcc cttcaacagg cacatttatt |
| 2041 | gaaaaacatg agtcactgca ggcagccttc tgacagcagt gttgataaat ttgtgttgag |
| 2101 | agatgaagct actgaaccgg gtgatcaaga aaacaagcct tgccgcatca aaggtgacat |
| 2161 | tggacagtcc actgatgatg actctgcacc tcttgtccat tgtgtccgcc ttttatctgc |
| 2221 | ttcgtttttg ctaacagggg gaaaaaatgt gctggttccg gacagggatg tgagggtcag |
| 2281 | cgtgaaggcc ctggccctca gctgtgtggg agcagctgtg gccctccacc cggaatcttt |
| 2341 | cttcagcaaa ctctataaag ttcctcttga caccacggaa taccctgagg aacagtatgt |
| 2401 | ctcagacatc ttgaactaca tcgatcatgg agacccacag gttcgaggag ccactgccat |
| 2461 | tctctgtggg accctcatct gctccatcct cagcaggtcc cgcttccacg tgggagattg |
| 2521 | gatgggcacc attagaaccc tcacaggaaa tacattttct ttggcggatt gcattccttt |
| 2581 | gctgcggaaa acactgaagg atgagtcttc tgttacttgc aagttagctt gtacagctgt |
| 2641 | gaggaactgt gtcatgagtc tctgcagcag cagctacagt gagttaggac tgcagctgat |
| 2701 | catcgatgtg ctgactctga ggaacagttc ctattggctg gtgaggacag agcttctgga |
| 2761 | aacccttgca gagattgact tcaggctggt gagctttttg gaggcaaaag cagaaaactt |
| 2821 | acacagaggg gctcatcatt atacagggct tttaaaactg caagaacgag tgctcaataa |
| 2881 | tgttgtcatc catttgcttg gagatgaaga ccccagggtg cgacatgttg ccgcagcatc |
| 2941 | actaattagg cttgtcccaa agctgtttta taaatgtgac caaggacaag ctgatccagt |
| 3001 | agtggccgtg gcaagagatc aaagcagtgt ttacctgaaa cttctcatgc atgagacgca |
| 3061 | gcctccatct catttctccg tcagcacaat aaccagaata tatagaggct ataacctact |
| 3121 | accaagcata acagacgtca ctatggaaaa taacctttca agagttattg cagcagtttc |
| 3181 | tcatgaacta atcacatcaa ccaccagagc actcacattt ggatgctgtg aagctttgtg |
| 3241 | tcttctttcc actgccttcc cagtttgcat ttggagttta ggttggcact gtggagtgcc |
| 3301 | tccactgagt gcctcagatg agtctaggaa gagctgtacc gttgggatgg ccacaatgat |
| 3361 | tctgaccctg ctctcgtcag cttggttccc attggatctc tcagcccatc aagatgcttt |
| 3421 | gattttggcc ggaaacttgc ttgcagccag tgctcccaaa tctctgagaa gttcatgggc |
| 3481 | ctctgaagaa gaagccaacc cagcagccac caagcaagag gaggtctggc cagccctggg |
| 3541 | ggaccgggcc ctggtgccca tggtggagca gctcttctct cacctgctga aggtgattaa |
| 3601 | catttgtgcc cacgtcctgg atgacgtggc tcctggaccc gcaataaagg cagccttgcc |
| 3661 | ttctctaaca aacccccctt ctctaagtcc catccgacga aaggggaagg agaaagaacc |
| 3721 | aggagaacaa gcatctgtac cgttgagtcc caagaaaggc agtgaggcca gtgcagcttc |
| 3781 | tagacaatct gatacctcag gtcctgttac aacaagtaaa tcctcatcac tggggagttt |
| 3841 | ctatcatctt ccttcatacc tcaaactgca tgatgtcctg aaagctacac acgctaacta |
| 3901 | caaggtcacg ctggatcttc agaacagcac ggaaaagttt ggagggtttc tccgctcagc |
| 3961 | cttggatgtt ctttctcaga tactagagct ggccacactg caggacattg ggaagtgtgt |
| 4021 | tgaagagatc ctaggatacc tgaaatcctg ctttagtcga gaaccaatga tggcaactgt |
| 4081 | ttgtgttcaa caattgttga agactctctt tggcacaaac ttggcctccc agtttgatgg |
| 4141 | cttatcttcc aaccccagca agtcacaagg ccgagcacag cgccttggct cctccagtgt |
| 4201 | gaggccaggc ttgtaccact actgcttcat ggccccgtac acccacttca cccaggccct |
| 4261 | cgctgacgcc agcctgagga acatggtgca ggcggagcag gagaacgaca cctcgggatg |
| 4321 | gtttgatgtc ctccagaaag tgtctaccca gttgaagaca aacctcacga gtgtcacaaa |
| 4381 | gaaccgtgca gataagaatg ctattcataa tcacattcgt ttgtttgaac ctcttgttat |
| 4441 | aaaagcttta aaacagtaca cgactacaac atgtgtgcag ttacagaagc aggttttaga |
| 4501 | tttgctggcg cagctggttc agttacgggt taattactgt cttctggatt cagatcaggt |
| 4561 | gtttattggc tttgtattga aacagtttga atacattgaa gtgggccagt tcagggaatc |
| 4621 | agaggcaatc attccaaaca totttttctt cttggtatta ctatcttatg aacgctatca |
| 4681 | ttcaaaacag atcattggaa ttcctaaaat cattcagctc tgtgatggca tcatggccag |
| 4741 | tggaaggaag gctgtgacac atgccatacc ggctctgcag cccatagtcc acgacctctt |
| 4801 | tgtattaaga ggaacaaata aagctgatgc aggaaaagag cttgaaaccc aaaaagaggt |
| 4861 | ggtggtgtca atgttactga gactcatcca gtaccatcag gtgttggaga tgttcattct |
| 4921 | tgtcctgcag cagtgccaca aggagaatga agacaagtgg aagcgactgt ctcgacagat |
| 4981 | agctgacatc atcctcccaa tgttagccaa acagcagaty cacattgact ctcatgaagc |
| 5041 | ccttggagtg ttaaatacat tatttgagat tttggcccct tcctccctcc gtccggtaga |
| 5101 | catgctttta cggagtatgt tcgtcactcc aaacacaatg gcgtccgtga gcactgttca |
| 5161 | actgtggata tcgggaattc tggccatttt gagggttctg atttcccagt caactgaaga |
| 5221 | tattgttctt tctcgtattc aggagctctc cttctctccg tatttaatct cctgtacagt |
| 5281 | aattaatagg ttaagagatg gggacagtac ttcaacgcta gaagaacaca gtgaagggaa |
| 5341 | acaaataaag aatttgccag aagaaacatt ttcaaggttt ctattacaac tggttggtat |
| 5401 | tcttttagaa gacattgtta caaaacagct gaaggtggaa atgagtgagc agcaacatac |
| 5461 | tttctattgc caggaactag gcacactgct aatgtgtctg atccacatct tcaagtctgg |
| 5521 | aatgttccgg agaatcacag cagctgccac taggctgttc cgcagtgatg gctgtggcgg |
| 5581 | cagtttctac accctggaca gcttgaactt gcgggctcgt tccatgatca ccacccaccc |
| 5641 | ggccctggtg ctgctctggt gtcagatact gctgcttgtc aaccacaccg actaccgctg |
| 5701 | gtgggcagaa gtgcagcaga ccccgaaaag acacagtctg tccagcacaa agttacttag |
| 5761 | tccccagatg tctggagaag aggaggattc tgacttggca gccaaacttg gaatgtgcaa |
| 5821 | tagagaaata gtacgaagag gggctctcat tctcttctgt gattatgtct gtcagaacct |
| 5881 | ccatgactcc gagcacttaa cgtggctcat tgtaaatcac attcaagatc tgatcagcct |
| 5941 | ttcccacgag cctccagtac aggacttcat cagtgccgtt catcggaact ctgctgccag |
| 6001 | cggcctgttc atccaggcaa ttcagtctcg ttgtgaaaac ctttcaactc caaccatgct |
| 6061 | gaagaaaact cttcagtgct tggaggggat ccatctcagc cagtcgggag ctgtgctcac |
| 6121 | gctgtatgtg gacaggcttc tgtgcacccc tttccgtgtg ctggctcgca tggtcgacat |
| 6181 | ccttgcttgt cgccgggtag aaatgcttct ggctgcaaat ttacagagca gcatggccca |
| 6241 | gttgccaatg gaagaactca acagaatcca ggaatacctt cagagcagcg ggctcgctca |
| 6301 | gagacaccaa aggctctatt ccctgctgga caggtttcgt ctctccacca tgcaagactc |
| 6361 | acttagtccc tctcctccag tctcttccca cccgctggac ggggatgggc acgtgtcact |
| 6421 | ggaaacagtg agtccggaca aagactggta cgttcatctt gtcaaatccc agtgttggac |
| 6481 | caggtcagat totgcactgc tggaaggtgc agagctggtg aatcggattc ctgctgaaga |
| 6541 | tatgaatgcc ttcatgatga actcggagtt caacctaagc ctgctagctc catgcttaag |
| 6601 | cctagggatg agtgaaattt ctggtggcca gaagagtgcc ctttttgaag cagcccgtga |
| 6661 | ggtgactctg gcccgtgtga gcggcaccgt gcagcagctc cctgctgtcc atcatgtctt |
| 6721 | ccagcccgag ctgcctgcag agccggcggc ctactggagc aagttgaatg atctgtttgg |
| 6781 | ggatgctgca ctgtatcagt ccctgcccac tctggcccgg gccctggcac agtacctggt |
| 6841 | ggtggtctcc aaactgccca gtcatttgca ccttcctcct gagaaagaga aggacattgt |
| 6901 | gaaattcgtg gtggcaaccc ttgaggccct gtcctggcat ttgatccatg agcagatccc |
| 6961 | gctgagtctg gatctccagg cagggctgga ctgctgctgc ctggccctgc agctgcctgg |
| 7021 | cctctggagc gtggtctcct ccacagagtt tgtgacccac gcctgctccc tcatctactg |
| 7081 | tgtgcacttc atcctggagg ccgttgcagt gcagcctgga gagcagcttc ttagtccaga |
| 7141 | aagaaggaca aataccccaa aagccatcag cgaggaggag gaggaagtag atccaaacac |
| 7201 | acagaatcct aagtatatca ctgcagcctg tgagatggtg gcagaaatgg tggagtctct |
| 7261 | gcagtcggtg ttggccttgg gtcataaaag gaatagcggc gtgccggcgt ttctcacgcc |
| 7321 | attgctaagg aacatcatca tcagcctggc ccgcctgccc cttgtcaaca gctacacacg |
| 7381 | tgtgccccca ctggtgtgga agcttggatg gtcacccaaa ccgggagggg attttggcac |
| 7441 | agcattccct gagatccccg tggagttcct ccaggaaaag gaagtcttta aggagttcat |
| 7501 | ctaccgcatc aacacactag gctggaccag tcgtactcag tttgaagaaa cttgggccac |
| 7561 | cctccttggt gtcctggtga cgcagcccct cgtgatggag caggaggaga gcccaccaga |
| 7621 | agaagacaca gagaggaccc agatcaacgt cctggccgtg caggccatca cctcactggt |
| 7681 | gctcagtgca atgactgtgc ctgtggccgg caacccagct gtaagctgct tggagcagca |
| 7741 | gccccggaac aagcctctga aagctctcga caccaggttt gggaggaagc tgagcattat |
| 7801 | cagagggatt gtggagcaag agattcaagc aatggtttca aagagagaga atattgccac |
| 7861 | ccatcattta tatcaggcat gggatcctgt cccttctctg tctccggcta ctacaggtgc |
| 7921 | cctcatcagc cacgagaagc tgctgctaca gatcaacccc gagcgggagc tggggagcat |
| 7981 | gagctacaaa ctcggccagg tgtccataca ctccgtgtgg ctggggaaca gcatcacacc |
| 8041 | cctgagggag gaggaatggg acgaggaaga ggaggaggag gccgacgccc ctgcaccttc |
| 8101 | gtcaccaccc acgtctccag tcaactccag gaaacaccgg gctggagttg acatccactc |
| 8161 | ctgttcgcag tttttgcttg agttgtacag ccgctggatc ctgccgtcca gctcagccag |
| 8221 | gaggaccccg gccatcctga tcagtgaggt ggtcagatcc cttctagtgg tctcagactt |
| 8281 | gttcaccgag cgcaaccagt ttgagctgat gtatgtgacg ctgacagaac tgcgaagggt |
| 8341 | gcacccttca gaagacgaga tcctcgctca gtacctggtg cctgccacct gcaaggcagc |
| 8401 | tgccgtcctt gggatggaca aggccgtggc ggagcctgtc agccgcctgc tggagagcac |
| 8461 | gctcaggagc agccacctgc ccagcagggt tggagccctg cacggcgtcc tctatgtgct |
| 8521 | ggagtgcgac ctgctggacg acactgccaa gcagctcatc ccggtcatca gcgactatct |
| 8581 | cctctccaac ctgaaaggga tcgcccactg cgtgaacatt cacagccagc agcacgtact |
| 8641 | ggtcatgtgt gccactgcgt tttacctcat tgagaactat cctctggacg tagggccgga |
| 8701 | attttcagca tcaataatac agatgtgtgg ggtgatgctg tctggaagtg aggagtccac |
| 8761 | cccctccatc atttaccact gtgccctcag aggcctggag cgcctcctgc tctctgagca |
| 8821 | gctctcccgc ctggatgcag aatcgctggt caagctgagt gtggacagag tgaacgtgca |
| 8881 | cagcccgcac cgggccatgg cggctctggg cctgatgctc acctgcatgt acacaggaaa |
| 8941 | ggagaaagtc agtccgggta gaacttcaga ccctaatcct gcagcccccg acagcgagtc |
| 9001 | agtgattgtt gctatggagc gggtatctgt tctttttgat aggatcagga aaggctttcc |
| 9061 | ttgtgaagcc agagtggtgg ccaggatcct gccccagttt ctagacgact tcttcccacc |
| 9121 | ccaggacatc atgaacaaag tcatcggaga gtttctgtcc aaccagcagc cataccccca |
| 9181 | gttcatggcc accgtggtgt ataaggtgtt tcagactctg cacagcaccg ggcagtcgtc |
| 9241 | catggtccgg gactgggtca tgctgtccct ctccaacttc acgcagaggg ccccggtcgc |
| 9301 | catggccacg tggagcctct cctgcttctt tgtcagcgcg tccaccagcc cgtgggtcgc |
| 9361 | ggcgatcctc ccacatgtca tcagcaggat gggcaagctg gagcaggtgg acgtgaacct |
| 9421 | tttctgcctg gtcgccacag acttctacag acaccagata gaggaggagc tcgaccgcag |
| 9481 | ggccttccag tctgtgcttg aggtggttgc agccccagga agcccatatc accggctgct |
| 9541 | gacttgttta cgaaatgtcc acaaggtcac cacctgctga gcgccatggt gggagagact |
| 9601 | gtgaggcggc agctggggcc ggagcctttg gaagtctgcg cccttgtgcc ctgcctccac |
| 9661 | cgagccagct tggtccctat gggcttccgc acatgccgcg ggcggccagg caacgtgcgt |
| 9721 | gtctctgcca tgtggcagaa gtgctctttg tggcagtggc caggcaggga gtgtctgcag |
| 9781 | tcctggtggg gctgagcctg aggccttcca gaaagcagga gcagctgtgc tgcaccccat |
| 9841 | gtgggtgacc aggtcctttc tcctgatagt cacctgctgg ttgttgccag gttgcagctg |
| 9901 | ctcttgcatc tgggccagaa gtcctccctc ctgcaggctg gctgttggcc cctctgctgt |
| 9961 | cctgcagtag aaggtgccgt gagcaggctt tgggaacact ggcctgggtc tccctggtgg |
| 10021 | ggtgtgcatg ccacgccccg tgtctggatg cacagatgcc atggcctgtg ctgggccagt |
| 10081 | ggctgggggt gctagacacc cggcaccatt ctcccttctc tcttttcttc tcaggattta |
| 10141 | aaatttaatt atatcagtaa agagattaat tttaacgtaa ctctttctat gcccgtgtaa |
| 10201 | agtatgtgaa tcgcaaggcc tgtgctgcat gcgacagcgt ccggggtggt ggacagggcc |
| 10261 | cccggccacg ctccctctcc tgtagccact ggcatagccc tcctgagcac ccgctgacat |
| 10321 | ttccgttgta catgttcctg tttatgcatt cacaaggtga ctgggatgta gagaggcgtt |
| 10381 | agtgggcagg tggccacagc aggactgagg acaggccccc attatcctag gggtgcgctc |
| 10441 | acctgcagcc cctcctcctc gggcacagac gactgtcgtt ctccacccac cagtcaggga |
| 10501 | cagcagcctc cctgtcactc agctgagaag gccagccctc cctggctgtg agcagcctcc |
| 10561 | actgtgtcca gagacatggg cctcccactc ctgttccttg ctagccctgg ggtggcgtct |
| 10621 | gcctaggagc tggctggcag gtgttgggac ctgctgctcc atggatgcat gccctaagag |
| 10681 | tgtcactgag ctgtgttttg tctgagcctc tctcggtcaa cagcaaagct tggtgtcttg |
| 10741 | gcactgttag tgacagagcc cagcatccct tctgcccccg ttccagctga catcttgcac |
| 10801 | ggtgacccct tttagtcagg agagtgcaga tctgtgctca tcggagactg ccccacggcc |
| 10861 | ctgtcagagc cgccactcct atccccaggc caggtccctg gaccagcctc ctgtttgcag |
| 10921 | gcccagagga gccaagtcat taaaatggaa gtggattctg gatggccggg ctgctgctga |
| 10981 | tgtaggagct ggatttggga gctctgcttg ccgactggct gtgagacgag gcaggggctc |
| 11041 | tgcttcctca gccctagagg cgagccaggc aaggttggcg actgtcatgt ggcttggttt |
| 11101 | ggtcatgccc gtcgatgttt tgggtattga atgtggtaag tggaggaaat gttggaactc |
| 11161 | tgtgcaggtg ctgccttgag acccccaagc ttccacctgt ccctctccta tgtggcagct |
| 11221 | ggggagcagc tgagatgtgg acttgtatgc tgcccacata cgtgaggggg agctgaaagg |
| 11281 | gagcccctcc tctgagcagc ctctgccagg cctgtatgag gottttccca ccagctccca |
| 11341 | acagaggcct cccccagcca ggaccacctc gtcctcgtgg cggggcagca ggagcggtag |
| 11401 | aaaggggtcc gatgtttgag gaggccctta agggaagcta ctgaattata acacgtaaga |
| 11461 | aaatcaccat tccgtattgg ttgggggctc ctgtttctca tcctagcttt ttcctggaaa |
| 11521 | gcccgctaga aggtttggga acgaggggaa agttctcaga actgttggct gctccccacc |
| 11581 | cgcctcccgc ctcccccgca ggttatgtca gcagctctga gacagcagta tcacaggcca |
| 11641 | gatgttgttc ctggctagat gtttacattt gtaagaaata acactgtgaa tgtaaaacag |
| 11701 | agccattccc ttggaatgca tatcgctggg ctcaacatag agtttgtctt cctcttgttt |
| 11761 | acgacgtgat ctaaaccagt ccttagcaag gggctcagaa caccccgctc tggcagtagg |
| 11821 | tgtcccccac ccccaaagac ctgcctgtgt gctccggaga tgaatatgag ctcattagta |
| 11881 | aaaatgactt cacccacgca tatacataaa gtatccatgc atgtgcatat agacacatct |
| 11941 | ataattttac acacacacct ctcaagacgg agatgcatgg cctctaagag tgcccgtgtc |
| 12001 | ggttcttcct ggaagttgac tttccttaga cccgccaggt caagttagcc gcgtgacgga |
| 12061 | catccaggcg tgggacgtgg tcagggcagg gctcattcat tgcccactag gatcccactg |
| 12121 | gcgaagatgg tctccatatc agctctctgc agaagggagg aagactttat catgttccta |
| 12181 | aaaatctgtg gcaagcaccc atcgtattat ccaaattttg ttgcaaatgt gattaatttg |
| 12241 | gttgtcaagt tttgggggtg ggctgtgggg agattgcttt tttttcctg ctggtaatat |
| 12301 | cgggaaagat tttaatgaaa ccagggtaga attgtttggc aatgcactga agcgtgtttc |
| 12361 | tttcccaaaa tgtgcctccc ttccgctgcg ggcccagctg agtctatgta ggtgatgttt |
| 12421 | ccagctgcca agtgctcttt gttactgtcc accctcattt ctgccagcgc atgtgtcctt |
| 12481 | tcaaggggaa aatgtgaagc tgaaccccct ccagacaccc agaatgtagc atctgagaag |
| 12541 | gccctgtgcc ctaaaggaca cccctcgccc ccatcttcat ggagggggtc atttcagagc |
| 12601 | cctcggagcc aatgaacagc tcctcctctt ggagctgaga tgagccccac gtggagctcg |
| 12661 | ggacggatag tagacagcaa taactcggtg tgtggccgcc tggcaggtgg aacttcctcc |
| 12721 | cgttgcgggg tggagtgagg ttagttctgt gtgtctggtg ggtggagtca ggcttctctt |
| 12781 | gctacctgtg agcatccttc ccagcagaca tcctcatcgg gctttgtccc tcccccgctt |
| 12841 | cctccctctg cggggaggac ccgggaccac agctgctggc cagggtagac ttggagctgt |
| 12901 | cctccagagg ggtcacgtgt aggagtgaga agaaggaaga tcttgagagc tgctgaggga |
| 12961 | ccttggagag ctcaggatgg ctcagacgag gacactcgct tgccgggcct gggcctcctg |
| 13021 | ggaaggaggg agctgctcag aatgccgcat gacaactgaa ggcaacctgg aaggttcagg |
| 13081 | ggccgctctt cccccatgtg cctgtcacgc tctggtgcag tcaaaggaac gccttcccct |
| 13141 | cagttgtttc taagagcaga gtctcccgct gcaatctggg tggtaactgc cagccttgga |
| 13201 | ggatcgtggc caacgtggac ctgcctacgg agggtgggct ctgacccaag tggggcctcc |
| 13261 | ttgtccaggt ctcactgctt tgcaccgtgg tcagagggac tgtcagctga gcttgagctc |
| 13321 | ccctggagcc agcagggctg tgatgggcga gtcccggagc cccacccaga cctgaatgct |
| 13381 | tctgagagca aagggaagga ctgacgagag atgtatattt aattttttaa ctgctgcaaa |
| 13441 | cattgtacat ccaaattaaa ggaaaaaaat ggaaaccatc aaaaaaaaaa aaaaaaaa |
| Human HTT protein sequence | |
| SEQ ID NO: 5 | |
| 1 | matleklmka feslksfqqq qqqqqqqqqq qqqqqqqqqq pppppppppp pqlpqpppqa |
| 61 | qpllpqpqpp ppppppppgp avaeeplhrp kkelsatkkd rvnhcltice nivaqsvrns |
| 121 | pefqkllgia melfllcsdd aesdvrmvad eclnkvikal mdsnlprlql elykeikkng |
| 181 | aprslraalw rfaelahlvr pqkcrpylvn llpcltrtsk rpeesvqetl aaavpkimas |
| 241 | fgnfandnei kvllkafian lksssptirr taagsavsic qhsrrtqyfy swllnvllgl |
| 301 | lvpvedehst llilgvlltl rylvpllqqq vkdtslkgsf gvtrkemevs psaeqlvqvy |
| 361 | eltlhhtqhq dhnvvtgale llqqlfrtpp pellqtltav ggigqltaak eesggrsrsg |
| 421 | siveliaggg sscspvlsrk qkgkvllgee ealeddsesr sdvsssalta svkdeisgel |
| 481 | aassgvstpg saghdiiteq prsqhtlqad svdlascdlt ssatdgdeed ilshsssqvs |
| 541 | avpsdpamdl ndgtqasspi sdssqttteg pdsavtpsds seivldgtdn qylglqigqp |
| 601 | qdedeeatgi lpdeaseafr nssmalqqah llknmshcrq psdssvdkfv lrdeatepgd |
| 661 | qenkpcrikg digqstddds aplvhcvrll sasflltggk nvlvpdrdvr vsvkalalsc |
| 721 | vgaavalhpe sffsklykvp ldtteypeeq yvsdilnyid hgdpqvrgat ailcgtlics |
| 781 | ilsrsrfhvg dwmgtirtlt gntfsladci pllrktlkde ssvtcklact avrncvmslc |
| 841 | sssyselglq liidvltlrn ssywlvrtel letlaeidfr lvsfleakae nlhrgahhyt |
| 901 | gllklqervl nnvvihllgd edprvrhvaa aslirlvpkl fykcdqgqad pvvavardqs |
| 961 | svylkllmhe tqppshfsvs titriyrgyn llpsitdvtm ennlsrviaa vshelitstt |
| 1021 | raltfgccea lcllstafpv ciwslgwhcg vpplsasdes rksctvgmat miltllssaw |
| 1081 | fpldlsahqd alilagnlla asapkslrss waseeeanpa atkqeevwpa lgdralvpmv |
| 1141 | eqlfshllkv inicahvldd vapgpaikaa lpsltnppsl spirrkgkek epgeqasvpl |
| 1201 | spkkgseasa asrqsdtsgp vttskssslg sfyhlpsylk lhdvlkatha nykvtldlqn |
| 1261 | stekfggflr saldvlsqil elatlqdigk cveeilgylk scfsrepmma tvcvqqllkt |
| 1321 | lfgtnlasqf dglssnpsks qgraqrlgss svrpglyhyc fmapythftq aladaslrnm |
| 1381 | vqaeqendts gwfdvlqkvs tqlktnltsv tknradknai hnhirlfepl vikalkqytt |
| 1441 | ttcvqlqkqv ldllaqlvql rvnyclldsd qvfigfvlkq feyievgqfr eseaiipnif |
| 1501 | fflvllsyer yhskqiigip kiiqlcdgim asgrkavtha ipalqpivhd lfvlrgtnka |
| 1561 | dagkeletqk evvvsmllrl iqyhqvlemf ilvlqqchke nedkwkrlsr qiadiilpml |
| 1621 | akqqmhidsh ealgvlntlf eilapsslrp vdmllrsmfv tpntmasvst vqlwisgila |
| 1681 | ilrvlisqst edivlsrige lsfspylisc tvinrlrdgd ststleehse gkqiknlpee |
| 1741 | tfsrfllqlv gilledivtk qlkvemseqq htfycqelgt llmclihifk sgmfrritaa |
| 1801 | atrlfrsdgc ggsfytldsl nlrarsmitt hpalvllwcq illlvnhtdy rwwaevqqtp |
| 1861 | krhslsstkl lspqmsgeee dsdlaaklgm cnreivrrga lilfcdyvcq nlhdsehltw |
| 1921 | livnhiqdli slsheppvqd fisavhrnsa asglfiqaiq srcenlstpt mlkktlqcle |
| 1981 | gihlsqsgav ltlyvdrllc tpfrvlarmv dilacrrvem llaanlqssm aqlpmeelnr |
| 2041 | iqeylqssgl aqrhqrlysl ldrfristmq dslspsppvs shpldgdghv sletvspdkd |
| 2101 | wyvhlvksqc wtrsdsalle gaelvnripa edmnafmmns efnlsllapc Islgmseisg |
| 2161 | gqksalfeaa revtlarvsg tvqqlpavhh vfqpelpaep aaywsklndl fgdaalyqsl |
| 2221 | ptlaralaqy lvvvsklpsh lhlppekekd ivkfvvatle alswhliheq iplsldlqag |
| 2281 | ldccclalql pglwsvvsst efvthacsli ycvhfileav avqpgeqlls perrtntpka |
| 2341 | iseeeeevdp ntqnpkyita acemvaemve slqsvlalgh krnsgvpafl tpllrniiis |
| 2401 | larlplvnsy trvpplvwkl gwspkpggdf gtafpeipve flqekevfke fiyrintlgw |
| 2461 | tsrtqfeetw atllgvlvtq plvmeqeesp peedtertqi nvlavqaits lvlsamtvpv |
| 2521 | agnpavscle qqprnkplka ldtrfgrkls iirgiveqei qamvskreni athhlyqawd |
| 2581 | pvpslspatt galishekll lqinperelg smsyklgqvs ihsvwlgnsi tplreeewde |
| 2641 | eeeeeadapa psspptspvn srkhragvdi hscsqfllel ysrwilpsss arrtpailis |
| 2701 | evvrsllvvs dlfternqfe lmyvtltelr rvhpsedeil agylvpatck aaavlgmdka |
| 2761 | vaepvsrlle stlrsshlps rvgalhgvly vlecdllddt akqlipvisd yllsnlkgia |
| 2821 | hcvnihsqqh vlvmcatafy lienypldvg pefsasiiqm cgvmlsgsee stpsiiyhca |
| 2881 | lrglerllls eqlsrldaes lvklsvdrvn vhsphramaa lglmltcmyt gkekvspgrt |
| 2941 | sdpnpaapds esvivamerv svlfdrirkg fpcearvvar ilpqflddff ppqdimnkvi |
| 3001 | geflsnqqpy pqfmatvvyk vfqtlhstgq ssmvrdwvml slsnftqrap vamatwslsc |
| 3061 | ffvsastspw vaailphvis rmgkleqvdv nlfclvatdf yrhqieeeld rrafqsvlev |
| 3121 | vaapgspyhr lltclrnvhk vttc |

CYP46A1 variant
SEQ ID NO: 109
MSPGLLLLGSAVLLAFGLCCTFVHRARSRYEHIPGPPRPSFLLGH
LPCFWKKDEVGGRVLQDVFLDWAKKYGPVVRVNVFHKTSVIVTSPESVKKFLMSTKYNK
DSKMYRALQTVFGERLFGQGLVSECNYERWHKQRRVIDLAFSRSSLVSLMETFNEKAEQ
LVEILEAKADGQTPVSMQDMLTYTAMDILAKAAFGMETSMLLGAQKPLSQAVKLMLEGI
TASRNTLAKFLPGKRKQLREVRESIRFLRQVGRDWVQRRREALKRGEEVPADILTQILK
AEEGAQDDEGLLDNFVTFFIAGHETSANHLAFTVMELSRQPEIVARLQAEVDEVIGSKR
YLDFEDLGRLQYLSQVLKESLRLYPPAWGTFRLLEEETLIDGVRVPGNTPLLFSTYVMG
RMDTYFEDPLTFNPDRFGPGAPKPRFTYFPFSLGHRSCIGQQFAQMEVKVVMAKLLQRL
EFRLVPGQRFGLQEQATLKPLDPVLCTLRPRGWQPAPPPPPC
CYP46A1 variant CDS
SEQ ID NO: 110
atgagccccgggctgctgctgctcggtagcgccgtcctgctcgccttcggcctctgctgcaccttcgtgcaccgcgctcgcagccgct
acgagcacatccccgggccgccgcggcccagtttccttctaggacacctcccctgcttttggaaaaaggatgaggttggtggccgtgt
gctccaagatgtgtttCtAgattgggctaagaagtatggacctgtAgtgcgggtcaacgtcttccacaaaacctcagtcatcgtcacg
agtcctgagtcggttaagaagttcctgatgtcaaccaagtacaacaaggactccaagatgtaccgtgcgctccagactgtgtttggtga
gagactcttcggccaaggcttggtgtccgaatgcaactatgagcgctggcacaagcagcggagagtGatagacctggccttcagcc
ggagctccttggttagcttaatggaaacattcaacgaAaaggctgagcagctggtggagattctagaagccaaggcagatgggcag
accccTgtGAGCatgcaggacatgctgacctacaccgccatggacatcctggccaaggcagcttttgggatggagaccagtatg
ctgctgggtgcccagaagcctctgtcccaggcagtgaaacttatgttggagggaatcactgcgtcccgcaacactctggcaaagttcct
gccagggaagaggaagcagctccgggaggtccgggagagcattcgcttcctgcgccaggtgggcagggactgggtccagcgcc
gccgggaagccctgaagaggggcgaggaggttcctgccgacatcctcacacagattctgaaagctgaagagggagcccaggacg
acgagggtctgctggacaacttcgtcaccttcttcattgctggtcacgagacctctgccaaccacttggcgttcacagtgatggagctgt
ctcgccagccagagatcgtggcaaggctgcaggccgaggtggatgaAgtGattggttctaagaggtacctggatttcgaggacctg
gggagactgcagtacctgtcccaggtcctcaaagagtcgctgaggctgtacccaccagcatggggcacctttAGGctgctggaag
aggagaccttgattgatggggtGagagtccccggcaacaccccgctcttgttcagcacctatgtGatggggcggatggacacatact
ttgaggacccgctgactttcaaccccgatcgcttcggccctggagcacccaagccacggttcacctacttccccttctccctgggccac
cgctcctgcatcgggcagcagtttgctcagatggaggtgaaggtggtcatggcaaagctgctgcagaggctggagttccggctggtg
cccgggcagcgcttcgggctgcaggagcaggccacactcaagccactggaccccgtgctgtgcaccctgcggccccgcggctgg
cagcccgcacccccaccacccccctgc

### Example 2

The synthetic NS-specific promoters according to the present invention were designed through reviewing scientific literature to identify genes and their respective promoters which are highly active in NS cells.

During the design of these promoters, particular shortcomings of known NS-specific promoters were considered. First of all, known NS-specific promoters which are specific for a NS cell type (e.g. Synapsin-1, CAMKIIa and GFAP) are not expressed in the whole cellular population (e.g. not expressed in all neurones/astrocytes). This has been shown for GFAP by (Zhang et al., 2019) and can be seen from distribution of Syn-1 in neurones from the Allen brain atlas. Secondly, the majority of the known CREs, promoter elements and promoters are too large to be included in a self-complementary AAV vector (scAAV) (depending on the size of the transgene, the size of the promoter may need to be less than 1000 bp, preferably less than 900 bp, more preferably less than 800 bp, most preferably less than 700 bp). Additionally, expression may be required in a specific cell type or a combination of cell types across the entire NS, suitably the entire CNS or the entire brain.

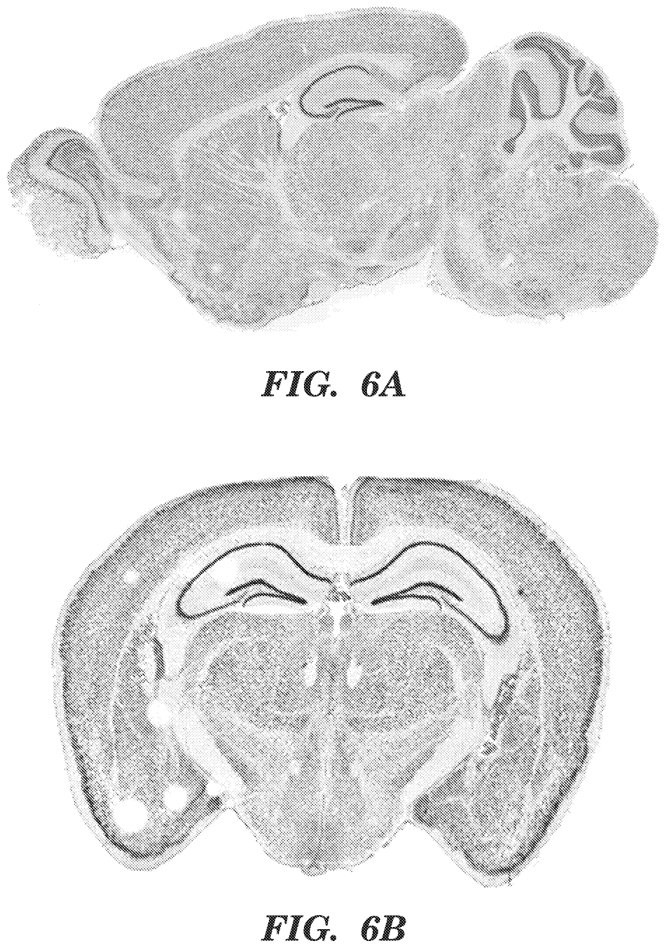

Currently known promoters are not able to address these shortcomings and there is a need in gene therapy to develop short, cell-type NS-specific promoters both with targeted localised expression and also with expression across the entire NS. For example, the requirement for an expression across the entire NS (e.g. the entire brain) is highlighted by the expression pattern of the HTT (huntingtin) and CYP46A1 genes in the adult mouse brain shown in FIG. 6A and FIG. 6B. Since the HTT (huntingtin) gene is expressed throughout the brain, it may be beneficial for any potential expression product suppressing the faulty huntingtin gene and/or counteracting or alleviating the detrimental effects of the faulty huntingtin to be expressed throughout the brain. Similarly, since the CYP46A1 gene is expressed throughout the brain, it may be beneficial for any potential supplementary CYP46A1 expression to be throughout the brain.

Gene expression in all neurons as well as astrocytes and/or oligodendrocytes across the CNS may be desirable in treatment of some diseases such as Huntington's disease. Expression in astrocytes and oligodendrocytes may be beneficial as glial cells are implicated in Huntington's disease (Shin et al., 2005).

Therefore, the present invention sets out to design tandem NS promoters which are active in multiple NS cell types while addressing some of the shortcomings listed above. For example, the promoter design involved combination of one or more CRE together with a promoter element in order to broaden the cell tropism compared to the individual CRE/promoter element in order to create promoters active in multiple NS cell types and also to address the drawback of known promoters not being expressed in the whole cellular population. Additionally, in order to address the drawback of known CREs, promoter elements and promoters being too large to be included in an AAV vector such as self-complementary AAV vector (scAAV), some of the CREs and promoter elements of the present invention have been shortened using bioinformatic analysis, literature searching and publicly available genomic databases but are still expected to be active CREs and promoter elements.

The synthetic NS-specific promoters according to the present invention are operably linked to a nucleic acid sequence encoding the CYP46A1 transgene and a Human influenza hemagglutinin (HA) tag and experimentally tested in wildtype C57BL6/J mice. The synthetic NS-specific promoters according to the present invention operably linked to a nucleic acid sequence encoding the CYP46A1 transgene and a HA tag are administered intravenously in a viral vector. Vector copy number will be assessed in brain and spinal cord tissue sections by qPCR analysis of the viral transgene CYP46A1 normalised to internal genomic DNA copy number control to confirm equivalent injected doses. Western blot will be performed to assess the protein expression of the HA tagged transgene in the brain and spinal cord tissue. Finally, immunofluorescent staining will be performed on brain and spinal cord tissue sections to assess the expression of the transgene within CNS cell types. Similarly, immunofluorescent staining can be performed on PNS tissue sections to assess the expression of the transgene within PNS cell types. Specifically, double staining will be performed using the HA tag to mark CYP46A1 gene expression and standard markers for neurones, astrocytes, oligodendrocytes and microglia.

SP0013 (SEQ ID NO: 74) is predicted to be active in neurones and astrocytes. SP0014 (SEQ ID NO: 75) is predicted to be active in neurones and astrocytes. SP0026 (SEQ ID NO: 76) is predicted to be active in excitatory neurones and astrocytes. SP0027 (SEQ ID NO: 77) is predicted to be active in excitatory neurones and astrocytes. SP0030 (SEQ ID NO: 78) is predicted to be active in neurones and astrocytes. SP0031 (SEQ ID NO: 79) is predicted to be active in neurones and astrocytes. SP0032 (SEQ ID NO: 80) is predicted to be active in neurones, astrocytes and oligodendrocytes. SP0033 (SEQ ID NO: 81) is predicted to be active in neurones, astrocytes and oligodendrocytes. SP0019 (SEQ ID NO: 82) is predicted to be active in neurones, astrocytes and oligodendrocytes. SP0020 (SEQ ID NO: 83) is predicted to be active in neurones, astrocytes and oligodendrocytes. SP0021 (SEQ ID NO: 84) is predicted to be active in neurones, astrocytes and oligodendrocytes. SP0022 (SEQ ID NO: 85) is predicted to be active in neurones, astrocytes and oligodendrocytes. SP0028 (SEQ ID NO: 86) is predicted to be active in excitatory neurones, astrocytes and oligodendrocytes. SP0029 (SEQ ID NO: 87) is predicted to be active in excitatory neurones, astrocytes and oligodendrocytes. SP0011 (SEQ ID NO: 88) is predicted to be active in neurones and astrocytes. SP0034 (SEQ ID NO: 89) is predicted to be active in neurones and astrocytes. SP0035 (SEQ ID NO: 90) is predicted to be active in neurones and astrocytes. SP0036 (SEQ ID NO: 154) is predicted to be active in neurones and astrocytes.

Bioinformatic analysis of RNA sequencing data predicts that some of the genes associated with the CREs and/or promoter elements of the present invention (aqp4, cend1, eno2, gfap, s100B, syn1) are expressed in the dorsal root ganglion and tibial nerve. Therefore, CREs and/or promoter elements associated with these genes are predicted to be expressed in cells of the PNS. CRE0001_S100B (SEQ ID NO: 106), CRE0002_S100B (SEQ ID NO: 108), CRE0005_GFAP (SEQ ID NO: 103), CRE0007_GFAP (SEQ ID NO: 104), CRE0009_S100B (SEQ ID NO: 107), CRE0006_GFAP (SEQ ID NO: 99), CRE0008_GFAP (SEQ ID NO: 100), CRE0006_AQP4 (SEQ ID NO: 101), CRE0008 AQP4 (SEQ ID NO: 102), or functional variants thereof are predicted to be active in cells of the PNS.

Bioinformatic analysis of single cell RNA sequencing data predicts that some of the genes associated with the CREs and/or promoter elements of the present invention (aqp4, cend1, eno2, gfap, s100B, syn1) are expressed in sensory neurones, PNS sympathetic neurones and PNS enteric neurones. Therefore, CREs and/or promoter elements associated with these genes are predicted to be expressed in sensory neurones, PNS sympathetic neurones and PNS enteric neurones. CRE0001_S100B (SEQ ID NO: 106), CRE0002_S100B (SEQ ID NO: 108), CRE0005_GFAP (SEQ ID NO: 103), CRE0007_GFAP (SEQ ID NO: 104), CRE0009_S100B (SEQ ID NO: 107), CRE0006_GFAP (SEQ ID NO: 99), CRE0008_GFAP (SEQ ID NO: 100), CRE0006_AQP4 (SEQ ID NO: 101), CRE0008 AQP4 (SEQ ID NO: 102), or functional variants thereof are predicted to be active in sensory neurones, PNS sympathetic neurones and/or PNS enteric neurones.

### Example 3

Described herein in is a method of manufacturing viral vectors from Pro10/HEK293 cells that have been engineered to stably integrate the CYP46A1 gene.

The stable cell line, Pro10/HEK293, as described in U.S. Pat. No. 9,441,206, is ideal for scalable production of AAV vectors. This cell line can be contacted with an expression vector comprising CYP46A1 gene operatively linked to any NS-specific promoter described herein, for example as described in Tables 10-15, or variants thereof. Clonal populations having CYP46A1 integrated into their genome are selected using methods well known in the art. Pro 10/HEK293 cells stably encompassing CYP46A1 gene are transfected with a Packaging plasmid encoding Rep2 and serotype-specific Cap2: AAV-Rep/Cap, and the Ad-Helper plasmid (XX680: encoding adenoviral helper sequences).

Transfection. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The cocktail is inverted to mix prior to being incubated at room temperature. The transfection cocktail is pipetted into the flasks and placed back in the shaker/incubator. All optimization studies are carried out at 30 mL culture volumes followed by validation at larger culture volumes. Cells are harvested 48 hours post-transfection.

Production of rAAV Using Wave Bioreactor Systems. Wave bags are seeded 2 days prior to transfection. Two days post-seeding the wave bag, cell culture counts are taken and the cell culture is then expanded/diluted before transfection. The wave bioreactor cell culture is then transfected. Cell culture is harvested from the wave bio-reactor bag at least 48 hours post-transfection.

Titer. AAV titers are calculated after DNase digestion using qPCR against a standard curve (AAV ITR specific) and primers specific to CYP46A1 gene. Harvesting Suspension Cells from Shaker Flasks and 60 Wave Bioreactor Bags. 48 hours post-transfection, cell cultures are collected into 500 mL polypropylene conical tubes (Corning) either by pouring from shaker flasks or pumping from wave bioreactor bags. The cell culture is then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 min. At this point, the pellet can either be stored in NLT −60° C. or continued through purification.

Titering rAAV from Cell Lysate Using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl2), 5 mM MgCl2, 50 mM Tris-HCl pH 8.0) followed by sonication to lyse the cells efficiently. 300 μL is then removed and placed into a 1.5 mL microfuge tube. 140 units of DNase I is then added to each sample and incubated at 37° C. for 1 hour. To determine the effectiveness of the DNase digestion, 4-5 mg of CYP46A1 plasmid is spiked into a non-transfected cell lysate with and without the addition of DNase. 50 L of EDTA/Sarkosyl solution (6.3% sarkosyl, 62.5 mM EDTA pH 8.0) is added to each tube and incubated at 70° C. for 20 minutes. 50 μL of Proteinase K (10 mg/mL) is then added and incubated at 55° C. for at least 2 hours. Samples are boiled for 15 minutes to inactivate the Proteinase K. An aliquot is removed from each sample to be analyzed by qPCR. Two qPCR reactions are carried out in order to effectively determine how much rAAV vector is generated per cell. One qPCR reaction is set up using a set of primers designed to bind to a homologous sequence on the backbones of plasmids XX680, pXR2 and CYP46A1. The second qPCR reaction is set up using a set of primers to bind and amplify a region within the CYP46A1 gene. qPCR is conducted using Sybr green reagents and Light cycler 480 from Roche. Samples are denatured at 95° C. for 10 minutes followed by 45 cycles (90° C. for 10 sec, 62° C. for 10 sec and 72° C. for 10 sec) and melting curve (1 cycle 99° C. for 30 sec, 65° C. for 1 minute continuous).

Purification of rAAV from Crude Lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art can use as mechanical methods such as microfluidization or chemical methods such as detergents, etc., followed by a clarification step using depth filtration or Tangential Flow Filtration (TFF).

AAV Vector Purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc.), which are incorporated herein by reference in their entireties.

### Example 4

A selection of the NS-specific promoters according to the present invention were tested in neuroblastoma-derived SH-SY5Y cells.

### Materials and Methods

Cell maintenance and transfection. SH-SY5Y cells were cultured in HAM F12 media with 1 mM L-Glutamine (Gibco 11765-054), 15% heat-inactivated FBS (ThermoFisher 10500064), 1% non-essential amino acids (Merck M1745—100 ML), and 1% penicillin/streptomycin (ThermoFisher 15140122). The cells were passaged twice a week between 1:3 and 1:4 to maintain a healthy cell density of between 70-80%. The cells were kept under passage number 20. For transfections, the cells were seeded at 10⁵ cells/well into an adherent 48 well plate. 24 hours post-seeding, 300 ng plasmid was transfected into the cells using Lipofectamine3000 reagent (ThermoFisher L3000008).

The plasmid which was transfected into the SHSY5Y cell line comprises SP0013, SPOO14, SP0030, SP0031, SP0032, SP0019, SP0020, SP0021, SP0033, SP0011, SP0034, SP0035 or SP0036 operably linked to GFP.

Flow Cytometry. 48 hours after transfection, SH-SY5Y cells were washed with PBS before dissociation with 0.05% trypsin. The cells were collected and resuspended in 90% PBS, 10% FBS solution. The GFP expression of the cells was assessed by flow cytometry using the Attune Nxt Acoustic Focusing Cytometer. The cell viability dye 7-AAD (ThermoFisher 00-6993-50) was mixed with the control cell population to identify and exclude the dead cells. The expression of GFP was measured in the living, single cell population using a blue 488 nm laser at the band-pass filter 510/10 nm. Untransfected cells were used to set the gates for GFP-negative and GFP-positive cells. The number of GFP-positive single cells and the median GFP fluorescence of all GFP-positive cells was calculated by the Attune Nxt Software.

### Results

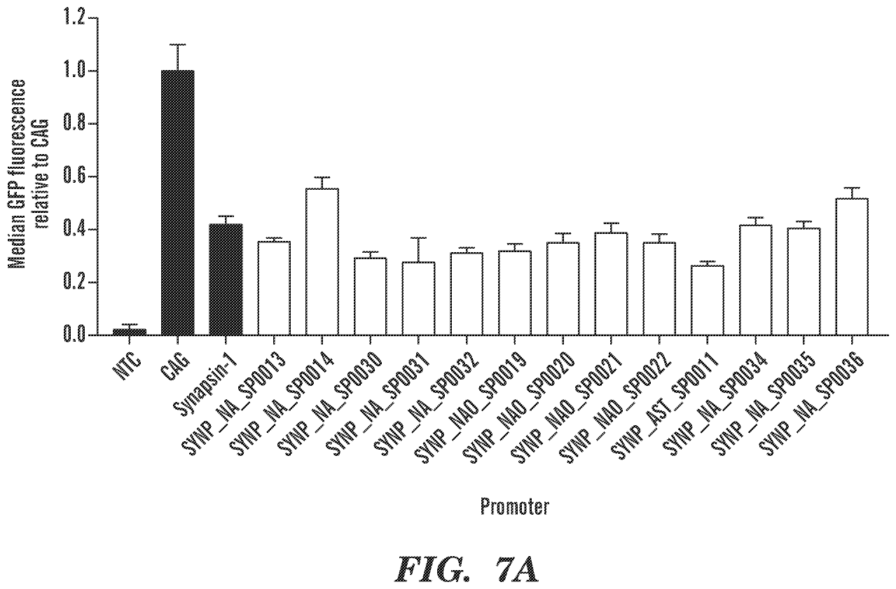

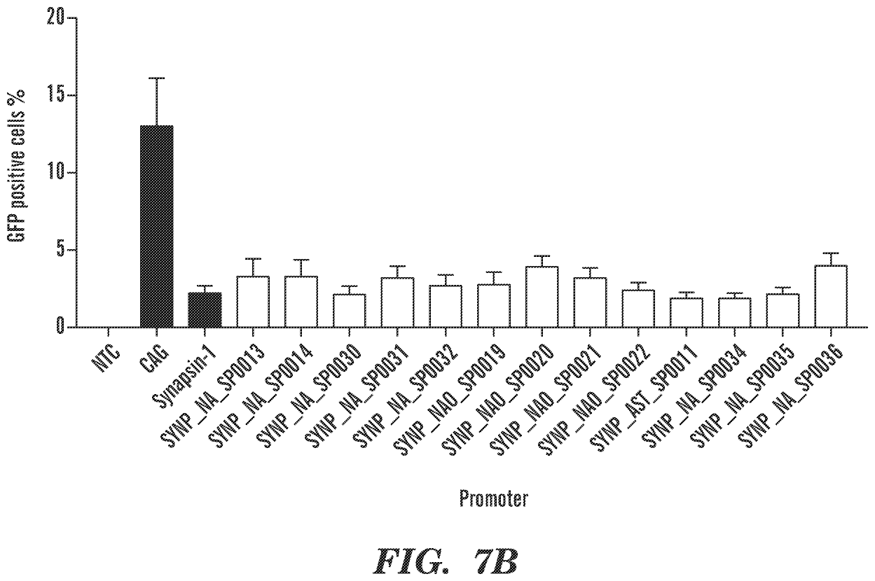

The results of this experiment are shown in FIGS. 7A and 7B. Neuroblastoma-derived SH-SY5Y cells transfected with expression cassette comprising SP0013, SP0014, SP0030, SP0031, SP0032, SP0019, SP0020, SP0021, SP0022, SP0011, SP0034, SP0035 or SP0036 operably linked to GFP were assessed for median GFP expression and percentage of GFP positive cells by flow cytometry. Expression cassettes comprising known promoters Synapsin-1 and CAG operably linked to GFP were used as controls. All tested promoters have comparable transfection efficiency and median GFP expression to the neuronal-specific control promoter Synapsin-1 (see FIGS. 7A and 7B). Control promoter CAG showed 2 to 3 times higher transfection efficiency (FIG. 7B) and around 2.5 higher median GFP expression compared to control promoter Synapsin-1 and the tested synthetic NS-specific promoters (FIG. 7A).

Synthetic NS-specific promoter SP0028 (SEQ ID NO: 86) is a similar design to synthetic NS-specific promoter SP0019 (SEQ ID NO: 82) as both comprise identical elements. As such, SP0028 (SEQ ID NO: 86) may be expected to perform similarly to SP0019 (SEQ ID NO: 82).

Synthetic NS-specific promoter SP0029 (SEQ ID NO: 87) is a similar design to synthetic NS-specific promoter SP0021 (SEQ ID NO: 84) as both comprise identical elements. As such, SP0029 (SEQ ID NO: 87) may be expected to perform similarly to SP0021 (SEQ ID NO: 84).

Synthetic NS-specific promoter SP0026 (SEQ ID NO: 76) is a similar design to synthetic NS-specific promoter SP0013 (SEQ ID NO: 74) as both comprise identical elements. As such, SP0026 (SEQ ID NO: 76) may be expected to perform similarly to SP0013 (SEQ ID NO: 74).

Synthetic NS-specific promoter SP0027 (SEQ ID NO: 77) is a similar design to synthetic NS-specific promoter SP0014 (SEQ ID NO: 75) as both comprise identical elements. As such, SP0027 (SEQ ID NO: 77) may be expected to perform similarly to SP0014 (SEQ ID NO: 75).

Synthetic NS-specific promoter SP0033 (SEQ ID NO: 81) is a similar design to SP0021 (SEQ ID NO: 84) as both comprise identical and similar elements. Therefore, SP0033 (SEQ ID NO: 81) is a shorter version of SP0021 (SEQ ID NO: 84) and, as such, may be expected to perform similarly.

### Example 5

Modified vector comprising-CYP46A1 or GFP and covalent mannosylation of the vector will be compared to Parental unmodified rAAV. Delivery of CYP46A1 by rAAV drives abundant secretion of CYP46A1 from transduced neurons that can be visually detected by immunohistochemistry and quantified by ELISA of tissue extracts. After infusion of Modified AAV-CYP46A1 into the thalamus, e.g., by convection-enhanced delivery described in U.S. patent application Ser. No. 13/146,640 or catheter delivery in monkey, the extent of CYP46A1-immunopositive staining will be assessed in the frontal cortex ipsilateral to the infusion site. The expression of CYP46A1 delivered with modified vector will be significantly enhanced as compared to un-modified vector and significantly extended from prefrontal association cortical areas (Cortical Areas 9 and 10) through the frontal eye-fields (Area 8), pre-motor cortex (Area 6), primary Somatosensory cortical areas (Areas 3, 1 and 2) to primary motor cortex (Area 4), and included expression in the cingulate cortex (Areas 23, 24, 32) and Broca's area (Area 44, 45). In addition to the intense staining of individual neuronal cell bodies and cellular processes, CYP46A1 staining will be observed across multiple layers of the frontal cortex with an intensity gradient that was highest in cortical Layers III and IV, as compare to the same dose of unmodified vector.

Delivery of modified vector comprising GFP as compared to parent will also be tested in monkey model as describe in U.S. patent application Ser. No. 13/146,640. The relative amount of modified vector in the AN anterior nucleus; MD medio-dorsal nucleus; VA ventral anterior nucleus; VL ventral lateral nucleus; VP ventral posterior nucleus will be significantly higher than that of un-modified vector. In addition, modified vector is distributed widely and more efficiently throughout cortex as compared to un-modified vector. The percent of positive cells is significantly higher in each area and region as compared to parental vector. More efficient transduction of cortical layers 1-6 is also expected. Delivery to multiple lobes of the cerebral cortex or all of cortical areas 1-4, 6 and 8-10 can be achieved.

| Region | Area |
|---|---|
| Pre-Frontal Cortex | 9/10/46 |
| Broca's Area | 44/45 |
| Frontal Eye Field | 8 |
| Secondary Motor Cortex | 6 |
| Anterior Cingulate Cortex | 24/32 |
| Somatosensory Cortex | 1.2/03 |
| Primary Motor Cortex | 4 |
| Posterior Cingulate Cortex | 23/31 |

Surgical Delivery. Modified and un-modified rAAV vectors GFP) under the control of cytomegalovirus promoter were infused into the right thalamus of six adult Rhesus monkeys by convection enhanced delivery (CED) protocol. All experimentation is performed according to the National Institutes of Health guidelines and to the protocols approved by the Institutional Animal Care and Use Committee at the University of California San Francisco.

Immunostaining with antibodies against CYP46A1 (1:500, AF-212-NA, R&D Systems) and GFP (1:500, AB3080, Chemicon) is performed on Zamboni fixed 40-um coronal sections covering the entire frontal cortex and extending in a posterior direction to the level of the thalamus. The localization of CYP46A1 and GFP immunopositive neurons is analyzed with reference to The Rhesus Monkey Brain in Stereotactic Coordinates to identify specific areas of immunostaining in the cortex and thalamus.

CYP46A1 Protein ELISA. Tissue punches from 3-mm coronal blocks of fresh frozen tissue are taken from a number of cortical, thalamic. Methods and Materials and striatal regions of a modified vector infused monkey. Surgical Delivery expressed is quantified by ELISA assay with a commercial ELISA kit (Emax ELISA, Promega, Wis.) human CYP46A1 c1DNA or GFP cDNA.

### Example 6

Next, to determine if changing the modification of a capsid enables re-administration, the modified vector comprising—CYP46A1 of Example 5 is redesigned to have a different chemical modification, but consists of the same capsid and comprises the same payload (i.e., CYP46A1) of the capsid of Example 5. An adult Rhesus monkeys are administered the first modified vector comprising—CYP46A1 of Example 2, and at 14 days post-administration, administered either a second dose of the same vector, or the redesigned modified capsid. CYP46A1 expression is assessed using the ELISA assay described above in Example 5. It was found that re-administration of the same vector has significantly reduced expression, likely due to neutralizing antibodies generated against vector following the first administration. Strikingly, expression of the redesigned vector was high and widespread, indicating that the change in modification of the capsid enabled expression of the redesigned vector.

1. A method for treating a neurological disease or disorder in a subject in need thereof, the method comprising administering to a subject having or at risk of developing the neurological disease or disorder a therapeutically effective amount of
(a) an isolated nucleic acid encoding a transgene encoding one or more miRNAs; and
(b) an isolated nucleic acid encoding a CYP46A1 protein.

2. A method for treating a neurological disease or disorder in a subject in need thereof, the method comprising administering to a subject having or at risk of developing the neurological disease or disorder a therapeutically effective amount of
(a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and
(b) a recombinant viral vector comprising an isolated nucleic acid encoding the CYP46A1 protein.

3. The method of any of claims 1-2, wherein the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

4. The method of any of claims 1-3, wherein the neurological disease or disorder is a central nervous system (CNS) disease or disorder.

5. The method of any of claims 1-4, wherein the CNS disease or disorder is selected from Huntington's disease, Alzheimer's disease, Polyglutamine repeat spinocerebellar ataxias, Amyotrophic lateral sclerosis and Parkinson's disease.

6. The method of any of claims 1-5, wherein the CNS disease or disorder is Alzheimer's disease and the at least one miRNA comprises a seed sequence complementary to Amyloid Precursor Protein (APP), Presenilin 1, Presenilin 2, ABCA7, SORL1, and disease-associated alleles thereof.

7. The method of any of claims 1-5, wherein the CNS disease or disorder is Parkinson's disease and the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

8. The method of any of claims 1-5, wherein the CNS disease is Huntington's disease and at least one miRNA comprises a seed sequence complementary to SEQ ID NO: 4, or wherein at least one miRNA comprises the sequence of any one of SEQ ID NOs: 6-17, 40-44, or 50-66 flanked by a miRNA backbone sequence.

9. The method of any of claims 1-8, wherein the CNS disease is Huntington's disease and at least one miRNA comprises the sequence of any one of SEQ ID NOs: 6-17, 40-44, or 50-66.

10. The method of any of claims 8-9, wherein at least one of the miRNAs hybridizes with and inhibits expression of human huntingtin.

11. The method of any of claims 8-10, wherein the subject comprises a huntingtin gene having more than 36 CAG repeats, more than 40 repeats, or more than 100 repeats.

12. The method of any of claims 8-11, wherein the subject is less than 20 years of age.

13. The method of any of claims 1-12, wherein the recombinant viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector.

14. The method of any of claims 2-13, wherein the recombinant viral vector comprising (a) is the same as the recombinant viral vector comprising (b).

15. The method of any of claims 1-13, wherein the isolated nucleic acid of (a) and (b) are comprised in separate recombinant viral vectors.

16. The method of any of claims 1-14, wherein the isolated nucleic acid of (a) and (b) are comprised in the same recombinant viral vector.

17. The method of any one of claims 1-16, wherein (a) and (b) are administered at substantially the same time.

18. The method of any one of claims 1-13 and 15, wherein (a) and (b) are administered at different time points.

19. The method of claim 18, wherein the different time points are spaced by at least 1 min, at least 1 hour, at least 1 day, at least 1 week, at least 1 month, at least 1 year, or more.

20. The method of any of claims 18-19, wherein (a) is administered prior to the administration of (b).

21. The method of any of claims 18-19, wherein (b) is administered prior to the administration of (a).

22. The method of any of claims 1-21, wherein the administration of (a), (b), or (a) and (b) is repeated at least once.

23. The method of any of claims 1-22, wherein the transgene comprises two miRNAs in tandem that are flanked by introns.

24. The method of claim 23, wherein the flanking introns are identical.

25. The method of claim 23, wherein the flanking introns are from the same species.

26. The method of claim 23, wherein the flanking introns are hCG introns.

27. The method of any one of claims 1-26, wherein the transgene comprises a promoter.

28. The method of claim 27, wherein the promoter is a synapsin (Syn1) promoter, or a promoter of Tables 10-13.

29. The method of any one of claims 1-28, wherein the one or more miRNAs are located in an untranslated portion of the transgene.

30. The method of claim 29, wherein the untranslated portion is an intron.

31. The method of claim 30, wherein the untranslated portion is between the last codon of the nucleic acid sequence encoding a protein and a poly-A tail sequence, or between the last nucleotide base of a promoter sequence and a poly-A tail sequence.

32. The method of any one of claims 1-31, further comprising a third region comprising a second adeno-associated virus (AAV) inverted terminal repeat (ITR), or a variant thereof.

33. The method of any one of claims 1-33, wherein the ITR variant lacks a functional terminal resolution site (TRS), optionally wherein the ITR variant is a ATRS ITR.

34. The method of any of claims 1-33, wherein the administration results in delivery of the viral vector or isolated nucleic acid to the central nervous system (CNS) of the subject.

35. The method of any of claims 1-34, wherein the administration is via injection, optionally intravenous injection or intrastriatal injection.

36. The method of any of claims 2-35, wherein the viral vector is AAV1, AAV2, AAV3b, AAV4, AAV5, AAV6, AAV7, AAV8, AAV9, AAV10, AAV 11, or, AAV12, or a chimera thereof.

37. The method of any of claims 2-36, the viral vector comprises a capsid protein from AAV serotype AAV1, AAV2, AAV3b, AAV4, AAV5, AAV6, AAV7, AAV8, AAV9, AAV10, AAV 11, or, AAV12, or a chimera thereof

38. The method of claim 37, wherein the capsid protein is an AAV9 capsid protein.

39. The method of any of claims 2-38, wherein the viral vector is a self-complementary AAV (scAAV).

40. The method of any of claims 2-39, wherein the viral vector is formulated for delivery to the central nervous system (CNS).

41. A composition or combination comprising:
(a) an isolated nucleic acid encoding a transgene encoding one or more miRNAs; and
(b) an isolated nucleic acid encoding a CYP46A1 protein.

42. A composition or combination comprising:
(a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and
(b) a recombinant viral vector comprising an isolated nucleic acid encoding the CYP46A1 protein.

43. The composition or combination of any of claims 41-42, for use in a method for treating a neurological disease or disorder in a subject in need thereof, the method comprising administering to a subject having or at risk of developing the neurological disease or disorder a therapeutically effective amount of the composition or combination.

44. The composition or combination of claim 43, wherein the neurological disease or disorder is Alzheimer's disease, Parkinson's disease, Huntington's disease, Canavan disease, Leigh's disease, spinal cerebral ataxia, Krabbe's disease, polyglutamine repeat spinocerebellar ataxia, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

45. The composition or combination of claim 44, wherein the neurological disease or disorder is a central nervous system (CNS) disease or disorder.

46. The composition or combination of claim 45, wherein the CNS disease or disorder is selected from Huntington's disease, Alzheimer's disease, Polyglutamine repeat spinocerebellar ataxias, Amyotrophic lateral sclerosis and Parkinson's disease.

47. The composition or combination of any of claims 41-46, wherein the at least one miRNA comprises a seed sequence complementary to Amyloid Precursor Protein (APP), Presenilin 1, Presenilin 2, ABCA7, SORL1, and disease-associated alleles thereof.

48. The composition or combination of any of claims 41-46, wherein the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

49. The composition or combination of any of claims 41-46, wherein the at least one miRNA comprises a seed sequence complementary to SEQ ID NO: 4, or wherein the at least one miRNA comprises the sequence of any one of SEQ ID NOs: 6-17, 40-44, or 50-66 flanked by a miRNA backbone sequence.

50. The composition or combination of any of claims 41-46, wherein the at least one miRNA comprises the sequence of any one of SEQ ID NOs: 6-17, 40-44, or 50-66.

51. The composition or combination of any of claims 49-50, wherein at least one of the miRNAs hybridizes with and inhibits expression of human huntingtin.

52. The composition or combination of any of claims 49-51, wherein the subject comprises a huntingtin gene having more than 36 CAG repeats, more than 40 repeats, or more than 100 repeats.

53. The composition or combination of any of claims 49-52, wherein the subject is less than 20 years of age.

54. The composition or combination of any of claims 42-53, wherein the recombinant viral vector is selected from the group consisting of: an AAV vector, an adenovirus vector, a lentivirus vector, a retrovirus vector, a herpesvirus vector, an alphavirus vector, a poxvirus vector a baculovirus vector, and a chimeric virus vector.

55. The composition or combination of any of claims 42-54, wherein the recombinant viral vector comprising (a) is the same as the recombinant viral vector comprising (b).

56. The composition or combination of any of claims 41-54, wherein the isolated nucleic acid of (a) and (b) are comprised in separate recombinant viral vectors.

57. The composition or combination of any of claims 41-55, wherein the isolated nucleic acid of (a) and (b) are comprised in the same recombinant viral vector.

58. The composition or combination of any of claims 41-57, wherein (a) and (b) are administered at substantially the same time.

59. The composition or combination of any of claims 41-54 and 56, wherein (a) and (b) are administered at different time points.

60. The composition or combination of claim 59, wherein the different time points are spaced by at least 1 min, at least 1 hour, at least 1 day, at least 1 week, at least 1 month, at least 1 year, or more.

61. The composition or combination of any of claims 59-60, wherein (a) is administered prior to the administration of (b).

62. The composition or combination of any of claims 59-60, wherein (b) is administered prior to the administration of (a).

63. The composition or combination of any of claims 59-60, wherein the administration of (a), (b), or (a) and (b) is repeated at least once.

64. The composition or combination of any of claims 41-65, wherein the transgene comprises two miRNAs in tandem that are flanked by introns.

65. The composition or combination of claim 64, wherein the flanking introns are identical.

66. The composition or combination of claim 64, wherein the flanking introns are from the same species.

67. The composition or combination of claim 64, wherein the flanking introns are hCG introns.

68. The composition or combination of any of claims 41-67, wherein the transgene comprises a promoter.

69. The composition or combination of claim 68, wherein the promoter is a synapsin (Syn1) promoter or a promoter of Tables 10-13.

70. The composition or combination of any of claims 41-69, wherein the one or more miRNAs are located in an untranslated portion of the transgene.

71. The composition or combination of claim 70, wherein the untranslated portion is an intron.

72. The composition or combination of claim 70, wherein the untranslated portion is between the last codon of the nucleic acid sequence encoding a protein and a poly-A tail sequence, or between the last nucleotide base of a promoter sequence and a poly-A tail sequence.

73. The composition or combination of any of claims 41-72, further comprising a third region comprising a second adeno-associated virus (AAV) inverted terminal repeat (ITR), or a variant thereof.

74. The composition or combination of any of claims 41-73, wherein the ITR variant lacks a functional terminal resolution site (TRS), optionally wherein the ITR variant is a ATRS ITR.

75. The composition or combination of any of claims 41-74, wherein the administration results in delivery of the viral vector or isolated nucleic acid to the central nervous system (CNS) of the subject.

76. The composition or combination of any of claims 41-75, wherein the administration is via injection, optionally intravenous injection or intrastriatal injection.

77. The composition or combination of any of claims 42-76, wherein the viral vector is an AAV1, AAV2, AAV3b, AAV4, AAV5, AAV6, AAV7, AAV8, AAV9, AAV10, AAV 11, AAV12, or a chimera thereof.

78. The composition of any of claims 42-77, the viral vector comprises a capsid protein from AAV serotype AAV1, AAV2, AAV3b, AAV4, AAV5, AAV6, AAV7, AAV8, AAV9, AAV10, AAV11, or, AAV12, or a chimera thereof

79. The composition or combination of claim 78, wherein the capsid protein is an AAV9 capsid protein.

80. The composition or combination of any of claims 42-79, wherein the viral vector is a self-complementary AAV (scAAV).

81. The composition or combination of any of claims 42-80, wherein the viral vector is formulated for delivery to the central nervous system (CNS).

82. A composition comprising an isolated nucleic acid encoding a CYP46A1 protein, the nucleic acid comprising a sequence at least 80% identical to SEQ ID NO: 110, or, at least 80% identical to SEQ ID No: 111, or, at least 80% identical to SEQ ID NO: 153.

83. A composition comprising a recombinant viral vector comprising an isolated nucleic acid encoding a CYP46A1 protein, the nucleic acid comprising a sequence at least 80% identical to SEQ ID NO: 110. or, at least 80% identical to SEQ ID No: 111, or, at least 80% identical to SEQ ID NO: 153.

84. A method for treating a neurological disease or disorder in a subject in need thereof, the method comprising administering to a subject having or at risk of developing the neurological disease or disorder a therapeutically effective amount of a composition of claim 82 or 83.

85. The method of claim 84, wherein the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

86. The method of any of claims 84-85, wherein the neurological disease or disorder is a central nervous system (CNS) disease or disorder.

87. The method of any of claims 84-86, wherein the CNS disease or disorder is selected from Huntington's disease, Alzheimer's disease, Polyglutamine repeat spinocerebellar ataxias, Amyotrophic lateral sclerosis and Parkinson's disease.

88. The composition or method of any of claims 83-87, wherein the recombinant viral vector is selected from the group consisting of: an AAV vector, an adenovirus vector, a lentivirus vector, a retrovirus vector, a herpesvirus vector, an alphavirus vector, a poxvirus vector a baculovirus vector, and a chimeric virus vector.

89. The method of any of claims 84-88, wherein the administration is repeated at least once.

90. The method of any of claims 84-89, wherein the administration results in delivery of the viral vector or isolated nucleic acid to the central nervous system (CNS) of the subject.

91. The method of any of claims 84-90, wherein the administration is via injection, optionally intravenous injection or intrastriatal injection.

92. The composition or method of any of claims 83-91, wherein the viral vector is AAV1, AAV2, AAV3b, AAV4, AAV5, AAV6, AAV7, AAV8, AAV9, AAV10, AAV 11, or, AAV12, or a chimera thereof.

93. The composition or method of any of claims 83-92, viral vector comprises a capsid protein from AAV serotype AAV1, AAV2, AAV3b, AAV4, AAV5, AAV6, AAV7, AAV8, AAV9, AAV10, AAV11, or, AAV12, or a chimera thereof

94. The composition or method of claim 93, wherein the capsid protein is an AAV9 capsid protein.

95. The composition or method of any of claims 83-94, wherein the viral vector is a self-complementary AAV (scAAV).

96. The composition or method of any of claims 83-95, wherein the viral vector is formulated for delivery to the central nervous system (CNS).

97. The composition or method of any of claims 82-96, wherein the nucleic acid comprises a sequence at least 90% identical to SEQ ID NO: 110.

98. The composition or method of any of claims 82-96, wherein the nucleic acid comprises a sequence at least 95% identical to SEQ ID NO: 110.

99. The composition or method of any of claims 82-96, wherein the nucleic acid comprises a sequence identical to SEQ ID NO: 110.

100. The composition or method of any of claims 2-40, 42-81, 83-99, where the viral vector comprises a modified viral capsid.

101. The composition or method of any of claims 2-40, 42-81, 83-99, where the viral vector comprises a modification to a viral capsid.

102. The composition or method of claim 100 or 101, wherein the modification is a chemical, non-chemical or amino acid modification of the viral capsid.

103. The composition or method of claim 100 or 101, wherein at least one of the capsid modifications preferentially targets cells in the CNS or PNS.

104. The composition or method of claim 100 or 101, wherein the chemical modification comprises a chemically-modified tyrosine residue modified to comprise a covalently-linked mono- or polysaccharide moiety.

105. The composition or method of claim 104, wherein the chemically-modified tyrosine residue comprises a mono-saccharide selected from galactose, mannose, N-acetylgalactosamine, bridge GalNac, and mannose-6-phosphate.

106. The composition or method of claim 100 or 101, wherein the chemical modification comprises a ligand covalently linked to a primary amino group of a capsid polypeptide via a —CSNH-bond.

107. The composition or method of claim 106, wherein the ligand comprises an arylene or heteroarylene radical covalently bound to the ligand.

108. The composition or method of any of claims 100-107, wherein the modified viral capsid is a chimeric capsid or a haploid capsid.

109. The composition or method of any of claims 100-107, wherein the modified viral capsid is a haploid capsid.

110. The composition or method of any of claims 100-107, wherein the modified viral capsid is a chimeric or haploid capsid further comprising a modification.

111. The composition or method of any of claims 100-110, wherein the modified viral capsid is from an AAV serotype AAV1, AAV2, AAV3b, AAV4, AAV5, AAV6, AAV7, AAV8, AAV9, AAV10, AAV 11, AAV12, or a mutant modified from, a chimera, a mosaic, or a rational haploid thereof.

112. The composition or method of any of claims 100-111, wherein the modification changes the antigenic profile of the modified viral capsid as compared to the unmodified viral capsid.

113. The composition or method of any of claims 100-112, wherein the modified viral capsid can be used for repeat administration.


SEQUENCE LISTING (157 sequences)

SEQ ID NO: 1 - DNA, 2138 bp, Homo sapiens
  CDS (1)..(1500)
atgagccccgggctgctgctgctcggcagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttttggattgggctaagaagtatggacctgttgtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtcatagacctggccttcagccggagctccttggttagcttaatggaaacattcaacgagaaggctgagcagctggtggagattctagaagccaaggcagatgggcagaccccagtgtccatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaggtcattggttctaagaggtacctggatttcgaggacctggggagactgcagtacctgtcccaggtcctcaaagagtcgctgaggctgtacccaccagcatggggcacctttcgcctgctggaagaggagaccttgattgatggggtcagagtccccggcaacaccccgctcttgttcagcacctatgtcatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctgagggggcctccaggcaggacgagactcctcgggcaagggccgtgcccgcccacctctgctgcccacggccacccacccttctcccctgccccgtcccctgggccacccttcacgctggcttccagcgggccctctgccgaccgcctgcttcacacccctcagcgctccctgtcgcctgcggactccatggcccttcctggactggcccttgcccaactcccagccaccaccactgtccctaccactgagcccttgcacaggccacttgctcagacgagacaccctaactcttgctcactccctaaagccctcttcaggggtcacctcctccaagaagccctccttgccaccccccgccggcaggggcccctcctctgtgctccctcggtcacctgtgctacctctaacaccacactgaccacactgtatcgtgagtgtccgttgacgtgaccaattgccctgccaggctgtcagcgcctcaagggtagggtctgcgtgtgatttgtctctgagccccctgtgcccacccagggcccggcacagagtcgatgctcaataaatgtgtgttgactgcaaaaaaaaaaaaaaaaaaaaaaaaaaaaaaaaaaaaaaaaaaaaaaaaaaaaaaaaaaaaaa

SEQ ID NO: 2 - protein, 500 aa, Homo sapiens
MSPGLLLLGSAVLLAFGLCCTFVHRARSRYEHIPGPPRPSFLLGHLPCFWKKDEVGGRVLQDVFLDWAKKYGPVVRVNVFHKTSVIVTSPESVKKFLMSTKYNKDSKMYRALQTVFGERLFGQGLVSECNYERWHKQRRVIDLAFSRSSLVSLMETFNEKAEQLVEILEAKADGQTPVSMQDMLTYTAMDILAKAAFGMETSMLLGAQKPLSQAVKLMLEGITASRNTLAKFLPGKRKQLREVRESIRFLRQVGRDWVQRRREALKRGEEVPADILTQILKAEEGAQDDEGLLDNFVTFFIAGHETSANHLAFTVMELSRQPEIVARLQAEVDEVIGSKRYLDFEDLGRLQYLSQVLKESLRLYPPAWGTFRLLEEETLIDGVRVPGNTPLLFSTYVMGRMDTYFEDPLTFNPDRFGPGAPKPRFTYFPFSLGHRSCIGQQFAQMEVKVVMAKLLQRLEFRLVPGQRFGLQEQATLKPLDPVLCTLRPRGWQPAPPPPPC

SEQ ID NO: 3 - RNA, 614 nt, Homo sapiens
uugcugugugaggcagaaccugcgggggcaggggcgggcugguucccuggccagccauuggcagaguccgcaggcuagggcugucaaucaugcuggccggcguggccccgccuccgccggcgcggccccgccuccgccggcgcacgucugggacgcaaggcgccgugggggcugccgggacggguccaagauggacggccgcucagguucugcuuuuaccugcggcccagagccccauucauugccccggugcugagcggcgccgcgagucggcccgaggccuccggggacugccgugccgggcgggagaccgccauggcgacccuggaaaagcugaugaaggccuucgagucccucaaguccuuccagcagcagcagcagcagcagcagcagcagcagcagcagcagcagcagcagcagcagcagcagcaacagccgccaccgccgccgccgccgccgccgccuccucagcuuccucagccgccgccgcaggcacagccgcugcugccucagccgcagccgcccccgccgccgcccccgccgccacccggcccggcuguggcugaggagccgcugcaccgaccgugaguuugggcccgcugcagcucccuguc

SEQ ID NO: 4 - DNA, 13498 bp, Homo sapiens
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcaaaaaaaaaaaaaaaaaa

SEQ ID NO: 5 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 6 - RNA, 19 nt, Artificial Sequence
  Description of Artificial Sequence Synthetic oligonucleotide
aaggacuugagggacucga

SEQ ID NO: 7 - RNA, 20 nt, Artificial Sequence
  Description of Artificial Sequence Synthetic oligonucleotide
aaggacuugagggacucgaa

SEQ ID NO: 8 - RNA, 21 nt, Artificial Sequence
  Description of Artificial Sequence Synthetic oligonucleotide
aaggacuugagggacucgaag

SEQ ID NO: 9 - RNA, 22 nt, Artificial Sequence
  Description of Artificial Sequence Synthetic oligonucleotide
aaggacuugagggacucgaagg

SEQ ID NO: 10 - RNA, 23 nt, Artificial Sequence
  Description of Artificial Sequence Synthetic oligonucleotide
aaggacuugagggacucgaaggc

SEQ ID NO: 11 - RNA, 19 nt, Artificial Sequence
  Description of Artificial Sequence Synthetic oligonucleotide
ucgagucccucaaguccuu

SEQ ID NO: 12 - RNA, 20 nt, Artificial Sequence
  Description of Artificial Sequence Synthetic oligonucleotide
uucgagucccucaaguccuu

SEQ ID NO: 13 - RNA, 21 nt, Artificial Sequence
  Description of Artificial Sequence Synthetic oligonucleotide
cuucgagucccucaaguccuu

SEQ ID NO: 14 - RNA, 22 nt, Artificial Sequence
  Description of Artificial Sequence Synthetic oligonucleotide
ccuucgagucccucaaguccuu

SEQ ID NO: 15 - RNA, 23 nt, Artificial Sequence
  Description of Artificial Sequence Synthetic oligonucleotide
gccuucgagucccucaaguccuu

SEQ ID NO: 16 - RNA, 19 nt, Artificial Sequence
  Description of Artificial Sequence Synthetic oligonucleotide
cuucgagucucaaguccuu

SEQ ID NO: 17 - RNA, 19 nt, Artificial Sequence
  Description of Artificial Sequence Synthetic oligonucleotide
acgagucccucaaguccuc

SEQ ID NO: 18 - RNA, 20 nt, Homo sapiens
aaggacuugagggacucgaa

SEQ ID NO: 19 - DNA, 21 bp, Homo sapiens
tccaagatggacggccgctca

SEQ ID NO: 20 - DNA, 21 bp, Homo sapiens
ccaagatggacggccgctcag

SEQ ID NO: 21 - DNA, 21 bp, Homo sapiens
agatggacggccgctcaggtt

SEQ ID NO: 22 - DNA, 21 bp, Homo sapiens
atggacggccgctcaggttct

SEQ ID NO: 23 - DNA, 21 bp, Homo sapiens
gacggccgctcaggttctgct

SEQ ID NO: 24 - DNA, 21 bp, Homo sapiens
cggccgctcaggttctgcttt

SEQ ID NO: 25 - DNA, 21 bp, Homo sapiens
gtgctgagcggcgccgcgagt

SEQ ID NO: 26 - DNA, 21 bp, Homo sapiens
cgccgcgagtcggcccgaggc

SEQ ID NO: 27 - DNA, 21 bp, Homo sapiens
accgccatggcgaccctggaa

SEQ ID NO: 28 - DNA, 21 bp, Homo sapiens
ccgccatggcgaccctggaaa

SEQ ID NO: 29 - DNA, 21 bp, Homo sapiens
gaaggccttcgagtccctcaa

SEQ ID NO: 30 - DNA, 21 bp, Homo sapiens
cttcgagtccctcaagtcctt

SEQ ID NO: 31 - DNA, 21 bp, Homo sapiens
ccgccgccgcctcctcagctt

SEQ ID NO: 32 - DNA, 21 bp, Homo sapiens
gccgcctcctcagcttcctca

SEQ ID NO: 33 - DNA, 21 bp, Homo sapiens
tcagccgccgccgcaggcaca

SEQ ID NO: 34 - DNA, 21 bp, Homo sapiens
gccgcaggcacagccgctgct

SEQ ID NO: 35 - DNA, 21 bp, Homo sapiens
ggcacagccgctgctgcctca

SEQ ID NO: 36 - DNA, 21 bp, Homo sapiens
gccgctgctgcctcagccgca

SEQ ID NO: 37 - DNA, 21 bp, Homo sapiens
cggcccggctgtggctgagga

SEQ ID NO: 38 - DNA, 21 bp, Homo sapiens
ctgtggctgaggagccgctgc

SEQ ID NO: 39 - DNA, 21 bp, Homo sapiens
tgtggctgaggagccgctgca

SEQ ID NO: 40 - RNA, 72 nt, Artificial Sequence
  Description of Artificial Sequence Synthetic oligonucleotide
cuugggaauggcaaggaaggacuugagggacucgaagacgagucccucaaguccucucuugcuauacccaga

SEQ ID NO: 41 - RNA, 69 nt, Artificial Sequence
  Description of Artificial Sequence Synthetic oligonucleotide
ugcugaaggacuugagggacucgaagguuuuggccacugacugaccuucgagucucaaguccuucagga

SEQ ID NO: 42 - RNA, 272 nt, Artificial Sequence
  Description of Artificial Sequence Synthetic polynucleotide
gcuaagcacuucguggccgucgaucguuuaaagggagguagugagucgaccaguggauccuggaggcuugcugaaggcuguaugcugaaggacuugagggacucgaagguuuuggccacugacugaccuucgagucucaaguccuucaggacacaaggccuguuacuagcacucacauggaacaaauggcccagaucuggccgcacucgagauaucuagacccagcuuucuuguacaaagugguugaucuagagggcccgcgguucgcugau

SEQ ID NO: 43 - RNA, 605 nt, Artificial Sequence
  Description of Artificial Sequence Synthetic polynucleotide
gcuccugggcaacgugcugguuauugugcugucucaucauuuuggcaaagaauuaagggcgaauucgagcucgguaccucgcgaaugcaucuagauaucggcgcuaugcuuccugugcccccaguggggcccuggcugggauuucaucauauacuguaaguuugcgaugagacacuacaguauagaugauguacuaguccgggcacccccagcucuggagccugacaaggaggacaggagagaugcugcaagcccaagaagcucucugcucagccugucacaaccuacugacugccagggcacuugggaauggcaaggaaggacuugagggacucgaagacgagucccucaaguccucucuugcuauacccagaaaacgugccaggaagagaacucaggacccugaagcagacuacuggaagggagacuccagcucaaacaaggcaggggugggggcgugggauuggggguaggggagggaauagauacauuuucucuuuccuguuguaaagaaauaaagauaagccaggcacaguggcucacgccuguaaucccaccacuuucagaggccaaggcgcuggauccagaucucgagcggccgcccg

SEQ ID NO: 44 - RNA, 819 nt, Artificial Sequence
  Description of Artificial Sequence Synthetic polynucleotide
agucucgugcagauggacagcaccgcugagcaauggaagcggguaggccuuuggggcagcggccaauagcagcuuugcuccuucgcuuucugggcucagaggcugggaagggguggguccgggggcgggcucaggggcgggcucaggggcggggcgggcgcccgaagguccuccggaggcccggcauucugcacgcuucaaaagcgcacgucugccgcgcuguucuccucuuccucaucuccgggccuuucgacccggaucccccgggcugcaggaauucgagcucgguaccucgcgaaugcaucuagauaucggcgcuaugcuuccugugcccccaguggggcccuggcugggauuucaucauauacuguaaguuugcgaugagacacuacaguauagaugauguacuaguccgggcacccccagcucuggagccugacaaggaggacaggagagaugcugcaagcccaagaagcucucugcucagccugucacaaccuacugacugccagggcacuugggaauggcaaggaaggacuugagggacucgaagacgagucccucaaguccucucuugcuauacccagaaaacgugccaggaagagaacucaggacccugaagcagacuacuggaagggagacuccagcucaaacaaggcaggggugggggcgugggauuggggguaggggagggaauagauacauuuucucuuuccuguuguaaagaaauaaagauaagccaggcacaguggcucacgccuguaaucccaccacuuucagaggccaaggcgcuggauccagaucucgagcggccgcccg

SEQ ID NO: 45 - RNA, 40 nt, Homo sapiens
ccacgccugcucccucauccacugugugcacuucauccug

SEQ ID NO: 46 - RNA, 40 nt, Homo sapiens
ccacgccugcucccucaucuacugugugcacuucauccug

SEQ ID NO: 47 - RNA, 37 nt, Homo sapiens
uaagagauggggacaguaauucaacgcuagaagaaca

SEQ ID NO: 48 - RNA, 37 nt, Homo sapiens
uaagagauggggacaguacuucaacgcuagaagaaca

SEQ ID NO: 49 - DNA, 27 bp, Homo sapiens
cagatgccatggcctgtgctgggccag

SEQ ID NO: 50 - RNA, 21 nt, Artificial Sequence
  Description of Artificial Sequence Synthetic oligonucleotide
ucccucauccacugugugaac

SEQ ID NO: 51 - RNA, 21 nt, Artificial Sequence
  Description of Artificial Sequence Synthetic oligonucleotide
gcacacaguggaugagggagc

SEQ ID NO: 52 - RNA, 21 nt, Artificial Sequence
  Description of Artificial Sequence Synthetic oligonucleotide
ucccucaucuacugugugaac

SEQ ID NO: 53 - RNA, 21 nt, Artificial Sequence
  Description of Artificial Sequence Synthetic oligonucleotide
cgagggaguagaugacacacg

SEQ ID NO: 54 - RNA, 21 nt, Artificial Sequence
  Description of Artificial Sequence Synthetic oligonucleotide
gggacaguaauucaacgcguc

SEQ ID NO: 55 - RNA, 21 nt, Artificial Sequence
  Description of Artificial Sequence Synthetic oligonucleotide
agcguugaauuacugucccca

SEQ ID NO: 56 - RNA, 21 nt, Artificial Sequence
  Description of Artificial Sequence Synthetic oligonucleotide
gggacaguacuucaacgcguc

SEQ ID NO: 57 - RNA, 21 nt, Artificial Sequence
  Description of Artificial Sequence Synthetic oligonucleotide
accccugucaugaaguugcga

SEQ ID NO: 58 - RNA, 21 nt, Artificial Sequence
  Description of Artificial Sequence Synthetic oligonucleotide
ugccauggccugugcuggucc

SEQ ID NO: 59 - RNA, 20 nt, Artificial Sequence
  Description of Artificial Sequence Synthetic oligonucleotide
cccagcacaggccauggcac

SEQ ID NO: 60 - RNA, 21 nt, Artificial Sequence
  Description of Artificial Sequence Synthetic oligonucleotide
ugccauggcaugugcuggucc

SEQ ID NO: 61 - RNA, 21 nt, Artificial Sequence
  Description of Artificial Sequence Synthetic oligonucleotide
cccagcacaugccuaggcauc

SEQ ID NO: 62 - DNA, 20 bp, Artificial Sequence
  Description of Artificial Sequence Synthetic oligonucleotide
ctcagtaacattgacaccac

SEQ ID NO: 63 - DNA, 20 bp, Artificial Sequence
  Description of Artificial Sequence Synthetic oligonucleotide
ctcgactaaagcaggatttc

SEQ ID NO: 64 - DNA, 20 bp, Artificial Sequence
  Description of Artificial Sequence Synthetic oligonucleotide
ccttccctgaaggttcctcc

SEQ ID NO: 65 - DNA, 20 bp, Artificial Sequence
  Description of Artificial Sequence Synthetic oligonucleotide
gcagggttaccgccatcccc

SEQ ID NO: 66 - DNA, 20 bp, Artificial Sequence
  Description of Artificial Sequence Synthetic oligonucleotide
cgagacagtcgcttccactt

SEQ ID NO: 67 - DNA, 20 bp, Artificial Sequence
  Description of Artificial Sequence Synthetic oligonucleotide
cgaggccggggcggggcaca

SEQ ID NO: 68 - DNA, 20 bp, Artificial Sequence
  Description of Artificial Sequence Synthetic oligonucleotide
cggggcggggccgtggaggg

SEQ ID NO: 69 - DNA, 20 bp, Artificial Sequence
  Description of Artificial Sequence Synthetic oligonucleotide
actgtgccactatgttttca

SEQ ID NO: 70 - DNA, 20 bp, Artificial Sequence
  Description of Artificial Sequence Synthetic oligonucleotide
gccttcatcagcttttccag

SEQ ID NO: 71 - DNA, 20 bp, Artificial Sequence
  Description of Artificial Sequence Synthetic oligonucleotide
gctgctgctgctgctgctgc

SEQ ID NO: 72 - DNA, 20 bp, Artificial Sequence
  Description of Artificial Sequence Synthetic oligonucleotide
tgctggaaggacttgaggga

SEQ ID NO: 73 - DNA, 20 bp, Artificial Sequence
  Description of Artificial Sequence Synthetic oligonucleotide
tgttgctgctgctgctgctg

SEQ ID NO: 74 - DNA, 795 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 75 - DNA, 810 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 76 - DNA, 916 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 77 - DNA, 931 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 78 - DNA, 617 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 79 - DNA, 632 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcactgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 80 - DNA, 701 bp, Artificial Sequence
  Description of Artificial Sequence Synthetic polynucleotide
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 81 - DNA, 716 bp, Artificial Sequence
  Description of Artificial Sequence Synthetic polynucleotide
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 82 - DNA, 792 bp, Artificial Sequence
  Description of Artificial Sequence Synthetic polynucleotide
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 83 - DNA, 686 bp, Artificial Sequence
  Description of Artificial Sequence Synthetic polynucleotide
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggccgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 84 - DNA, 807 bp, Artificial Sequence
  Description of Artificial Sequence Synthetic polynucleotide
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 85 - DNA, 701 bp, Artificial Sequence
  Description of Artificial Sequence Synthetic polynucleotide
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcctgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 86 - DNA, 913 bp, Artificial Sequence
  Description of Artificial Sequence Synthetic polynucleotide
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 87 - DNA, 928 bp, Artificial Sequence
  Description of Artificial Sequence Synthetic polynucleotide
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 88 - DNA, 529 bp, Artificial Sequence
  Description of Artificial Sequence Synthetic polynucleotide
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatgagctctccccatagctgggctgcggcccaaccccaccccctcaggctatgccagggggtgttgccaggggcacccgggcatcgccagtctagcccactccttcataaagccctcgcatcccaggagcgagcagagccagagc

SEQ ID NO: 89 - DNA, 646 bp, Artificial Sequence
  Description of Artificial Sequence Synthetic polynucleotide
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacccgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 90 - DNA, 614 bp, Artificial Sequence
  Description of Artificial Sequence Synthetic polynucleotide
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatcgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 91 - DNA, 696 bp, Artificial Sequence
  Description of Artificial Sequence Synthetic polynucleotide
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgcttctg

SEQ ID NO: 92 - DNA, 709 bp, Artificial Sequence
  Description of Artificial Sequence Synthetic polynucleotide
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggcgggctgggcataaaagtcagggcagagccatctattgcttacatttgcttctg

SEQ ID NO: 93 - DNA, 768 bp, Artificial Sequence
  Description of Artificial Sequence Synthetic polynucleotide
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggggggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 94 - DNA, 736 bp, Artificial Sequence
  Description of Artificial Sequence Synthetic polynucleotide
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcccgagcggagaggcggcccgggagcaggggggcggcccccactccggccgggtgcccggcccctggcccctgcctgccctctagatcgccgccgcagccgccgctactgggagtctg

SEQ ID NO: 95 - DNA, 1744 bp, Artificial Sequence
  Description of Artificial Sequence Synthetic polynucleotide
ttgtaatgggaataagggcaggactcctgggtataagtagctcagctgatcccaccctgcttctatgtgttaattcatttattcattcattcaacaagcatttgttgaaatgctctttgtgtcaggctcagcaggaagcagtggcaataaaatggtgaacaagaaagactcggggtttcttcatctatgttgatgtctgcagagaacagtatcagccttctaggaagtttgtaatcagatacattgttagagagatacttatctagtaaattcctactcatcctataaggctcaaaacaaatgcctctatgaaaccttccgtgattccctcaggcagagttaagagcttcctttcctgggcctctatctccttccattagtattataactgtttaccagtttcccctctagactaaatttctcaaaagagagaatgaggtctctttcagtcttctttgcatctttaaactagcctgggtcccagcctgtttgatgaaagaaacaagaacactgatacaagccacagccccttggcaaaaaagatacccaatagcaatggcaatgtaaaatcagttttagtaaatgaatcaagaattctgatgctttagggaaagtaatgtgaacctggcaccattaacaaattcagaactcttcttcttaggagctctctaactgaacagacagagggatgtcaacccctaattcagcttgatcgtatctcagcaactacatttaatgagacagtgggaaaaagagagctgtccacttttaaatcagcatatttctaactaaacaatggcaatggctaaatctttaaaatgcctatttctctcaagaacactgcaatggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaaacatgagatgccaggaaaacacacttaaaaaaaaaattcccactttaagctttagactgaatgtgagaaaggagatgataaaaagagtatcacaagagaatcttcaggctgtggg

SEQ ID NO: 96 - DNA, 1104 bp, Artificial Sequence
  Description of Artificial Sequence Synthetic polynucleotide
tttggcactgtgagcagtttacttgacaaattctgtcaaatatttgctttctgaaatctcgagaattggttgaatataattgtacttaatgtttgcaaaataaataaatatgggactaaggacgttctatcattaatttgtcagaaaagagagttgtcatttctgaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatttccagcagagttctattaaagggaggacagaatctgtctcttccaaggtggaaaatcgtgaatattccctgcattaatgaaccaagttaacactttaattgcttatagaaccgagttctccaatgacagcattaaaagatagggaggctctgatttatggtcaacacagatttgtaaccc

SEQ ID NO: 97 - DNA, 1910 bp, Artificial Sequence
  Description of Artificial Sequence Synthetic polynucleotide
tcaacatggataaccaaagttcttaaaactacgctttcaatgaacacatatcctttgagcaagactaataatgaggaatgggagccagctcctgtgatatttatgcaactactaaattctcactgaagtcaatgggagtttgcttacgtaagggctgcaaactttagcctccagagattaaaggggaaaaaaatccttaaactctttcaacattaatattgcctgtaaggaatccagccatgacctaagccatggagctttctgaacctagcaagtagaagggtaaacagtaaacaccagttattttaagcacaatctaatcagagttcaatgagaagcaatattatatttgatctctaaggtattaatacttgtatatcactattagacatctttatgtagtccattatccaaacaatggcttaagtctgtggtatttaataaatcaagtttccatggccgtgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagtgatatttaaaaaagaaactttggcaaatgcttgcaaaaaacacaccttttacaattttaaatgtgatttactgatggccagaacttgttaaacatagtaggaaattaaatatttattcatcttatttcattttcagggccgtaaacgctccttctgagtcattcccaataacaagaatttctaccagtaaagctattaacaggcatcaaaataggggagtgctaaattaagatgagattgtaaaagcaaataagaacatacgcagactcgcataggagtgcaaatgatcgtttctgattgaaatgtttatagctaaatgagtttggctgaattaaacacaaatgttccaaaagataagccgtagctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgctatgcttcaattttcatctaggtgatcaagattcattttcttgttctgtcatccaaataggcagacagaaaagtgattgaaatacattatggagatgtgtcattgcaca

SEQ ID NO: 98 - DNA, 540 bp, Artificial Sequence
  Description of Artificial Sequence Synthetic polynucleotide
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgc

SEQ ID NO: 99 - DNA, 359 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgcca

SEQ ID NO: 100 - DNA, 463 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 101 - DNA, 386 bp, Artificial Sequence
  Description of Artificial Sequence Synthetic polynucleotide
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacat

SEQ ID NO: 102 - DNA, 418 bp, Artificial Sequence
  Description of Artificial Sequence Synthetic polynucleotide
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacc

SEQ ID NO: 103 - DNA, 502 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 104 - DNA, 324 bp, Artificial Sequence
  Description of Artificial Sequence Synthetic polynucleotide
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcac

SEQ ID NO: 105 - DNA, 121 bp, Artificial Sequence
  Description of Artificial Sequence Synthetic polynucleotide
agcgcgcagagtctgcatgcgtgaggaagctcctgggcgcgtcacagccgcgctattctcagcgtctctccttttatggctccggaagtgagctggggttgctggcagcctggctggcact

SEQ ID NO: 106 - DNA, 393 bp, Artificial Sequence
  Description of Artificial Sequence Synthetic polynucleotide
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcc

SEQ ID NO: 107 - DNA, 408 bp, Artificial Sequence
  Description of Artificial Sequence Synthetic polynucleotide
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaa

SEQ ID NO: 108 - DNA, 499 bp, Artificial Sequence
  Description of Artificial Sequence Synthetic polynucleotide
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaa

SEQ ID NO: 109 - protein, 500 aa, Artificial Sequence; identical to SEQ ID NO: 2
  Description of Artificial Sequence Synthetic polypeptide

SEQ ID NO: 110 - DNA, 1500 bp, Artificial Sequence
  Description of Artificial Sequence Synthetic polynucleotide
atgagccccgggctgctgctgctcggtagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttctagattgggctaagaagtatggacctgtagtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtgatagacctggccttcagccggagctccttggttagcttaatggaaacattcaacgaaaaggctgagcagctggtggagattctagaagccaaggcagatgggcagacccctgtgagcatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaagtgattggttctaagaggtacctggatttcgaggacctggggagactgcagtacctgtcccaggtcctcaaagagtcgctgaggctgtacccaccagcatggggcacctttaggctgctggaagaggagaccttgattgatggggtgagagtccccggcaacaccccgctcttgttcagcacctatgtgatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgc

SEQ ID NO: 111 - DNA, 4036 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgtgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattccttaagctatcataggaatgagccccgggctgctgctgctcggtagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttctagattgggctaagaagtatggacctgtagtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtgatagacctggccttcagccggagctccttggttagcttaatggaaacattcaacgaaaaggctgagcagctggtggagattctagaagccaaggcagatgggcagacccctgtgagcatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaagtgattggttctaagaggtacctggatttcgaggacctggggagactgcagtacctgtcccaggtcctcaaagagtcgctgaggctgtacccaccagcatggggcacctttaggctgctggaagaggagaccttgattgatggggtgagagtccccggcaacaccccgctcttgttcagcacctatgtgatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctgagtgatagcttggtaccgagctcgatccaattgcaatgatcatcatgacagatctgcgcgcgatcgatatcagcgctttaaatttgcgcatgcagctatagttctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 112 - DNA, 696 bp, Artificial Sequence; identical to SEQ ID NO: 91
  Description of Artificial Sequence Synthetic polynucleotide

SEQ ID NO: 113 - DNA, 709 bp, Artificial Sequence; identical to SEQ ID NO: 92
  Description of Artificial Sequence Synthetic polynucleotide

SEQ ID NO: 114 - DNA, 768 bp, Artificial Sequence; identical to SEQ ID NO: 93
  Description of Artificial Sequence Synthetic polynucleotide

SEQ ID NO: 115 - DNA, 736 bp, Artificial Sequence; identical to SEQ ID NO: 94
  Description of Artificial Sequence Synthetic polynucleotide

SEQ ID NO: 116 - DNA, 1744 bp, Artificial Sequence; identical to SEQ ID NO: 95
  Description of Artificial Sequence Synthetic polynucleotide

SEQ ID NO: 117 - DNA, 1104 bp, Artificial Sequence; identical to SEQ ID NO: 96
  Description of Artificial Sequence Synthetic polynucleotide

SEQ ID NO: 118 - DNA, 1910 bp, Artificial Sequence; identical to SEQ ID NO: 97
  Description of Artificial Sequence Synthetic polynucleotide

SEQ ID NO: 119 - DNA, 540 bp, Artificial Sequence; identical to SEQ ID NO: 98
  Description of Artificial Sequence Synthetic polynucleotide

SEQ ID NO: 120 - DNA, 982 bp, Artificial Sequence
  Description of Artificial Sequence Synthetic polynucleotide
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagatgccacc

SEQ ID NO: 121 - DNA, 876 bp, Artificial Sequence
  Description of Artificial Sequence Synthetic polynucleotide
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagatgccacc

SEQ ID NO: 122 - DNA, 835 bp, Artificial Sequence
  Description of Artificial Sequence Synthetic polynucleotide
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaagggctgggcataaaagtcagggcagagccatctattgcttacatttgcttctg

SEQ ID NO: 123 - DNA, 812 bp, Artificial Sequence
  Description of Artificial Sequence Synthetic polynucleotide
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatgggctgggcataaaagtcagggcagagccatctattgcttacatttgcttctg

SEQ ID NO: 124 - DNA, 487 bp, Artificial Sequence
  Description of Artificial Sequence Synthetic polynucleotide
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagggctgggcataaaagtcagggcagagccatctattgcttacatttgcttctg

SEQ ID NO: 125 - DNA, 593 bp, Artificial Sequence
  Description of Artificial Sequence Synthetic polynucleotide
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgcgggctgggcataaaagtcagggcagagccatctattgcttacatttgcttctg

SEQ ID NO: 126 - DNA, 53 bp, Artificial Sequence
  Description of Artificial Sequence Synthetic oligonucleotide
gggctgggcataaaagtcagggcagagccatctattgcttacatttgcttctg

SEQ ID NO: 127 - DNA, 553 bp, Artificial Sequence
  Description of Artificial Sequence Synthetic polynucleotide
gggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 128 - DNA, 643 bp, Artificial Sequence
  Description of Artificial Sequence Synthetic polynucleotide
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatg

SEQ ID NO: 129 - DNA, 656 bp, Artificial Sequence
  Description of Artificial Sequence Synthetic polynucleotide
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggc

SEQ ID NO: 130 - DNA, 215 bp, Artificial Sequence
  Description of Artificial Sequence Synthetic polynucleotide
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggg

SEQ ID NO: 131 - DNA, 782 bp, Artificial Sequence
  Description of Artificial Sequence Synthetic polynucleotide
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaa

SEQ ID NO: 132 - DNA, 759 bp, Artificial Sequence
  Description of Artificial Sequence Synthetic polynucleotide
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgat

SEQ ID NO: 133 - DNA, 434 bp, Artificial Sequence
  Description of Artificial Sequence Synthetic polynucleotide
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccata

SEQ ID NO: 134 - DNA, 540 bp, Artificial Sequence; identical to SEQ ID NO: 98
  Description of Artificial Sequence Synthetic polynucleotide

SEQ ID NO: 135 - DNA, 20 bp, Artificial Sequence
  Description of Artificial Sequence Synthetic oligonucleotide
tgctgctgctgctgctgctg

SEQ ID NO: 136 - DNA, 20 bp, Artificial Sequence
  Description of Artificial Sequence Synthetic oligonucleotide
ggcggcggcggcggcggcgg

SEQ ID NO: 137 - DNA, 20 bp, Artificial Sequence
  Description of Artificial Sequence Synthetic oligonucleotide
gaggggtggggaggctgggg

SEQ ID NO: 138 - DNA, 20 bp, Artificial Sequence
  Description of Artificial Sequence Synthetic oligonucleotide
tccttgacctgctgctgcag

SEQ ID NO: 139 - DNA, 20 bp, Artificial Sequence
  Description of Artificial Sequence Synthetic oligonucleotide
ccttccactggccatgatgc

SEQ ID NO: 140 - DNA, 20 bp, Artificial Sequence
  Description of Artificial Sequence Synthetic oligonucleotide
actgtgccactatgttttca

SEQ ID NO: 141 - DNA, 20 bp, Artificial Sequence
  Description of Artificial Sequence Synthetic oligonucleotide
tgaggtatcagattgtctag

SEQ ID NO: 142 - DNA, 20 bp, Artificial Sequence
  Description of Artificial Sequence Synthetic oligonucleotide
aaattaatctcttacctgat

SEQ ID NO: 143 - DNA, 20 bp, Artificial Sequence
  Description of Artificial Sequence Synthetic oligonucleotide
cccagggctagcaaggaaca

SEQ ID NO: 144 - DNA, 20 bp, Artificial Sequence
  Description of Artificial Sequence Synthetic oligonucleotide
aattcagtagcttcccttaa

SEQ ID NO: 145 - DNA, 20 bp, Artificial Sequence
  Description of Artificial Sequence Synthetic oligonucleotide
ctgggcccgcagcggaaggg

SEQ ID NO: 146 - DNA, 20 bp, Artificial Sequence
  Description of Artificial Sequence Synthetic oligonucleotide
ttattgctgtctactatccg

SEQ ID NO: 147 - DNA, 20 bp, Artificial Sequence
  Description of Artificial Sequence Synthetic oligonucleotide
tcagtccttcccaaagctct

SEQ ID NO: 148 - DNA, 20 bp, Artificial Sequence
  Description of Artificial Sequence Synthetic oligonucleotide
taatctctttactgatataa

SEQ ID NO: 149 - DNA, 20 bp, Artificial Sequence
  Description of Artificial Sequence Synthetic oligonucleotide
tcagcagtgttatttcttac

SEQ ID NO: 150 - DNA, 22 bp, Artificial Sequence
  Description of Artificial Sequence Synthetic oligonucleotide
aaaccgttaccattactgagtt

SEQ ID NO: 151 - DNA, 21 bp, Artificial Sequence
  Description of Artificial Sequence Synthetic oligonucleotide
aaatcgctgatttgtgtagtc

SEQ ID NO: 152 - DNA, 467 bp, Artificial Sequence
  Description of Artificial Sequence Synthetic polynucleotide
gagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcg

SEQ ID NO: 153 - protein, 13 aa, Artificial Sequence
  Description of Artificial Sequence Synthetic peptide
SQAGASDIRDQSR

SEQ ID NO: 154 - protein, 13 aa, Artificial Sequence
  Description of Artificial Sequence Synthetic peptide
  MOD_RES (2)..(2): Any amino acid
  MOD_RES (5)..(5): Any amino acid
  MOD_RES (7)..(10): Any amino acid
  MOD_RES (12)..(12): Any amino acid
SXAGXSXXXXQXR

SEQ ID NO: 155 - protein, 4 aa, Artificial Sequence
  Description of Artificial Sequence Synthetic peptide
EYSW

SEQ ID NO: 156 - DNA, 120 bp, Artificial Sequence
  Description of Artificial Sequence Synthetic polynucleotide
  See specification as filed for detailed description of substitutions and preferred embodiments
cagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 157 - DNA, 117 bp, Artificial Sequence
  Description of Artificial Sequence Synthetic polynucleotide
  SITE (1)..(117): This sequence may encompass 36-39 “cag” repeating units
cagcagcagcagcagcagcagcagcagcagcagcagcagcagcagcagcagcagcagcagcagcagcagcagcagcagcagcagcagcagcagcagcagcagcagcagcagcagcag